SEQUENCE LISTING (179 sequences)

SEQ ID NO: 1 - DNA, 51 bp, synthetic construct
  source 1..51: mol_type = other DNA
  misc_difference 24: note = a, c, t, g, unknown or other
  misc_difference 25: note = a, c, t, g, unknown or other
  misc_difference 26: note = a, c, t, g, unknown or other
  misc_difference 27: note = a, c, t, g, unknown or other
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
cctacacgacgctcttccgatctnnnnnnnnctggaagtgccagtagacgg

SEQ ID NO: 2 - DNA, 55 bp, synthetic construct
  source 1..55: mol_type = other DNA
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
  misc_difference 32: note = a, c, t, g, unknown or other
  misc_difference 33: note = a, c, t, g, unknown or other
  misc_difference 34: note = a, c, t, g, unknown or other
  misc_difference 35: note = a, c, t, g, unknown or other
gagttcagacgtgtgctcttccgatctnnnnnnnnaaatacttcgagtccgcccg

SEQ ID NO: 3 - DNA, 51 bp, synthetic construct
  source 1..51: mol_type = other DNA
  misc_difference 24: note = a, c, t, g, unknown or other
  misc_difference 25: note = a, c, t, g, unknown or other
  misc_difference 26: note = a, c, t, g, unknown or other
  misc_difference 27: note = a, c, t, g, unknown or other
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
cctacacgacgctcttccgatctnnnnnnnnggttcatgccggatggtttg

SEQ ID NO: 4 - DNA, 55 bp, synthetic construct
  source 1..55: mol_type = other DNA
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
  misc_difference 32: note = a, c, t, g, unknown or other
  misc_difference 33: note = a, c, t, g, unknown or other
  misc_difference 34: note = a, c, t, g, unknown or other
  misc_difference 35: note = a, c, t, g, unknown or other
gagttcagacgtgtgctcttccgatctnnnnnnnngatcgagcaaggtgatcgca

SEQ ID NO: 5 - DNA, 51 bp, synthetic construct
  source 1..51: mol_type = other DNA
  misc_difference 24: note = a, c, t, g, unknown or other
  misc_difference 25: note = a, c, t, g, unknown or other
  misc_difference 26: note = a, c, t, g, unknown or other
  misc_difference 27: note = a, c, t, g, unknown or other
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
cctacacgacgctcttccgatctnnnnnnnncaagtactgcgtacatgggc

SEQ ID NO: 6 - DNA, 55 bp, synthetic construct
  source 1..55: mol_type = other DNA
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
  misc_difference 32: note = a, c, t, g, unknown or other
  misc_difference 33: note = a, c, t, g, unknown or other
  misc_difference 34: note = a, c, t, g, unknown or other
  misc_difference 35: note = a, c, t, g, unknown or other
gagttcagacgtgtgctcttccgatctnnnnnnnngcatcgaggatcgactgctt

SEQ ID NO: 7 - DNA, 51 bp, synthetic construct
  source 1..51: mol_type = other DNA
  misc_difference 24: note = a, c, t, g, unknown or other
  misc_difference 25: note = a, c, t, g, unknown or other
  misc_difference 26: note = a, c, t, g, unknown or other
  misc_difference 27: note = a, c, t, g, unknown or other
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
cctacacgacgctcttccgatctnnnnnnnncgacggtaccaaggtcgatt

SEQ ID NO: 8 - DNA, 55 bp, synthetic construct
  source 1..55: mol_type = other DNA
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
  misc_difference 32: note = a, c, t, g, unknown or other
  misc_difference 33: note = a, c, t, g, unknown or other
  misc_difference 34: note = a, c, t, g, unknown or other
  misc_difference 35: note = a, c, t, g, unknown or other
gagttcagacgtgtgctcttccgatctnnnnnnnncgttgcctgtcggtcgatta

SEQ ID NO: 9 - DNA, 51 bp, synthetic construct
  source 1..51: mol_type = other DNA
  misc_difference 24: note = a, c, t, g, unknown or other
  misc_difference 25: note = a, c, t, g, unknown or other
  misc_difference 26: note = a, c, t, g, unknown or other
  misc_difference 27: note = a, c, t, g, unknown or other
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
cctacacgacgctcttccgatctnnnnnnnnttggaatcggtcaatccggc

SEQ ID NO: 10 - DNA, 55 bp, synthetic construct
  source 1..55: mol_type = other DNA
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
  misc_difference 32: note = a, c, t, g, unknown or other
  misc_difference 33: note = a, c, t, g, unknown or other
  misc_difference 34: note = a, c, t, g, unknown or other
  misc_difference 35: note = a, c, t, g, unknown or other
gagttcagacgtgtgctcttccgatctnnnnnnnncaacgacttctcccttggca

SEQ ID NO: 11 - DNA, 51 bp, synthetic construct
  source 1..51: mol_type = other DNA
  misc_difference 24: note = a, c, t, g, unknown or other
  misc_difference 25: note = a, c, t, g, unknown or other
  misc_difference 26: note = a, c, t, g, unknown or other
  misc_difference 27: note = a, c, t, g, unknown or other
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
cctacacgacgctcttccgatctnnnnnnnnatcatcggcgtggaaacctt

SEQ ID NO: 12 - DNA, 55 bp, synthetic construct
  source 1..55: mol_type = other DNA
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
  misc_difference 32: note = a, c, t, g, unknown or other
  misc_difference 33: note = a, c, t, g, unknown or other
  misc_difference 34: note = a, c, t, g, unknown or other
  misc_difference 35: note = a, c, t, g, unknown or other
gagttcagacgtgtgctcttccgatctnnnnnnnnctcggcatgaattgtggcag

SEQ ID NO: 13 - DNA, 51 bp, synthetic construct
  source 1..51: mol_type = other DNA
  misc_difference 24: note = a, c, t, g, unknown or other
  misc_difference 25: note = a, c, t, g, unknown or other
  misc_difference 26: note = a, c, t, g, unknown or other
  misc_difference 27: note = a, c, t, g, unknown or other
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
cctacacgacgctcttccgatctnnnnnnnnctggtggacctgtcgatgat

SEQ ID NO: 14 - DNA, 55 bp, synthetic construct
  source 1..55: mol_type = other DNA
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
  misc_difference 32: note = a, c, t, g, unknown or other
  misc_difference 33: note = a, c, t, g, unknown or other
  misc_difference 34: note = a, c, t, g, unknown or other
  misc_difference 35: note = a, c, t, g, unknown or other
gagttcagacgtgtgctcttccgatctnnnnnnnnatggatgtgcagcaggttct

SEQ ID NO: 15 - DNA, 51 bp, synthetic construct
  source 1..51: mol_type = other DNA
  misc_difference 24: note = a, c, t, g, unknown or other
  misc_difference 25: note = a, c, t, g, unknown or other
  misc_difference 26: note = a, c, t, g, unknown or other
  misc_difference 27: note = a, c, t, g, unknown or other
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
cctacacgacgctcttccgatctnnnnnnnncgctctttccctgctgttga

SEQ ID NO: 16 - DNA, 55 bp, synthetic construct
  source 1..55: mol_type = other DNA
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
  misc_difference 32: note = a, c, t, g, unknown or other
  misc_difference 33: note = a, c, t, g, unknown or other
  misc_difference 34: note = a, c, t, g, unknown or other
  misc_difference 35: note = a, c, t, g, unknown or other
gagttcagacgtgtgctcttccgatctnnnnnnnnggataccagtggccagatcc

SEQ ID NO: 17 - DNA, 50 bp, synthetic construct
  source 1..50: mol_type = other DNA
  misc_difference 24: note = a, c, t, g, unknown or other
  misc_difference 25: note = a, c, t, g, unknown or other
  misc_difference 26: note = a, c, t, g, unknown or other
  misc_difference 27: note = a, c, t, g, unknown or other
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
cctacacgacgctcttccgatctnnnnnnnncgaagcggatcgcgtagtc

SEQ ID NO: 18 - DNA, 55 bp, synthetic construct
  source 1..55: mol_type = other DNA
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
  misc_difference 32: note = a, c, t, g, unknown or other
  misc_difference 33: note = a, c, t, g, unknown or other
  misc_difference 34: note = a, c, t, g, unknown or other
  misc_difference 35: note = a, c, t, g, unknown or other
gagttcagacgtgtgctcttccgatctnnnnnnnntgctgtgtgactccttcgac

SEQ ID NO: 19 - DNA, 51 bp, synthetic construct
  source 1..51: mol_type = other DNA
  misc_difference 24: note = a, c, t, g, unknown or other
  misc_difference 25: note = a, c, t, g, unknown or other
  misc_difference 26: note = a, c, t, g, unknown or other
  misc_difference 27: note = a, c, t, g, unknown or other
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
cctacacgacgctcttccgatctnnnnnnnncttccagctcttcctcaccg

SEQ ID NO: 20 - DNA, 55 bp, synthetic construct
  source 1..55: mol_type = other DNA
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
  misc_difference 32: note = a, c, t, g, unknown or other
  misc_difference 33: note = a, c, t, g, unknown or other
  misc_difference 34: note = a, c, t, g, unknown or other
  misc_difference 35: note = a, c, t, g, unknown or other
gagttcagacgtgtgctcttccgatctnnnnnnnnacctgaaacgaaaaacgccg

SEQ ID NO: 21 - DNA, 51 bp, synthetic construct
  source 1..51: mol_type = other DNA
  misc_difference 24: note = a, c, t, g, unknown or other
  misc_difference 25: note = a, c, t, g, unknown or other
  misc_difference 26: note = a, c, t, g, unknown or other
  misc_difference 27: note = a, c, t, g, unknown or other
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
cctacacgacgctcttccgatctnnnnnnnncttggcatcgaactggaggg

SEQ ID NO: 22 - DNA, 55 bp, synthetic construct
  source 1..55: mol_type = other DNA
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
  misc_difference 32: note = a, c, t, g, unknown or other
  misc_difference 33: note = a, c, t, g, unknown or other
  misc_difference 34: note = a, c, t, g, unknown or other
  misc_difference 35: note = a, c, t, g, unknown or other
gagttcagacgtgtgctcttccgatctnnnnnnnnagcaacaccaggaacgtcat

SEQ ID NO: 23 - DNA, 51 bp, synthetic construct
  source 1..51: mol_type = other DNA
  misc_difference 24: note = a, c, t, g, unknown or other
  misc_difference 25: note = a, c, t, g, unknown or other
  misc_difference 26: note = a, c, t, g, unknown or other
  misc_difference 27: note = a, c, t, g, unknown or other
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
cctacacgacgctcttccgatctnnnnnnnnggcttgtagtggccgaacag

SEQ ID NO: 24 - DNA, 54 bp, synthetic construct
  source 1..54: mol_type = other DNA
  misc_difference 28: note = a, c, t, g, unknown or other
  misc_difference 29: note = a, c, t, g, unknown or other
  misc_difference 30: note = a, c, t, g, unknown or other
  misc_difference 31: note = a, c, t, g, unknown or other
  misc_difference 32: note = a, c, t, g, unknown or other
  misc_difference 33: note = a, c, t, g, unknown or other
  misc_difference 34: note = a, c, t, g, unknown or other
  misc_difference 35: note = a, c, t, g, unknown or other
gagttcagacgtgtgctcttccgatctnnnnnnnnctgctgcaacgcatcactg

SEQ ID NO: 25 - DNA, 58 bp, synthetic construct
  source 1..58: mol_type = other DNA
aatgatacggcgaccaccgagatctacactctttccctacacgacgctcttccgatct

SEQ ID NO: 26 - DNA, 60 bp, synthetic construct
  source 1..60: mol_type = other DNA
caagcagaagacggcatacgagattatccagtgtgactggagttcagacgtgtgctcttc

SEQ ID NO: 27 - DNA, 60 bp, synthetic construct
  source 1..60: mol_type = other DNA
caagcagaagacggcatacgagatgagataacgtgactggagttcagacgtgtgctcttc

SEQ ID NO: 28 - DNA, 60 bp, synthetic construct
  source 1..60: mol_type = other DNA
caagcagaagacggcatacgagatcatggaatgtgactggagttcagacgtgtgctcttc

SEQ ID NO: 29 - DNA, 60 bp, synthetic construct
  source 1..60: mol_type = other DNA
caagcagaagacggcatacgagatcagttccagtgactggagttcagacgtgtgctcttc

SEQ ID NO: 30 - DNA, 60 bp, synthetic construct
  source 1..60: mol_type = other DNA
caagcagaagacggcatacgagatccgtatatgtgactggagttcagacgtgtgctcttc

SEQ ID NO: 31 - DNA, 60 bp, synthetic construct
  source 1..60: mol_type = other DNA
caagcagaagacggcatacgagatgctgaagagtgactggagttcagacgtgtgctcttc

SEQ ID NO: 32 - DNA, 60 bp, synthetic construct
  source 1..60: mol_type = other DNA
caagcagaagacggcatacgagatcgcaagctgtgactggagttcagacgtgtgctcttc

SEQ ID NO: 33 - DNA, 60 bp, synthetic construct
  source 1..60: mol_type = other DNA
caagcagaagacggcatacgagattatctgtggtgactggagttcagacgtgtgctcttc

SEQ ID NO: 34 - DNA, 60 bp, synthetic construct
  source 1..60: mol_type = other DNA
caagcagaagacggcatacgagatgccgaatggtgactggagttcagacgtgtgctcttc

SEQ ID NO: 35 - DNA, 60 bp, synthetic construct
  source 1..60: mol_type = other DNA
caagcagaagacggcatacgagattactgcaggtgactggagttcagacgtgtgctcttc

SEQ ID NO: 36 - DNA, 60 bp, synthetic construct
  source 1..60: mol_type = other DNA
caagcagaagacggcatacgagatcatgttgagtgactggagttcagacgtgtgctcttc

SEQ ID NO: 37 - DNA, 60 bp, synthetic construct
  source 1..60: mol_type = other DNA
caagcagaagacggcatacgagatatagagtcgtgactggagttcagacgtgtgctcttc

SEQ ID NO: 38 - DNA, 60 bp, synthetic construct
  source 1..60: mol_type = other DNA
caagcagaagacggcatacgagattaacctcagtgactggagttcagacgtgtgctcttc

SEQ ID NO: 39 - DNA, 60 bp, synthetic construct
  source 1..60: mol_type = other DNA
caagcagaagacggcatacgagatattagctggtgactggagttcagacgtgtgctcttc

SEQ ID NO: 40 - DNA, 60 bp, synthetic construct
  source 1..60: mol_type = other DNA
caagcagaagacggcatacgagattcacgctcgtgactggagttcagacgtgtgctcttc

SEQ ID NO: 41 - DNA, 60 bp, synthetic construct
  source 1..60: mol_type = other DNA
caagcagaagacggcatacgagatgcatcacggtgactggagttcagacgtgtgctcttc

SEQ ID NO: 42 - DNA, 60 bp, synthetic construct
  source 1..60: mol_type = other DNA
caagcagaagacggcatacgagataggacggtgtgactggagttcagacgtgtgctcttc

SEQ ID NO: 43 - DNA, 60 bp, synthetic construct
  source 1..60: mol_type = other DNA
caagcagaagacggcatacgagatgaacgtaggtgactggagttcagacgtgtgctcttc

SEQ ID NO: 44 - DNA, 60 bp, synthetic construct
  source 1..60: mol_type = other DNA
caagcagaagacggcatacgagataccttgaagtgactggagttcagacgtgtgctcttc

SEQ ID NO: 45 - DNA, 60 bp, synthetic construct
  source 1..60: mol_type = other DNA
caagcagaagacggcatacgagatgtggttccgtgactggagttcagacgtgtgctcttc

SEQ ID NO: 46 - DNA, 60 bp, synthetic construct
  source 1..60: mol_type = other DNA
caagcagaagacggcatacgagattatggtgtgtgactggagttcagacgtgtgctcttc

SEQ ID NO: 47 - DNA, 60 bp, synthetic construct
  source 1..60: mol_type = other DNA
caagcagaagacggcatacgagatcgactgctgtgactggagttcagacgtgtgctcttc

SEQ ID NO: 48 - DNA, 60 bp, synthetic construct
  source 1..60: mol_type = other DNA
caagcagaagacggcatacgagatctggctctgtgactggagttcagacgtgtgctcttc

SEQ ID NO: 49 - DNA, 60 bp, synthetic construct
  source 1..60: mol_type = other DNA
caagcagaagacggcatacgagatggctcttggtgactggagttcagacgtgtgctcttc

SEQ ID NO: 50 - DNA, 60 bp, synthetic construct
  source 1..60: mol_type = other DNA
caagcagaagacggcatacgagatcgaacagtgtgactggagttcagacgtgtgctcttc

SEQ ID NO: 51 - DNA, 60 bp, synthetic construct
  source 1..60: mol_type = other DNA
caagcagaagacggcatacgagatggttagcagtgactggagttcagacgtgtgctcttc

SEQ ID NO: 52 - DNA, 60 bp, synthetic construct
  source 1..60: mol_type = other DNA
caagcagaagacggcatacgagatagacgctggtgactggagttcagacgtgtgctcttc

SEQ ID NO: 53 - DNA, 60 bp, synthetic construct
  source 1..60: mol_type = other DNA
caagcagaagacggcatacgagatttgctagggtgactggagttcagacgtgtgctcttc

SEQ ID NO: 54 - DNA, 60 bp, synthetic construct
  source 1..60: mol_type = other DNA
caagcagaagacggcatacgagatctccggttgtgactggagttcagacgtgtgctcttc

SEQ ID NO: 55 - DNA, 60 bp, synthetic construct
  source 1..60: mol_type = other DNA
caagcagaagacggcatacgagataccgcatcgtgactggagttcagacgtgtgctcttc

SEQ ID NO: 56 - DNA, 60 bp, synthetic construct
  source 1..60: mol_type = other DNA
caagcagaagacggcatacgagatattaccgagtgactggagttcagacgtgtgctcttc

SEQ ID NO: 57 - DNA, 60 bp, synthetic construct
  source 1..60: mol_type = other DNA
caagcagaagacggcatacgagatagttgttggtgactggagttcagacgtgtgctcttc

SEQ ID NO: 58 - DNA, 60 bp, synthetic construct
  source 1..60: mol_type = other DNA
caagcagaagacggcatacgagatacgctgtcgtgactggagttcagacgtgtgctcttc

SEQ ID NO: 59 - DNA, 60 bp, synthetic construct
  source 1..60: mol_type = other DNA
caagcagaagacggcatacgagatattggcaagtgactggagttcagacgtgtgctcttc

SEQ ID NO: 60 - DNA, 60 bp, synthetic construct
  source 1..60: mol_type = other DNA
caagcagaagacggcatacgagattagcacgcgtgactggagttcagacgtgtgctcttc

SEQ ID NO: 61 - DNA, 60 bp, synthetic construct
  source 1..60: mol_type = other DNA
caagcagaagacggcatacgagattgaaggatgtgactggagttcagacgtgtgctcttc

SEQ ID NO: 62 - DNA, 60 bp, synthetic construct
  source 1..60: mol_type = other DNA
caagcagaagacggcatacgagatttcagtaggtgactggagttcagacgtgtgctcttc

SEQ ID NO: 63 - DNA, 60 bp, synthetic construct
  source 1..60: mol_type = other DNA
caagcagaagacggcatacgagatcgatacgcgtgactggagttcagacgtgtgctcttc

SEQ ID NO: 64 - DNA, 60 bp, synthetic construct
  source 1..60: mol_type = other DNA
caagcagaagacggcatacgagatctgtaagtgtgactggagttcagacgtgtgctcttc

SEQ ID NO: 65 - DNA, 60 bp, synthetic construct
  source 1..60: mol_type = other DNA
caagcagaagacggcatacgagatacgtagccgtgactggagttcagacgtgtgctcttc

SEQ ID NO: 66 - DNA, 60 bp, synthetic construct
  source 1..60: mol_type = other DNA
caagcagaagacggcatacgagatggcttcaagtgactggagttcagacgtgtgctcttc

SEQ ID NO: 67 - DNA, 60 bp, synthetic construct
  source 1..60: mol_type = other DNA
caagcagaagacggcatacgagattccacaccgtgactggagttcagacgtgtgctcttc

SEQ ID NO: 68 - DNA, 60 bp, synthetic construct
  source 1..60: mol_type = other DNA
caagcagaagacggcatacgagatgttgctcagtgactggagttcagacgtgtgctcttc

SEQ ID NO: 69 - DNA, 60 bp, synthetic construct
  source 1..60: mol_type = other DNA
caagcagaagacggcatacgagatctcacttagtgactggagttcagacgtgtgctcttc

SEQ ID NO: 70 - DNA, 60 bp, synthetic construct
  source 1..60: mol_type = other DNA
caagcagaagacggcatacgagatgcactagagtgactggagttcagacgtgtgctcttc

SEQ ID NO: 71 - DNA, 60 bp, synthetic construct
  source 1..60: mol_type = other DNA
caagcagaagacggcatacgagattacaacacgtgactggagttcagacgtgtgctcttc

SEQ ID NO: 72 - DNA, 60 bp, synthetic construct
  source 1..60: mol_type = other DNA
caagcagaagacggcatacgagatgcgaacacgtgactggagttcagacgtgtgctcttc

SEQ ID NO: 73 - DNA, 60 bp, synthetic construct
  source 1..60: mol_type = other DNA
caagcagaagacggcatacgagatagagtgcggtgactggagttcagacgtgtgctcttc

SEQ ID NO: 74 - protein, 281 aa, Homo sapiens
MKFEGTQSYVATDDLKLAVNAAITLQRPLLVKGEPGTGKTMLAEQLAESFGAKLITWHIKSTTKAHQGLYEYDAVSRLRDSQLGVDKVHDVRNYIKKGKLWEAFEAEERVILLIDEIDKADIEFPNDLLQELDKMEFYVYETNETIKAKQRPIIIITSNNEKELPDAFLRRCFFHYIAFPDRETLQKIVDVHYPNIKQSLVSEALDIFFDVRKVPGLKKKPSTSELVDWLKLLMADEIGEAVLRERDPTKAIPPLAGALVKNEQDVQLLERLAFMSRRASR

SEQ ID NO: 75 - protein, 499 aa, Homo sapiens
MNILYDERLDGPLPQVDKDGLLAELRLRLPDLELLHAAEDLRPYECDGLSAYRCTPLLVALPERIEQVQGLLALCHRLKVPVVARGAGTGLSGGALPLENGVLLVMARFRRILEIDPLGRFARVQPGVRNLAISQAAAPHGLYYAPDPSSQIACSIGGNVAENAGGVHCLKYGLTVHNLLQVDIVTLEGERLSLGSSALDSAGFDLLALFTGSEGLLGVVVEVTVRLLPRPPVAKVLLASFDDVESAGRAVADLIGAGIVPAGLEMMDNLSIRAAEDFIHAGYPVDAAAILLCELDGVEADVHEDCERVRELFEAAGATSVRQAQDEAERQRFWAGRKNAFPAVGRISPDYYCMDGSIPRRELPRVLHGIAELSREYGLRVANVFHAGDGNMHPLILFDANLPGELERAEALGGRILELCVAVGGSITGEHGVGREKINQMCAQFNADELTLFHAVKAAFDPAGLLNPGKNVPTLHRCAEFGAMHVHHGRLPFPELERF

SEQ ID NO: 76 - protein, 595 aa, Homo sapiens
MSMPLPMKLRTRLFLSISALITVSLFGLLLGLFSVMQLGRAQEQRMSHHYATIEVSQQLRQLLGDQLVILLRETPDGQALERSQNDFRRVLEQGRANTVDSAEQAALDGVRDAYLQLQAHTPALLEAPMADNDGFSEAFNGLRLRLQDLQQLALAGISEAETSARHRAYLVAGLLGLVGVAILLIGFVTAHSIARRFGAPIETLARAADRIGEGDFDVTLPMTNVAEVGQLTRRFGLMAEALRQYRKTSVEEVLSGERRLQAVLDSIDDGLVIFDNQGRIEHANPVAIRQLFVSNDPHGKRIDEILSDVDVQEAVEKALLGEVQDEAMPDLVVDVAGESRLLAWSLYPVTHPGGHSVGAVLVVRDVTEQRAFERVRSEFVLRASHELRTPVTGMQMAFSLLRERLDFPAESREADLIQTVDEEMSRLVLLINDLLNFSRYQTGMQKLELASCDLVDLLTQAQQRFIPKGEARRVSLQLELGDELPRLQLDRLQIERVIDNLLENALRHSSEGGQIHLQARRQGDRVLIAVEDNGEGIPFSQQGRIFEPFVQVGRKKGGAGLGLALCKEIIQLHGGRIAVRSQPGQGARFYMLLPV

SEQ ID NO: 77 - protein, 573 aa, Homo sapiens
MPLARWVPGLDSLLHYRRAWFRPDVQAGLSVAAIQIPTAIAYAQIAGFPPQVGLYACILPMLIYALIGSSRQLMVGPDAATAAMVAAAITPLAAGDPQRLVDLSMIVAIMVGLFSIVAGLARAGFIASFLSRPILVGYLNGIGLSLLVGQLGKLFGYEAATSGFVAGILALLENLLHIHWPTLILGSLSLLLMVLLPRRFPQLPGALCGVLLASLAAALLGLDRYGVELLGEVPAGLPQLSWPQTSLEELKSLLRDATGITVVSFCSAMLTARSFAARHGYSINPNHEFVALGLANIGAGVSQGFAISGADSRTAVNDMVGGKTQLVGVVAALVIAATLLLLNKPLGWVPMPALGAVLLLAGWGLIDVQALKGFWKLSRFEFSLCLLTTVGVLSVGVLPGIFVAVSIAVLRLLYYTYRPSDAVLGWMHGIDGQVELAKYPQATTLPGLVIYRFDAPLLFFNADYFKQRVLAVVDGSERPNAVLLNAEAMTNLDISGLATLHEVQQILKAQGVHLSLARVTGQTLDLLQRSSMLGEIKPPLVFSSVRSGVSAYRYWLRQQERLAAQAAATSGNA

SEQ ID NO: 78 - protein, 221 aa, Homo sapiens
MKPKEFVRRLSAVSTKNSFNPYSQVCSTFDVKSADKIRFQLLLDMLEKASRVEVDAIWIGRDLGYRGGRRTGLALTDEVHAKEYAERWSLCAQRTTKGDPCKERTASVIWDALRCIEDNIFLWNVFPLHPHEAGDPFSNRSHNAAERKIGEEILKDLVSMIKPRRLIAVGNDAVSSIGKIAPNIPSAKVRHPSYGGQNIFLQQIEGLYGVVCQPVIQRELF

SEQ ID NO: 79 - protein, 346 aa, Homo sapiens
MMNLWLLLPAVAALSLLLTAGLRRYAIARSLIDVPNARSSHQVPTPRGGGVAIVLSFLLAVLLAAILGAVKPDLATGILGAGIGIALLGFLDDHGHIAARWRLLGHFAGACWLLYWLGGLPALAFFGLVVDLGWVGHIAAAFYLVWMLNLYNFMDGIDGIASVEAVCVCVGAALLVVVSGVGSDEASQGVWLAALLAAAVTGFLFWNFPPARIFMGDAGSGFLGVIIGGLSLQAAWVSPQLFWGWLILLGVFIVDATLTLLRRLLRGDKVYEAHRSHAYQYASRHYGRHLPVTLAVGGINIFWLLPLALLVAAGKIDGMLALLIGYLPLAFLALRFKAGVLESRAA

SEQ ID NO: 80 - protein, 313 aa, Homo sapiens
MKIKAALIVDDLSLSEWQKRAIEDSSEYLDIQLVLSCRNSATKKSVIKHCGYYFLNILSLKNDMTRRVQLDSRGSEVIHFDSDYEGAWQRIPEDVCARILDKGIKLVIKFGMSLLRIDGGLQRLDILSYHHGDPEYYRGRPAGFYEIYENADSVGIIVQKLSNKLDAGEVLVRGYSKVHHHSYKKTSRNFYLNSVVLLRKALVNYSRGEQVVLEKLGKNYRLPSNFTVFKFFCKTIFRGLARLSYGAFFEKKWNVVALPYNDIPSLQELSVSAGKIPKVEKGYTFYADPFFSADGKLIRLEAQGNRMNACRFE

SEQ ID NO: 81 - protein, 229 aa, Homo sapiens
MHNVLIVGASRGIGLGLADAFLQRGAQVFAVARRPQGSPGLQALAERAGERLQAVTGDLNQHDCAERIGEMLGERRIDRLIVNAGIYGPQQQDVAEIDAEQTAQLFLTNAIAPLRLARALSGRVSRGGVVAFMSSQMASLALGLSATMPLYGASKAALNSLVRSWEGEFEELPFSLLLLHPGWVRTEMGGDSAPLSVEESAAGLVAAVEDAAGVNACRFVDYRNQPLPW

SEQ ID NO: 82 - protein, 257 aa, Homo sapiens
MLDRIRVVLVNTSHPGNIGGAARAMKNMGLSQLVLVQPESFPHGDAVARASGATDILDAARVVDTLEEALSGCSVVLGTSARDRRIPWPLLDPRECATTCLEHLEANGEVALVFGREYAGLTNEELQRCQFHVHIPSDPEFGSLNLAAAVQVLTYEVRMAWLAAQGKPTKMEKFESTSMLNTELVTADELELYYAHLERTLIDIGFLDPEKPRHLMSRLRRLYGRSAISKLEMNILRGILTETQKVARGLSYKRSDD

SEQ ID NO: 83 - protein, 152 aa, Homo sapiens
MTDNLLSISAACLFDDQGNLLLVRKRGTQAFMLPGGKREPGETPLAALQRELLEELRLPMGASTFEHLGSFQAPAANEANTRVDADIYVARLPHAVCAQAELEELAWLVPGQAQPDNLAPLLRDHVLPALARRAAENPETQAEHRTRPDHVR

SEQ ID NO: 84 - protein, 140 aa, Homo sapiens
MRALWFCVALMPVLAQADIYRWTDAQGKVHFSATPPAGAQRVEVRPQVVERDAATRQREQRTQEYFDARREERTAAAERAGQRQAALAEECGRLRQQLSQLERGGRFYRQDAGGGPVYLSDAELDAIRRELASRESERCR

SEQ ID NO: 85 - protein, 603 aa, Homo sapiens
MSDSPQNPGPSSLKIYFRLLGYVKPYIGMFLLSIVGFLIFASTQPMLAGILKYFVDGLSNPDAALFPNVQWPWLRDLHLVYAVPLLIILIAAWQGLGSFLGNFFLAKVSLGLVHDLRVALFNKLLVLPNRYFDTHSSGHLISRITFNVTMVTGAATDAIKVVIREGLTVVFLFLYLLWMNWKLTLVMLAILPVIAVMVTTASRKFRKQSKKIQVAMGDVTHVASETIQGYRVVRSFGGEAYEEKRFLDASQSNTDKQLRMTKTGAVYTPMLQLVIYVAMAILMFLVLWLRGDASAGDLVAYITAAGLLPKPIRQLSEVSSTVQRGVAGAESIFEQLDEAAEEDQGTVEKERVSGRLEVRNLSFRYPGTDKQVLDDISFIAEPGQMIALVGRSGSGKSTLANLVPRFYQHNNGKILLDGVEVEDYRLRNLRRHIALVTQQVTLFNDSVANNIAYGDLAGAPREEIERAAKAANAKEFIDNLPQGFDTEVGENGVLLSGGQRQRLAIARALLKDAPLLILDEATSALDTESERHIQAALDEVMKGRTTLVIAHRLSTIEKADLILVMDQGQIVERGSHAELLAQNGHYARLHAMGLDEQAPAPVG

SEQ ID NO: 86 - protein, 355 aa, Homo sapiens
MTPEGFNGAVLDWYDRHGRKDLPWQQGITPYRVWVSEIMLQQTQVSTVLGYFDRFMAALPDVEALAAAAEDEVLHLWTGLGYYSRARNLHKTAQIVVERHAGEFPRDVEQLAELPGIGRSTAGAIASLSMGLRAPILDGNVKRVLARYLAQDGYPGEPKVARALWEAAERFTPHARVNHYTQAMMDLGATLCTRSKPSCLLCPLVSGCRAHLLGREADYPQPKPRKALPQKRTLMPILANRDGAILLYRRPSSGLWGGLWSLPELDDLDGLEPLAARHSLALGERRELSGLTHTFSHFQLAIEPWLVAVESAPRAVAEGDWLWYNLATPPRLGLAAPVKKLLKRAEQELGRGTAA

SEQ ID NO: 87 - protein, 441 aa, Homo sapiens
MKVMKWSAIALAVSAGSTQFAVADAFVSDQAEAKGFIEDSSLNLLLRNYYFNRDGKEGRGDRVDWTQGFLTTYESGFTQGTVGFGVDAFGYLGLKLDGTSDKTGTGNLPVMNDGKPRDDYSRAGGAVKVRISKTMLKWGEMQPTAPVFAAGGSRLFPQTATGFQLQSSEFEGLDLEAGHFTEGKEPTTVKSRGELYATYAGQTAKSADFAGGRYAITDNLSASLYGAELKDIYRQYYLNTNYTIPLASDQSLGFDFNIYRTTDEGKSKAGDISNTTWSLAGAYTLDAHTFTLAYQQVHGDEPFDYIGFGGNGSGAGGDSIFLANSVQYSDFNGPGEKSWQARYDLNLASYGVPGLTFMLRYINGKDIDGTKVDSSSSYAGLYGEDGKHHETNLEAKYVVQSGPAKDLSFRIRQAWHRANADQGEGDQNEFRLIVDYPLSIL

SEQ ID NO: 88 - protein, 188 aa, Homo sapiens
MHFDSVTGWVRGVRHCPSPNFNLRPQGDAVSLLVIHNISLPPGQFGTGKVQAFFQNRLDPNEHPYFEEIRHLTVSAHFLIERDGAITQFVSCHDRAWHAGVSCFDGREACNDFSLGIELEGTDTEPYTDAQYTALAGLTRLLRAAFPAITPERIQGHCDIAPERKTDPGEAFDWSRYRAGLTDSKEET

SEQ ID NO: 89 - protein, 336 aa, Homo sapiens
MQVLRTWAARGVQWVGVAGVIGLSGAAQAGDYDGSPQVAEFVSEMTRDYGFAGEQLMGLFRDVNRKQSILDAISRPAERVKQWKEYRPIFISDARISRGVDFWNKHAEDLARAEKEYGVPAEIIVSIIGVETFFGRNTGSYRVMDALSTLGFDYPPRADFFRKELREFLLLAREQQVDPLSLTGSYAGAMGLPQFMPSSFRAYAVDFDGDGHINIWSDPTDAIGSVASYFKQHGWVTGEPVVSVAEINDESAESAVTRGVDPTMSLGELRARGWRTHDALRDDQKVTAMRFVGDKGIEYWVGLPNFYVITRYNRSAMYAMAVYQLAGEIARARGAH

SEQ ID NO: 90 - protein, 2472 aa, Homo sapiens
MGDRHDYVALEWVKGEIAETLKQARQALEAFVENPQDPTRMRFCLTYVHQVQGTLQMVEFYGAALLAEEMEQLVQALLDGRVPNQGEALEVLMQAILQLPVYLDRIQTARRDLPMVVLPLLNDLRAARGEKLLSETSLFAPDLSQRQPQLDGEAIAQLRTDELGGLLRKLRQTQQMALVGLLRNQDVATSLGYLARVYARLEGLCREAPLGPLWSIASGLVEGLANGSVVNSASVRTLLRQLDRELKRLVEQGADGLNQAAPDELVKNLLFYVAKAPSQSPRIRALKEQYRFDEALPDHETVDAERARLAGPDRDAMRSVVGALCEELVRIKDSLDLFVRSDRGHPSELDALLAPLKQIADTLAVLGFGQPRKVILDQLDVIHALAQGRREPSDAILMDVAGALLYVEATLAGMAGPGDERNSEESRLPTTDVAQIHQLVIKEARNGLEQAKDAIIEFIASQWNHEHLARVPELLTQVRGGLAMIPLERAATLLETCNRYIQEQLLARKAVPDWQSLDTLADAITSVEYYLERLSEDHASQSDLILDVAEDSLANLGYTLKPNSSAPAEPGLSGPAAIESPAAEPERPEAVVEVAETAEQPPADTASAEAAREDAPLLASDDNWTLGEVVPDAGEPSLDLALDLPLDDSAEVPPALPEVVEESGQPQSTPAPARSLDDFSLDEIDLSGLDLPADAAPASGPAALADWSLPEQWGLGDDLAQPAQAGETLDLSLEEPALSFDAPLESLEPLPALEPFDGSAEQELVLDALDPLPLDVALPESEGEVSAWEGSSLEELDLSDLDLPEVQLPEAEAEAPPAAEALASEAPALSLAEVMAAPVQPINPPAQNVPVSLLPPPADEEPVDEELREVFIEEAGEVLETIGRYLPAWKADHDDREALTEVRRAFHTLKGSGRMVRALVIGELAWSIENLFNRVLDRSIAASEPVQRVVDQVVALLPELVEEFAANAQRQRDDVDLLAATAHALAKGEPLPEPPAPDDGGVPPEAGAEQPSSLDNGVQAPPLADAPQAAAEAQSDVELLDPQLLEIFTNEAETHLEALVGFLADCARELPQPVTDALQRALHTLKGSAHMAGILPIAEIATPLEKLVKEYKSNLLAFDLREAELLHDAEQLFRIGLEQVGAQRPLNPIPGSDALLERIEALHQERIASLEAERYSDAGERRDPLLIEAFLVEGMDILLDAEDLLERWHEHPQERQELSALREELSTLDRGARHAELPQVEELCQALLALYDAVEEGRLAVSPAFFEEARQAHEALIGMMDQVAAGLQVTPRPERVAALQELLEAPAAEAVPFIDPESLGADDFPPEDEEPALPEAVFEEAGTPAEETVPAAPAPAPGRELDEEMVSIFLEEAVDILESAGQALAQWQAEPGALSSLSALQRDLHTLKGGARMAEIAEIGDLAHELEALYEGLVDRRYQHSPQLAGLLQACHDRLAEQLDQLSAGQPLADPHDLIQAIRRFRQGPVAEAATPGEAESPVEELVAPAVEEPAAPAAEAFEERDPELVEIFLEEGFDILDSAAAALQRWIDDVDNTIELEALQRDLHTLKGGARMAEIGEIGDLAHELEFLYEGLCGGRLRASPALFGLLQRCHDELAEMLEAVRGHRSLPDGQALIAEIRRLRSDPDEQLSVPTSVSLKPLAAKGAAADESEILDIFLEEADDLLENLELALGRWDGGNGDAQPLDDLLRILHTLKGGARLAGQTELGNLAHDLEQHLTDAQQQGAPWPDSLLLDAQSGLEGLQRQVDLLRERLAEDDEAGERPEPAQALVQADDTDRAVASALAELTRLAPAAGAIMAAEAAPPAAPATTLPFVRKAQEAAQEAASRRAPQELVKVPAELLENLVNLAGETSIFRGRVEQQVSDVGFTLGEMESTIERVRDQLRRLDTETQAQILSRHQADAERAGYEEFDPLEMDRYSQLQQLSRALFESASDLLDLKETLAAKNRDAETLLLQQARVNTELQEGLMRTRMVPFDRLVPRLRRIVRQVAGELGKQVEFVVGNADGEMDRTVLERIVAPLEHMLRNAVDHGIESGETRRAAGKPEHGTIRLNIGREGGDILLTLSDDGAGIRLDAVRRKAIERGLMSADSDLSDHEVLQFVLESGFSTAEKVTQISGRGVGLDVANSEVKQLGGSVSIQTEPGQGTRFNVRLPFTVSVNRALMVLSGEDLYAVPLNTIEGIVRVSPYELEALYDQRGEAGLDTPSFEYAGQSYELKYLGELLNNGQEPKLVGQSLPLPVILVRSSEHAVAVQVDSLAGSREIVVKSLGPQFAGVAGISGATLLGDGRVVVILDLLATIRSRHALLGQESRRERLALRQEMAASESEQQRPPLVMVVDDSVTVRKVTTRLLERNGMNVLTAKDGVDAIAQLQEHRPDILLLDIEMPRMDGFEVATLVRHDERLGNLPIIMITSRTGEKHRERALGIGVNQYLGKPYQETELLEAIQSLVGQHE

SEQ ID NO: 91 - protein, 214 aa, Homo sapiens
MVAITHTPKLKHLDKLLAHCHRRRYTAKSTIIYAGDRCETLFFIIKGSVTILIEDDDGREMIIGYLNSGDFFGELGLFEKEGSEQERSAWVRAKVECEVAEISYAKFRELSQQDSEILYTLGSQMADRLRKTTRKVGDLAFLDVTGRVARTLLDLCQQPDAMAHPDGMQIKITRQEIGRIVGCSREMVGRVLKSLEEQGLVHVKGKTMVVFGTR

SEQ ID NO: 92 - protein, 1039 aa, Homo sapiens
MIAKLIRWSVANRFLVLLATAMLTAWGVWGVRSTPVDALPDLSDVQVIIRTNYPGQAPQIVENQVTYPLATTMLSVPGAKTVRGFSFFGDSFVYVLFEDGTDLYWARSRVLEYLNQVQGRLPATAKPALGPDATGVGWIFQYALVDRTGKNDLAQLRALQDWFLKFELKSLPNVAEVASVGGMVKQYQVVLDPIKLASYGLSQAQVRDALMGANQETGGSVLELSGAEYMVRASGYLKTLDEFREIPLTARGGVPVRLGDVATLQIGPEMRRGIAELDGEGEVAGGVVVLRSGKNAQETIAAVKAKLAELQGSLPPGVEIVTTYDRSALIERAIRNLTTKLGEEFLVVALVCALFLWHLRSALVAIISLPLGVMTAFLVMRYQGINANIMSLGGIAIAVGAMVDAAVVMIENAHKKLEAWQHAHPDQRLQGKERWDVITQAAEEVGPALFFSLLIITLSFIPVFTLEAQEGRLFGPLAFTKTYAMAAAAGLSVTLIPVLMGYWIRGRIPDEQKNPITRILIAAYRPALEWVLRRPKATLLIAVLALATTAWPLARLGGEFLPRLDEGDLLYMPSALPGLSAQRATELLQLSNRMIKTVPEVDKVFGKAGRAETATDPAPLEMFETTVKLKPREQWRPGMTPEKLVEELDRAVKIPGLSNIWIPPIRNRIDMLATGIKSPIGVKVTGNDLGVIDRIAAEVEQVAKGIPGVTSSLAERLTGGRYVDVQIDRVAAGRYGLNIADVQAVIAGAVGGENVSETVEGLARFPINLRYAREWRDSPQRLAELPIFTPMGQQITLGTVARIAITDGPPMLKSENARPSGWVYVDVRGRDLASVANELRDAIGQQVKLEPGVSITYSGQFEYMERANARLKVVVPATLLIIFVLLYLTFARVDEAGLIMATLPFALTGGIWFLYLLNYNLSIATGVGFIALAGVAAEFGVVMLIYLKQALAERCPDGRNPTREELLDAIREGAVLRVRPKAMTVAVILAGLVPIVWSSGTGSEVMSRIAAPMLGGMVTAPLLSLFVIPAAYLLMRKPR

SEQ ID NO: 93 - protein, 226 aa, Homo sapiens
MSHYLEVLTPQNSQIIFIDQQPQMAFGVQSIDRQTLKNNVVGLAKAAKVFDIPVTITTVETDSFSGPTYPELLAVFPEQKILERTSMNSWDDQNVRDSLAAAGRKKVVVAGLWTEVCNTTFALCAMLEGGYEIYMVADASGGTSSDAHKYAMDRMVQAGVVPVTWQQVLLEWQRDWARRDTYDAVMAIAKEHSGAYGMGVDYAYTMVHKAPERVTHGERIGPNPAK

SEQ ID NO: 94 - protein, 293 aa, Homo sapiens
MSSNSRSNSTFTGRLSALANRLRLGAARSDGRELRERAAALELPFQPLRRPEPSVWWQAGPPLHTLVDLPRGALSGPVQEDKAEAHAVLKRLVRVSHSTLESVDLKTIEGVCSREILVQAPCPRLEDLATAEVCRGVRIISYKDFVKALSLALPRFTNGDSIRLRQAAWHGERLFWAGERQACAFAAAIVYARRRELELKLPAHLERYELEPGALDELEQRYHMLRIPTEAWSEPTFMSLLLDTGLPYARLALFTPETPECLLLPRNDERADALGEGLRAAGAADVVKYLKQL

SEQ ID NO: 95 - protein, 422 aa, Homo sapiens
MKDLKVAVVGLGYVGLPLAVEFGKKRTVVGFDINQGRIAELRQGIDSTLEVDAAELKEASELSFTFNLQDLQKCNVFIVTVPTPIDEHKQPDLTPLVKASESIGKVLKKGDIVIYESTVYPGATEEDCVPVLEKFSGLRFNEDFFAGYSPERINPGDKEHRVSSIKKVTSGSTPEIAELVDSLYREIITAGTHKASSIKVAEAAKVIENTQRDLNIALINELAIIFNRMGIDTEAVLKAAGTKWNFMPFRPGLVGGHCIGVDPYYLTHKAQSIGYHPEIILAGRRLNDGMGAYVVSQLVKAMLKRRIHVDGARVLLMGLTFKENCPDLRNTKVVDIVRELAEYNIQVDVFDPWVSAEDAMHEYGITPVGTPSHGAYDGIILAVAHSEFKNMGAENIRKLGKAEHVLYDLKYLLDEDKSDLRL

SEQ ID NO: 96 - protein, 446 aa, Homo sapiens
MREYYSRVLAYIACGASIAAGTYTQYFSYGILWMVPYALLYPHLAYHLGQRFRQHDPRKVTRALLAVDAVHCGLGMALLGFSVVPSLMFLLVLSFTALVIGGLRLLGMALLVSASSALLVAVLVAPPLLGNTPVEVAAVSILFCGLYICITAFFGHQQGLRLAQVRQEIAREQEKAARLARNLAKYLSPQVWEMIFSGKKSVRLETQRKKLTVFFSDIRGFTELSEELEAEALTDLLNNYLNEMSKIALKYGGTIDKFVGDCVMVFFGDPSTQGAKKDAVAAVSMGIAMRKHMKVLRQQWRAQGITKPLEIRMGINTGYCTVGNFGADTRMDYTIIGREVNLASRLESASEAGEILISHETYSLIKDVIMCRDKGQIAVKGFSRPVQIYQVVDSRRDLGAAPSYVEHELPGFSMYLDTNNIQNYDKERVIQALQQAAERLRDKVIL

SEQ ID NO: 97 - protein, 525 aa, Homo sapiens
MKIKAALIVDDLSLSEWQKRAIEDSSEYLDIQLVLSCRNSATKKSVIKHCGYYFLNILSLKNDMTRRVQLDSRGSEVIHFDSDYEGAWQRIPEDVCARILDKGIKLVIKFGMSLLRIDGGLQRLDILSYHHGDPEYYRGRPAGFYEIYENADSVGIIVQKLSNKLDAGEVLVRGYSKVHHHSYKKTSRNFYLNSVVLLRKALVNYSRGEQVVLEKLGKNYRLPSNFTVFKFFCKTIFRGLARLSYGAFFEKKWNVVALPYNDIPSLQELSVSAGKIPKVEKGYTFYADPFFSADGKLIRLEALNASNGLGEIIELKAQSLDFSRVILKGNHFSYPYSFEASGVEYLIPEVASHSAPCLLPPPFALESKKLFQGMEGERILDGTLFEHGGRYYLFCGQAVSGSDNLYLYVGESLEGPYTSHPCNPVVMNPGSARMGGRIFKEGGKLYRFGQNNSYGYGSSLAVNEIEVLDPEHYSEKRVANLAFQDARGPHTIDIHGQTMILDFYQDRFSLLAGYRRLVARLLSRG

SEQ ID NO: 98 - protein, 135 aa, Homo sapiens
MEQQSDGLKVMVIDDSKTIRRTAETLLKKVGCDVITAIDGFDALAKIADTHPNIIFVDIMMPRLDGYQTCALIKNNSAFKSTPVIMLSSKDGLFDKAKGRIVGSDQYLTKPFSKEELLGAIKAHVPSFTPVDAVS

SEQ ID NO: 99 - protein, 682 aa, Homo sapiens
MKKINAGNLFAGMRSSSVIAGLFIVLIVSIVLLFANFAYLNTQSNHDKQYIGHAGELRVLSQRIAKNATEAAAGKGEAFKLLKDARNDFEKRWNILVNGDESTSLPPSPEAVKPQMDVVQQDWDGLRKNADSILASEQTVLSLHQVASTLAETIPQLQVEYEEVVDILLENGAPADQVAVAQRQSLLAERILGSVNKVLAGDENSVQAADSFGRDASLFGRVLKGMQEGNAAMSISKVTNAEAVDRLNEIAELFEFVSGSVDEILETSPDLFQVREAANNIFSVSQTLLDKASQLADGFENLAGGRSINLFAGYALGALALASIILIGLVMVRETNRRLAETAEKNDRNQAAILRLLDEIADLADGDLTVAATVTEDFTGAIADSINYSIDQLRELVETINQTAVQVAAAAQETQSTAMHLAEASEHQAQEIAGASAAINEMAVSIDQVSANASESSAVAERSVAIANKGNEVVHNTITGMDNIREQIQDTSKRIKRLGESSQEIGDIVSLINDIADQTNILALNAAIQASMAGDAGRGFAVVADEVQRLAERSSAATKQIEALVKTIQTDTNEAVISMEQTTSEVVRGARLAQDAGVALEEIEKVSKTLAALIQNISNAARQQASSAGHISNTMNVIQEITSQTSAGTTATARSIGNLAKMASEMRNSVSGFKLPEGVEQA

SEQ ID NO: 100 - protein, 296 aa, Homo sapiens
MVRPHLPLNALRAFEASARHLSFTRAAIELCVTQAAVSHQVKSLEERLGVALFKRLPRGLMLTHEGESLLPVLCDSFDRIAGLLERFEGGHYRDVLTVGAVGTFTVGWLLPRLEDFQARHPFIDLRLSTHNNRVDIAAEGLDYAIRFGGGAWHGTEALALFEAPLTVLCCPEVAAQLHSPADLLQHTLLRSYRADEWPLWFQAAGLPAHAPLTRSIVFDTSLAMLEAARQGVGVALAPAAMFARQLASESIRRPFATEVSTGSYWLTRLQSRGETSAMLAFRGWLLEMAAVEARGR

SEQ ID NO: 101 - protein, 147 aa, Homo sapiens
MNYPVNPDLMPALMAVFQHVRTRIQSELDCQRLDLTPPDVHVLKLIDEQRGLNLQDLGRQMCRDKALITRKIRELEGRNLVRRERNPSDQRSFQLFLTDEGLAIHQHAEAIMSRVHDELFAPLTPVEQATRVHLLDQCLAAQPLEDI

SEQ ID NO: 102 - protein, 479 aa, Homo sapiens
MLIPVVLSGGAGTRLWPVSREGQPKPFMRLPDGQTLLGKTYRRAAGLLAGHGEIVTVTNREHYFQSKDQFQAARLGRHRGHFILEPTGRNTAPAIAVAALALQAEHGDAAVLVVMPADHLIRNEEAFREAVGHAARLAVAGHLVTFGVVPDAAETGFGYIELGDRLDEQGAAKVRRFVEKPDEETARRYVESGGFLWNSGMFCFTASTLVDELAQHAPALLEQARACLAASAAVKMADGIQHELAGEAFAALPDISIDYALMERSARVAVVPAAFDWSDIGSWGAMSALLDADAEGNRGSGDTLFVDTRNTFVQSDGRLVATVGVDDLVVVDTSDALLIARADRVQEVRRVVQRLKDERHEAYRLHRTVNRPWGSYTVLEEGPRFKIKRIVVRPGERLSLQMHHHRSEHWIVVQGMARVTNGDGARLVNSNESTYIPAGHRHRLENPGVIDLVMIEVQSGEYLGEDDIVRFEDQYGRVV

SEQ ID NO: 103 - protein, 899 aa, Homo sapiens
MKSHPDAASRSAAEVVTQLPVPSRLGLLRFERLNEPSWALLFLDPACERQLGLPATTLCALLDAPYASLMEPEARHRLHEQIQQQLVKRPHYQVSYKLHTPNGVLTMLEFGEAFQQHGRQLLHGYLMVEERAESAERSEQLLDLESQNLRLKASLDLYQRSQDDHLQHLLRSRTQQNLIVRLARHRYLSSDPLLEAAQLITQAACEAYGTARAGIWRLLDDQRLEAVTVYRRDLDQYEKPQSIDASRYPAYLEAVHSGRAIDAHNAQRDPRTQELYKDYLRPLGVNALLDATIRIGGEVVGVLCLEHAGENRMWQSDEIAFAGELADQYAQVLMNHERRNVSSALHLFQRAVEQSASAFLLIDRDGVVEYVNPSFTSITQYSADEVRNRRLSELPALENLSELLFDARSALTQQNSWQGEFRSRRKNHEPYWGQLSLSKVYDDLGELTHYIGIYEDITQNKLAQQHIEKLAYRDNLTGLANRHYFIGALEERLESSGDRPLSLLLVDIDNFKRINDSLGHQTGDKLLVSLARRLRSCLGDGATLARFASNEFAVLLDDTAVEKGESIAAQVLHMLDKPLFVDNQLINITGSIGLASAPQHGCDPQTLMKYAGLALHKAKANGKHQVQVFTEALTAEASYKLFVESNLRRALAQNELAVHYQPKLCLRSGQLLGLEALLRWQHPEKGMIRPDRFISVAEETGLIVPIGKWVIREACRQARELAEAGLGELQIAINLSPKQFTDPDLVGSIAAILHEENIPASQLELELTESLLLDATDDTRQQLERLKSLGLTLAMDDFGTGYSSLSYLKKFPIDVIKIDRSFIKDIPDSQDDMEITSAVIAMAHNLKLKVVAEGVESAEQLAFLRRNRCDIGQGYLFDRPIPSDLLNTSLLRYPCRTLH

SEQ ID NO: 104 - protein, 942 aa, Homo sapiens
MVRLRIAIGLLVSFLLLLLGPMSPAVADDAGVSSVPLQTTATTPSVNQNWRLLRDESAQLRIADVLQRKEQFRPLAKRSFIFPASPQAVWLQVQLPAQKVPSWLWIFAPRVQYLDYYLVQDGQLVRDQHTGESRPFQERPLPSRSYLFSLPVDGKPMTLYVRMTSNHPLMAWFDQIDEAGLVGLEKPAYAFGMLLGGMLLLLMYNLIRFAYSRSASSLWLAAVHAALAVCAAANLGLVAFWLPGLKFNQSLTADLGALGAAVSLLWFACSFFRGTAESRLNRLLQGEALLILAVGAIIAFTQQLWFSWLIYLLVILSSLSVPLIAAWHWYRGYQPARLIVAGMIVFNAGFMVFLPVLFGTKQLDPGWLVLGVFSFATLAGLVLSVSLTERQRLIQQLNLQQRTSEAAHTAELQTKAEFLAKISHEIRTPMNGVLGMTELLLGTPLSAKQRDYVQTIHSAGNELLTLINEILDISKLESGQIELDEVQFDLNALIEDCLDIFRVKAEQQRIELISFTQPQVPRVIGGDPTRLRQVVLSLLDNAFKQTEEGEILLVVALDDQGETPRLRIAVRDSGHPFDAKEREALLTAELHSGDFLSASKLGSHLGLIIARQLVRLMGGEFGIQSGSSQGTTLSLTLPLDPQQLENPTADLDGPLQGARLLVVDDNETCRKVLVQQCSGWGLNVSAVSSGKEALAQLRTKAHLREYFDVVLLDQDMPGMTGMQLAAKIKEDPNLNHDILLIMLTGISNAPSKIIARNAGIKRILAKPVAGYTLKATLADELAQRGVSGVTNYLQPAKEAQAPSLPSDFRILVAEDNSISTKVIRGMLNKLNLQPDTASNGQEALSAMKATQYDLVLMDCEMPVLDGFSATEQLRAWEAHEQRPHTPVVALTAHILSEHKERARLVGMDGHMAKPVELSQLRELIAYWVGERDRRRQGDALPS

SEQ ID NO: 105 - protein, 465 aa, Homo sapiens
MDDNKNKVLRLRDVVLYTVSAMLFMDQIALASSLGPSSLFWWLYVLVLLFLPMAMMTSELGTAFPANGGVYHWVRSAFGFRWGARVSWMYWVNNALWMPSVYTLFGSMLGAFYFPELSLWGKIAIGIALALLTAAFNVVALRLGKWLPNLGALLKLLAVLALGVGGLHFGWNHGFANDFSLDSIVPSSPGQMAALGVMVYGIMGTELACCSAAEMRNARRDIPRAVLISGLIVGAFNIFGTLGVLAAVPAEETDVTRIFAHTLYNIYGHDGAGGMLADLVGAFVLFTLFTNMVTWSMGTNRAAVEAAKAGELPALFGVVHSRHGTPIGSAVLASAVSIVLLLLYGLVAHTAEELFWTLLSIFAMVFMMPYVLMCLAFVRLRRADPRPRPYRMPLGDRLASLWALFVALHVLAGICLFVVTPGAPMDWAYAGKIVGGVALALAVGELLIRQAARRRGVMSLRGAYG

SEQ ID NO: 106 - protein, 268 aa, Homo sapiens
MRRIAVVGAAGRMGKNLIEAVQQTGGAAGLTAAVDRPDSTLVGADAGELAGLGRIGVPLSGDLGKVCEEFDVLIDFTHPSVTLKNIEQCRKARRAMVIGTTGFSADEKLLLAEAAKDIPIVFAANFSVGVNLCLKLLDTAARVLGDEVDIEIIEAHHRHKVDAPSGTALRMGEVVAQALGRDLQEVAVYGREGQTGARARETIGFATVRAGDVVGDHTVLFAAEGERVEITHKASSRMTFARGAVRAALWLEGKENGLYDMQDVLGLR

SEQ ID NO: 107 - protein, 188 aa, Homo sapiens
MHFDSVTGWVRGVRHCPSPNFNLRPQGDAVSLLVIHNISLPPGHFGTGKVQAFFQNRLDPNEHPYFEEIRHLTVSAHFLIERDGAITQFVSCHDRAWHAGVSCFDGREACNDFSLGIELEGTDTEPYTDAQYTALAGLTRLLRAAFPAITPERIQGHCDIAPERKTDPGEAFDWSRYRAGLTYSKEET

SEQ ID NO: 108 - protein, 403 aa, Homo sapiens
MNKFMAWVDARFPATKMWEDHLSKYYAPKNFNFWYFFGSLALLVLVNQILTGIWLTMSFTPSAEEAFASVEYIMRDVDYGWIIRYMHSTGASAFFIVVYLHMFRGLLYGSYQKPRELVWIFGMLIYLALMAEAFMGYLLPWGQMSYWGAQVIISLFGAIPVVGEDLAQWIRGDFLISGITLNRFFALHVIALPIVLLGLVVLHILALHEVGSNNPDGVDIKKKKDENGVPLDGIAFHPYYTVKDIVGVVVFLFIFCTVIFFFPEMGGYFLEKPNFEMANQFKTPEHIAPVWYFTPFYAILRAVPDKLMGVVAMGAAIAVLFVLPWLDRSPVRSIRYKGWLSKLWLVIFAVSFVILGYYGAQAPSPLGTTLSRVCTVLYFAFFILMPFYTRMEKTKPVPERVTG

SEQ ID NO: 109 - protein, 687 aa, Homo sapiens
MKLDSRHSLSLKLLRVVLLAALAVGVVLSCAQIVFDAYKAKQAVSSDAQRILAMVRDPSTQAVYSLDREMAMQVLEGLFQHEAVRQASIGHPGEPMLAEKSRPLLDLPTRWLTDPILGQERTFSIRLIGRPPYSEYYGDLKITLDTAPYGENFVTTSEIIFISGILRALAMGLVLFLVYHWMLTKPLSKIIEHLVSINPDRPSQHQLPLLKGHERNELGLWVTTANQLLASIESNSHLRREAEDNLLRISQYDFLTGLPNRQLLQQQLDQILDGAGRQQRRVAVLCLGLDDFKGINEQYTYQLGDQLLIALADRLRGHSARLGSLARLGGDQFALVQADIEQPYEAAELAQSILDGLEAPFEIDQHEVRLRATIGITLFPEDGETTEKLLQKAEQTMTLAKTRSRNRYQFYIASVDSEMRRRRELEKDLRDALQRHELHLVYQPQVDYRDHRVVGVEALLRWQHPLHGFVPPDLFIPLAEQNGSIFSIGEWVLDQACRQLREWHDQGFDDLRMAVNLSTVQLHHNALPRVVSNLLQVYRLPARSLELEVTETGLMEDISTAAQHLLSLRRAGALIAIDDFGTGYSSLSYLKSLPLDKIKIDKSFVQDLLQDEDDATIVRAIIQLGKSLGMQVIAEGVETAEQEAYIIAEGCNEGQGYLYSKPLPARELTQYLKQARRLSQATSSERP

SEQ ID NO: 110 - protein, 233 aa, Homo sapiens
MRAILFDVFGTLVDWRSSLIEQFQALERELGGTLPCVELTDRWRQQYKPAMDRVRNGQAPWQHLDQLHRQSLEALAGEFGLALDEALLQRITGFWHRLRPWPDTLAGMHALKADYWLAALSNGNTALMLDVARHAGLPWDMLLCADLFGHYKPDPQVYLGACRLLDLPPQEVMLCAAHNYDLKAARALGLKTAFIARPLEYGPGQSQDLAAEQDWDLIASDLLDLHRQLAASA

SEQ ID NO: 111 - protein, 499 aa, Homo sapiens
MSHWLILPILLPLFAGSLLLLPLAERWQRGLSLLAALALIPLSLLLIRTAASGDLSVYALGNWAAPFGIVLMLDRLAALMLLATAVLGSAALIYALRGDDRLGKHFHALFQFQLLGINGAFLTGDLFNLFVFFEILLIASYALLLHGGGAERVRSGLHYVILNLVGSAFFLIAVGTLYGLTGTLNMADMAQKIAMADAERAPLLAAAGLLLLVVFALKAALLPLYFWLPRAYAAASAPVAALFAIMTKVGIYSILRVYTLVFGDAAGELANLAQAWLWPLALATLGLGAIGALAARTLQSLLAYLVVVSAGTLLAGVALGSERALAASLYYLLHSTWIAGGLFLLADLVARQRGDKAGDLVQGPALQNPRLLGGAFFIGAIAVAGLPPLSGFFGKVMLLQSVAPGSQALALWSVVLGSGLVALVALSRAGSTLFWRTGHTVLGSAELDHGRLFACILLLSAGPLLVFAAKPLLAYVQATAAQLHDLDLYRQIITRGGAA

SEQ ID NO: 112 - protein, 239 aa, Homo sapiens
MALVDGFLELERSSGKLEWSAILQKMASDLGFSKILFGLLPKDSQDYENAFIVGNYPAAWREHYDRAGYARVDPTVSHCTQSVLPIFWEPSIYQTRKQHEFFEEASAAGLVYGLTMPLHGARGELGALSLSVEAENRAEANRFMESVLPTLWMLKDYALQSGAGLAFEHPVSKPVVLTSREKEVLQWCAIGKTSWEISVICNCSEANVNFHMGNIRRKFGVTSRRVAAIMAVNLGLITL

SEQ ID NO: 113 - protein, 290 aa, Homo sapiens
MDIGLREWLIVIGLIVIAGILFDGWRRMRGGKGKLKFKLDRSFANLPDDDGDSAELLGPARVVEHREPSFDEQDLPSVSAREAKERKGGKRQEEPRQGDLDLDDEGLALEADPSDAAETVEPRKGKSKGRKEKEREKAPAVAAEPAPVDEVLIINVIARDESGFKGPALLQNILESGLRFGDMDIFHRHESMAGNGEILFSMANAVKPGTFDLDDIDNFSTRAVSFFLGLPGPRHPKQAFDVMVAAARKLAHELNGELKDEQRSVLTAQTIEHYRQRIIDHERRSLMQKR

SEQ ID NO: 114 - protein, 496 aa, Homo sapiens
MHGLFLGIDCGTQGSKALLLDAGSGRTLGLGSAAQRPPEGRDGRREQDPADWLEAMASAVRMALEEAAVDGREVRALAVSAQQHGLLLLDAEGRALRPAKLWCDTESAAENRELLEALGGPAGSLERLGLVLAPGYTLSKLLWSRRRFPELFARVAHILLPHDYLNHWLTSRVCSEAGDASGSGYFDVRRRTWASDVLELVEPGGRLAAALPELIEPGACIGNLRPEAAAALGLAPHTRVACGGGDNMLAAIGTGNIRPGLLTASLGTSGTLSAYAERPLVSPHGELATFCASSGGWLPLACTMNLTGACGLVQDLLHLDLDEFSRLAAQAPVGAEGLLMLPFFDGERVPALPHASASLHGMTAANLSRANLCRAVLEGTAFGLRYGLDLLRASGLPGEEVRLVGGAAKNPLWRRTLADLLGLPLVCPRQTEAAALGAALQAAWSLGRESGAGESLEALCRRCVALDESTRTQPQARQQAAYEQAYRRYLELLPPR

SEQ ID NO: 115 - protein, 287 aa, Homo sapiens
MFALNKSALAGLASTSLFVLLWSSGAIASKWGLAHSSPFAFLVFRFGIALACLLPLAPLLRLRAPRSARERGKALLTGLVMLGVYPIFYIFSLKLQVTPGMMATILGVQPILTAVILERRQSPARLFGLLLGLAGLVLVVYQGIGLAGMSTAGILCALLALAGVTGGSIMQKGIRENPLGTLPLQYLAGLGLCLAFVPFQPFEFEWNAGFLVPALWMGVVVSVGATLLLYRLIAQGNLVNVTSLFYLVPAVTAIMDFMVFGNRLGWLSLLGMGLIVVGLMFVFRKAG

SEQ ID NO: 116 - protein, 379 aa, Homo sapiens
MMVMAPGANTALGAAQCSWTLECGNPSAFGDYAAVALLPLDDKRHPKGEAALFQVSQAWMQWSGGQEKICCNLNLGQLPTGADRVLLVVYTFSAMGPVSDLRLLRLQIDSQIEFNLNLSDNGESAIIVGEFYCRNHQWKFRALAEGSAYGLAALGRRIGLKIDDAHPHRRSSSSEQSRPASGATGTGFAVTSTHILTCAHVIEDMKEIHIASFEGRHRAEAVVVDQRNDLALLRVQGAPAFKPVAFRDGVGCDLGEPVVALGFPLAGLAGGGVHVTQGGVSALFGLHNDSSLLQFTAPIQPGSSGSPLFDAAGSVVGMVTSTIPDAQNMNFAVKAGLALAFLDACGIEPARTPTGKTFSTSQMAREAQQSLWKIEARNP

SEQ ID NO: 117 - protein, 147 aa, Homo sapiens
MLTDWAAYAGLFLSAFGSATLLPLQSETVLAALLLRGGQSVAWLLALAIVGNVLGSWVNWWLGRYLEHFRGRRWFPVGEVQLLRAQRHYRRYGRWTLLLSWVPVIGDPLTLVAGIMREPCWSFLLIVGLAKTLRYLALAALVLGWAG

SEQ ID NO: 118 - protein, 474 aa, Homo sapiens
MLGKHSLVYFLFKSFPAILTLVGLSVFTRLLSPGEYGVYSLTIIVVGFLNTVFLQWVALGVGRYLPECSDDQARARLLGTARAISFLVSLVIIFVTFLLWEWREEIGFSILYYMVGFLCLAQAWHDLNLKIQNAILQPLTYGKMLLIKGAGSFFIGVLLVYFGFGVDGLLLGTLVSLVLATIFFQDAWRGVSWALVDKEQLTRLFAYGAPLTLTFLFAFIVNASDRFFIGAFLGDAAVGVYSVSYDLAQYSVGTVASVVHLAAFPLVMEKLSKSGLPQTQDQLRKTFIFIFAVVSPAACGLAMVAPEISGSIMGEEFREGALKIIPLISLSAFLGALKSFYFDYSFQLASATRVQVVTVAVSAVVDVVFNLILIPEFGIVGAAVSSVMAFSSAILISIFLGRRVFPMPALPGKDAMKIALSVLLMAVSVASFSLESAFFGLVVKVVLGGGVYLAAMIALDVSGMRTFLKSKLIR

SEQ ID NO: 119 - protein, 417 aa, Homo sapiens
MYAMLTGATLLIFAVAARLLARSAIHPSVAMPITWGLGLIGVSLASLIGFYRVESDALLIFLFGVMSFSLSAGCFSFLYNGYFRAPSSNFLFDSELRTRALVIFFCLAHIVFLTVIYRDLSSIAPTLREAAYMARAQSVSGEPVLSSLSMNYLQLGQTVIPLVVLLYLRGKCGVLGFLAISVPWMGVILLASGRASLMQMLVGLFFIYILVKGSPSLKSLLVIGLAMFLVIAVGAVATSKIQFHEGDGISTLFIELYRHVAGYALQGPVLFDRYYQGSIHLEPYWSPLNGFCSILATVGLCQKPPLHLDFYEYAPGELGNVYSMFFSMYPHYGALGVIGVMALYGMLCSYAYCKAKKGSLYFTVLSSYLFSAIVFSLFSDQISTSWWFYVKMTIILGILCFVFRRDRMFVIRLPQAG

SEQ ID NO: 120 - protein, 228 aa, Homo sapiens
MSLDKQALFWLAATVGGYLLSRQLYRRVKWYWLSPIVFVPVLLYALAIPTHTRYADYARDTNWLVALLGPATVAFAIPIWQQRELLMRHWPALLAGMFAGTAVAIGSSWALAQALALDGQVTLSLLPRSITTPFAMEMSHDLGGVPELTAAFVMITGVFGAVIGGTLLRVLRLRTPLARGALFGVGAHGAGTSRAYEFGGEEGSAAGLLMVLTGLFNLLVAPLVAHCL

SEQ ID NO: 121 - protein, 563 aa, Homo sapiens
MNLKPLIASFLLVATGGCTITNVNDTMRRAEVASESAEGLAASMRSRQDTPARPTVRYSDTPWVSTRPIDLKIDGIPDALNCDITYSPTVDVDIFQVGQEITKWCGIPVRVTPDVTLTGSSTSAISLPSLNDGAQSSAANPASSMGLPPLPALPQGGSALGSSGGRNLTISGLKWKGGPAKGLLDMATVRLGLSWKYSAAENLVTIFYVDTKTFRFYAIPSVTDMTSVVQSGTTTAAGVSNSGTSSSSSGGGISGNSGSSQSTGVTINTDITKDIGNSVQSMLTPGVGRMSMSSSTGTMTVTDTPEVLARVGDFLNGENSNITKQVLLNVKVLSVTLTDKDDLGIDWNLVYKAVNGKWGLGWKNVTQTDAAAVQGSVSILDTSSQWAGSNLLVKALAQQGRVSTITSPSVTTLNLQPVPVQVARQTSYLASIQTTNTADVGSTTSLTPGTVTSGFNMNLLPYVMPGKELLLRYSINLSALKQIRQVSSGDNTIEIPEVDNRIFSQMVKLRSGETLVLSGFEQSVDNGSKAGVGSASNWLMGGSLKRDNSKDVIVVLITPIVEG

SEQ ID NO: 122 - protein, 393 aa, Homo sapiens
MTRIGTPLSPSATRVLLCGSGELGKEVAIELQRLGCEVIAVDRYGNAPAMQVAHRSHVISMLDGAALRAVIEQEKPHYIVPEIEAIATATLVELEAEGYTVVPTARAAQLTMNREGIRRLAAEELGLPTSPYHFADTFEDYRRGVERVGYPCVVKPIMSSSGKGQSVLKGPDDLQAAWDYAQEGGRAGKGRVIVEGFIDFDYEITLLTVRHVDGTTFCAPIGHRQVKGDYHESWQPQAMSAQALAESERVARAVTEALGGRGLFGVELFVKGDQVWFSEVSPRPHDTGLVTLISQDLSEFALHARAILGLPIPVIRQLGPSASAVILVEGKSRQVAFANLGAALSEADTALRLFGKPEVDGQRRMGVALARDESIDAARAKATRAAQAVRVEL

SEQ ID NO: 123 - protein, 458 aa, Homo sapiens
MSVPQRAVQLTEPSEFLKEHPEVQFVDLLIADMNGVVRGKRIERNSLNKVFEKGINLPASLFALDITGSTVESTGLGLDIGDADRICYPIPGTLSMEPWQKRPTAQLLMTMHELEGEPFFADPREVLRQVVARFTEMELTIVAAFELEFYLIDQENVNGRPQPPRSPISGKRPQSVQVYSIDDLDEYVECLQDIIDGARAQGIPADAIVAESAPAQFEVNLNHVNDALKACDHAVLLKRLVKNIAYDHEMDTTFMAKPYPGQAGNGLHVHISLLDKHGNNIFTSEDPEQNAALRHAIGGVLETLPASMAFLCPNVNSYRRFGSQFYVPNAPSWGLDNRTVALRVPTGSPDAVRLEHRVAGADANPYLLLAAVLAGVHHGLTNKVEPGAPIEGNSYEQMEPSLPNNLRDALRELDESEIMAKYIDPKYIDIFVACKESELEEFEHSISDLEYNWYLHTV

SEQ ID NO: 124 - protein, 182 aa, Homo sapiens
MSDTTLESAGLSRRSLMKVGLIGGAFLATAGVTASLTGCSAEKPASGLEKVRESDLPFLRALLPVMLLGAVSAEQMPKAVEGAIQSLDHNLARLSPEMFKLTQQLFDVLALPLTRGPLTGIWGSWENASGDDVRAFLSRWENSFIGLLRMGHSSLMQLAMMAWYARPEAWAHCGYPGPPKIA

SEQ ID NO: 125 - protein, 121 aa, Homo sapiens
MARILIVDDSPTEMYKLTAMLEKHGHQVLKAENGGDGVALARQEKPDVVLMDIVMPGLNGFQATRQLTKDAETSAIPVIIVTTKDQETDKVWGKRQGARDYLTKPVDEETLLKTINAVLAG

SEQ ID NO: 126 - protein, 363 aa, Homo sapiens
MPRYLGLMSGTSLDGMDIVLIEQGDRTTLLASHYLPMPAGLREDILALCVPGPDEIARAAEVEQRWVALAAQGVRELLLQQQMSPDGVRAIGSHGQTIRHEPARHFTVQIGNPALLAELTGIDVVADFRRRDVAAGGQGAPLVPAFHQALFGDGDASRAVLNIGGFSNVSLLSPGKPVRGFDCGPGNVLMDAWIHHQRGEHFDRDGAWAASGQVNHALLASLLADEFFAARGPKSTGRERFNLPWLQEHLARHPALPAADIQATLLELSARSISESLLDAQPDCEEVLVCGGGAFNTALMKRLAMLMPEARVASTDEYGIPPAWMEGMAFAWLAHRFLERLPGNCPDVTGALGPRTLGALYPA

SEQ ID NO: 127 - protein, 156 aa, Homo sapiens
MPRRRVAAKREVLADPKYGSQILAKFMNHVMESGKKAVAERIVYGALDKVKERGKADPLETFEKALDAIAPLVEVKSRRVGGATYQVPVEVRPSRRNALAMRWLVDFARKRGEKSMALRLAGELLDAAEGKGAAVKKREDVHRMAEANKAFSHYRF

SEQ ID NO: 128 - protein, 244 aa, Homo sapiens
MTAIGTIFRRELGSYFATPLAYVFTLVFLVLSGVATFYLGDFFERGQADLAPFFSSLPWLYLLLIPALAMRLWAEERKSGSIEMLMTLPVSRATLVTGKFLAAWFCAGLALLLTFPMPLTVNYLGSPDNGAIIAGYLAGWLLSGGYLAIGSCMSALAKNQIIAFALTVLVCLLFVGAGTPHVQQALSGWLPQWLLDGIASLSVLVRFEALGRGVLDVRDLAYFCSLIVAWLVATTIVIDLKKAA

SEQ ID NO: 129 - protein, 795 aa, Homo sapiens
MRHWLILFLIALPCLAGAVSFNEQVERLPLGQSIDVFEDVRGSADINDITSRAIDSSFRRHDKDVLNAGYSRSVFWLRLDLDYRPVASSDPRTWLLELAYPPLDKLDLYLPDGQGGYRLAQRTGDTLPFASRPIRQNNYLFELGLEPNKPQRVYLRLESQGSIQAPLTLWSPKAYLEEQPERIYVLGIIYGVLLVMLIYNLFIFLSVRDTSYLYYILYIASFGLYQVSVNGAGIEYFWPDSPWWANAATPFLIGSAALFGCQFARSFLHTRDHSVWVDRGLLALMAVGALVMLMALTMSYAVALRLATYLALAFTGLIFAAGILAWLRGMRVARYFIIAWTAFLLGGIVNTLMVLGYLPNMFLTMYASQIGSALEVGLLSLALADRINAMKEERARILQESSRKLEALNQELANSNRLKDEFLATVTHELRTPMSGVIGSLELMQTVPMDVELAEYQRTAAGSARDMMRMVNDILALIELQAGKLYPRREPFSLRGLFDSLRAQYAPRAEEKGLRFALQLDDSLPDTLEGDAGKLAQALGYLVDNAIKFTARGSVTLRVAAGRTHDGVALRVEVIDTGIGFDMAAGSDLYQRFVQADSSLTRGYGGLGIGLALCRKLVELLGGELTHESRPGQGSRFLLRLQLTQPAQGLAPPPRRAGGQAVRRPEECTVLVVEDNAINQLVTRGMLLKLGYRVRTADNGSEALELLARERPDGVLLDCQMPVMDGFATCRAIRALPGCAELPVLALTAHSHSGDRERCLAAGMSDYMAKPVKFEELQTLLHDWLLCQPIVTKSA

SEQ ID NO: 130 - protein, 299 aa, Homo sapiens
MSDAPTSPRFSAATSTRLLDHAQLEALCADYDGFLLDLWGVVMDGTEAFPGALAWLARRHAEGRPVWFLSNSSSSVVEMSAGLERLGIRRDWFAGITTSGQLTIDALLQTAEYRRGGIYLAGVGLAQQSWPAEIRERFVEDIAQAALIVGVGSFPQDELEQRFAPLRGATDLPFLCANPDRVVVSGGRTVYGAGMLAELFSEEGGQVSWYGKPDPAAFRIAQRQLEARGARHILFVGDSLVTDVPGALAARIDTLWLGATGIHREALGAEFNGALDEERVRSLLHGYPIRPHFAAPGLV

SEQ ID NO: 131 - protein, 769 aa, Homo sapiens
MTPDQMRDASLLELFRLEAEAQTQVLNAGLMALERSPTQADQLEACMRAAHSLKGAARIVGLDAGVRVAHVMEDCLVEAQDGRLLLQSEHIDALLQGCDLLLRIGTPPAGDAGWAEGAGREEIDGLVLRLEGLVRSGLPLARAELPATTPGLPEAVPEAPPAASAAASDDNDEEPAGQAGGEQAEERRSRVLRVTAERLDRLLDISSKSLVEFQRIKPLADSLQRLRRLQSSASRALDVVRETVQETALDPQAQAMLGEARQLIGECQQMLVQHIADLDEFAWQGGQRAQVLYDAALASRMRPFADVLSGQARMVRDLGRSLGKQVRLLVEGESTQVDRDVLEKLEAPLTHLLRNAVDHGIEAPETRLAAGKPAEGRITIRARHHAGMLVLELSDDGGGIDLQRLRETVLNRQFATAETVAQLSEEELLAFLFLPGFSMREQVTEVSGRGVGLDAVQHMVRQLRGGVRMEQRQGQGALFHVEVPLTLSVVRSLVVEIGEEAYAFPLAHIERMCELEAEEIVQLEGRQHFWYEGRHVGLVSAAQLLQRPESSRTEGAIPVVVVRDRDAVYGVAVERFVGERTLVVMPLDPRLGKVRDVSAGALLDDGSPVLILDVEDLLHSVGKLLSSGRLERIDRSRRQAGGAQRKRILVVDDSLTVRELERKLLLGRGYDVAVAVDGMDGWNALRSEHFDLLITDIDMPRMDGIELVTLVRRDSRLQSLPVMVVSYKDREEDRRRGLDAGADYYLAKASFHDEALLDAVVVLIGEAQG

SEQ ID NO: 132 - protein, 212 aa, Homo sapiens
MRRTKEDSEKNRTAILLAAEELFLEKGVSHTSLEQIARAAGVTRGAVYWHFQNKAHLFNEMLNQVRLPPEQLTERLSGCDGSDPLRSLYDLCLEAVQSLLTQEKKRRILTILMQRCEFTEELREAQERNNAFVQMFIELCEQLFARDECRVRLHPGMTPRIASRALHALILGLFNDWLRDPRLFDPDTDAEHLLEPMFRGLVRDWGQASSAP

SEQ ID NO: 133 - protein, 224 aa, Homo sapiens
MRILIIEDEVKTADYLHQGLTESGYIVDRANDGIDGLHMALQHPYELVILDVNLPGIDGWDLLRRLRERSSARVMMLTGHGRLTDKVRGLDLGADDFMVKPFQFPELLARVRSLLRRHDQAPMQDVLRVADLELDASRHRAFRGRVRINLTTKEFALLHLLMRRNGDVITRTQIISLIWDMNFDNDSNVVEVAICRLRAKIDDGFDLKLIHTIRGVGYVLEARR

SEQ ID NO: 134 - protein, 370 aa, Homo sapiens
MERIDHLLPWSTLGSEKRLSVFRFGCGARKVYIQSSLHADELPGMRTAWELKQRLRLLEAEGRLRGTVELVPVANPVGLGQMIQALHQGRFEMSSGRNFNRDFPDLLDAVIDSVGERLGSDPAANVALVRQTLRAALDALPPATSELEGMQRLLYRHACDADLVLDLHCDFEAAIHLYTLPQQWPAFASLAARLGAAVGLLAEESGGGSFDEACSVPWLRLSRLYPRAELPLACLATTVELGGQADTTVQQAEANAAAILAFLAEQGFVEGEWPAAPEACCEGLPFEGTEYVHAPHTGVVSFLRRPGEWVEAGEPLFQVIDPLADRASTVCAGVSGVLFAIERMRYAQPGLWLAKVAGRQPIRQGRLLSD

SEQ ID NO: 135 - protein, 687 aa, Homo sapiens
MKLDSRHSLSLKLLRVVLLAALAVGVVLRCAQIVFDAYKAKQAVSSDAQRILAMVRDPSTQAVYSLDREMAMQVLEGLFQHEAVRQASIGHPGEPMLAEKSRPLLDLPTRWLTDPILGQERTFSIRLIGRPPYSEYYGDLKITLDTAPYGENFVTTSEIIFISGILRALAMGLVLFLVYHWMLTKPLSKIIEHLVSINPDRPSQHQLPLLKGHERNELGLWVTTANQLLASIESNSHLRREAEDNLLRISQYDFLTGLPNRQLLQQQLDQILDGAGRQQRRVAVLCLGLDDFKGINEQYTYQLGDQLLIALADRLRGHSARLGSLARLGGDQFALVQADIEQPYEAAELAQSILDGLEAPFEIDQHEVRLRATIGITLFPEDGETTEKLLQKAEQTMTLAKTRSRNRYQFYIASVDSEMRRRRELEKDLRDALQRHELHLVYQPQVDYRDHRVVGVEALLRWQHPLHGFVPPDLFIPLAEQNGSIFSIGEWVLDQACRQLREWHDQGFDDLRMAVNLSTVQLHHNALPRVVSNLLQVYRLPARSLELEVTETGLMEDISTAAQHLLSLRRAGALIAIDDFGTGYSSLSYLKSLPLDKIKIDKSFVQDLLQDEDDATIVRAIIQLGKSLGMQVIAEGVETAEQEAYIIAEGCNEGQGYLYSKPLPARELTQYLKQARRLSQATSSERP

SEQ ID NO: 136 - protein, 439 aa, Homo sapiens
MNARVHQPVHTAQHAPSYYAATLNRRIECPPLAGEEQADVCVVGGGFSGVNTALELAQRGFSVVLLEAHRIGWGASGRNGGQLIRGVGHDVEQFLPVIGADGVKALKLMGLEAVEIVRRRVEQYAIDCDLRWGYCDLANKPGDYQGFREDMEELQALGYRHEMRLVPAAEMRSVVGSDRYVGGLVDMGSGHLHPLNLVLGEAAAAQSLGVRLFERSPVTRIDYGTEVQVHTATGKVRAKTLVLGCNAYMNDLNPLLGGKVLPAGSYVIATEPLDEKLARQLLPQNMAVCDQRVALDYYRLSADNRLLFGGACHYSGRDPSDIAAYMRPKMLEVFPQLANVRIDYQWGGMIGIGANRLPQIGRLPGQPNVYFAQAYSGHGVNATHLAGQLLAEAIGGQQSDGFDLFAKVPHITFPGGKLLRSPLLALGMAWYRLKEKLGS

SEQ ID NO: 137 - protein, 280 aa, Homo sapiens
MAEWNRNETLWRQGLLLASDAVEALGLHHPESPERTLVIVASHDCDLAQSPEKEPDIEVVIGRLALEKDGNSTHAKNARKLHIEFTGADTFWAEFEATAKSKVGKLELNRHAPRSGATLSPECHAVFQMWLASRYRRSAFPDEFERRLTSKDFKLHERISKAVKPHGDLIAGVFFDVDEGVEINRNGADDTYTLDIIIMHSADPNFEEAEKAAESAAATITQAFKEKLFSPTSTWQHIELRSCDAVSESVLTYQQFKQLKRWRLEHLSLAADPQQPVLAE

SEQ ID NO: 138 - protein, 525 aa, Homo sapiens
MKIKAALIVDDLSLREWQKRAIEDSSEYLDIQLVLSCRNSATKKSVIKHCGYYFLNILSLKNDMTRRVQLDSRGSEVIHFDSDYEGAWQRIPEDVCARILDKGIKLVIKFGMSLLRIDGGLQRLDILSYHHGDPEYYRGRPAGFYEIYENADSVGIIVQKLSNKLDAGEVLVRGYSKVHHHSYKKTSRNFYLNSVVLLRKALVNYSRGEQVVLEKLGKNYRLPSNFTVFKFFCKTIFRGLARLSYGAFFEKKWNVVALPYNDIPSLQELSVSAGKIPKVEKGYTFYADPFFSADGKLIRLEALNASNGLGEIIELKAQSLDFSRVILKGNHFSYPYSFEASGVEYLIPEVASHSAPCLLPPPFALESKKLFQGMEGERILDGTLFEHGGRYYLFCGQAVSGSDNLYLYVGESLEGPYTSHPCNPVVMNPGSARMGGRIFKEGGKLYRFGQNNSYGYGSSLAVNEIEVLDPEHYSEKRVANLAFQDARGPHTIDIHGQTMILDFYQDRFSLLAGYRRLVARLLSKG

SEQ ID NO: 139 - protein, 908 aa, Homo sapiens
MSRSPIPRHRALLAGFCLAGALSAQAATQEEILDAALVSGDSSQLTDSHLVALRLQQQVERIRQTRTQLLDGLYQNLSQAYDPGAASMWVLPANPDNTLPFLIGDKGRVLASLSLEAGGRGLAYGTNVLTQLSGANAAHAPLLKRAVQWLVNGDPGAATAKDFKVSVVGVDKTATLNGLKSAGLQPADAACNALTDASCASASKLLVLGNGASAASLSATVRARLQAGLPILFVHTNGWNQSSTGQQILSGLGLQEGPYGGNYWDKDAVPSSRTRARSVELGGAYGQDPALVQQIVDGSWRTDYDWSKCTSYVGRTTCDDVPGLSDFSKRVDVLKGALDAYNQKAQNLFALPGTTSLRLWLLWADAVRQNIRYPMDKAADTARFQETFVADAIVGYVREAGAAQKELGSYAGQRQQSMPVSGSEETLTLTLPSAQGFTAIGRMAAPGKRLSIRIEDAGQASLAVGLNTQRIGSTRLWNTRQYDRPRFLKSPDIKLQANQSVALVSPYGGLLQLVYSGATPGQTVTVKVTGAASQPFLDIQPGEDSSQAIADFIQALDADKADWLEIRSGSVEVHAKVEKVRGSIDKDYGGDVQRFIRELNEVFIDDAYTLAGFAIPNQAKTPAIQQECAVRGWDCDSETLHKLPGTQHINVDQYAQCGGGCSGNPYDQTWGLNPRGWGESHELGHNLQVNRLKVYGGRSGEISNQIFPLHKDWRVLREFGQNLDDTRVNYRNAYNLIVAGRAEADPLAGVYKRLWEDPGTYALNGERMAFYTQWVHYWADLKNDPLQGWDIWTLLYLHQRQVDKSDWDANKAALGYGTYAQRPGNSGDASSTDGNDNLLLGLSWLTQRDQRPTFALWGIRTSAAAQAQVAAYGFAEQPAFFYANNRTNEYSTVKLLDMSQGSPAWPFP

SEQ ID NO: 140 - protein, 211 aa, Homo sapiens
MALTIVIGNRNDSSWSLRGWLALRMSGAAFDEILVPLGRPDSRERILQYSPTGKVPLLKSEDGDIWDSLAIAEYLAERFPEAHLWPRGEAARALARSVCAEMHSGFAALRGEPPMDLRRQQPLVELSEATRQDIQRICEAWADCRRRFGQDGPFLFGHASLADAFYAPVAARFRSYAVELPDIARTYVETIYQWPAFRAWYDAALREQAGS

SEQ ID NO: 141 - protein, 230 aa, Homo sapiens
MTLLDIEKLLDMTHHMSVHIPIELIISWEQSEEAWSVNDNCLNFVYVNRRYTELITPRFGQKNSLLSPFSASIEEHDKLVIQTGKRIEALALLRPDDYPAPCCLYFERMPLYDRRGNRTGVIAHAKTLTSVAPRGFIATDGIGTFTFTPPSELFTSREWDVIYLLLSGLSEKEIAEQISRSLSTVKFHKSNIFQKVGCSCIGAFKALARQKKWNFYIPPTFASAKYIINH

SEQ ID NO: 142 - protein, 4342 aa, Homo sapiens
MMDAFELPTTLVQALRRRAVQEPERLALRFLAEDDGEGVVLSYRDLDLRARSIAAALQAHAQLGDRAVLLFPSGPDYVAAFFGCLYAGVIAVPAYPPESARRHHQERLLSIIADAEPRLVLTTADLREPLLQMNAQLSAANAPQLLCVDQLDPAVAEAWDEPQVRPEHIAFLQYTSGSTALPKGVQVSHGNLVANEVLIRRGFGIGADDVIVSWLPLYHDMGLIGGLLQPIFSGVPCVLMSPRYFLERPVRWLEAISQYGGTVSGGPDFAYRLCSERVAESALQRLDLSGWRVAFSGSEPIRQDSLERFAEKFAASRFDASSFFACYGLAEATLFVTGGQRGQGIPALAVDGEALARNRIAEGAGSVLMCCGRSQPEHAVLIVDAVSGEALGDDNVGEIWAAGPSIAHGYWRNPEASAKTFVERDGRTWLRTGDLGFLRDGELFVTGRLKDMLIVRGHNLYPQDIERTVESEVPSARKGRVAAFAVTVDGEEGIGIAAEIGRGVQKSVPAQELIDSIRQAVAEAYQEAPKVVALLNPGALPKTSSGKLQRSACRLRLEDGSLDSYALFPGLQAVQEAQPPAGDDELLARIGEIWKARLGVAQVAPRDHFFLLGGNSIGAAQVVAQVRDSLGVALDLRQLFEAPTLHAFSATVARQLAAGLPAEAPMAHLPRGVDLPQSAAQQRLWLTWQIDPQSAAYNIPGGLRLRGELDEAALRASFQRLVERHEALRTRFLERDGAALQRIDERGEFAWQFVDLAALAEHERAAAAAQRREAEAQQPFDLEKGPLLRISLVRLDEQEHQLWVTLHHIVADGWSLNLLLDEFSRLYAEACGGQPADLAPLELHYAEFAAWQRQWLDAGEGARQLAYWRERLGDAAPVLELATDHPRTARQASPAARYSLRVDEALARAIREAALDHEASVFMWLLAAFQALLHRHSGQGEIRIGVPSANRQRLDTQGLVGFFINTLVLRGTPRARQPFAALLGEAREATLGAQANQDLPFDQVLAACGQGGQLFQVLFNHQQRDLSALRRLPGLLADELPWHSREAKFDLQLQSEEDARGRLTLNFDYAADLFDEASIRRFAAQYLELLRQVAEDPQRCLGDIALVDAEQAARLAEWGSAPCEPARAWLPELLERQLAQSAERVALEWDGGSLGYAELHARANRLAHYLRDKGVGPDVRVAICAERSPQLLVGLLAIVKAGGAYVPLDPDYPSERLAYMLADSGVELLLTQAHLFERLPGAEGVTPICLDSLKLDNWPSQAPGLHLHGDNLAYVIYTSGSTGQPKGVGNTHAALAERLQWMQATYALDGDDVLMQKAPVSFDVSVWECFWPLVTGCRLVLAAPGEHRDPARLVELVRQFGVTTLHFVPPLLQLFIDEPGVAACGSLRRLFSGGEALPAELRNRVLQRLPAVALHNRYGPTETAINVTHWQCRAEDGERSPIGRPLGNVVCRVLDAEFNLLPAGVAGELCIGGLGLARGYLGRPALSAERFVADPFSADGERLYRTGDRARWNADGVLEYLGRLDQQVKLRGFRIEPEEIQARLLAQPGVAQAVVVIREGVAGSQLVGYYTGAVGAEAEAEQNQRLRAALQAELPEYMVPAQLMRLAQMPLGPSGKLDTRALPEPVWQQREHVEPRTELQRRIAAIWSEVLGLPRVGLRDDFFELGGHSLLATRIVSRTRQACDVELPLRALFEASELEAFCEQVRAAQAAGRTDSHGAIRRIDREQPVPLSYSQQRMWFLWQLEPDSPAYNVGGLARLSGPLDVARFEAALQALVQRHETLRTTFPSVDGVPVQRVHGDGGLHMDWQDFSALDRDSRQQHLQTLADSEAHRPFDLESGPLLRVCMVKMAEREHYLVVTLHHIVTEGWAMDIFARELGALYEAFLDDRESPLEPLPVQYLDYSVWQREWLESGERQRQLDYWKAQLGNEHPLLELPGDRPRPPVQSHQGDLYRFDLSPELAERVRRFNAARGLTMFMTMTATLAALLYRYSGQQDLRIGAPVANRIRPESEGLIGAFLNTQVLRCRLDGQMSVGELLEQVRQTVIDGQSHQDLPFDHLVEALQPPRSAAYNPLFQVMCNVQRWEFQQTRQLAGMTVEYIANDARATKFDLNLEVTDLDQRLGCCLTYSRDLFDEPRIARMAGHWQNLLEALLGDPQRRIAELPLFAAEERKQLLLAGTAGEAGLQDTLHGLFAARVAASPQAPALTFAGQTLSYAELDARSNRLARVLRSHGVGPEVRVGLALERSLEMVVGLLAILKAGGAYVPLDPEYPLERLQYMIEDSGVRLLLSHAALFEALGELPAGVARWCLEEDGPALDAEDPAPLAALSGPQHQAYLIYTSGSTGKPKGVAVSHGEIAMHCAAVIERFGMRAEDCELHFYSINFDAASERLLAPLLCGARVVLRAQGQWGAEEICELIRAEGVSILGFTPSYGSQLAQWLESQGRQLPVRMCITGGEALTGEHLQRIRQAFAPASFFNAYGPTETVVMPLACLAPERLEEGAASVPIGSVVGARVAYILDADLALVPQGASGELYVGGAGLARGYHERPALSAERFVPDPFAAEGGRLYRTGDLVRLCDNGQVEYVGRIDHQVKIRGFRIELGEIEARLLEHPQVREALVLALDSPSGKQLAGYVASAVAEQDEDAQAALREALKTHLKQQLPDYMVPAHLLLLASLPLTANGKLDRRALPAPDPALNRQAYEAPRSVLEQQLAGVWREVLNVERVGLGDNFFELGGDSILSIQVVSRARQLGIHFSPRDLFQHQTVQSLAAVARHSQASQAEQGPVQGDSALTPIQHWFFDLPLARREHWNQSLLLQPRQALDLGLLRKSLQRLVEQHDALRLAFRQVDGEWLAQHRPLREQELLWHVPVQSFDECAELFAKAQRSLDLEQGPLLRAVLVDGPAGEQRLLLAIHHLVVDGVSWRVLLEDLQQVYRQFAEGAEPALPAKTSAFRDWAGRLQAYAGSESLREELGWWQARLGGQSAEWPCDRPQGDNREALAESVSLRLDPQRTRQLLQQAPAAYRTQVNDLLLAALARVLCRWSGQPSTLVQLEGHGREALFDDIDLTRSVGWFTSAYPLRLTPAQSPGESIKAIKEQLRAVPHKGLGYGVLRYLADPAVRQAMAALPTAPITFNYLGQFDQSFADALFQPLDQPTGPIHDEQAPLPNELSVDGQVYGGELVLRWTYSRERYDARTVNELAQAYLAELQALIEHCLEDGAGGLTPSDFPLAQLSQAQLDALAVPVGEIEDVYPLTPMQEGLLLHTLLEPGTGIYYMQDRYRIDSPLDPERFAAAWQAVVARHEALRASFVWNAGETMLQVIHKPGRTRIEFLDWSELPEDGHEERLQALHKREREAGFDLLEQPPFHLRLIRLGEARYWFMMSNHHILIDAWCRGLLMNDFFEIYGALGEGRPANLPTPPRYRDYIAWLQRQDLEQSRRWWSESLRGFERPTLVPSDRPFLREHAGESGGMIVGDRYTRLDAADGARLRELAQRYQLTVNTFAQAAWALTLRRFSGERDVLFGVTVAGRPVGMPEMQRTVGLFINSIPLRVQMPAAGQRCTVREWLNRLFERNLELREHEHLPLVAIQESSELPKGQPLFDSLFVFENAPVEVSVLDRAQSLNASSDSGRTHTNFPLTVVCYPGDDLGLHLSYDQRYFEAPTVERLLGEFKRLLLALADGFHGELEALPLLGEDERDFLLDGCNRSARDYPLEQGYVRLFEAQVAAHPQRIAASCLEQRWSYAELNRRANRLGHALRAAGVGIDQPVALLAERGLDLLGMIVGSFKAGAGYLPLDPGHPTQRLTRIVELSRTPVLVCTQACREQALALFDELGCVDRPRLLVWDEIQQGEGAEHDPQVYSGPQNLAYVIYTSGSTGLPKGVMVEQASMLNNQLSKVPYLELDENDVIAQTASQSFDISVWQFLAAPLFGARVAIVPNAIAHDPQGLLAHVGEQGITVLESVPSLIQGMLAEERQALDGLRWMLPTGEAMPPELARQWLKRYPRIGLVNAYGPAECSDDVAFFRVDLASTESTYLPIGSPTDNNRLYLLGAGADDAFELVPLGAVGELCVAGTGVGRGYVGDPLRTAQAFVPHPFGAPGERLYRTGDLARRRADGVLEYVGRIDHQVKIRGFRIELGEIEARLHERADVREAAVAVQEGANGKYLVGYLVPGETPRSSADSPAGLMVEQGAWFERIKQQLRADLPDYMVPLHWLVLDRMPLNANGKLDRKALPALDIGQMQNQAYQAPRNELEETLARIWAEVLKVERVGVFDNFFELGGHSLLATQIASRVQKALQRNVPLRAMFECTTVEELASYIESLAPSEISEQKAERLNDLMSKLEML

SEQ ID NO: 143 - protein, 187 aa, Homo sapiens
MSEQLSTRRCDTPLLQAFVDNRTILVKIAARITGCRSRAEDVVQDAFFRLQSAPQITSSFKAQLSYLFQIVRNLAIDHYRKQALEQKYSGPEEEGLNVVIQGASPETSHINYATLEHIADALTELPKRTRYAFEMYRLHGVPQKDIAKELGVSPTLVNFMIRDALVHCRKVTAERQGDNVTHLSARR

SEQ ID NO: 144 - protein, 501 aa, Homo sapiens
MKTSFAFTRPARTLAPLALAAALAGCSMAPKYDRPAAPIDTAYPSGAAYVELAAATPDDAITAEIGWRDFFRDPLLQQLIGISLENNRDMHKAALNVEAAQALYRIQRAEMLPNLGVSARGASERVPADLSTTGQSDVLRRYDVAGVTAAWELDLWGRIRSLNDRALASYLALDETRIATQMSLVSEVASAYLTLRADQELLRLTSDTLATQKRSYDLTTQLVEAGNSTQLDLRRAEIALRTAEANRAAYTRQAAKDRNALVLLLGQPLTPELSRQLDEAVALPDDIVPTDLPSGLPSELLARRPDIRAAEQMLIGANANIGAARAAFFPTISLTGSAGTASASLDGLFDSGSRAWSFLPQITLPIFRGGALRANLDVAQVQKRIEIANYEKSIQAAFAEVADGLAGKRTLDEQIRSEQLLVAASQKAYQLAEQRFQEGVDDNLTLLDAQRTQYGAQQTLVRTRLTRLNNLIHLYKALGGGWTEHTVQSGATGQPSARSPG

SEQ ID NO: 145 - protein, 241 aa, Homo sapiens
MRNDGGFLLWWDGLRSEMQPIHDSQGVFAVLEKEVRRLGFDYYAYGVRHTIPFTRPKTEVHGTYPKAWLERYQMQNYGAVDPAILNGLRSSEMVVWSDSLFDQSRMLWNEARDWGLCVGATLPIRAPNNLLSVLSVARDQQNISSFEREEIRLRLRCMIELLTQKLTDLEHPMLMSNPVCLSHREREILQWTADGKSSGEIAIILSISESTVNFHHKNIQKKFDAPNKTLAAAYAAALGLI

SEQ ID NO: 146 - protein, 359 aa, Homo sapiens
MSANKKPVTTPLHLLQQLSHSLVEHLEGACKQALVDSEKLLAKLEKQRGKAQEKLHKARTKLQDAAKAGKTKAQAKARETISDLEEALDTLKARQADTRTYIVGLKRDVQESLKLAQGVGKVKEAAGKALESRKAKPATKPAAKAAAKPAVKTVAAKPAAKPVAKTAAAKPAAKPAAKPAAKPAAKPAAKTAAAKPAAKPVAKPAAKPAAKPAAKTAAAKPAAKSAAKPVAKPAAKPAAKTAAAKPAAKPAVKPVAKPAAKPAAKTAAAKPAAKPAAKPVAKPAPAAAKPAAKPAAKPAAKPVAKPAAKPVAAKPAAAKPATAPAAKPAATPSAPAAASSAASATPAAGSNGAAPTSAS

SEQ ID NO: 147 - protein, 652 aa, Homo sapiens
MGGKAKGQFNLFHSHFVFAMHPPKILLWLLPLVCAFSLGAIADTAVDPSQALHLLSYLAADYPPTVADGKIVDPSEYQEQVEFVGNLQALVLTLPMRPERAELERGGASLRQAIEQRLPGRDVALQARNLEARVADIYQVVQTPAITPDPSRAAPIYAQQCAICHGDAGKGDGPAGIGLEPPPANLTDRQRLDHLSLYDLRNVIGLGVAGTDMPAFADQLDERQRWDLASYVAGLSAGSAQPDKAHAYPLATLATQTPAEVAEHDGEAAAESFRALRAHPPLEQRGPGQLIDYTAATLDKSFAVYREGDRDQAYDLSVAAYLEGFELVESSLDNVDADLRRSTEKQLMAYRQALRDGLPETQVAQQLELAKGKLAEAAKQLGGDSLSFSISFVSALLILLREGVEAILVLAAILAFLRNTGQESAVRGVHVGWGLAFVAGFATWALAAYVIDIGGAQRELMEGFTSLFACVMVLWLGVWMHDRRHAAAWQDYIRSSLVGGGGRFGFAVLAFFSVYRELFEVILFYETLWLQAGPAGHNAVIGGAATAVVLLIGLAWVILRGSAKLPLGLFFSINAALLCALSVVFAGHGVIALQEAGVIGTRPVPFFDFDWLGIKADAYSLSAQAMALVAIALLYGRSRIVERRRAAANAAD

SEQ ID NO: 148 - protein, 181 aa, Homo sapiens
MSDTPAEVCENLLARALTNDDLVAFLVGEQPYFIESHSGEEEPQDVCRAIERCLLPCWQSGRFPYLPRRFADALLKILATYPDRNRAIYVAQNWIWYYRFCLSKKRANPRGPYGDLFEVDLGAVAVALKRQLEANKDELIRDARWAGATWNSSNGLWGPLLRTSTTVRDKLDGPDFVPDTP

SEQ ID NO: 149 - protein, 397 aa, Homo sapiens
MAKEKFERNKPHVNVGTIGHVDHGKTTLTAALTKVCSDTWGGSARAFDQIDNAPEEKARGITINTSHVEYDSAVRHYAHVDCPGHADYVKNMITGAAQMDGAILVCSAADGPMPQTREHILLSRQVGVPYIVVFLNKADMVDDAELLELVEMEVRDLLNTYDFPGDDTPIIIGSALMALEGKDDNGIGVSAVQKLVETLDSYIPEPVRAIDQPFLMPIEDVFSISGRGTVVTGRVERGIIKVQEEVEIVGIKATTKTTCTGVEMFRKLLDEGRAGENVGILLRGTKREDVERGQVLAKPGTIKPHTKFECEVYVLSKEEGGRHTPFFKGYRPQFYFRTTDVTGNCELPEGVEMVMPGDNIKMVVTLIAPIAMEDGLRFAIREGGRTVGAGVVAKIIE

SEQ ID NO: 150 - protein, 627 aa, Homo sapiens
MNALPTSLLQRLLERPAPFALLYRPESNGPGLLDVIRGETLELHGLADLPLDEPGPGLPRHDLLALIPYRQIAERGFEALDDGTPLLALKVLEQELLPLEQALALLPNQALELSEEAFDLDDEAYAEVVGRVIADEIGRGEGANFVIKRRFQARIDGYATASALSFFRQLLLREKGAYWTFIVHTGERTLVGASPERHISVRDGLAVMNPISGTYRYPPAGPNLAEVMEFLDNRKEADELYMVVDEELKMMARICEDGGRVLGPYLKEMAHLAHTEYFIEGQTSRDVREVLRETLFAPTVTGSPLESACRVIRRYEPQGRGYYSGVAALIGGDGQGGRTLDSAILIRTAEIEGDGRLRIGVGSTIVRHSDPLGEAAESRAKASGLIAALKSQAPQRLGSHPHVVAALASRNAPIADFWLRGASERQQLQADLSGREVLIVDAEDTFTSMIAKQLKSLGLTVTVRGFQEPYSFDGYDLVIMGPGPGNPTEIGQPKIGHLHLAIRSLLSERRPFLAVCLSHQVLSLCLGLDLQRRQEPNQGVQKQIDLFGAAERVGFYNTFAARALQDRIEIPEVGPIEISRDRETGEVHALRGPRFASMQFHPESVLTREGPRIIADLLRHALVERRP

SEQ ID NO: 151 - protein, 728 aa, Homo sapiens
MTSITPVTLANCEDEPIHVPGAIQPHGALIALRADGMVLAASENIHALLGFVASPGSYLAPEQVGPEVLRMLEEGLTGNGPWSNSVETRIGEHLFDVIGHSYKEVFYLEFEIRTADTLSITSFTLNAQRIIAQVQLHNDTASLLSNVTDELRRMTGYDRVMAYRFRHDDSGEVVAESRREDLESYLGQRYPASDIPAQARRLYIQNPIRLIADVAYTPMRVFPALNPETNESFDLSYSVLRSVSPIHCEYLTNMGVRASMSISIVVGGKLWGLFSCHHMSPKLIPYPVRMSFQIFSQVCSAIVERLEQGRIAELLRVPTERRLALARRARDADDLFGALAHPDDGIAALIPCDGALVMLGGRTLSIRGDFERQAGNVLQRLQRDPERDIYHTDNWPQPSEDSPDGGDCCGVLAIRFHRQESGWIFWFRHEEVHRIRWGGKPEKLLTIGPSGPRLTPRGSFEAWEEVVRGHSTPWSETDLAIAEKLRLDLMELCLNHAAEVDRMRQRLIAVLGHDLRNPLQSISMAAALLSSSDTRTTELRQHISASSSRMERLVSQILDMSRLQSGIGLTVNPVDTDVSQLVQQIVCETDVAYPGLVIEIAIDPQVRAVVDPDRYAQVAANLLSNARHHGLPGRPVLVTLTRQGDEVCLSVLNETSGLSEAQLANLFEPFKRESADNQRNRNGLGIGLYISQAIAQAHQGRIDVDCRDDVITFCLRLPVRQAETGSSS

SEQ ID NO: 152 - protein, 464 aa, Homo sapiens
MARFTHSVGGETYRFDSLKDVMAKASPARSGDFLAGVAASNDGERVAAQMALADIPLKHFLDEALIPYEDDEVTRLIIDTHQRDAFAPVSHLTVGGFRDWLLGDAADEASLRALAPGLTPEMAAAVSKIMRVQDLVLVAQKIRVVTRFRNTLGLRGRLSTRLQPNHPTDDPAGIAASILDGLLFGNGDAMLGINPATDSMASICALLEMLDAIIQRYEIPTQACVLTHVTSSIEAINRGVPLDLVFQSIAGTEAANASFGISLKILQEGYEAGLSQKRGTLGNNLMYFETGQGSALSANAHHGVDQQTCETRAYAVARHFKPFLVNTVVGFIGPEYLYNGKQIIRAGLEDHFCGKLLGVPMGCDICYTNHAEADQDDMDMLLTLLGVAGINFIMGIPGSDDVMLNYQTTSFHDALYARQTLGLKPAPEFEDWLQRMGIFTQADGRIRFGDELPPAFRQALAQLA

SEQ ID NO: 153 - protein, 619 aa, Homo sapiens
MALLQIAEPGQSPKPHERRLAVGIDLGTTNSLVAAVRSGVAEPLPDAQGRLILPSAVRYHAERAEVGESARAAAAKDPFNTIISVKRLMGRGLEDVKQLGEQLPYRFRQGESHMPFIETVQGLKSPVEVSADILRELRQRAETTLGGELVGAVITVPAYFDDAQRQATKDAARLAGLNVLRLLNEPTAAAVAYGLDKGAEGLVAIYDLGGGTFDISILRLTRGVFEVLATGGDTALGGDDFDHAIAGWVIEQAGLSADLDPGSQRQLLQIACAAKERLTDEASVRVAYGDWSGELSRATLDELIEPFVARSLKSCRRAVRDSGVDLEEIRSVVMVGGSTRVPRVRTAVGELFGCEPLTDIDPDQVVAIGAAIQADALAGNKRGEELLLLDVIPLSLGLETMGGLMEKVIPRNTTIPVARAQEFTTYKDGQTAMMIHVLQGERELVKDCRSLARFELRGIPPMVAGAAKIRVTFQVDADGLLGVSARELSSGVEASIQVKPSYGLTDGEIARMLKDSFDYAGDDKAARALREQQVEAQRLLEAVQSALDVDGERLLDEEERLAIAAQMDTLRELAGGSDTAAIENQIKRLSQVTDAFAARRMDATVKAALSGRRLNEIEE

SEQ ID NO: 154 - protein, 277 aa, Homo sapiens
MDKSDDSQDKYIVPGLERGLLLLCEFSRKDRTLTAPELARRLKLPRSTIFRLLTTLEAMGFVTRNGNEYRLGMAVLRLGFEYLASLELTELGQPLLNRLCDEIRYPCNLVVRDGRSIVYVAKVSPSTPLSSSVNVGTRLPAHATVLGRILLQDLSLGELRELYPEEQLEQFSPNTPRSVLELFDMVQGDRQRGFVQGEGFFEASISTVAAPVRDHSGRVIAAMGATIAAGHIDPERIEGLVSRVRSSADELSYLLDYRADGQDNVTPIFRSRSHETV

SEQ ID NO: 155 - protein, 571 aa, Homo sapiens
MLQSVSPKHDLPLKPEGQAAKPERTGTWAPFSIQAFRIIWICNLFANLGTWAQSVAAAWVVTDAHASPLMVAMIQVAAALPLVLLSILSGVIADNHDRRKIMLWGLSFEMTGAMFATLLAFLGYLDPVLLIISILWISLGGSVTIPAWQAAVNEQVPARMVSDAVLLNSVNYNVARAAGPALGGLLLSAVGPAWVFLFNSFCYMALIWAIWQWRRDVPKRSLPPEGILEGVTAALRFTQYSTVTRLVMMRSFAFGLSASAVWALLRLLAHRNPDGDAAIYGYMLGALGLGAILGSTQVSRLRQRIGSSRLISLAGFTLALILLTLGLVDNLWVLFPVLILGGGCWIGALATYNSAVQILVPDWIKARALALYQTALYGGLALGSFLWGHLAETMTVHGALLAAGCLLLASVILLYNSRLPEMDAASISRAPASMPGQPSFVFNTRRGMVLVSIEYRIPAERTRDFVRAAQPLRRLRLRNGAERWSLFRDVSNPEVWQELFLVDNWIQHLRMLDRMTLADKIVIDNVTALHAGDGPPQIRHCVSYEASSYDTPLVKSATPPANDEEGATAGN

SEQ ID NO: 156 - protein, 108 aa, Homo sapiens
MRPLKQATPTYSSRTADKFVVRLPEGMREQIAEVARSHHRSMNSEIIARLEQSLLQEGALQDNLGVRLDSPELSLHERELLQRFRQLTHRQQNALVALIAHDAELAQA

SEQ ID NO: 157 - protein, 372 aa, Homo sapiens
MRKLIFLILITLFAAYAGWAERRPVGHYLSDLRSQVSVVQGQPGERGNLLAVQPELFTPDYQSAERLQLKFHAYLENARRQGLLNERTVVVFPEHVVTWLVASGEKPEVYAAADWPTAMDWMAASNPLKVARGWITARGEQRLTDTLFRMKAVDMAHDYQTLFGGLAHDFKVTVVAGSIVLPDPEVEDGELRPGTGQLYNVSLTFGPDGRPLGQPQRKVFPTRHELSYLNNGRGERLQVLDTPAGRLGVLIGTDSWYPDTYATLVEQRVELLAIPAALNQSGRWQQPWPGFDAELVPGDVRLAPNSLSNAEAWQRLAVGERLLASGARGAAVAFAHSRLWNVTEDGQSLLGSPQGMRQANPGGGAQLVNLWL

SEQ ID NO: 158 - protein, 507 aa, Homo sapiens
MTEHTAPTAPVADVCLLLEGTWPYVRGGVSSWVNQLILGLPDLTFSVFFIGGQKDAYSKRHYPIPDNVLHIEEHFLETAWSSPNPQTRQGSSETEKALRDLHRFFHYPETPDVEEGDALLDLLAEGRIGREDFLHSKASWEAITVGYERYCTDPSFVNYFWTLRSMQAPVFMLAEAARRMPRARMLHSISTGYAGLLGCILQRRWGCRYLLSEHGIYTKERKIDLAQASWIAENPDEQLSTGLDAEVSYIRRLWIRFFERVGLLTYRAANPIVALYEGNRQRQVLDGAEPWRTRVIPNGIDLDAWAGALERRPPGIPPVVGLVGRVVPIKDVKTFIRAMRGVVSAMPEAEGWIVGPEEEDPDYASECRSLVASLGLQDKVKFLGFRRIGEVLPQLGLMVLTSISEAQPLVILEAWAAGTPVVSSDVGSCRELIEGADAEDRALGRAGEVVAIADPQATSRAILALLRNPQRWQVAQAVGLQRVERYYTEALMLGRYRGLYREATEIA

SEQ ID NO: 159 - protein, 4136 aa, Homo sapiens
MNAEDSLKLARRFIELPVEKRRVFLETLRGEGIDFSLFPIPAGVSSAERDRLSYAQQRMWFLWHLEPQSGAYNLPSAVRLNGPLDRQALERAFASLVQRHETLRTVFPRGADDSLAQAPLQRPLEVAFEDCSGLPEAEQEARLREEAQRESLQPFDLCEGPLLRVRLIRLGEERHVLLLTLHHIVSDGWSMNVLIEEFSRFYSAYATGAEPGLPALPIQYADYALWQRSWLEAGEQERQLEYWRGKLGERHPVLELPTDHPRPAVPSYRGSCYEFSIEPALAEALRGTARRQGLTLFMLLLGGFNILLQRYSGQTDLRVGVPIANRNRAEVEGLIGLFVNTQVLRSVFDGRTSVATLLAGLKDTVLGAQAHQDLPFERLVEAFKVERSLSHSPLFQVMYNHQPLVADIEALDSVAGLSFGQLDWKSRTTQFDLSLDTYEKGGRLYAALTYATDLFEARTVERMARHWQNLLRGMLENPQASVDSLPMLDAEERYQLLEGWNATAAEYPLQRGVHRLFEEQVERTPTAPALAFGEERLDYAELNRRANRLAHALIERGVGADRLVGVAMERSIEMVVALMAILKAGGAYVPVDPEYPEERQAYMLEDSGVELLLSQSHLKLPLAQGVQRIDLDRGAPWFEDYSEANPDIHLDGENLAYVIYTSGSTGKPKGAGNRHSALSNRLCWMQQAYGLGVGDTVLQKTPFSFDVSVWEFFWPLMSGARLVVAAPGDHRDPAKLVALINREGVDTLHFVPSMLQAFLQDEDVASCTSLKRIVCSGEALPADAQQQVFAKLPQAGLYNLYGPTEAAIDVTHWTCVEEGKDAVPIGRPIANLGCYILDGNLEPVPVGVLGELYLAGRGLARGYHQRPGLTAERFVASPFVAGERMYRTGDLARYRADGVIEYAGRIDHQVKLRGLRIELGEIEARLLEHPWVREAAVLAVDGRQLVGYVVLESESGDWREVLAAHLATSLPEYMVPAQWLALERMPLSPNGKLDRKALPAPEVSVAQAGYSAPRNAVERTLAEIWQDLLGVERVGLDDNFFSLGGDSIVSIQVVSRARQAGLQLSPRDLFQHQNIRSLALAAKAGAATTEQGPASGEVALAPVQRWFFERAIPNRQHWNQSLLLQARQPLDGDRLGRALERLQAQHDALRLRFREERGAWHQAYAEQAGEPLWRRQAGSEEALLALCEEAQRSLDLEQGPLLRALLVDMADGSQRLLLVIHHLAVDGVSWRILLEDLQRLYADLDADLGPRSSSYQAWSRHLHEQAGARLDELDYWQAQLHDAPHALPCENPHGALENRHERKLVLTLDAERTRQLLQEAPAAYRTQVNDLLLTALARATCRWSGDASVLVQLEGHGREDLGEAIDLSRTVGWFTSLFPLRLTPAADLGESLKAIKEQLRGVPDKGVGYGLLRYLAGEEAAARLAALPQPRITFNYLGRFDRQFDGAALLVPATESAGAAQDPCAPLANWLSIEGQVYGGELSLHWSFSREMFAEATVQRLVDDYARELHVLIEHCCQEGNVGATPSDFPLATLRQEQLDRLPLALIEDIYPLSPMQHGMLFHSLYEQASGDYLNQLRVDVHGLDPARFRAAWQAALDSHDILRAGFLWQGDLEQPLQVIHKHLELPFAEHDWRGREALAEALDELAASERRRGFELEQAPLLRLVLVRMDEERYHLVYTHHHILLDGWSSAQLLGEVLARYTGEQAERTGGRYRDYIAWLQAQDKRVSEAFWKEQLAELLEPTRLAQAVAAEREQVGSGQFQRSLPPARTARLKTFAQRHAVTLNTLVQAAWSLLLQRYTGQDTVVFGATVAGRPAELAGIERQIGLFINTLPVVATPQPGMRLTDWLQEVQARSLALREQEHTPLFEIQRWAGLGEALFDSLLVFENYPVAEALEKGSPGGVRFGPVSNHEQTNYPLTVALGVGDSLSLQYSYDRQAFSDAAVEQLDRHLLNLLEGFVDNAERTLVELSLLDAEERALIDSLWNRSESGFPASPLIHQRVAERARLAPDAPAVLFDDQVLSFAELDSRANRLAHALIARGVGPEVRVAIAMQRSAEIMIAFLAVLKSGGAYVPLDIEYPRERLLYMMQDSRAHLLLTQSHLLDRLPIPDGLSCLCLDREQEWAGFPAHDPEVALHGDNLAYVIYTSGSTGMPKGVAVSHGPLAAHIVATGERYEMTPADCELHFMSFAFDGSHEGWMHPLINGARVLIRDDSLWLPEQTYAQMHRHGVTVAVFPPVYLQQLAEHAERDGNPPAARVYCFGGDAVAQASYDLAWRALRPQYLFNGYGPTETVVTPLLWKARPDDPCGAAYMPIGTLLGNRSGYILDAQLNLLPVGVAGELYLGGEGVARGYLERPALTAERFVPDPFGAPGSRLYRSGDLTRGRADGVVDYLGRVDHQVKIRGFRIELGEIEARLREQAAVREAVVVAQAGASGQQLVGYVVPQDPALAEDVGAQAACRDALRKALKERLPEYMLPAHLLFLACMPLTPNGKLDRKGLPKPSADQQQRDYQAPRSEVERQLATIWAEVLKLEQVGLADNFFEIGGDSIISLQVVSRARQLGIHFTPKMLFEAQTIGALAPLAESGTQVLAIDQGPVTGVTPLLPIQQGFFAEEVAERHWWNQSVLLEAREPLDARLLEQALRGVLAHHDALRLSFTREAAGWTARHRGVEEGAAALLRVARVADLAALRALADEVQRSLDLADGPLLRALLATFDDGSQRLLLVIHHLVVDGVSWRILFEDLQTAYRQLLAGQAVELPAKTSAFRDWAERLQAFAGDGGLDGELAYWQGQLQGASSDLPCLDPQGDQSNRHARSVSCGLDAEATRQLLQEAPAAYRTQVNDLLLTALARVICRWTGQVDALIQLEGHGREELFAEIDLTRTVGWFTSLFPLRLTPAEGIAASIKGIKEQLRAVPNKGIGFGALRYLGSAASQAALAGLPVPRITFNYLGQFDGSFAMEEGALFAPAGERAGDDQSPDAPLANWLALNGRIYGGELRIDWSFSGECFEIASIQRLADAYRDELLALIAHCRVAEGQGLTPSDFPLARLDQARLDQLPLAPCEVEDLYPLSPMQQGMLFHSLYQQEAGDYINQLRVDIDGLHPESFRAAWQAALDEHDVLRSGFLWQGAFETPLQVVRKRVEVPFSVLDWRGREDLAAALDELAAGEGRLGFDLSEAPLLRLVLVRTDEERYHLIYTNHHILMDGWSNSQLLGEVLQRYRGETPPRSGGRYRDYIAWLQRQDAALAEAFWLPRLRQLDEPTRLGQSIAQAKQRGKGYAERLRELDGEQTRRLAELAREQKITVNTLVQAAWLMLLQRYTGQDSVAFGATVAGRPAELNGIEEQIGLFINTLPVIASPLPQQSLASWLQAVQGENLALREFEHTPLYDIQRWAGQGGEALFDNILVFENYPVSQMLQQQASQGLAFGAVGNHEQTNYPLTLSVSLGQRLELQFAYDREHFDDASVARLDRHLTHLLAQMVERPASTCLAEFQLLEAAERRQAIFDWGRNPGRYPDERSVEQLFASRAAMEPERVALLFEERQLSYGELNAQANRLAHRLIELGVGPDVLVGIAVERGLEMIVSLLAVLKAGGAYVPLDPEYPQERLGYMIEDSGIALLLSQSHLLQRLPAASGIACLALDQARDWQDRPASDPQLRAHPQNLAYVMFTSGSTGRPKGVGISRESLSRHTHVSLEFFGIGPDDRVLQFSTFNFDGFVEQLYPPLACGASVVLRGTEIWDSETLYREIVERRITTVDLTTAYWNMLAKDFANQGVRDYGALRQVHAGGEAMPPESLVAWKAAGLEHVRLLNTYGPTEATVTVTTLDCAPYVDGSKAIPATMPIGKVLPGRAIYLLDDAGQPAPVGAVGELVIGAELLARGYFKRPDLTAARFIPDPFDEQGGGRLYRTGDLARYGADGVIEYVGRVDHQVKVRGFRIELGEIEACLGEHPAVREALVIAIEGTAGAQLVAYLVPQAEALASATLEVQAALRNELKALLRDSLPEYMVPAHLLFLERLPLSPNGKVDRKALPAPDASLLQEAYVAPRSELECQVAAIWQEVLKLQRVGLDDHFFELGGHSLLAINVISRIQLELGMKLTPQLLFQFPTLGLFVSNLEKAGGQVDTSKLNKLEALLDEMEEV

SEQ ID NO: 160 - protein, 139 aa, Homo sapiens
MRIVCLLVLLSLIGGCSHSVTLQERAEQEAIFIQEFRQEFAKRLRYPLFAPGDEIPEADVVVFFRFASSSGRISSCRVQYGEGNVKSRSTLDEVFARRVIATCREPDLPVAPPALLDGGGGFGFKQRVMFRKEVERPRF

SEQ ID NO: 161 - protein, 169 aa, Homo sapiens
MDSQGSIKNTLIAQLFQQNYDWLCKKLSYQTGCSHSAEDLAAEAFLQVWMLPDPASIRSPRAFLATIAQRLMYESWRRKDLERAYLQILAEAPEAVQPSPHEQWMLIESLQAIDRLLDGLSGQARAVFLMSQLEGLTYVQIGERLGLSLGRIHQLMKDALHCCYRGFQE

SEQ ID NO: 162 - protein, 624 aa, Homo sapiens
MPRHPFSPSFLAASVFCAATPALAALQPIAEAPLAGETEFRCQFNADNSTDCVSTYSFTILKPSGREMLSRIDRSYAETDSLIVEKAELTQPGGKPVPLDQSQIDTRTAPNPDQGFLRERQTSLAFPNLRVGTRISYTLREHFTAKPLSTQFHYILSRPPMPVRDDRFVAEFKAERPIFVRSELMDAYRIEQSADKKTLKVSLKKPQYTNYINEAGNAYLRHTPRLELGSSLDLQDNFGPFAARYNEILAAELPKGAAAAVAAVKGKPAREQVAGLMQYINDTYRYLGDWRASERGYVPFSLAEIERNGYGDCKDLAILLAAMLKAAGIKAEPTLVSRGDVVWDLLVPGMYAPNHAIVRAEVDGKTWWLDPTNPVFAPGRTMPDIQQRWALVLGADGKVRRDEIPLEAPGDTLRVTRSEHYTHDGEARVESRVELSNAPLMQLSVADRQRGRTSTDQDLCRNFAKEGSDCVLERDDSQFVLPPSYTISARLTDRRALDRLGGEYFYNRQDLASQWDAFAKYRSEGQLAELYLGEPQITSYDISLSGGKTDEPAHSCEIRSPWFDIDLQAEPAKDGGYHYRYREVQKTSWLNHDEINSAEFGKLIEQSRGCVEQLRLVVKLDKRP

SEQ ID NO: 163 - protein, 345 aa, Homo sapiens
MSKQVALVLGSGGARGYAHIGVIEELEARGYEITCIAGCSMGSVIGGIYAAGKLREYREWVESLDYLDVLRLLDVSFRLGAIRGERVFGKIHEILGEVNIEDLSIPYTAVATDLTNQQEIWFQEGCLHQAMRASAAIPSLFTPVMQGSRMLVDGGLLNPLPIVPVVSAHSDLIIAVNLNATNQKQYHLPVIERPAALKGRFDSMIESLGHKLSFFRRHGDDNTPPELSADALLHPVPAESEEPAEPQLQQPAAAPQGKDSPKSASGTTVVESSGPASLLELVNQSFEVMQTSLAQYKIAGYPPDILINVPKRVCRFFEFYKAPELIALGRQIASDTLDRYEEDNA

SEQ ID NO: 164 - protein, 302 aa, Homo sapiens
MQQLLNEILDEVRPLIGRGKVADYIPALAGVEPNQLGIAVYSRDGELFHAGDALRPFSIQSISKVFSLVQAIQHSGEDIWQRLGHEPSGQPFNSLVQLEFERGKPRNPFINAGALVICDINQSRFAAPAQSMRDFVRRLCGNPEVVSDSVVARSEYQHRSRNAAAAYLMKSFGNFHNDVEAVLLSYFHHCALRMSCVDLARAFCFLADKGFCKHSGEQVLNERQTKQVNAIMATSGLYDEAGNFAYRVGLPGKSGVGGGIIAVVPGRFTVCVWSPELNAAGNSLAGIAALEKLSERIGWSIF

SEQ ID NO: 165 - protein, 150 aa, Homo sapiens
MRRYLFAGFLPALASLSAPLCAAEGTTGAAAPTVGAASGAAAQLAQLVLGLGLVIGLIFLLAWLVRRVQQAGPRGNRLIRTLASQPLGPRDRLVLVQVGEEQILLGLTPGRITPLHVLKEPVHLPDGEPATPEFAQRLLELLNKDPKGKP

SEQ ID NO: 166 - protein, 616 aa, Homo sapiens
MNRVFLTPAAVALCGASSLSLAEPVSLADQVVTATRTAQTASQSLAAVSVIDREDIERSQARSVPELLRQVPGVSLANNGGFGKNTTLFLRGTESDHVLVLIDGIKVGSASAGLTAFQDLPVELIERIEVVRGPRSSLYGSEAIGGVIQIFTRRGDGQGAKPFFSAGYGTHQTLEGSAGVSGGAGNGWYSLGVSSFDTAGINTKRAGTAGYEPDRDGYRNLSGNLRGGYRFDNGLELDGTLLRAKSHNDYDQVFGNSGFNANADGEQNLVGGRARFTPFDPWLVTLQAGRSEDKADAYQDGRFYSRFDTRRDSLSWQNDLTLAEGHVLTLGYDWQKDEISSSEAFSVDSRLNKGWFAQYLGQYGRQDWQLSLRRDDNQQFGVHDTGSAAWGYALSDALRFTVSYGTAFKAPTFNELYYPDYGNPDLDAETSRSLEVGLSGTHGWGHWAVNAFRTNVDDLIGNDPRPAPGRPWGQPNNIDEARIRGVELVLGSQWLGWDWNANATFLDPQNRSGGVSDGNELPRRARRMFNLELDRRFERLSLGASVHAEGRRYDDPANKVRLGGYATLDLRSEYRLNDEWRLQGRIANLFGADYETAYGYNQPGQAVYLSVRYQAL

SEQ ID NO: 167 - protein, 233 aa, Homo sapiens
MLSFDKVSTYYGKIQALHDVSVEVKKGEIVTLIGANGAGKSTLLMTLCGSPQAASGSIRYEGEELVGLPSSTIMRKSIAVVPEGRRVFSRLTVEENLAMGGFFTDKDDYQVQMDKVLELFPRLKERYEQRAGTMSGGEQQMLAIGRALMSKPKLLLLDEPSLGLAPIIIQQIFEIIEQLRREGVTVFLVEQNANQALKLADRAYVLENGRIVMHDTGAALLTNPKVRDAYLGG

SEQ ID NO: 168 - protein, 213 aa, Homo sapiens
MAIRSEDSDGFWQAFSRQEQDSTNRLLPTTLAAPFILCASSATPCRSASSSNSANRVRSSRLLANKVSSNRRNAAGSPPVMASSERMSKRLSIGDSEPSAFIQTPSICLKEGHCALRCPAYGTPGIRRTTRPYMPSGPVIRKQHKGIASASAVQTGEHRLLRLIERPQTALETLMAGTESTFASRPQPARPLVSGRETPTVSLCEEASSVATA

SEQ ID NO: 169 - protein, 61 aa, Homo sapiens
MLILTRRVGETLMVGDDVTVTVLGVKGNQVRIGVNAPKEVAVHREEIYQRIQKEKDQEPNH

SEQ ID NO: 170 - protein, 450 aa, Homo sapiens
MTDYTQQLAGFLAGLRYQDLPPAVLARMEELFLDWLGSALAGKGQHPIPLFERYAERMGPADGSAQILPSRRRSSPYFAALVNGAASHVVEQDDLHNSSVLHPAAVVFPAALAAAQDLGRSGAELILAAVAGYEAGIRIGEFLGRSHYRVFHTTATVGTLAAAVAVGKLMDFDRERFVDLLGSAGTQAAGLWEFLRDAADSKQLHTAKAAADGLLAAYLTADGLSGARHILEGEQGMAAGMSSDADPERLVDRLGSRWALLETSFKFHASCRHTHPAADALLALMQREGLDHSQIAAVTARVHQGAIDVLGRVVEPQTVHQAKFSMGTVLGLIAVYGKAGLGEFHRHALSDPRVAAFRERVEMRLDPEVDAAYPQRWLGRVEVLDREGRRHTAAIDEPKGDPGNTLSRDELADKFRRLLAFSGAATGAEAEILIQRAWGLRQAPSVTPLI

SEQ ID NO: 171 - protein, 362 aa, Homo sapiens
MEQPFDLAAELAKQPHLLEIAGNLLMKGGPEDYIGAVLCLRGTLYFKEAHTPLVRESLCQCFDEFKRLAEPHLTRLWREEPAQGKPLTAYRDTQPLREMMGAMDEDDHLSFCYTSGKKSRDAGAWLFDIYGKRSWQAKMGHDLSVLEFSVPLLYQEQQPLDFLQLFIDFARRLEPEQGYAGHAYNLSPTSWDNDEPSEAFMAARMPGLDVGTACLLANTPEFKPTRIKTVSWLTLLNNERLALAGGLDALRAQLPSSHFAFYRYGDGVVIQAGAYPYIAGDAEDSRPAPYVLLNHALKGIRYETIGSLHGGSHDGELRLVGWAADQWLKRLDVEDSEIPRWRDKLLSAEPCLDATNTLPERL

SEQ ID NO: 172 - protein, 168 aa, Homo sapiens
MSYRSNSTGFTLIELLIIVVLLAIMASFAIPSFKQLTERNELQSAAEELNAMLQYARSEAVSQRRAISIQALKDKDWGKGLSIGVLASGSIAAPLRKHDGFRAATLTAKEKSAVEHLTFTANGTLVPPTERTFAICQNGKTDGGRVLSISQAGRIQLEPSSKAPQSCY

SEQ ID NO: 173 - protein, 346 aa, Homo sapiens
MQSSQLLPLGSLLLSFATPLAQADALHDQASALFKPIPEQVTELRGQPISEQQRELGKKLFFDPRLSRSHVLSCNTCHNVGTGGADNVPTSVGHGWQKGPRNSPTVFNAVFNAAQFWDGRAKDLGEQAKGPIQNSVEMHSTPQLVEQTLGSIPEYVDAFRKAFPKAGKPVSFDNMALAIEAYEATLVTPDSPFDLYLKGDDKALDAQQKKGLKAFMDSGCSACHNGINLGGQAYFPFGLVKKPDASVLPSGDKGRFAVTKTQSDEYVFRAAPLRNVALTAPYFHSGQVWELKDAVAIMGNAQLGKQLAPDDVENIVAFLHSLSGKQPRVEYPLLPASTETTPRPAE

SEQ ID NO: 174 - protein, 345 aa, Homo sapiens
MRILIVGPSWVGDMVMAQTLFQCLRQRHPECVIDVLAPEWSRPILERMPEVRQALSFPLGHGVMDVATRRRIGRGLRGQYEQAILLPNSLKSALVPWFAGIPKRTGWRGEMRYGLLNDIRKLDKQRYPLMIERFMALAFEPGVELPKPYPQPRLRIDDGSRQAALDKFALSLDRPVLALCPGAEFGEAKRWPAEHYAAVAEAKIRAGWQVWLFGSKNDHPGGEEIRQRLIPGLREESFNLAGETSLAEAIDLMSCAGAVVSNDSGLMHVAAALDRPLVGVYGSTSPQFTPPLADRVEIVRLGLECSPCFERTCRFGHYNCLRELPPGLVLQALERLVGDPAEVAG

SEQ ID NO: 175 - protein, 357 aa, Homo sapiens
MKQESKRWLSRALIVAALLGVGVLVWQVSRPTGLGEGFASGNGRIEATEVDVAAKLPGRVAEIKVDEGDFVKAGEIVARMDTQVLEAQLAQAQAQVRQAENAKLTATSLVAQRESEKSTAQAVVAQRQAELTAAQKRFTRTEALVKRNALPQQQLDDDRATLQSAQAALSAARSQVISAQAAIEAGRSQVIEAQSAIEAAKASVARLQADIDDSLLKAPRNGRVQYRVAQPGEVLPAGGKLLNMVDLADVYMTFFLPSMQAGRVGLGQEVRLVIDAVPDYVIPAKVSYVASVAQFTPKTVETANEREKLMFRVKARLDPALLEKYITYVKTGVPGMAYLRLDPEVEWPANLQIKVPQ

SEQ ID NO: 176 - protein, 595 aa, Homo sapiens
MSMPLPMKLRTRLFLSISALITVSLFGLLLGLFSVMQLGRAQEQRMSHHHATIEVSQQLRQLLGDQLVILLREKPDGQALERSQNDFRRVLEQGRANTVDGAEQAALDGVRDAYLQLQAHTPALLKAPMADNDGFSEAFNTLRLRLQDLQQLALAGISDAETSARHRAYLVAGLLGLVGVAILLIGFVTAHSIARRFGAPIETLARAADRIGEGDFDVTLPMTNVAEVGQLTRRFGLMAEALRQYRKTSVEEVLSGERRLQAVLDSIDDGLVIFDNQGRIEHANPVAIRQLFVSNDPHGKRIDEILSDVDVQEAVEKALLGEVQDEAMPDLVVDVAGESRLLAWSLYPVTHPGGHSVGAVLVVRDVTEQRAFERVRSEFVLRASHELRTPVTGMQMAFSLLRERLDFPAESREADLIQTVDEEMSRLVLLINDLLNFSRYQTGMQKLELASCDLVDLLTQAQQRFTPKGEARRVSLQLELGDELPRLQLDRLQIERVIDNLLENALRHSSEGGQIHLQARRQGDRVLIAVEDNGEGIPFSQQGRIFEPFVQVGRKKGGAGLGLALCKEIIQLHGGRIAVRSQPGQGARFYMLLPV

SEQ ID NO: 177 - protein, 831 aa, Homo sapiens
MTEDQLEQETLGWLTELGYAYLYGPDIAHDGDNPERESYRDVLLTMRLRTAIARLNPQIPLAAREDALRQVLELGVPVQLSANRLLHRLLVGGVPVQYQKDGETRGDFVRLIDWVDVQANEWLAVNQFSIQGPKHTRRPDIILFVNGLPLVLLELKNPADVNADLVKAFDQLQTYKEQIPDVFHYNEILVISDGSEARMGSLSADIERFTRWRTIDGATVDPLGEFNELETLVRGVLQPAMLLDYLRYFVLFEDDGRLVKKIAGYHQFHAVRAAIQQVVSASRPGGTHKGGVVWHTQGSGKSITMTCFAARVMQEAAMENPTIVVITDRNDLDGQLFGVFSLSQDLLREQPVQVATRGDLREKLANRPSGGIVFATIQKFMPGEDEDSFPVLSTRSNIVVVADEAHRTQYGFSASLKVPDLKVAEASARYQVGYAQHLRDALPNATFVAFTGTPVSSEDRDTRAVFGDYIHVYDMQQAKEDGATVAIYYESRLAKLSLKDSELAHIDDEVDELAEDEEEDQQSRLKSRWAALEKVVGAEPRIKSVAADLVAHFEERNQAQNGKAMVVAMSREICVHLYNEIIALRPEWHAEDPEKGAVKIVMTGSASDKALLRPHIYPGQVKKRLEKRFKDPADPLQLVIVRDMWLTGFDAPCVHTLYVDKPMKGHNLMQAIARVNRVFKDKQGGLVVDYIGIANELKAALKEYTASKGRGRPTVDAHEAYAVLEEKLDVLRSLLYGFDYGDFLTGGHKLLAGAANHVLGLEDGKKRFADNALAMSKAFTLCCTLDEAKAVREEVAFLQAIKVLLIKRDISAQKKPTKNVSWRSARSSAMP

SEQ ID NO: 178 - protein, 177 aa, Homo sapiens
MRQDKRAQPKPPINENISAREVRLIGADGQQVGVVSIDEAIRLAEEAKLDLVEISADAVPPVCRIMDYGKHLFEKKKQAAVAKKNQKQAQVKEIKFRPGTEEGDYQVKLRNLVRFLSEGDKAKVSLRFRGREMAHQELGMELLKRVEADLVEYGTVEQHPKLEGRQLMMVIAPKKKK

SEQ ID NO: 179 - protein, 405 aa, Homo sapiens
MADKALKTCVFVWEGTDKKGAKVKGELAGQNTMLVKAQLRKQGINPLKVRKKGITLLGKGKRVKPMDIALFTRQMATMMGAGVPLLQSFDIISEGFDNPNMRKLVDEIKQEVSAGNSLANSLRKKPLYFDDLYCNLVDAGEQSGALETLLDRVATYKEKTESLKAKIKKAMTYPIAVVLVAIIVSAILLIKVVPQFQSVFSSFGAELPAFTMMVINLSNLLQEWWLVVLIGLFSASFAIKESHKRSVNFRNTVDRYMLKIPIIGGILYKSAVARYARTLSTTFAAGVPLVEALDSVSGATGNVVFRNAVSKIKQDVSTGMQLNFSMRTTNVFPNMAIQMTAIGEESGSLDDMLGKVAAFYEEEVDNAVDNLTTLMEPMIMAVLGVLVGGLIIAMYLPIFQLGSVV

(19) United States
(12) Patent Application Publication
(10) Pub. No.: US 2024/0425938 A1
(43) Pub. Date: Dec. 26, 2024

(54) COMPOSITIONS AND METHODS FOR CHARACTERIZING LOW FREQUENCY MUTATIONS

(71) Applicants: The Broad Institute, Inc., Cambridge, MA (US); The Children's Medical Center Corporation, Boston, MA (US); Technion Research & Development Foundation Limited, Haifa (IL)

(72) Inventors: Gregory P. PRIEBE, Boston, MA (US); Roy KISHONY, Haifa (IL); Hattie CHUNG, Cambridge, MA (US); Matthew M. SCHAEFERS, Boston, MA (US)

(73) Assignees: The Broad Institute, Inc., Cambridge, MA (US); The Children's Medical Center Corporation, Boston, MA (US); Technion Research & Development Foundation Limited, Haifa (IL)

(21) Appl. No.: 18/799,888

(22) Filed: Aug. 9, 2024

Related U.S. Application Data
(63) Continuation of application No. PCT/US2023/062210, filed on Feb. 8, 2023.
(60) Provisional application No. 63/309,368, filed on Feb. 11, 2022.

(51) Int. Cl. C12Q 1/689; C12Q 1/6806; C12Q 1/6844; C12Q 1/6869

(52) U.S. Cl. CPC C12Q 1/689; C12Q 1/6806; C12Q 1/6844; C12Q 1/6869; C12Q 2600/106; C12Q 2600/156

(57) ABSTRACT

This disclosure relates to methods and compositions useful for detecting and monitoring low-frequency mutations. Methods and compositions described herein can be used to guide clinical decisions, for example, by informing on which antibiotics should be avoided, or conversely, which antibiotics should be actively used in the case of compounds that select against a specific type of resistance.

### CROSS-REFERENCE TO RELATED APPLICATION

This application is a continuation of International Application No. PCT/US2023/062210, which claims the benefit of U.S. Provisional Application No. 63/309,368, filed Feb. 11, 2022, the entire contents of each of which are incorporated herein by reference.

### STATEMENT OF RIGHTS TO INVENTIONS MADE UNDER FEDERALLY SPONSORED RESEARCH

This invention was made with government support under grant No. R01 GM081617 awarded by the National Institutes of Health. The government has certain rights in the invention.

### SEQUENCE LISTING

The present application contains a Sequence Listing which has been submitted electronically in XML format. The content of the electronic XML Sequence Listing, (Date of creation: Mar. 17, 2023; Size: 285,032 bytes; Name: 167741-051400PCT_SL.xml), is herein incorporated by reference in its entirety.

### BACKGROUND OF THE INVENTION

Antibiotic treatment selects for resistance mutations, posing a major threat to effective treatment of bacterial infections. The selection of resistance mutations during chronic infections as a result of antibiotic treatment over months to years is well known. However, it is not well-understood how short-term changes in antibiotic therapy affect the dynamics of resistance mutations in acute infections, especially in a newly colonizing infection that is thought to start from a clonal population.

Emerging resistance is of particular concern in the treatment of acute respiratory tract infections that are common in intensive care units (ICUs) worldwide, particularly in mechanically ventilated patients who are at high risk for ventilator-associated pneumonia (VAP), septic shock, and infection-associated mortality. VAP and other lower respiratory tract infections are of major concern in the SARS-CoV-2 pandemic given the large number of hospitalized COVID-19 patients requiring ventilation. Pseudomonas aeruginosa is one of the most common bacterial pathogens causing respiratory infections in ventilated patients, and is associated with increased mortality and low treatment efficacy due to high rates of antibiotic resistance that can occur within days of antibiotic treatment.

Shallow profiling of pathogen populations using cultured isolates have shown that the frequencies of antibiotic resistance mutations can fluctuate over days to weeks during infection, but whether changes reflect drift, sampling bias, or treatment-induced selection at short timescales is unknown. Current clinical methods for detecting resistance variants are largely culture-based, where isolates with visually distinct morphology (by size, shape, color) are selected for resistance phenotyping. However, these methods are susceptible to bias from culture-based growth and are limited in their sampling resolution, especially for detecting low-frequency mutations. While molecular surveillance methods such as rapid PCR tests and real-time genome sequencing can identify the presence of known resistance genes, e.g. efflux pumps, for the rapid identification of resistant strains, they are not suitable for monitoring within-population pathogen diversity. Furthermore, it is not well-understood whether resistance mutations can contract and be reversed during the course of treatment in acute infection. A molecular, culture-free diagnostic could determine the role of low-frequency resistance variants at short time scales, and possibly inform which antibiotics should be avoided.

Accordingly, compositions and methods for rapidly detecting low-frequency resistance variants are urgently required.

### SUMMARY OF THE INVENTION

As described below, the present disclosure features compositions and methods for detecting low-frequency antimicrobial resistance mutations, and methods of using such mutations to select effective therapies for patients.

In one aspect, this disclosure provides a method for characterizing low-frequency mutations associated with resistance in a pathogen. The method includes (a) contacting a nucleic acid molecule derived from a biological sample from a subject with a primer pair, wherein at least one member of the primer pair comprises a unique molecular identifier, and wherein the primer pair binds a complementary sequence within or adjacent to an antimicrobial resistance gene, or a regulator of the antimicrobial resistance gene, present in a pathogen genome; (b) amplifying at least a portion of the antimicrobial resistance gene, or the regulator of the antimicrobial resistance gene, to obtain an amplicon; (c) deep sequencing the amplicon to identify an alteration in the antimicrobial resistance gene or the regulator of the antimicrobial resistance gene; and (d) determining the change in frequency of occurrence of the alteration in a population of pathogens over the course of time.

In another aspect, this disclosure provides a method for characterizing low-frequency mutations associated with resistance to selection in a nucleic acid molecule derived from an organism. The method includes (a) contacting the nucleic acid molecule with a primer pair, wherein at least one member of the primer pair comprises a unique molecular identifier, and wherein the primer pair binds a complementary sequence within or adjacent to a gene, or a regulator of the gene, associated with resistance to selection present in the nucleic acid molecule; (b) amplifying at least a portion of the gene, or the regulator of the gene, to obtain an amplicon; and (c) deep sequencing the amplicon to identify an alteration in the gene, or the regulator of the gene.

In another aspect, this disclosure provides a method of characterizing a bacterial infection in a subject. The method includes (a) contacting a biological sample derived from the subject with a primer pair, wherein at least one member of the primer pair comprises a unique molecular identifier, and wherein the primer pair binds a complementary sequence within or adjacent to an antimicrobial resistance gene, or a regulator of the gene, present in a bacterial genome; (b) amplifying at least a portion of the antimicrobial resistance gene, or the regulator of the antimicrobial resistance gene, to obtain an amplicon; and (c) deep sequencing the amplicon to identify an alteration in the antimicrobial resistance gene, or the regulator of the antimicrobial resistance gene.

In some embodiments of the above aspects or any other aspect delineated herein, the methods of this disclosure include identifying an alteration in an antibiotic resistance gene, wherein the gene is a gene listed in Table 3. For example, in some embodiments the antimicrobial resistance gene is NalD, OprD, MexR, AnmK, AmpD, SltB1, or PA0810. In some embodiments, methods of this disclosure include identifying an alteration in a regulator of the antimicrobial resistance gene, wherein the regulator is a gene promoter or an enhancer. In some embodiments, the alteration is a missense mutation, insertion, or deletion.

In some embodiments of the above aspects or any other aspect delineated herein, the, the pathogen analyzed by methods of this disclosure is a bacteria, a virus, a fungus, or a protozoa. For example, the pathogen can be a bacteria selected from Helicobacter pylori, Borrelia burgdorferi, Legionella pneumophilia, Mycobacteria species, Staphylococcus aureus, Neisseria gonorrhoeae, Neisseria meningitidis, Listeria monocytogenes, Streptococcus pyogenes, Streptococcus agalactiae, Streptococcus, Enterococcus faecalis, Streptococcus bovis, Streptococcus, Streptococcus pneumoniae, pathogenic Campylobacter sp., Salmonella species, Shigella species, Yersinia species, Enterococcus species, Haemophilus influenzae, Bacillus anthracis, Erysipelothrix rhusiopathiae, Clostridium perfringers, Clostridium tetani, Clostridioides difficile, Pasteurella multocida, Bacteroides sp., Fusobacterium species, Streptobacillus moniliformis, Treponema pallidium, Treponema pertenue, Leptospira, Rickettsia, Actinomyces israelli, Pseudomonas aeruginosa, Escherichia coli, Klebsiella species, Enterobacter species, Acinetobacter species, Stenotrophomonas maltophilia, Burkholderia cepacia complex, Achromobacter species, Burkholderia pseudomallei. In some embodiments, the pathogen is a bacteria, and the bacteria is a gram negative bacteria selected from the group consisting of Pseudomonas aeruginosa, Escherichia coli, Klebsiella species, Enterobacter species, Acinetobacter species, Stenotrophomonas maltophilia, Burkholderia cepacia complex, Achromobacter species, and Burkholderia pseudomallei.

In some embodiments of the above aspects or any other aspect delineated herein, the methods of this disclosure make use of a biological sample, wherein the biological sample is blood, cerebrospinal fluid, phlegm, saliva, sputum, semen, vaginal secretion, or urine. In some embodiments, the biological sample is sputum. In some embodiments, the pathogen of the biological sample is not cultured (e.g., grown in a selection plate).

In some embodiments of the above aspects or any other aspect delineated herein, the methods of the disclosure use primers that include a unique molecular identifier (UMI).

In some embodiments of the above aspects or any other aspect delineated herein, the methods of this disclosure are performed on a biological sample taken from a subject who was previously treated with at least one antimicrobial. In some embodiments, the antimicrobial treatment was conducted over the course of 1-3 days, 1 week, 2 weeks, 1 month, 3 months, or 6 months.

In another aspect, this disclosure provides a method of treating a bacterial infection in a subject. The method includes administering to the subject an effective amount of an antimicrobial selected for efficacy in the subject, wherein the antimicrobial is selected by characterizing a bacteria present in a biological sample of the subject according any one of the methods described herein. In some embodiments, the bacteria comprises one or more antimicrobial resistance mutations.

In another aspect, this disclosure provides a method of monitoring antimicrobial therapy in a subject. The method including (a) collecting two or more biological samples from the subject prior to or during the course of antimicrobial therapy; (b) contacting the biological samples with a primer pair, wherein at least one member of the primer pair comprises a unique molecular identifier, and wherein the primer pair binds a complementary sequence within or adjacent to an antimicrobial resistance gene, or a regulator of the antimicrobial resistance gene, present in a bacterial genome; (c) amplifying at least a portion of the antimicrobial resistance gene, or the regulator of the antimicrobial resistance gene, to obtain an amplicon; and (d) deep sequencing the amplicon to identify an alteration in the antimicrobial resistance gene, or the regulator of the antimicrobial resistance gene, thereby monitoring the antimicrobial therapy.

In some embodiments of the above aspects or any other aspect delineated herein, the methods of the disclosure include collecting a first biological sample prior to commencing therapy. In some embodiments, a second biological sample is collected 1, 2, or 3 days after therapy is commenced. In some embodiments, methods of this disclosure include identifying an alteration in an antimicrobial resistance gene or a regulator of the antimicrobial resistance gene. In some embodiments, the gene is a gene listed in Table 3. In some embodiments, the regulator is a gene promoter or an enhancer. In some embodiments, the antimicrobial resistance gene is NalD, OprD, MexR, AnmK, AmpD, SltB1, or PA0810.

In some embodiments of the above aspects or any other aspect delineated herein, the methods of the disclosure include identifying an alteration present in a bacterial genome. In some embodiments, the bacteria is a Gram negative bacteria. In some embodiments, the Gram negative bacteria is selected from the group consisting of Helicobacter pyloris, Borelia burgdorferi, Legionella pneumophilia, Mycobacteria spsm Staphylococcus aureus, Neisseria gonorrhoeae, Neisseria meningitidis, Listeria monocytogenes, Streptococcus pyogenes, Streptococcus agalactiae (Group B Streptococcus), Streptococcus, Streptococcus faecalis, Streptococcus bovis, Streptococcus, Streptococcus pneumoniae, pathogenic Campylobacter sp., Enterococcus sp., Haemophilus influenzae, Bacillus antracis, Corynebacterium diphtheriae, corynebacterium sp., Erysipelothrix rhusiopathiae, Clostridium perfringers, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, Rickettsia, and Actinomyces israelli.

In some embodiments of the above aspects or any other aspect delineated herein, the methods of the disclosure are carried out on a biological sample. In some embodiments, the biological sample is blood, cerebrospinal fluid, phlegm, saliva, sputum, semen, vaginal secretion, or urine. In some embodiments, the biological sample contains an uncultured pathogen. In some embodiments, methods of this disclosure include performing a whole genome sequencing analysis on a population of microorganisms. In some embodiments, methods of this disclosure further include correlating an identified alteration with a change in the population of microorganisms.

In another aspect of the above aspects or any other aspect delineated herein, the, this disclosure provides a kit for characterizing antimicrobial resistance in a bacteria. The kit can include one or more primers from among those listed in Table 4. The kit can additionally include reagents and instructions for characterizing antimicrobial resistance. Other features and advantages of the disclosure will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant a peptide, nucleic acid molecule, or small compound. In embodiments, the agent is an antimicrobial (e.g., antibiotic, antifungal, antiviral), a chemotherapeutic, or any other agent useful in applying selective pressure on a cell (e.g., cancer cell) or organism (e.g., pathogen).

By “ameliorate” is meant decrease, suppress, attenuate, diminish, arrest, or stabilize the development or progression of a disease. In some embodiments, the disease is a bacterial, fungal, or viral infection. In other embodiments, the disease is cancer.

By “alteration” is meant a change (e.g., increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels. In some embodiments, the alteration is a change in the sequence of a polypeptide or polynucleotide associated with resistance to selective pressure.

By “amplicon” is meant a polynucleotide generated during amplification.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “antimicrobial” is meant an agent that inhibits the growth of a pathogen. Exemplary antimicrobials include antivirals, antibiotics, and antifungals.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

The term “clonal sequence” refers to a sequence that is derived from a single molecule or cell. In an embodiment, a clonal sequence is analyzed using massively parallel sequencing. In an embodiment, a clonal sequence that is generated by massively parallel sequencing is derived from a distinct DNA molecule within a sample that serves as the “input” for the sequencing workflow.

By “decreases” is meant a reduction by at least about 5% relative to a reference level. A decrease may be by 5%, 10%, 15%, 20%, 25% or 50%, or even by as much as 75%, 85%, 95% or more and any intervening percentages.

By “deep sequencing” is meant sequencing a region of a polynucleotide hundreds or even thousands of times. In embodiments, deep sequencing includes next-generation sequencing, high-throughput sequencing and massively parallel sequencing. Deep sequencing involves obtaining large numbers of sequences corresponding to relatively short, targeted regions of a genome. A targeted region can include, for example, an entire gene or a portion of a gene (such as a mutation hotspot), or a regulator of the gene (e.g., a promoter or enhancer). In some cases, many thousands of clonal sequences are obtained from a short targeted segment allowing identification and quantitation of sequence variants. In embodiments, a particular region of a polynucleotide is sequenced for example 100, 250, 500, 1,000, 2,500, 5,000, 7,500, 10,000, 25,000, 50,000, 100,000, 250,000, 500,000, 750,000, or even 1, 5, 10, 25, 50, 75, or 100 million times.

“Detect” refers to identifying the presence, absence or amount of the analyte to be detected. In some embodiments, the analyte is a polynucleotide derived from a cell or organism, wherein the polynucleotide comprises a genetic alteration that increases resistance to selective pressure.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. Examples of diseases include pathogen infections (e.g., bacterial, fungal, viral) and cancer.

By “effective amount” is meant the amount of a required compound to ameliorate the symptoms of a disease of a patient relative to an untreated patient having the disease. The effective amount of active compound(s) used to practice the present disclosure for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The disclosure provides a number of targets that are useful for the development of highly specific drugs to treat, or a disorder characterized by the methods delineated herein. In addition, the methods of the disclosure provide a facile means to identify therapies that are safe for use in subjects. In addition, the methods of the disclosure provide a route for analyzing any number of compounds for effects on a disease described herein with high-volume throughput, high sensitivity, and low complexity.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

The terms “isolated,” “purified,” or “biologically pure” refer to material that is free to varying degrees from components that normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this disclosure is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the disclosure is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding an additional polypeptide sequence.

By an “isolated polypeptide” is meant a polypeptide of the disclosure that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the disclosure. An isolated polypeptide of the disclosure may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder.

By “mutation” is meant a change in a polypeptide or polynucleotide sequence relative to a reference sequence. In some embodiments, the reference sequence is a wild-type sequence. Exemplary mutations include point mutations, missense mutations, amino acid substitutions, and frameshift mutations. A “loss-of-function mutation” is a mutation that decreases or abolishes an activity or function of a polypeptide. A “gain-of-function mutation” is a mutation that enhances or increases an activity or function of a polypeptide.

As used herein, “obtaining” as in “obtaining an agent” includes synthesizing, purchasing, or otherwise acquiring the agent.

By “operably linked” refers to a functional linkage between a regulatory sequence and a coding sequence, where a first polynucleotide is positioned adjacent to a second polynucleotide that directs transcription of the first polynucleotide when appropriate molecules (e.g., transcriptional activator proteins) are bound to the second polynucleotide. The described components are therefore in a relationship permitting them to function in their intended manner. For example, placing a coding sequence under regulatory control of a promoter means positioning the coding sequence such that the expression of the coding sequence is controlled by the promoter.

By “portion” is meant a fragment of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 5, 6, 7, 8, 9, 10, 11, 12, 13, 14, 15, 16, 17, 18, 19, 20, or 21 nucleotides.

By “positioned for expression” is meant that the polynucleotide of the disclosure (e.g., a DNA molecule) is positioned adjacent to a DNA sequence that directs transcription and translation of the sequence (i.e., facilitates the production of, for example, a recombinant microRNA molecule described herein).

“Primer set” means a set of oligonucleotides that may be used, for example, for PCR. A primer set would consist of at least 2, 4, 6, 8, 10, 12, 14, 16, 18, 20, 30, 40, 50, 60, 80, 100, 200, 250, 300, 400, 500, 600, or more primers.

By “reduces” is meant a negative alteration of at least 10%, 25%, 50%, 75%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

By “regulator” or “gene regulator” is meant a nucleic acid sequence involved in controlling the expression of one or more genes. The regulator can be a gene promoter. A gene promoter is a sequence that is involved in gene transcription and is generally located near the beginning of the gene. The regulator can be an enhancer. An enhancer is a cis-regulatory element that can cooperate with promoters to control target gene transcription. Unlike a promoter, an enhancer is not necessarily adjacent to the target genes and can exert their functions regardless of enhancer orientations, positions and spatial segregations from the target gene.

By “resistance to selection” is meant the acquisition of a genetic alteration that allows a pathogen, cell, or organism to escape the consequences of selection. In embodiments, resistance to selection arises during treatment with a therapeutic agent. Therapeutic agents include, but are not limited to, antifungals, antivirals, antibiotics, and chemotherapeutics.

By “resistance polynucleotide” is meant a nucleic acid molecule encoding a resistance polypeptide, as well as the introns, exons, and regulatory sequences associated with the expression of the resistance polypeptide, or fragments thereof. In embodiments, a resistance polynucleotide is the genomic sequence, mRNA, or gene associated with and/or required for resistance polypeptide expression.

By “specifically binds” is meant a compound or antibody that recognizes and binds a polypeptide of the disclosure, but which does not substantially recognize and bind other molecules in a sample, for example, a biological sample, which naturally includes a polypeptide of the disclosure.

Nucleic acid molecules useful in the methods of the disclosure include any nucleic acid molecule that encodes a polypeptide of the disclosure or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant to pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid level to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition, or symptoms associated therewith be completely eliminated.

By “unique molecular identifier” or “UMI” is meant a short nucleic acid sequence that is identifiable in, for example, high-throughput sequencing techniques, such as but not limited to single-cell RNA-seq. The UMIs may be used to not only detect, but also to quantify. In embodiments of the disclosure, the UMIs are not viral barcodes.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1C provide a prospective study of P. aeruginosa populations from mechanically ventilated patients during acute lower respiratory tract infection.

FIG. 1A provides a prospective study design describing the enrollment strategy of mechanically ventilated patients in the ICU. Of 87 patients screened, 49 eligible patients were identified, from which 31 consented to enrollment. The analysis focused on 2 pilot patients sampled at only day 1, and 7 patients sampled at two time points spanning 4-11 days, who had predominant P. aeruginosa growth in both samples.

FIG. 1B shows sampling sputum and stool across patients (y axis) over time (x axis) from the onset of symptoms. Day 1 sputum sample (white box) were collected in all patients. In 7 patients, a follow-up sputum (dark gray box) was collected between day 5 and day 12, or 4-11 days after day 1. Stool with confirmed P. aeruginosa growth was collected in 2 patients (light gray box). Asterisk: patients with documented prior P. aeruginosa infection. Anti-pseudomonal antimicrobial administered to each patient are indicated by horizontal lines, indicating the days treated. Piperacillin/tazobactam (weighted solid black), cefepime (thin solid black), ceftazidime (large dotted black), ciprofloxacin (dotted blue), meropenem (weighted solid pink).

FIG. 1C provides a workflow showing that samples (sputum or stool) were cultured on cetrimide agar, in serial dilutions, to select random isolates. One isolate from day 1 sputum of each patient was collected for long-read sequencing in order to construct patient-specific reference genome. In all other samples, 24 isolates were randomly selected from day 1 sputum, follow-up sputum, or stool sample for short-read sequencing, reads from which were aligned to the patient-specific reference genomes to identify within-population mutations (SNPs and short indels).

FIGS. 2A-E show that patients with a prior history of P. aeruginosa infection harbor bacterial populations with elevated genomic diversity at the onset of infection.

FIG. 2A show maximum parsimony trees of P. aeruginosa populations in two pilot patients, A and E*. Numbers (rows) correspond to tree leaves (gray), each representing an isolate from day 1 sputum. Phylogenies are rooted with Outgroup. Scale: mutational events (single nucleotide polymorphisms (SNPs) and indels) from the most recent common ancestor (MRCA) inferred in each patient. Select branches are labeled with mutated genes.

FIG. 2B shows a scatter plot comparing initial pathogen diversity in patients (dots), by patient history of P. aeruginosa infection. Number of unique polymorphic loci (SNPs and indels; y axis) in patients with no prior P. aeruginosa history vs. patients with clinically documented infection history (x axis), showing significant difference in means (P=0.007, two-sided t-test).

FIG. 2C is a graph showing the relation between the estimated colonization time of pathogen within each patient (days, y axis) and time to the last clinically documented infection from day 1 (days, x axis) in each patient (dots), calculated for patients with paired samples. Spearman correlation, r=0.93, P=0.003.

FIG. 2D is a graph showing pathways (y axis) found in pre-existing mutations of coding regions at day 1 (x axis) across all patients, with functions related to biofilm formation and motility, among others.

FIG. 2E is a graph showing altered twitching phenotype in isolates with point mutations in genes of the pil locus. Individual isolates (x axis) assayed for twitching diameter (cm, y axis), from left to right: PAO1 strain used as reference, E-11 wild type control, E-9 singleton pilG mutant, E-22 pilJ singleton mutant, each assayed across 3 technical replicates (dots); representative of 3 biologically independent replicates. Bars show median; error bars, standard error (s.e.). Significance: Tukey's multiple comparisons test (E-11 vs. E-9, P=0.0003; E-11 vs. E-22, P=0.0002; adjusted P values).

FIGS. 3A-3G show phylogenetic analyses of P. aeruginosa isolates within patients and their corresponding antibiotic resistance profiles.

FIG. 3A shows a phylogenic analysis of “Patient B”.

FIG. 3B shows a phylogenic analysis of “Patient C”.

FIG. 3C shows a phylogenic analysis of “Patient D”.

FIG. 3D shows a phylogenic analysis of “Patient F*”.

FIG. 3E shows a phylogenic analysis of “Patient G*”.

FIG. 3F shows a phylogenic analysis of “Patient H*”.

FIG. 3G shows a phylogenic analysis of “Patient I*”.

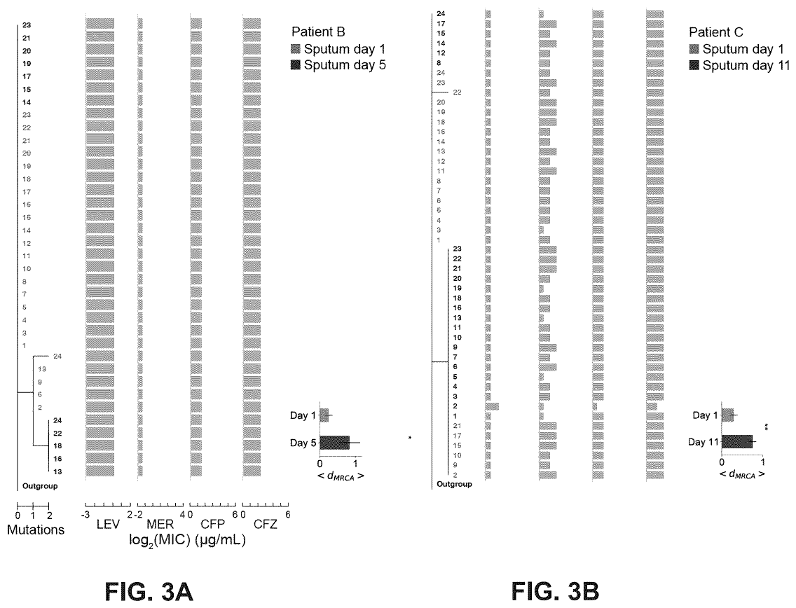

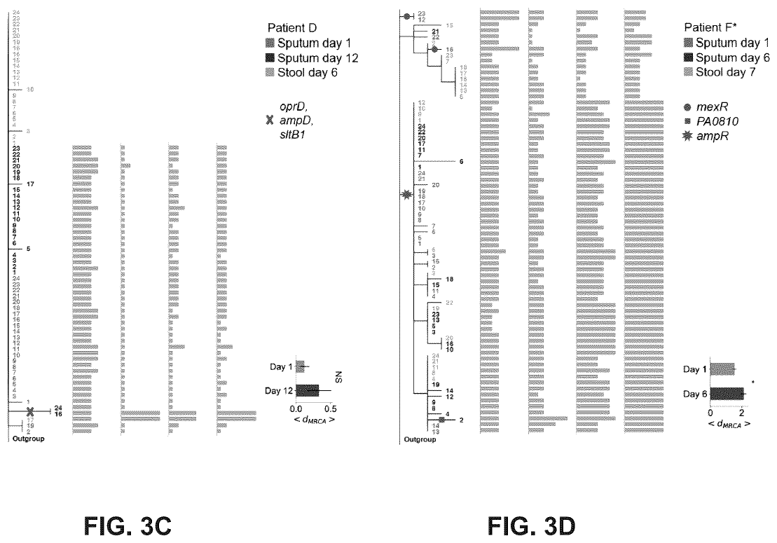

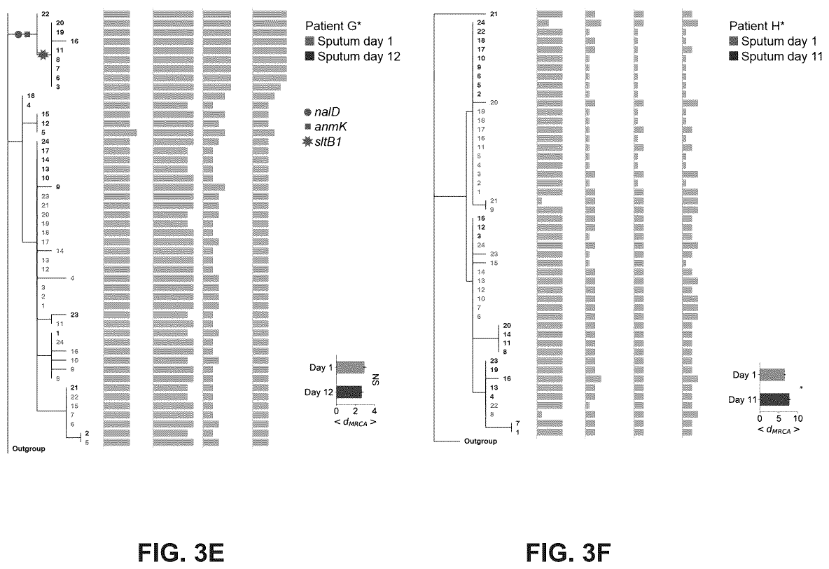

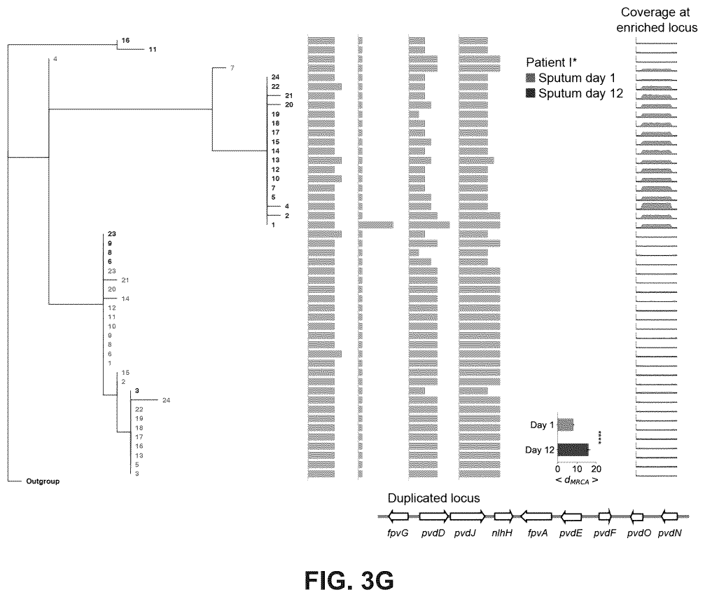

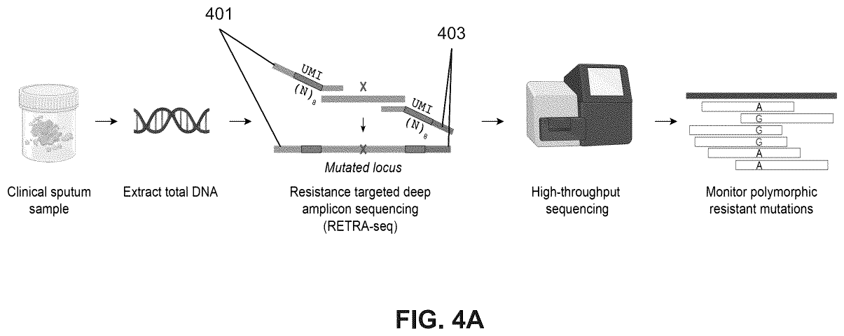

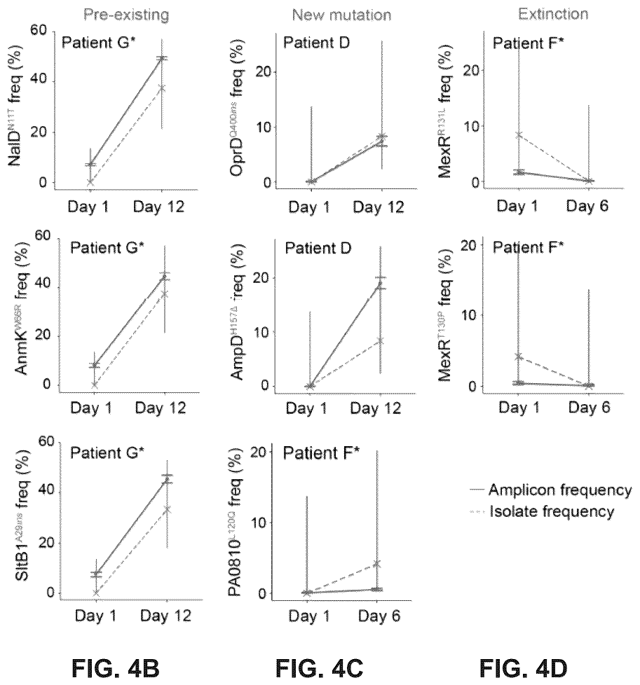

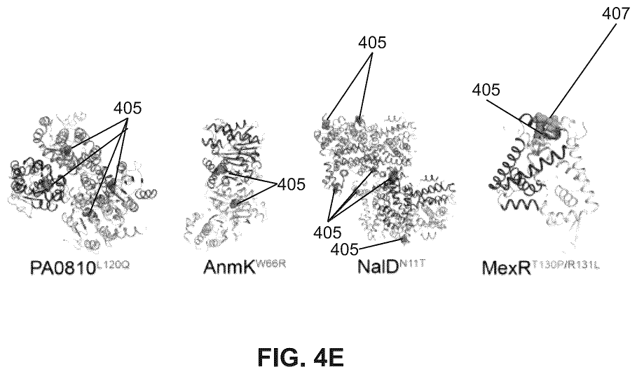

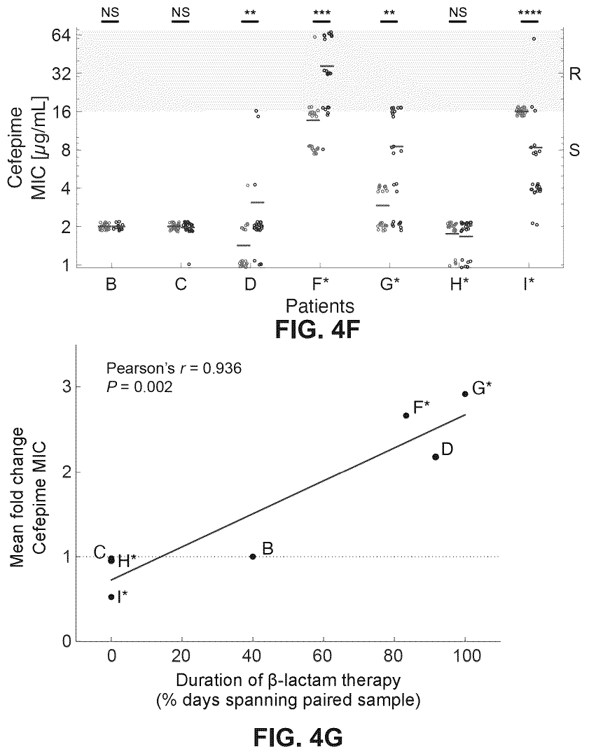

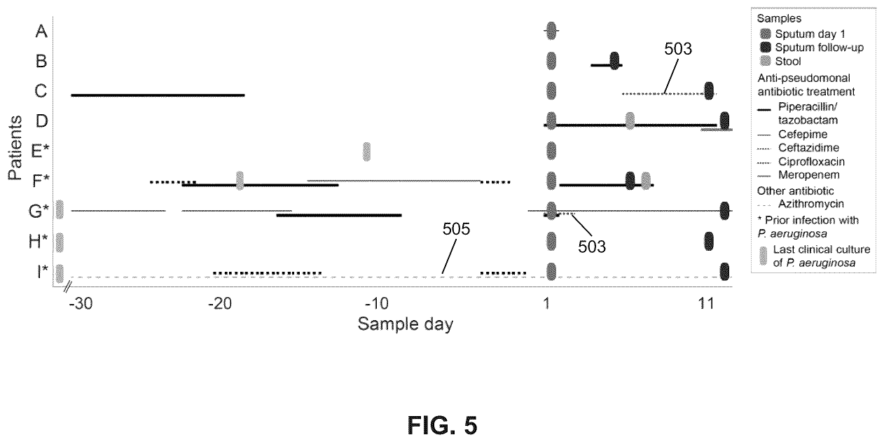

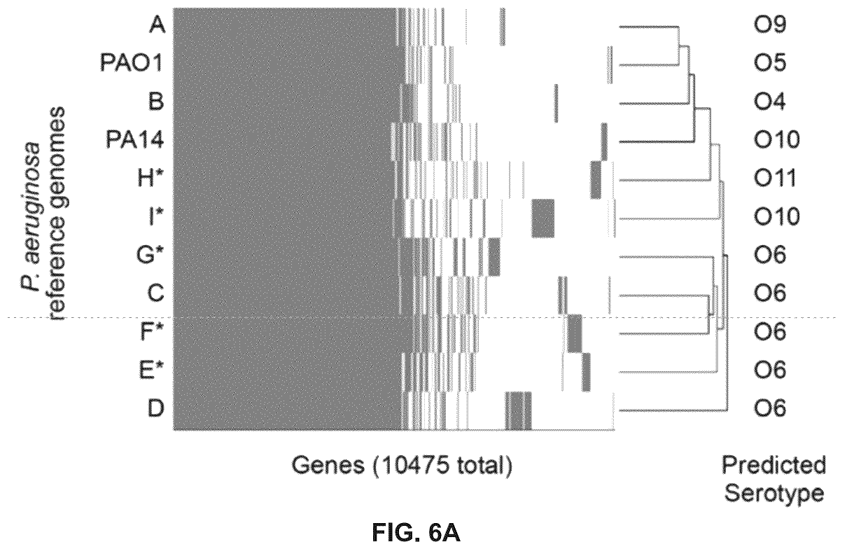

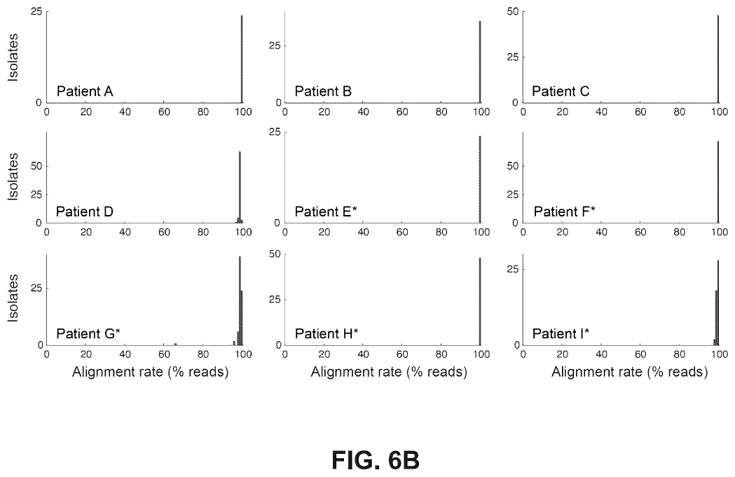

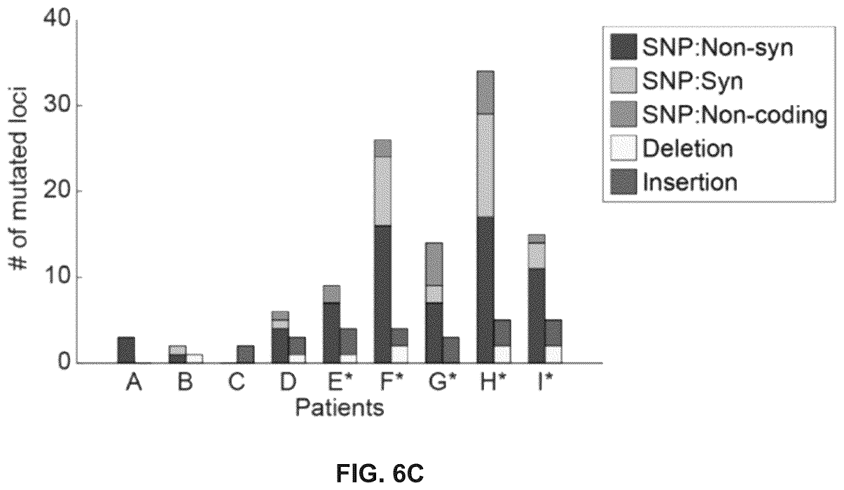

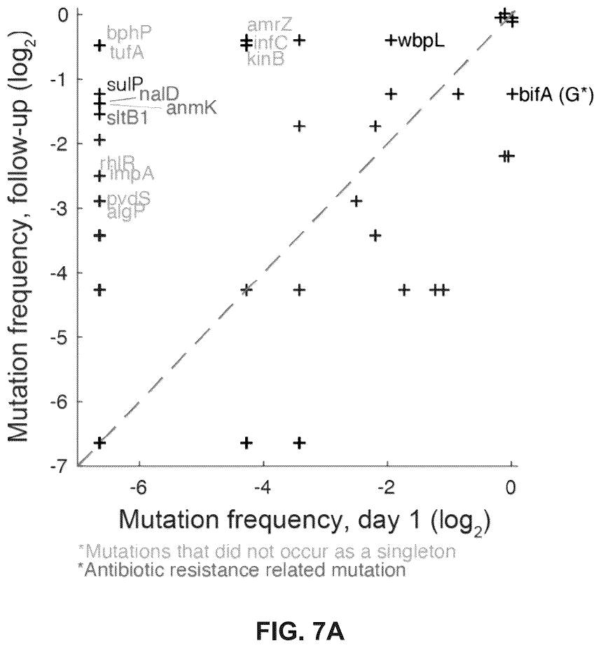

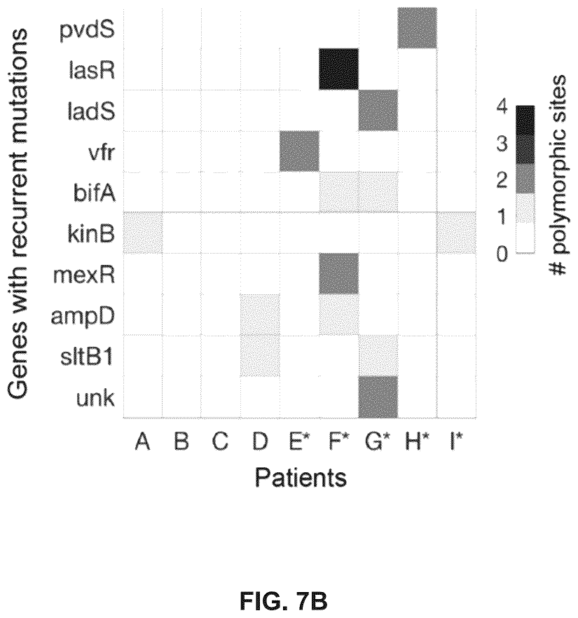

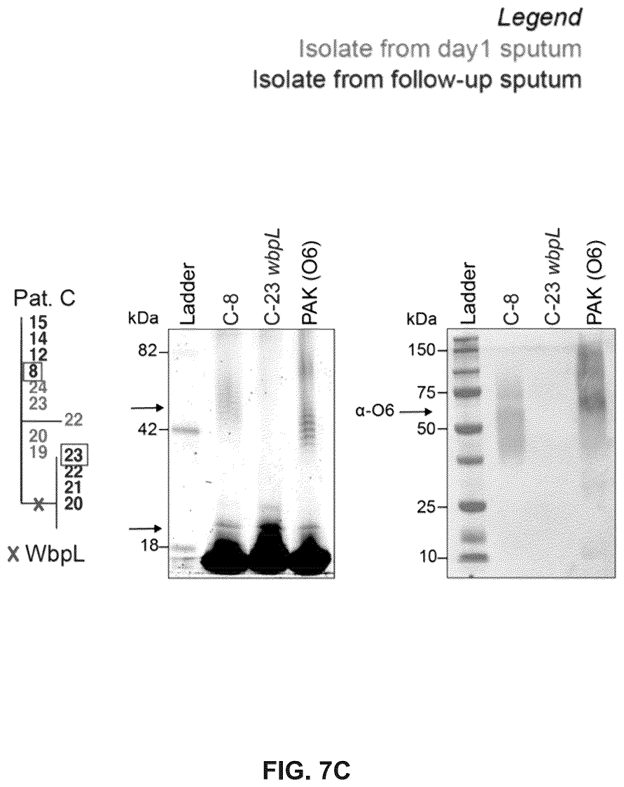

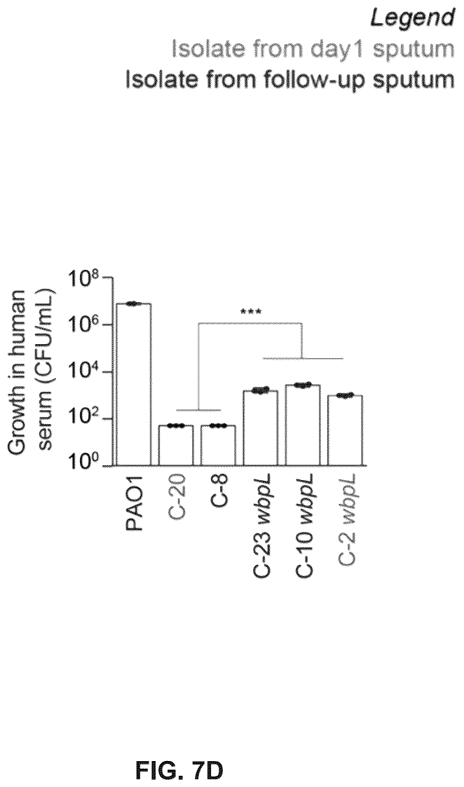

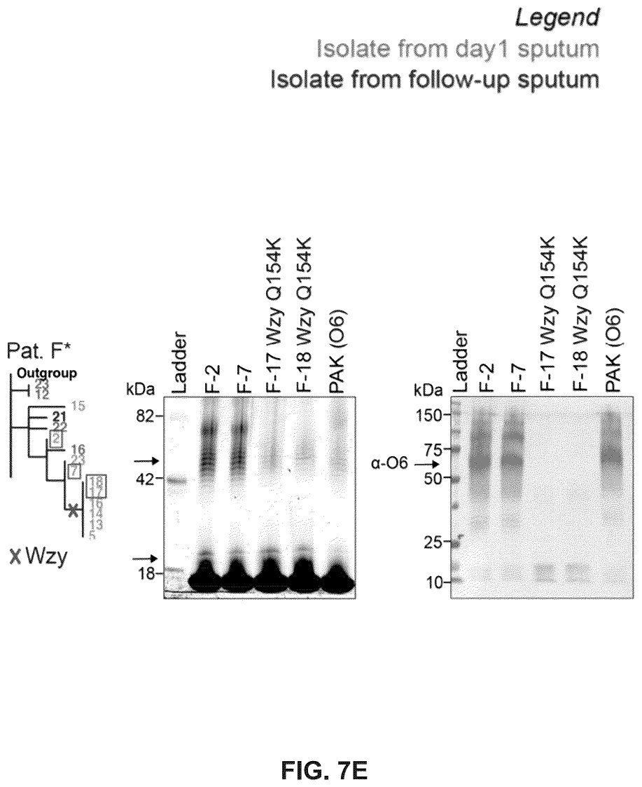

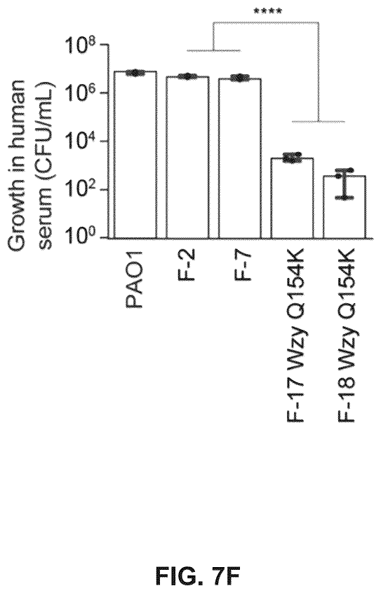

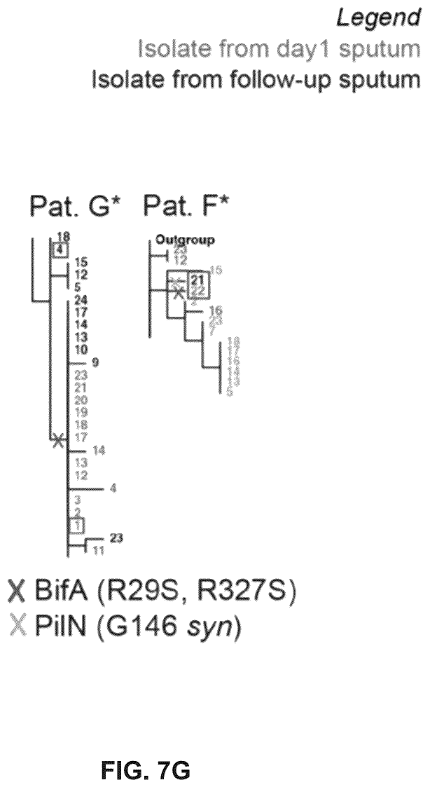

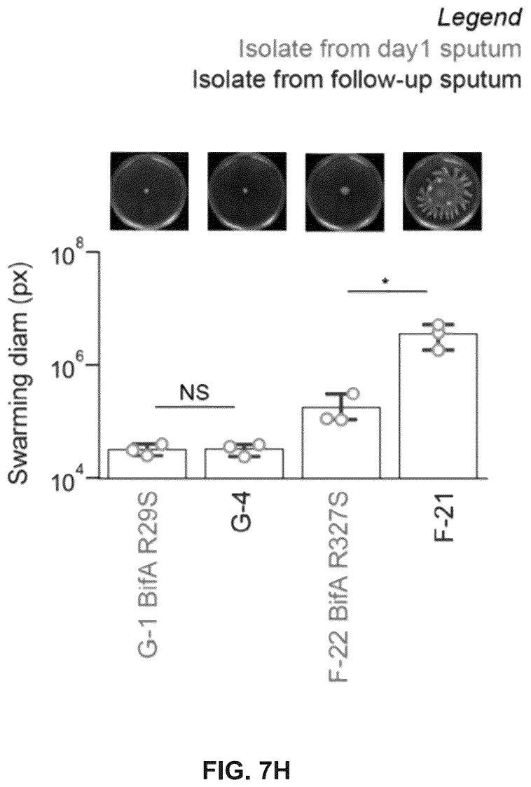

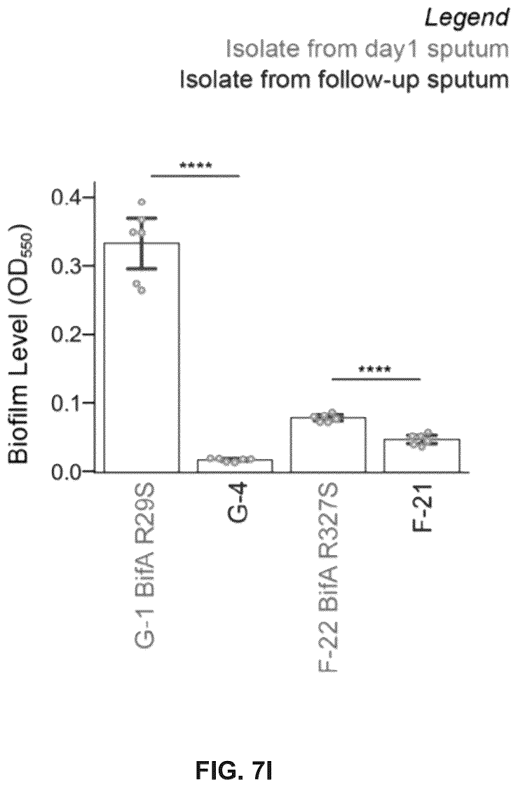

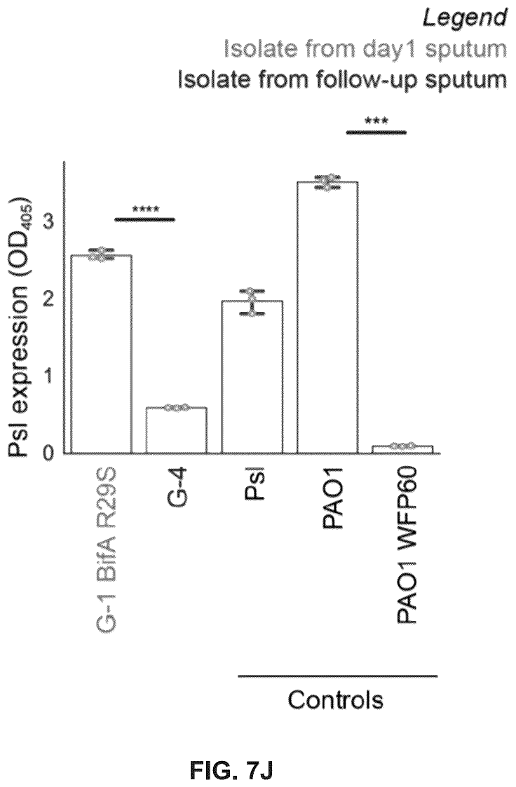

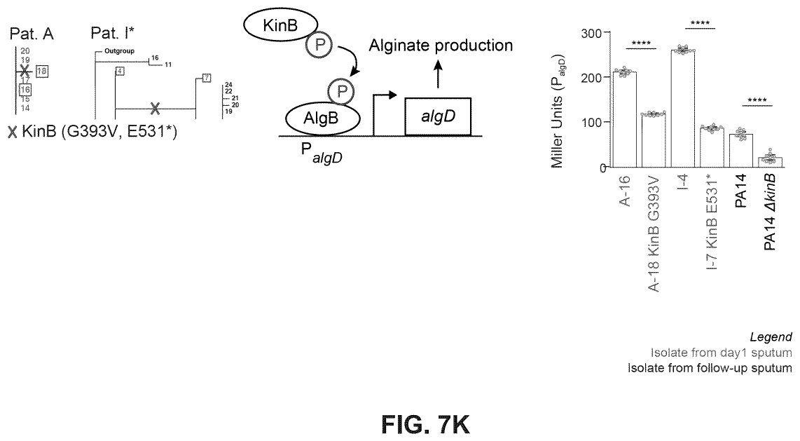

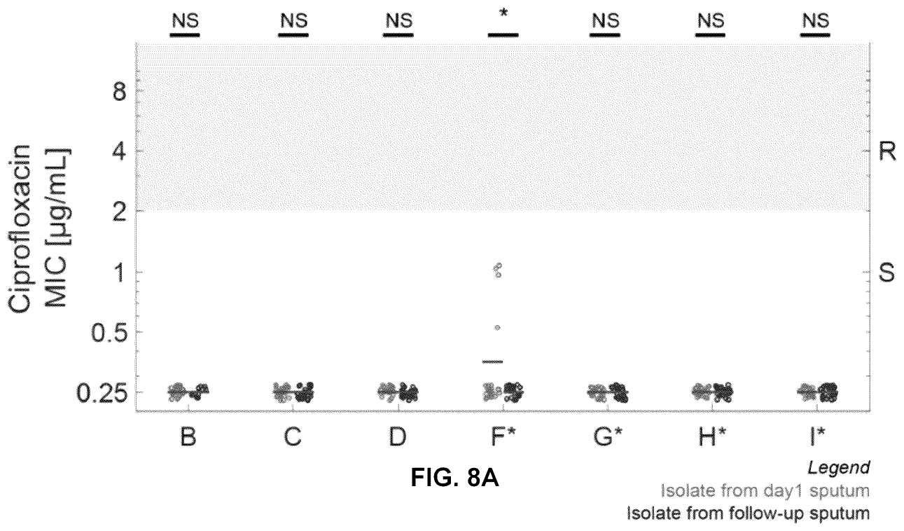

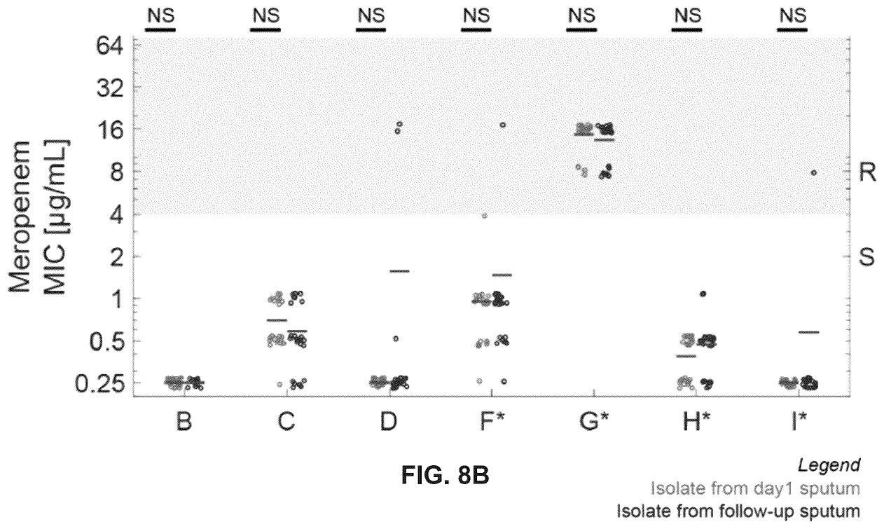

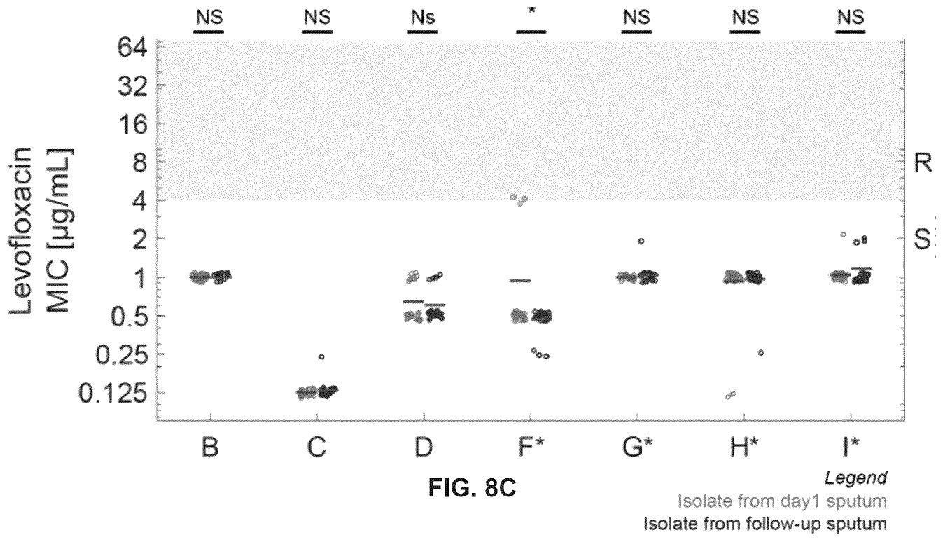

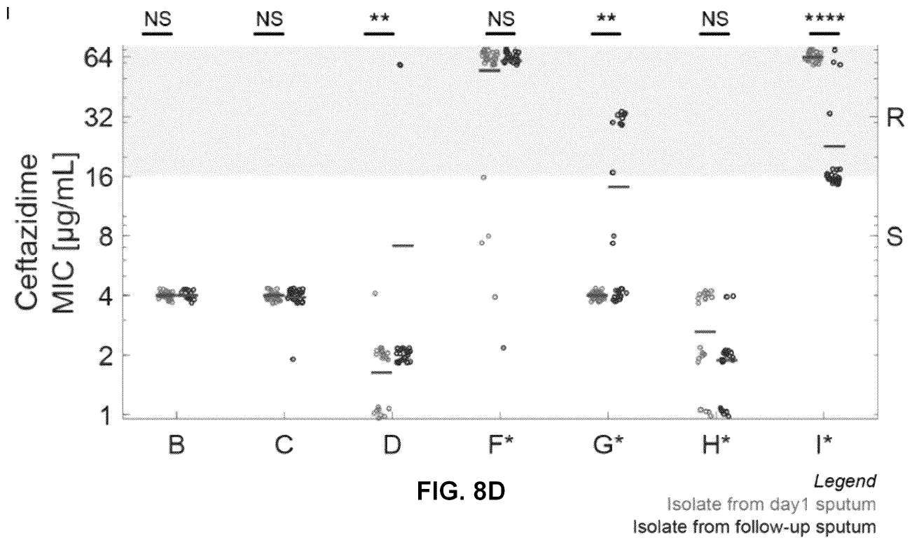

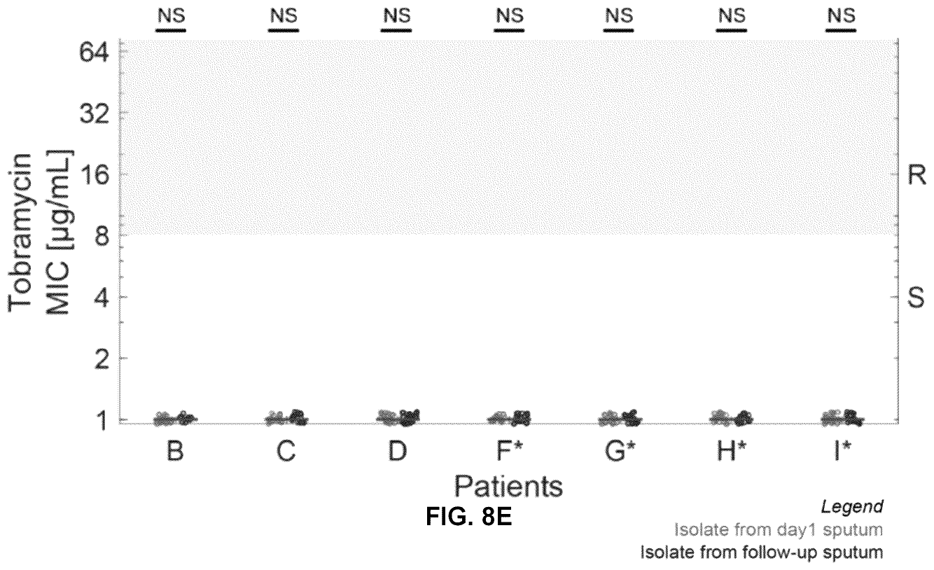

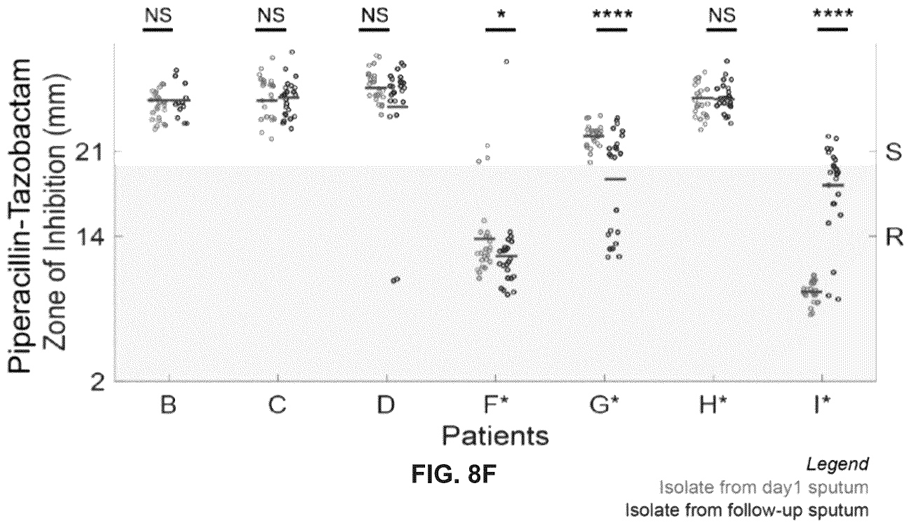

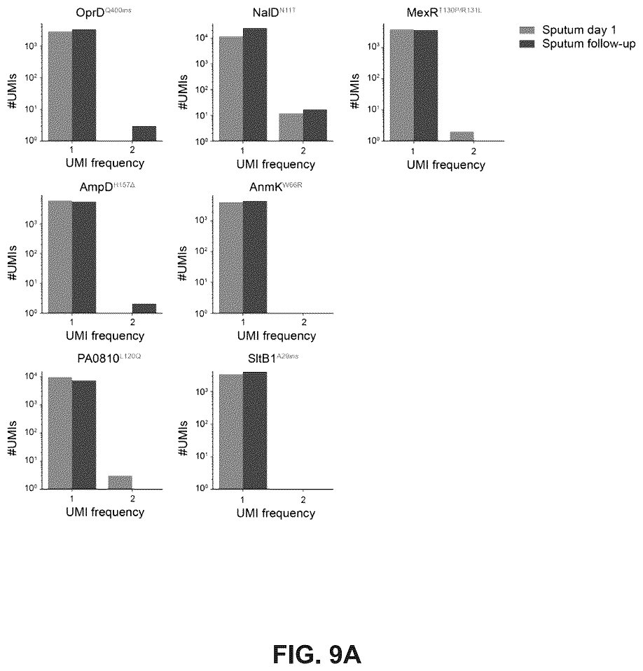

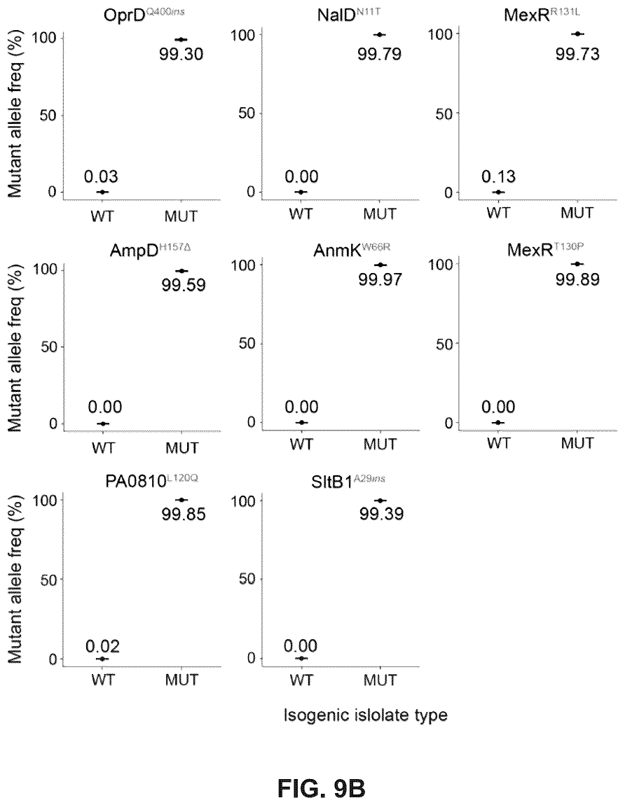

In FIGS. 3A-3G, the left side shows maximum parsimony trees of P. aeruginosa populations in each patient with paired sputum samples. Numbers (rows) correspond to tree leaves, each representing an isolate (teal: isolate from day 1 sputum, violet: isolate from follow-up sputum, light gray: isolate from stool). Phylogenies are rooted with Outgroup. Scale: mutational events (single nucleotide polymorphisms (SNPs) and indels) from the most recent common ancestor (MRCA) inferred in each patient. Select branches associated with increased resistance are marked with gray symbols that indicate non-synonymous or indel mutations in coding genes. In FIGS. 3A-3G, the middle shows Antibiotic resistance profiles (horizontal gray bars) in units of minimum inhibitory concentration (log 2(MIC); μg/mL) of individual isolates (rows) aligned to the isolate's position on the tree, shown for levofloxacin (LEV), meropenem (MER), cefepime (CFP), and ceftazidime (CFZ). In FIGS. 3A-3G, the right side shows horizontal bars showing the average distance to the MRCA (<dMRCA>, x axis) of isolates within each sputum sample (y axis, days of infection). Error bars, standard error of mean; significance, permutation test (one-tailed), *P<0.05, **P<0.005, ****P<10-5. NS—not significant. g. In FIGS. 3A-3G, the far right, bottom shows a schematic showing the relative copy number (y axis) of a duplicated chromosomal region (x axis) spanning ˜34 kb, encoding, among others, several genes of the pyoverdine pathway shown in gene block diagram (bottom).

FIGS. 4A-4F shows that low-frequency resistance mutations expand rapidly within days of infection by selection by treatment.

FIG. 4A provides a workflow diagram illustrating resistance-targeted deep amplicon sequencing (RETRA-seq) as a diagnostic for identifying resistance mutation frequencies in sputum samples. Total DNA is extracted from clinical sputum sample and prepared as sequencing libraries via PCR using primers with sequencing adapters (green 401, red 403) and unique molecular identifiers (UMIs; gray) composed of 8 degenerate nucleotides (N), sequenced on a next-generation sequencing platform, and aligned to a reference genome to determine polymorphic frequencies.

FIGS. 4B-D show mutation frequencies in pathogen populations (y axis) of day 1 and follow-up sputum samples (x axis) by patient (upper left). Frequencies at each time shown as measured by RETRA-seq (solid pink) and by the fraction of culture-based isolates (dashed gray). Axis labels (y axis) indicate mutated gene name and the mutation type (pink superscript) labeled with non-synonymous substitution, insertion (ins), or deletion (Δ). Error bars: Wilson Score interval of UMI counts (amplicon sequencing) or discrete counts (isolate sampling). Three types of changes in resistance mutation frequencies are shown: expansion of mutations that were pre-existing at day 1 but undetected by culture-based assay (b), expansion of de novo mutations emerging after day 1 (c), and extinction of mutations after day 1 (d).

FIG. 4E provides diagrams showing select non-synonymous mapped on protein structures of homologs of PA0810 (Protein Data Bank ID: 3UMC), AnmK NalD (5DAJ), and MexR (3ECH). Each color indicates a distinct monomer. Mutated residues shown by pink spheres 405, with the addition of another residue mutated in MexR shown in green 407.

FIG. 4F shows the distribution of susceptibility to cefepime determined by the minimum inhibitory concentration (MIC, μg/mL; y axis) of individual isolates (dots) in day 1 (teal) and follow-up (violet) sputum samples. Antibiotic susceptibility regimes indicated on the right and by background color, according to breakpoints defined by the Clinical Laboratory Standards Institute (CLSI), with resistant (R) or intermediate susceptibility in gray area and sensitivity (S) in white area. Significance in difference of means (horizontal gray line) across sputum samples within each patient (two-sided Mann-Whitney test): **P<0.005, ***P<10-4, ****P<10-5. NS—not significant.

FIG. 4G is a graph showing the relation between cefepime resistance and clinical history of patient therapy. Fold change in mean cefepime MIC (y axis) and the duration of β-lactam antibiotics administered to the patient, calculated as the fraction of days between the two sputum samples (x axis), shown for each serially sampled patient (dots). Pearson's correlation, r=0.936, P=0.002.

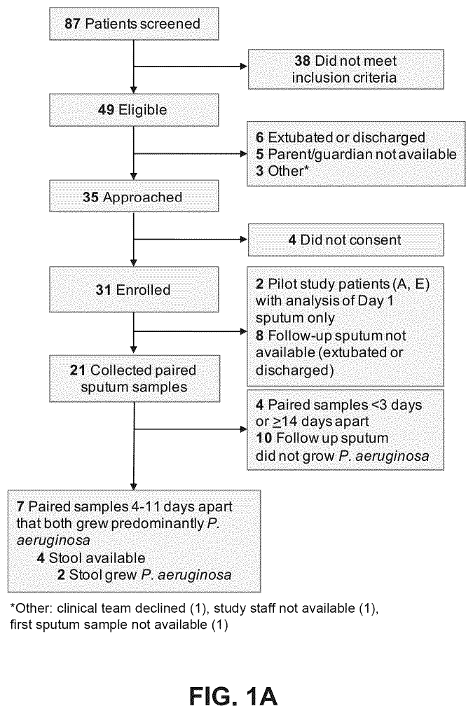

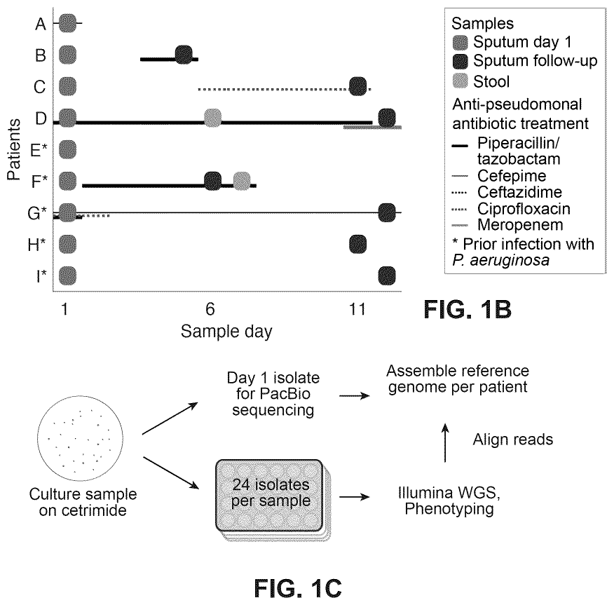

FIG. 5 illustrates the extended antibiotic treatment history of patients. Samples of sputum (day 1 in teal, follow-up in violet) and stool (orange) collected across patients (y axis) over time (x axis) from the onset of symptoms, as in FIG. 1B. Asterisk: patients with documented prior P. aeruginosa infection. Anti-pseudomonal and other antibiotics administered to each patient are indicated by horizontal lines, indicating the days treated, shown for 30 days prior to day 1 on the graph. For patients with prior documented infection of P. aeruginosa, the time of the last confirmed clinical culture of P. aeruginosa is shown by a gray box; cultures confirmed more than 30 days before day 1 shown to the left of the breakpoint (hatched black tracks, x axis). Antibiotics: Piperacillin/tazobactam (weighted solid black), cefepime (thin solid black), ceftazidime (dotted gray), ciprofloxacin (dotted blue, 503), meropenem (weighted solid pink), azithromycin (dashed orange, 505).

FIGS. 6A-C illustrate within-patient polymorphisms using patient-specific reference genomes.

FIG. 6A is a cluster map showing the presence (gray) or absence (white) of coding genes (x axis; 10,475 genes total) in each reference genome (y axis), for all genes of the pangenome constructed across patient strains and two published laboratory strains, PAO1 and PA14. The right side shows serotypes of each strain predicted in silico.

FIG. 6B is a graph showing the distribution of alignment rates across isolates, calculated as the percentage of short-reads from whole-genome sequencing of individual isolates aligned to patient-specific reference genomes.

FIG. 6C is a graph showing the distribution of the number of polymorphic mutation types (y axis) within each patient's population (x axis), shown by subtypes of single nucleotide polymorphisms (left bar: non-synonymous in maroon, synonymous in pale orange, non-coding in orange) and subtypes of short indels (right bar: deletions in light gray, insertions in dark gray).

FIGS. 7A-K characterizes clinically relevant phenotypic impacts of isolate variants.

FIG. 7A is a graph comparing the frequency of each mutation (points) in the pathogen population at day 1 (x axis) vs. in follow-up (y axis) sputum, based on the fraction of cultured isolates. Dotted gray line, y=x. Mutations of coding genes at >5% frequency in at least one time point are labeled with gene names (blue: antibiotic resistance associated mutations, suIP, naID, and anmK, as in FIG. 3; light gray: mutations that did not occur as a singleton).

FIG. 7B shows genes with recurrent mutations (rows), defined as those with two mutated polymorphic positions or more (color, grayscale), within or across patients (columns).

FIGS. 7C-F shows mutations disrupting lipopolysaccharide (LPS) and O antigen presentation (FIGS. 7C, 7E) lead to altered sensitivity to human serum (FIGS. 7D, 7F). In FIGS. 7C and 7E, the left side shows an inset of phylogenies (as shown in FIG. 3A-G) showing mutant and control isolates (gray box, 8 and 23) separated by the singleton mutation marked on the branch (gray x), used for phenotyping. Characterizing mutants of WbpL (single nucleotide frameshift deletion in the O antigen glycosyltransferase) in Patient C (FIG. 7C) or Wzy (non-synonymous substitution in a homolog of the O-polysaccharide polymerase) in Patient F* (FIG. 7E). c-f. Isolates: controls (C-8 in FIG. 7C; F-2 and F-7 in FIG. 7E), mutants (C-23 in FIG. 7C; F-17, F-18 in FIG. 7E), and PAK reference strain (serotype O6). Ladder indicates size (kDa). In FIGS. 7C and 7E, the middle section shows an LPS gel stain image (Pro-Q Emerald 300) showing truncated LPS banding patterns (rows) in mutant isolates compared to controls (columns). Top and bottom arrows indicate larger and truncated LPS banding patterns, respectively. In FIGS. 7C and 7E, the rights section shows a western blot detection of O antigen with anti-O6 antibody, showing intact recognition in controls (arrow) but absence in mutants as shown in FIGS. 7D, 7F. Altered sensitivity to human serum in mutants with disrupted O-antigen. Isolates (x axis) assayed for growth in human serum (CFU/mL, y axis), 3 technical replicates (dots); representative of 3 biologically independent replicates. Bars show median; error bars, standard error. Significance: ***P<0.001, ****P<0.0001, Tukey's multiple comparisons test (pairwise comparison between: C-20 or C-8 vs C-23 or C-10 or C-2; F-2 or F-7 vs F-17 or F-18), as shown in FIGS. 7G-7J. Phenotypic impact of BifA mutations.

FIG. 7G provides an inset of phylogenies (as shown in FIG. 3A-G, left: patient G*, right: patient F*) showing mutant and control isolates (dotted box) separated by KinB mutations labeled on the branch (dotted x; R29S singleton in Patient G*, R327S in patient F*). Control isolate of Patient F* harbored an additional synonymous G146 substitution in the gene PilN.

FIG. 7H-7J are graphs showing Control (G-4, F-21) and mutant (G-1 R29S mutant, F-22 R327S mutant) isolates (x axis) were phenotyped for swarming (FIG. 7H, diameter in corresponding images, pixels, y axis), biofilm production (FIG. 7I, OD550, y axis), and Psl expression measured by ELISA (FIG. 7J, OD405, y axis), each across 3, 6, or 3 technical replicates, respectively. Bars show median, error bars show standard error. Significance (FIGS. 7H-J): *P=0.0239, ****P<0.0001, two-sided t-test. NS—not significant.

FIG. 7K shows the phenotypic impact of KinB mutations. Inset of phylogenies (as in FIG. 3A-G, left: patient A, right: patient I*) showing mutant and control isolates, dotted box (controls: A-16, 1-4; mutants: A-18 G393V mutant, I-7 E531* mutant). KinB phosphorylates AlgB, which regulates algD and subsequent alginate production. Bar graph: isolates (x axis) have altered algD promoter activity (Miller units of R-gal expression, y axis); bars show median, error bars show standard error, for 8 technical replicates. Significance: ****P<0.0001, two-sided t-test.

FIGS. 8A-F show susceptibility measurements of all sputum isolates against anti-pseudomonal antibiotics. Distribution of antibiotic susceptibility determined by the minimum inhibitory concentration in liquid cultures (MIC, μg/mL y axis, a-e) or by the zone of inhibition via disk diffusion assay (mm, y axis, f) of individual isolates (dots) in day 1 (teal) and follow-up (violet) sputum samples. Antibiotic susceptibility regimes indicated on the right and by background color, according to breakpoints defined by the Clinical Laboratory Standards Institute (CLSI), with resistant (R) or intermediate susceptibility in gray and sensitivity (S) in white. Significance in difference of means (horizontal gray line) across sputum samples within each patient (two-sided Mann-Whitney test): **P<0.005, ***P<10-4, ****P<10-5. NS—not significant.

FIGS. 9A-9B are graphs assessing the unique number of genomes captured with deep amplicon sequencing. FIG. 9A shows a number of distinct unique molecular identifiers (UMIs, y axis) found in each amplicon sequencing library (individual plots, title), by the frequency of observed for each UMI (x axis) in raw sequencing data of each sputum sample (bar color; teal, day 1 sputum and violet, follow-up sputum). To account for amplification bias, primers barcoded with UMIs were used to amplify total DNA extracted from sputum. FIG. 9B shows mutant allele frequencies (y axis; exact frequencies labeled on plot) measured by deep amplicon sequencing in isogenic controls (x axis), left: WT colony, right: mutant colony, plots arranged as in FIG. 4B-D. Error bars: Wilson Score interval.

### DETAILED DESCRIPTION OF THE INVENTION

The disclosure features compositions and methods that are useful for characterizing low frequency resistance mutations and methods for selecting therapies for patients developing such resistance mutations. Exemplary resistance mutations include, but are not limited to, mutations that result in antibiotic, antifungal, antiviral, or chemotherapeutic resistance,

The invention is based, at least in part, on the discovery of a new method for characterizing rare resistance mutations using a new technique, termed Resistance-Targeted Deep Amplicon Sequencing (RETRA-Seq), which revealed that rare resistance mutations not detected by clinically used culture-based methods, can increase by nearly 40-fold over 5-12 days in response to antimicrobial changes. Acute bacterial infections are often treated empirically, with the choice of antimicrobial therapy (e.g., an antibiotic) updated during treatment. The effects of such rapid antimicrobial switching on the evolution of antimicrobial resistance in individual patients are poorly understood. As reported in detail below, it was found that low-frequency antimicrobial resistance mutations emerge, contract, and even go to extinction within days of changes in therapy. Pseudomonas aeruginosa populations were analyzed in sputum samples collected serially from 7 mechanically ventilated patients at the onset of respiratory infection. Combining short- and long-read sequencing and resistance phenotyping of 420 isolates revealed that while new infections are near-clonal, reflecting a recent colonization bottleneck, resistance mutations could emerge at low frequencies within days of therapy. The in vivo frequencies of select resistance mutations in intact sputum samples were measured with resistance-targeted deep amplicon sequencing (RETRA-Seq), which revealed that rare resistance mutations not detected by clinically used culture-based methods can increase by nearly 40-fold over 5-12 days in response to antimicrobial changes. Conversely, mutations conferring resistance to antimicrobials not administered diminish and even go to extinction. These findings underscore how therapy choice shapes the dynamics of low-frequency resistance mutations at short time scales, and provide a possibility for driving resistance mutations to extinction during early stages of infection by designing patient-specific antimicrobial cycling strategies informed by deep genomic surveillance.

### Detection of Low Frequency Mutations

The present disclosure provides compositions and methods useful for detecting one or more mutations (e.g., low frequency mutations) present in polynucleotides, including DNA (e.g., genomic DNA) or RNA. For example, methods described herein can be used to detect a mutation occurring at a frequency of less than 1%, e.g., less than 0.1%, in an individual's DNA or mixed DNA, such as from a mixture of microbial and patient genomic DNA. Such low-frequency mutations can include point mutations, base substitutions, deletions, insertions, and/or chromosomal rearrangements. The low frequency mutation identified by methods and compositions described herein can be present in a genic or an intergenic region of a nucleic acid, including a gene or a regulator of a gene, such as, a gene promoter or an enhancer. Since methods and compositions described herein can detect a mutation at the level of a single base pair, these methods and compositions may have particular applicability to clinical practices involving precision diagnostics and/or therapeutics.

Precision diagnostic and therapeutics often rely on sequencing of genes frequently mutated/amplified/deleted in certain diseases or conditions (e.g., bacterial infection) and believed to be associated with pathological progress. Recent studies, however, have revealed several limitations of these widely used approaches. For example, sequencing performed on a culture from a single cultured bacterial colony will not reveal heterogeneity. Clones evolving independently or minor clones with distinct mutations are often overlooked. In order to identify “low frequency” mutations, sequencing depth is important, yet most studies fail to identify mutations present in less than 15% of cells due to lack of deep sequencing.

In some embodiments, this disclosure describes methods and compositions that allow for the detection of low-frequency mutations by, in part, eliminating the biases that cause existing methodologies to overlook rare mutations. For example, in the context of antibacterial resistance mutations, current clinical methods for detecting resistance mutations are largely culture-based, where bacterial isolates with visually distinct morphology (by size, shape, color) are selected for profiling. However, these methods are susceptible to biases from culture-based growth and are limited in their sampling resolution, especially for detecting low-frequency mutations.

Compositions and methods described herein overcome those limitations by providing strategies for detecting mutations directly from a patient sample, such as sputum. Accordingly, in some embodiments, methods described herein can detect antimicrobial resistance directly from a clinical specimen and provide valuable information that can help clinicians make difficult decisions regarding patients, such as when to change antimicrobials and which antimicrobials to use to improve likelihood of a positive clinical outcome. As such, methods and compositions of this disclosure can be used guide treatment decisions during treatment of bacterial infections, including acute bacterial infections. For example, the methods described herein can be used to inform on which antimicrobials should be avoided, or conversely, which should be actively used in the case of compounds that select against a specific type of resistance.

Acute bacterial infections are often treated empirically, with the choice of antimicrobial therapy updated during treatment. The effects of such rapid antimicrobial switching on the evolution of antimicrobial resistance in individual patients are poorly understood. However, an insight of this disclosure is the discovery that low-frequency antimicrobial resistance mutations emerge, contract, and even go to extinction within days of changes in therapy. For example, disclosed herein are analyses of Pseudomonas aeruginosa populations in sputum samples collected serially from 7 mechanically ventilated patients at the onset of respiratory infection. Combining short- and long-read sequencing and resistance phenotyping of 420 isolates revealed that while new infections are near-clonal, reflecting a recent colonization bottleneck, resistance mutations could emerge at low frequencies within days of therapy. Measurements of in vivo frequencies of select resistance mutations in intact sputum samples were analyzed with resistance-targeted deep amplicon sequencing (RETRA-Seq), which revealed that rare resistance mutations not detected by clinically used culture-based methods can increase by nearly 40-fold over 5-12 days in response to antimicrobial changes. Conversely, mutations conferring resistance to antimicrobials not administered diminish and even go to extinction. The insights of this disclosure underscore how therapy choice shapes the dynamics of low-frequency resistance mutations at short time scales and provide a possibility for driving resistance mutations to extinction during early stages of infection by designing patient-specific antimicrobial cycling strategies informed by deep genomic surveillance. Antimicrobial treatment selects for resistance mutations, posing a major threat to effective treatment of bacterial infections. The selection of resistance mutations during chronic infections as a result of antimicrobial treatment over months to years is known. However, it is not well-understood how short-term changes in antimicrobial therapy affect the dynamics of resistance mutations in acute infections, especially in a newly colonizing infection that is thought to start from a clonal population.

Emerging resistance is of particular concern in the treatment of acute respiratory tract infections that are common in intensive care units (ICUs) worldwide, particularly in mechanically ventilated patients who are at high risk for ventilator-associated pneumonia (VAP), septic shock, and infection-associated mortality. VAP and other lower respiratory tract infections are of major concern in the SARS-CoV-2 pandemic given the large number of hospitalized COVID-19 patients requiring ventilation. Pseudomonas aeruginosa is one of the most common bacterial pathogens causing respiratory infections in ventilated patients and is associated with increased mortality and low treatment efficacy due to high rates of antimicrobial resistance that can occur within days of antimicrobial treatment.

Shallow profiling of pathogen populations using cultured isolates have shown that the frequencies of antimicrobial resistance mutations can fluctuate over days to weeks during infection, but whether changes reflect drift, sampling bias, or treatment-induced selection at short timescales is unknown. Current clinical methods for detecting resistance variants are largely culture-based, where isolates with visually distinct morphology (by size, shape, color) are selected for resistance phenotyping. However, these methods are susceptible to bias from culture-based growth and are limited in their sampling resolution, especially for detecting low-frequency mutations. While molecular surveillance methods such as rapid PCR tests and real-time genome sequencing can identify the presence of known resistance genes, e.g. efflux pumps, for the rapid identification of resistant strains, they are not suitable for monitoring within-population pathogen diversity. Furthermore, it is not well-understood whether resistance mutations can contract and be reversed during the course of treatment in acute infection. A molecular, culture-free diagnostic could determine the role of low-frequency resistance variants at short time scales, and possibly inform which antimicrobials should be avoided.

This disclosure provides methods and compositions that combine whole genome sequencing with resistance-targeted deep amplicon sequencing (RETRA-Seq). Using methods and compositions of the disclosure, provided herein are data that show that resistance mutations, either pre-existing or de novo, expand and contract rapidly within days of changes in therapy. By conducting a deep sampling study of P. aeruginosa populations and using long-read sequencing to construct patient-specific reference genomes in order to maximize the detection of within-population mutations, described herein are methods to construct a high-resolution view of pathogen evolution during acute respiratory infection. This disclosure then relates how changes in empirically administered antimicrobials impact resistance mutations in individual patients, and discover that resistance mutation frequencies change within days, depending on the duration and type of antimicrobial therapy.

This disclosure provides the insight that frequencies of within-population resistance mutations change rapidly with antimicrobial therapy, highlighting a potential for deep sequencing-guided, short-term cycling of antimicrobials within patients as a possible future therapeutic strategy. As resistance mutations can persist in the population for months following treatment, monitoring low-frequency mutations by deep population profiling can inform which antimicrobials should be avoided, or conversely, should be actively used in the case of compounds that select against a specific type of resistance. While antimicrobial cycling has been proposed as a strategy to limit the selective advantage of resistance mutations based on mathematical modeling and experimental evolution studies, to date, there are limited data on its clinical efficacy. This disclosure offers an approach to examine and treat acute infections, by identifying drugs likely to produce a positive clinical outcome within individual patients over short time scales.

To inform patient-specific antimicrobial cycling strategies, molecular diagnostics that deeply and accurately monitor pathogen diversity throughout infection, particularly at the start of infection, are needed. Current culture-based clinical microbiology practice risks missing low-frequency resistant variants. Furthermore, culture-based assays introduce growth bias that differs from the native context of the human lung, where spatial selection is known to occur on pathogens across different niches. Specific alleles encoding resistance could be detected with next-generation molecular assays, e.g. CRISPR-based diagnostics. To monitor known hotspots of mutated genes, this disclosure provides resistance targeted deep amplicon sequencing (RETRA-Seq), using primers that are designed to be suitable across multiple strains, as a highly sensitive method to monitor numerous loci across pathogen genomes.

In some embodiments, methods of the disclosure are useful for determining a rate of change in frequency of one or more resistance mutations. In some embodiments, determining a change in frequency of resistance mutations is carried out by performing a fluctuation assay. A fluctuation assay involves determining the distribution of mutant numbers of a microbial population at different time points. The time points can be 1, 2, 3, 4, 5, 6, or 7 days apart, or the time points can be 1, 2, 3, 4, or 5 weeks apart. Determining changes in frequency of resistance mutations can inform on certain changes in microbial populations, such as whether a particular clone that harbors a resistance mutation within the population is expanding (e.g., growing) or contracting. In some embodiments, methods described herein are useful for detection of mutations associated with antibiotic resistance. Resistance mutations that are detectable by compositions and methods described herein include any mutation in any one or more of the genes listed in Table 2, or Table 3, or in a regulator of any one or more of the genes listed. For example, the resistance mutation can be in a gene that has a sequence that is at least 75%, 80%, 85%, 90%, 95%, 99%, or 100% identical to one of the genes listed in Table 2. In some embodiments, methods of the disclosure involve targeted amplification of a gene, or a regulator of the gene, associated with bacterial resistance. For example, the gene can be any one or more of the genes listed in Table 2 or Table 3. The regulator can be a gene promoter or an enhancer. In some embodiments, methods of the disclosure involve the targeted amplification of a gene, such as a resistance gene. The resistance gene can be any one or more of the genes listed in Table 2, or Table 3. In some embodiments, compositions and methods described herein involve the use of primers that hybridize to a genomic DNA flanking a gene associated with a resistance mutation, including one or more of the genes listed in Table 2 or Table 3. After hybridization, the primer can be used to amplify the resistance mutation (e.g., by PCR) for downstream analysis. In some embodiments, the primer is selected from one or more of the primers listed in Table 4. In some embodiments, the gene comprises sequences or is flanked by a sequence that is at least 75%, 80%, 85%, 90%, 95%, 99%, or 100% identical to one of the sequences listed in Table 4. In some embodiments, the gene encodes a product that has a sequence that is at least 75%, 80%, 85%, 90%, 95%, 99%, or 100% identical to one of the amino acid sequences of the genes listed in Table 2.

In some embodiments, methods and compositions described herein are useful to detect and monitor subclinical reservoir mutations. For example, as discussed below, methods and compositions described herein can be used to detect microbes harboring one or more resistance mutations even before the pathogens present themselves clinically (e.g., give rise to an infection).

In general, antimicrobial resistance is characterized by detecting alterations in the sequence of a nucleic acid molecule derived from a pathogen present in a biological sample collected from a subject (e.g., patient having a bacterial infection).

### Pathogens

The methods described herein are ideally suited for characterizing genetic alterations in organisms subject to selective pressure. In particular embodiments, the organism is a pathogen. Pathogens include, but are not limited to, bacteria, viruses, fungi, and protozoa. Some exemplary pathogens include, but are not limited to, Helicobacter pylori, Borrelia burgdorferi, Legionella pneumophilia, Mycobacteria species (e.g. M. tuberculosis, M. avium, M. intracellulare, M kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Enterococcus faecalis, Streptococcus bovis, Streptococcus (anaerobic sps.), Streptococcus pneumoniae, pathogenic Campylobacter sp., Salmonella species, Shigella species, Yersinia species, Enterococcus species, Haemophilus influenzae, Bacillus anthracis, Erysipelothrix rhusiopathiae, Clostridium perfringers, Clostridium tetani, Clostridioides difficile, Pasteurella multocida, Bacteroides sp., Fusobacterium species, Streptobacillus moniliformis, Treponema pallidium, Treponema pertenue, Leptospira, Rickettsia, and Actinomyces israelli and Pseudomonas aeruginosa, Escherichia coli, Klebsiella species, Enterobacter species, Acinetobacter species, Stenotrophomonas maltophilia, Burkholderia cepacia complex, Achromobacter species, and Burkholderia pseudomallei, malaria, amoebiasis, giardiasis, toxoplasmosis, cryptosporidiosis, trichomoniasis, leishmaniasis, African trypanosomiasis, Acanthamoeba keratitis, primary amoebic meningoencephalitis, Orthopoxvirus, influenza, mumps, rubella, varicella, Ebola, HIV, Candida albicans, and Cryptococcus neoformans In some embodiments, the pathogen is a bacteria. In some embodiments, the pathogen is a gram negative bacteria. For example, in some embodiments, the pathogen is one of Pseudomonas aeruginosa, Escherichia coli, Klebsiella species, Enterobacter species, Acinetobacter species, Stenotrophomonas maltophilia, Burkholderia cepacia complex, Achromobacter species, and Burkholderia pseudomallei.

For example, in some embodiments the pathogen is a virus. Viruses are small particles, typically between 20 and 300 nanometers in length that contain RNA or DNA. Viruses require a host cell to replicate. Some of the diseases that are caused by viral pathogens include smallpox, influenza, mumps, measles, chickenpox, ebola, HIV, rubella, and COVID-19. Exemplary pathogenic viruses can be from any one of Adenoviridae, Coronaviridae, Picornaviridae, Herpesviridae, Hepadnaviridae, Flaviviridae, Retroviridae, Orthomyxoviridae, Paramyxoviridae, Papovaviridae, Polyomavirus, Rhabdoviridae, and Togaviridae. In some embodiments, the pathogen is a protozoan, which can cause a number of diseases including malaria, amoebiasis, giardiasis, toxoplasmosis, cryptosporidiosis, trichomoniasis, Chagas disease, leishmaniasis, African trypanosomiasis, Acanthamoeba keratitis, and primary amoebic meningoencephalitis. In some embodiments, the pathogen is a fungus, for example, the pathogen can be Candida albicans or Cryptococcus neoformans. In some embodiments, the pathogen is a bacteria, such as a gram positive bacteria or a gram negative bacteria.

Gram negative bacteria include Escherichia coli, Pseudomonas species, and Salmonella species. Specific examples of bacteria include but are not limited to, Helicobacter pyloris, Borelia burgdorferi, Legionella pneumophilia, Mycobacteria sps (e.g. M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus antracis, Corynebacterium diphtheriae, corynebacterium sp., Erysipelothrix rhusiopathiae, Clostridium perfringers, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, Rickettsia, and Actinomyces israelli. Gram positive bacteria include, but are not limited to, Pasteurella species, Staphylococci species, and Streptococcus species.

Antimicrobials are used to treat, destroy, or inhibit the growth of disease-causing pathogens. Antimicrobials described herein can include antibiotics, antifungals, antiparasitics, microbicides, antimicrobial chemotherapy agents, antimicrobial prophylaxis. Antimicrobials are frequently used to treat bacterial infections. Antibiotic therapies are used to reduce or inhibit the proliferation of bacteria. In one embodiments, the antibiotic is selected from the penicillins (e.g., penicillin G, ampicillin, methicillin, oxacillin, and amoxicillin), the cephalosporins (e.g., cefazolin, cefuroxime, cefotaxime, and ceftriaxone, ceftazidime), the carbapenems (e.g., imipenem, ertapenem, and meropenem), the tetracyclines and glycylclines (e.g., doxycycline, minocycline, tetracycline, and tigecycline), the aminoglycosides (e.g., amikacin, gentamycin, kanamycin, neomycin, streptomycin, and tobramycin), the macrolides (e.g., azithromycin, clarithromycin, and erythromycin), the quinolones and fluoroquinolones (e.g., gatifloxacin, moxifloxacin, sitafloxacin, ciprofloxacin, lomefloxacin, levofloxacin, and norfloxacin), the glycopeptides (e.g., vancomycin, teicoplanin, dalbavancin, and oritavancin), dihydrofolate reductase inhibitors (e.g., cotrimoxazole, trimethoprim, and fusidic acid), the streptogramins (e.g., synercid), the oxazolidinones (e.g., linezolid) and the lipopeptides (e.g., daptomycin).

In some embodiments, the pathogen (e.g., virus, bacteria, fungus), cell (e.g., cancer cell), or organism acquires resistance to a therapeutic agent (e.g., antibiotic, antiviral, antifungal, chemotherapeutic).

Other exemplary antibiotics include, but are not limited to, Aztreonam; Chlorhexidine Gluconate; Imidurea; Lycetamine; Nibroxane; Pirazmonam Sodium; Propionic Acid; Pyrithione Sodium; Sanguinarium Chloride; Tigemonam Dicholine; Acedapsone; Acetosulfone Sodium; Alamecin; Alexidine; Amdinocillin; Amdinocillin Pivoxil; Amicycline; Amifloxacin; Amifloxacin Mesylate; Amikacin; Amikacin Sulfate; Aminosalicylic acid; Aminosalicylate sodium; Amoxicillin; Amphomycin; Ampicillin; Ampicillin Sodium; Apalcillin Sodium; Apramycin; Aspartocin; Astromicin Sulfate; Avilamycin; Avoparcin; Azithromycin; Azlocillin; Azlocillin Sodium; Bacampicillin Hydrochloride; Bacitracin; Bacitracin Methylene Disalicylate; Bacitracin Zinc; Bambermycins; Benzoylpas Calcium; Berythromycin; Betamicin Sulfate; Biapenem; Biniramycin; Biphenamine Hydrochloride; Bispyrithione Magsulfex; Butikacin; Butirosin Sulfate; Capreomycin Sulfate; Carbadox; Carbenicillin Disodium; Carbenicillin Indanyl Sodium; Carbenicillin Phenyl Sodium; Carbenicillin Potassium; Carumonam Sodium; Cefaclor; Cefadroxil; Cefamandole; Cefamandole Nafate; Cefamandole Sodium; Cefaparole; Cefatrizine; Cefazaflur Sodium; Cefazolin; Cefazolin Sodium; Cefbuperazone; Cefdinir; Cefepime; Cefepime Hydrochloride; Cefetecol; Cefixime; Cefinenoxime Hydrochloride; Cefmetazole; Cefmetazole Sodium; Cefonicid Monosodium; Cefonicid Sodium; Cefoperazone Sodium; Ceforanide; Cefotaxime Sodium; Cefotetan; Cefotetan Disodium; Cefotiam Hydrochloride; Cefoxitin; Cefoxitin Sodium; Cefpimizole; Cefpimizole Sodium; Cefpiramide; Cefpiramide Sodium; Cefpirome Sulfate; Cefpodoxime Proxetil; Cefprozil; Cefroxadine; Cefsulodin Sodium; Ceftazidime; Ceftibuten; Ceftizoxime Sodium; Ceftriaxone Sodium; Cefuroxime; Cefuroxime Axetil; Cefuroxime Pivoxetil; Cefuroxime Sodium; Cephacetrile Sodium; Cephalexin; Cephalexin Hydrochloride, Cephaloglycin; Cephaloridine; Cephalothin Sodium; Cephapirin Sodium; Cephradine; Cetocycline Hydrochloride; Cetophenicol; Chloramphenicol; Chloramphenicol Palmitate; Chloramphenicol Pantothenate Complex; Chloramphenicol Sodium Succinate; Chlorhexidine Phosphanilate; Chloroxylenol; Chlortetracycline Bisulfate; Chlortetracycline Hydrochloride; Cinoxacin; Ciprofloxacin; Ciprofloxacin Hydrochloride; Cirolemycin; Clarithromycin; Clinafloxacin Hydrochloride; Clindamycin; Clindamycin Hydrochloride; Clindamycin Palmitate Hydrochloride; Clindamycin Phosphate; Clofazimine; Cloxacillin Benzathine; Cloxacillin Sodium; Cloxyquin; Colistimethate Sodium; Colistin Sulfate; Coumermycin; Coumermycin Sodium; Cyclacillin; Cycloserine; Dalfopristin; Dapsone; Daptomycin; Demeclocycline; Demeclocycline Hydrochloride; Demecycline; Denofungin; Diaveridine; Dicloxacillin; Dicloxacillin Sodium; Dihydrostreptomycin Sulfate; Dipyrithione; Dirithromycin; Doxycycline; Doxycycline Calcium; Doxycycline Fosfatex; Doxycycline Hyclate; Droxacin Sodium; Enoxacin; Epicillin; Epitetracycline Hydrochloride; Erythromycin; Erythromycin Acistrate; Erythromycin Estolate; Erythromycin Ethylsuccinate; Erythromycin Gluceptate; Erythromycin Lactobionate; Erythromycin Propionate; Erythromycin Stearate; Ethambutol Hydrochloride; Ethionamide; Fleroxacin; Floxacillin; Fludalanine; Flumequine; Fosfomycin; Fosfomycin Tromethamine; Fumoxicillin; Furazolium Chloride; Furazolium Tartrate; Fusidate Sodium; Fusidic Acid; Gentamicin Sulfate; Gloximonam; Gramicidin; Haloprogin; Hetacillin; Hetacillin Potassium; Hexedine; Ibafloxacin; Imipenem; Isoconazole; Isepamicin; Isoniazid; Josamycin; Kanamycin Sulfate; Kitasamycin; Levofuraltadone; Levopropylcillin Potassium; Lexithromycin; Lincomycin; Lincomycin Hydrochloride; Lomefloxacin; Lomefloxacin Hydrochloride; Lomefloxacin Mesylate; Loracarbef; Mafenide; Meclocycline; Meclocycline Sulfosalicylate; Megalomicin Potassium Phosphate; Mequidox; Meropenem; Methacycline; Methacycline Hydrochloride; Methenamine; Methenamine Hippurate; Methenamine Mandelate; Methicillin Sodium; Metioprim; Metronidazole Hydrochloride; Metronidazole Phosphate; Mezlocillin; Mezlocillin Sodium; Minocycline; Minocycline Hydrochloride; Mirincamycin lydrochloride; Monensin; Monensin Sodium; Nafcillin Sodium; Nalidixate Sodium; Nalidixic Acid; Natamycin; Nebramycin; Neomycin Palmitate; Neomycin Sulfate; Neomycin Undecylenate; Netilmicin Sulfate; Neutramycin; Nifuradene; Nifuraldezone; Nifuratel; Nifuratrone; Nifurdazil; Nifurimide; Nifurpirinol; Nifurquinazol; Nifurthiazole; Nitrocycline; Nitrofurantoin; Nitromide; Norfloxacin; Novobiocin Sodium; Ofloxacin; Ormetoprim; Oxacillin Sodium; Oximonam; Oximonam Sodium; Oxolinic Acid; Oxytetracycline; Oxytetracycline Calcium; Oxytetracycline Hydrochloride; Paldimycin; Parachlorophenol; Paulomycin; Pefloxacin; Pefloxacin Mesylate; Penamecillin; Penicillin G Benzathine; Penicillin G Potassium; Penicillin G Procaine; Penicillin G Sodium; Penicillin V; Penicillin V Benzathine; Penicillin V Hydrabamine; Penicillin V Potassium; Pentizidone Sodium; Phenyl Aminosalicylate; Piperacillin Sodium; Pirbenicillin Sodium; Piridicillin Sodium; Pirlimycin Hydrochloride; Pivampicillin Hydrochloride; Pivampicillin Pamoate; Pivampicillin Probenate; Polymyxin B Sulfate; Porfiromycin; Propikacin; Pyrazinamide; Pyrithione Zinc; Quindecamine Acetate; Quinupristin; Racephenicol; Ramoplanin; Ranimycin; Relomycin; Repromicin; Rifabutin; Rifametane; Rifamexil; Rifamide; Rifampin; Rifapentine; Rifaximin; Rolitetracycline; Rolitetracycline Nitrate; Rosaramicin; Rosaramicin Butyrate; Rosaramicin Propionate; Rosaramicin Sodium Phosphate; Rosaramicin Stearate; Rosoxacil; Roxarsone; Roxithromycin; Sancycline; Sanfetrinem Sodium; Sarmoxicillin; Sarpicillin; Scopafungin; Sisomicin; Sisomicin Sulfate; Sparfloxacin; Spectinomycin Hydrochloride; Spiramycin; Stallimycin Hydrochloride; Steffimycin; Streptomycin Sulfate; Streptonicozid; Sulfabenz: Sulfabenzamide; Sulfacetamide; Sulfacetamide Sodium; Sulfacytine; Sulfadiazine; Sulfadiazine Sodium; Sulfadoxine; Sulfalene; Sulfamerazine; Sulfameter; Sulfamethazine; Sulfamethizole; Sulfamethoxazole; Sulfamonomethoxine; Sulfamoxole; Sulfanilate Zinc; Sulfanitran; Sulfasalazine; Sulfasomizole; Sulfathiazole; Sulfazamet; Sulfisoxazole; Sulfisoxazole Acetyl; Sulfisoxazole Diolamine; Sulfomyxin; Sulopenem; Sultamicillin; Suncillin Sodium; Talampicillin Hydrochloride; Teicoplanin; Temafloxacin Hydrochloride; Temocillin; Tetracycline; Tetracycline Hydrochloridc; Tetracycline Phosphate Complex; Tetroxoprim; Thiamphenicol; Thiphencillin Potassium; Ticarcillin Cresyl Sodium: Ticarcillin Disodium; Ticarcillin Monosodium; Ticlatone; Tiodonium Chloride; Tobramycin; Tobramycin Sulfate; Tosufloxacin; Trimethoprim; Trimethoprim Sulfate; Trisulfapyrimidines; Troleandomycin; Trospectomycin Sulfate; Tyrothricin; Vancomycin; Vancomycin Hydrochloride; Virginiamycin; Zorbamycin; Difloxacin Hydrochloride; Lauryl Isoquinolinium Bromide; Moxalactam Disodium; Ornidazole; Pentisomicin; and Sarafloxacin Hydrochloride.

Exemplary anti-viral agents include, but are not limited to, acemannan, acyclovir, acyclovir sodium, adefovir, alovudine, alvircept sudotox, amantadine hydrochloride, aranotin, arildone, atevirdine mesylate, avridine, cidofovir, cipamfylline, cytarabine hydrochloride, delavirdine mesylate, desciclovir, didanosine, disoxaril, edoxudine, enviradene, enviroxime, famciclovir, famotine hydrochloride, fiacitabine, fialuridine, fosarilate, foscamet sodium, fosfonet sodium, ganciclovir, ganciclovir sodium, idoxuridine, kethoxal, lamivudine, lobucavir, memotine hydrochloride, methisazone, nevirapine, oseltamivir phosphate, penciclovir, pirodavir, ribavirin, rimantadine hydrochloride, saquinavir mesylate, somantadine hydrochloride, sorivudine, statolon, stavudine, tilorone hydrochloride, trifluridine, valacyclovir hydrochloride, vidarabine, vidarabine phosphate, vidarabine sodium phosphate, viroxime, zalcitabine, zanamivir, zidovudine, and zinviroxime immunomodulatory agents (e.g., interferon), anti-inflammatory agents (e.g., adrenocorticoids, corticosteroids (e.g., beclomethasone, budesonide, flunisolide, fluticasone, triamcinolone, methylprednisolone, prednisolone, prednisone, hydrocortisone), glucocorticoids, steroids, and non-steroidal anti-inflammatory drugs (e.g., aspirin, ibuprofen, diclofenac, and COX-2 inhibitors), pain relievers, leukotreine antagonists (e.g., montelukast, methyl xanthines, zafirlukast, and zileuton), beta2-agonists (e.g., albuterol, biterol, fenoterol, isoetharie, metaproterenol, pirbuterol, salbutamol, terbutalin formoterol, salmeterol, and salbutamol terbutaline), anticholinergic agents (e.g., ipratropium bromide and oxitropium bromide), sulphasalazine, penicillamine, dapsone, antihistamines, anti-malarial agents (e.g., hydroxychloroquine), anti-viral agents (e.g., nucleoside analogs (e.g., zidovudine, acyclovir, gangcyclovir, vidarabine, idoxuridine, trifluridine, and ribavirin), foscarnet, amantadine, rimantadine, saquinavir, indinavir, ritonavir, and AZT).

Exemplary anti-fungal agents include, but are not limited to, clotrimazole, ketoconazole, nystatin, amphotericin, miconazole, bifonazole, butoconazole, clomidazole, croconazole, eberconazole, econazole, fenticonazole, flutimazole, isoconazole, ketoconazole, lanoconazole, luliconazole, neticonazole, omoconazole, oxiconazole, setraconazole, sulconazole, tioconazole, fluconazole, itraconazole, terconazole, terbinafine, natrifine, amorolfine, amphotericin B, nystatin, natamaycin, flucytosine, griseofulvin, potassium iodide, butenafine, ciclopirox, ciloquinol (iodochlorhydroxyquin), haloprogin, tolnaftate, aluminum chloride, undecylenic acid, potassium permanganate, selenium sulphide, salicylic acid, zinc pyruthione, bromochlorsalicylanilide, methylrosaniline, tribromometacresol, undecylenic acid, polynoxylin, 2-(4-chlorphenoxy)-ethanol, chlorophensesin, ticlatone, sulbentine, ethyl hydroxybenzoate, dimazole, tolciclate, and sulphacetamide.

Exemplary chemotherapeutics include, but are not limited to, cisplatin, etoposide, abiraterone acetate, altretamine, anhydrovinblastine, auristatin, bexarotene, bicalutamide, bleomycin, cachectin, cemadotin, chlorambucil, cyclophosphamide, caleukoblastine, docetaxol, doxetaxel, cyclophosphamide, carboplatin, carmustine (BCNU), cryptophycin, cyclophosphamide, cytarabine, dacarbazine (DTIC), dactinomycin, daunorubicin, dolastatin, doxorubicin (adriamycin), 5-fluorouracil, finasteride, flutamide, hydroxyurea and hydroxyureataxanes, ifosfamide, liarozole, lonidamine, lomustine (CCNU), mechlorethamine (nitrogen mustard), melphalan, mivobulin isethionate, rhizoxin, sertenef, streptozocin, mitomycin, methotrexate, 5-fluorouracil, nilutamide, onapristone, paclitaxel, prednimustine, procarbazine, RPR109881, stramustine phosphate, tamoxifen, tasonermin, taxol, tretinoin, vinblastine, vincristine, vindesine sulfate, and vinflunine.

In some embodiments, the pathogen is a protozoa, helminths, or ectoparasitic arthropods (e.g., ticks, mites, etc.). Protozoa are single celled organisms that can replicate both intracellularly and extracellularly, particularly in the blood, intestinal tract or the extracellular matrix of tissues. Helminths are multicellular organisms which almost always are extracellular (the exception being Trichinella). Helminths normally require exit from a primary host and transmission into a secondary host in order to replicate. In contrast to these aforementioned classes, ectoparasitic arthropods form a parasitic relationship with the external surface of the host body.

In some embodiments, the pathogens can be classified based on whether they are intracellular or extracellular. An “intracellular pathogen” as used herein is a pathogen whose entire life cycle is intracellular. Examples of human intracellular pathogens include Leishmania, Plasmodium, Trypanosoma cruzi, Toxoplasma gondii, Babesia, and Trichinella spiralis. An “extracellular parasite” as used herein is a pathogen whose entire life cycle is extracellular. Extracellular pathogens capable of infecting humans include Entamoeba histolytica, Giardia lamblia, Enterocytozoon bieneusi, Naegleria and Acanthamoeba as well as most helminths. Yet another class of pathogens is defined as being mainly extracellular but with an obligate intracellular existence at a critical stage in their life cycles. Such pathogens are referred to herein as “obligate intracellular parasites”. These parasites may exist most of their lives or only a small portion of their lives in an extracellular environment, but they all have at least one obligate intracellular stage in their life cycles. This latter category of parasites includes Trypanosoma rhodesiense and Trypanosoma gambiense, Isospora, Cryptosporidium, Eimeria, Neospora, Sarcocystis, and Schistosoma. In one aspect, the disclosure relates to the prevention and treatment of infection resulting from intracellular parasites and obligate intracellular parasites, which have at least in one stage of their life cycle that is intracellular. In some embodiments, the disclosure is directed to the prevention of infection from obligate intracellular parasites, which are predominantly intracellular. An exemplary and non-limiting list of parasites for some aspects of the invention is provided herein.

In some embodiments, the pathogen is a blood-borne pathogen. Blood-borne pathogens include Plasmodium, Babesia microti, Babesia divergens, Leishmania tropica, Leishmania, Leishmania braziliensis, Leishmania donovani, Trypanosoma gambiense and Trypanosoma rhodesiense (African sleeping sickness), Trypanosoma cruzi (Chagas' disease), and Toxoplasma gondii.

In some embodiments, the pathogen is a fungi. Examples of pathogenic fungi include, without limitation, Altemaria, Aspergillus, Basidiobolus, Bipolaris, Blastoschizomyces, Candida, Candida albicans, Candida krusei, Candida glabrata (formerly called Torulopsis glabrata), Candida parapsilosis, Candida tropicalis, Candida pseudotropicalis, Candida guilliermondii, Candida dubliniensis, and Candida lusitaniae, Coccidioides, Cladophialophora, Cryptococcus, Cunninghamella, Curvularia, Exophiala, Fonsecaea, Histoplasma, Madurella, Malassezia, Plastomyces, Rhodotorula, Scedosporium, Scopulariopsis, Sporobolomyces, Tinea, and Trichosporon.

In some embodiments, the pathogen is a fungi, including, but not limited to Candida. There are approximately 200 species of the genus Candida, but nine cause the great majority of human infections. They are C. albicans, C. krusei, C. glabrata (formerly called Torulopsis glabrata), C. parapsilosis, C. tropicalis, C. pseudotropicalis, C. guilliermondii, C. dubliniensis, and C. lusitaniae. They cause infections of the mucous membranes, for example, thrush, esophagitis, and vagititis; skin, for example, intertrigo, balanitis, and generalized candidiasis; blood stream infections, for example, candidemia; and deep organ infections, for example, hepatosplenic candidiasis, urinary tract candidiasis, arthritis, endocarditis, and endophthamitis.

Exemplary bacterial pathogens include, but are not limited to, Aerobacter, Aeromonas, Acinetobacter, Actinomyces israelli, Agrobacterium, Bacillus, Bacillus antracis, Bacteroides, Bartonella, Bordetella, Bortella, Borrelia, Brucella, Burkholderia, Calymmatobacterium, Campylobacter, Citrobacter, Clostridium, Clostridium perfringers, Clostridium tetani, Cornyebacterium, Corynebacterium diphtheriae, corynebacterium sp., Enterobacter, Enterobacter aerogenes, Enterococcus, Erysipelothrix rhusiopathiae, Escherichia, Francisella, Fusobacterium nucleatum, Gardnerella, Haemophilus, Hafnia, Helicobacter, Klebsiella, Klebsiella pneumoniae, Lactobacillus, Legionella, Leptospira, Listeria, Morganella, Moraxella, Mycobacterium, Neisseria, Pasteurella, Pasteurella multocida, Proteus, Providencia, Pseudomonas, Rickettsia, Salmonella, Serratia, Shigella, Staphylococcus, Stentorophomonas, Streptococcus, Streptobacillus moniliformis, Treponema, Treponema pallidium, Treponema pertenue, Xanthomonas, Vibrio, and Yersinia.

Examples of viruses that have been found in humans include but are not limited to: Retroviridae (e.g. human immunodeficiency viruses, such as HIV-1 (also referred to as HDTV-III, LAVE or HTLV-III/LAV, or HIV-III; and other isolates, such as HIV-LP; Picomaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

### Types of Samples

Pathogens (e.g., bacteria) can be characterized when present in a biological sample from a patient having a pathogen infection. The biological samples are generally derived from a patient in the form of a bodily fluid (such as blood, cerebrospinal fluid, phlegm, saliva, sputum, semen, vaginal secretion, or urine) or tissue sample (e.g. a tissue sample obtained by biopsy).

In other embodiments, the sample is an environmental sample (e.g., water sample, such as waste water, or soil sample). Environmental samples are used, for example, to monitor the accumulation of genetic alterations in a population of pathogens present in a building, school, or city.

### Monitoring Antimicrobial Resistance in a Subject or Population

This disclosure provides methods of identifying a subject having an infection or condition (e.g., cancer) that is resistant or sensitive to a therapeutic agent (e.g., antimicrobial, chemotherapeutic). The method includes the step of characterizing the sequence of a polynucleotide (e.g., antimicrobial resistance gene) in a biological sample obtained from the subject. In some embodiments, a subject is identified as having a bacterial infection that is resistant to a therapeutic agent if a mutation in a polynucleotide or polypeptide relative to a reference sequence is detected. In some embodiments, a subject is identified as having a bacterial infection that is sensitive to a therapeutic agent if a mutation in an antimicrobial resistance gene (e.g., NalD, OprD, MexR, AnmK, AmpD, SltB1, or PA0810) or polypeptide relative to a reference sequence is detected.

Diagnostic analysis of resistance status should be performed in patients who are receiving, have received, or are expected to receive therapy, particularly patients who are receiving antimicrobial therapy and have developed resistance to the antimicrobial, or patients receiving chemotherapy for a cancer that is developing resistance to chemotherapy. A subject identified as sensitive to an antimicrobial agent can be administered such agent. Over time, many patients treated with an antimicrobial agent acquire resistance to the therapeutic effects of the agent. The early identification of resistance to an antimicrobial in a patient can be important to patient survival because it allows for the selection of alternative therapies. Subjects identified as having an infection resistant to a therapeutic agent are identified as in need of alternative treatment.

Methods of monitoring the sensitivity or resistance to a therapeutic agent are useful in managing subject treatment. The results presented here provide evidence for clonal dominance and resistance caused by insertion, deletion, truncating, missense, gain of function, or loss of function mutations.

Thus, in some embodiments, alterations in a polynucleotide or polypeptide (e.g., sequence, level, biological activity) are analyzed before and again after subject management or treatment. In these cases, the methods are used to monitor the status of sensitivity to a therapeutic agent. The level, biological activity, or sequence of a polypeptide or polynucleotide may be assayed before treatment, during treatment, or following the conclusion of a treatment regimen. In some embodiments, multiple assays (e.g., 2, 3, 4, 5) are made at one or more of those times to assay resistance to a therapeutic agent (e.g., antimicrobial).

In some embodiments, methods of the disclosure include selecting a subject for antimicrobial resistance monitoring. A subject can be selected for monitoring based on whether the subject is receiving a treatment that may impact the subject's immune system, e.g., a chemotherapy treatment. The subject can be selected for monitoring based on the subject being associated with a cohort of subjects identified as infectious. For example, a group of subjects sharing a contaminated water source.

### Amplification and Hybridization

Once a biological sample comprising a pathogen is collected from a subject, the sample comprising the target polynucleotide(s) of interest can be subjected to one or more preparative reactions. These preparative reactions can include in vitro transcription (IVT), labeling, fragmentation, amplification and other reactions.

By “amplification” is meant any process of producing at least one copy of a nucleic acid, and in many cases producing multiple copies. An amplification product can be RNA or DNA, and may include a complementary strand to the expressed target sequence. DNA amplification products can be produced initially through reverse translation and then optionally from further amplification reactions. The amplification product may include all or a portion of a target sequence, and may optionally be labeled. A variety of amplification methods are suitable for use, including polymerase-based methods and ligation-based methods. Exemplary amplification techniques include the polymerase chain reaction method (PCR), the lipase chain reaction (LCR), ribozyme-based methods, self sustained sequence replication (3SR), nucleic acid sequence-based amplification (NASBA), the use of Q Beta replicase, reverse transcription, nick translation, and the like.

The first cycle of amplification in polymerase-based methods typically involves a primer extension product complementary to the template strand. The primers for a PCR must, of course, be designed to hybridize to regions in their corresponding template that can produce an amplifiable segment; thus, each primer must hybridize so that its 3′ nucleotide is paired to a nucleotide in its complementary template strand that is located 3′ from the 3′ nucleotide of the primer used to replicate that complementary template strand in the PCR.

The target polynucleotide can be amplified by contacting one or more strands of the target polynucleotide with a primer and a polymerase having suitable activity to extend the primer and copy the target polynucleotide to produce a full-length complementary polynucleotide or a smaller portion thereof. Any enzyme having a polymerase activity that can copy the target polynucleotide can be used, including DNA polymerases, RNA polymerases, reverse transcriptases, or enzymes having more than one type of polymerase or enzyme activity. The enzyme can be thermolabile or thermostable. Mixtures of enzymes can also be used.

Suitable reaction conditions are chosen to permit amplification of the target polynucleotide, including pH, buffer, ionic strength, presence and concentration of one or more salts, presence and concentration of reactants and cofactors such as nucleotides and magnesium and/or other metal ions (e.g., manganese), optional cosolvents, temperature, thermal cycling profile for amplification schemes comprising a polymerase chain reaction, and may depend in part on the polymerase being used as well as the nature of the sample. Cosolvents include formamide (typically at from about 2 to about 10%), glycerol (typically at from about 5 to about 10%), and DMSO (typically at from about 0.9 to about 10%). Techniques may be used in the amplification scheme in order to minimize the production of false positives or artifacts produced during amplification. These include “touchdown” PCR, hot-start techniques, use of nested primers, or designing PCR primers so that they form stem-loop structures in the event of primer-dimer formation and thus are not amplified. Techniques to accelerate PCR can be used, for example centrifugal PCR, which allows for greater convection within the sample, and comprising infrared heating steps for rapid heating and cooling of the sample. One or more cycles of amplification can be performed. An excess of one primer can be used to produce an excess of one primer extension product during PCR; preferably, the primer extension product produced in excess is the amplification product to be detected. A plurality of different primers may be used to amplify different target polynucleotides or different regions of a particular target polynucleotide within the sample.

An amplification reaction can be performed under conditions that allow an optionally labeled sensor polynucleotide to hybridize to the amplification product during at least part of an amplification cycle. When the assay is performed in this manner, real-time detection of this hybridization event can take place by monitoring for light emission or fluorescence during amplification, as known in the art.

### Primers

Primers based on the nucleotide sequences of target sequences (e.g., antibiotic resistance genes) can be designed for use in amplification of the target sequences. For use in amplification reactions such as PCR, a pair of primers can be used. The exact composition of the primer sequences is not critical to the invention, but for most applications the primers may hybridize to specific sequences of the probe set under stringent conditions, particularly under conditions of high stringency, as known in the art. The pairs of primers are usually chosen so as to generate an amplification product of at least about 50 nucleotides, more usually at least about 100 nucleotides. Algorithms for the selection of primer sequences are generally known, and are available in commercial software packages. These primers may be used in standard quantitative or qualitative PCR-based assays to assess transcript expression levels of RNAs defined by the probe set. Alternatively, these primers may be used in combination with probes, such as molecular beacons in amplifications using real-time PCR.

As is known in the art, a nucleoside is a base-sugar combination and a nucleotide is a nucleoside that further includes a phosphate group covalently linked to the sugar portion of the nucleoside. In forming oligonucleotides, the phosphate groups covalently link adjacent nucleosides to one another to form a linear polymeric compound, with the normal linkage or backbone of RNA and DNA being a 3′ to 5′ phosphodiester linkage. Specific examples of polynucleotide probes or primers useful in this disclosure include oligonucleotides containing modified backbones or non-natural internucleoside linkages. As defined in this specification, oligonucleotides having modified backbones include both those that retain a phosphorus atom in the backbone and those that lack a phosphorus atom in the backbone. For the purposes of the present disclosure, and as sometimes referenced in the art, modified oligonucleotides that do not have a phosphorus atom in their internucleoside backbone can also be considered to be oligonucleotides.

Exemplary polynucleotide primers having modified oligonucleotide backbones include, for example, those with one or more modified internucleotide linkages that are phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′ amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2-5′ to 5′-2′. Various salts, mixed salts and free acid fauns are also included.

Other modifications may also be made at other positions on the polynucleotide probes or primers, particularly the 3′ position of the sugar on the 3′ terminal nucleotide or in 2-5′ linked oligonucleotides and the 5′ position of 5′ terminal nucleotide. Polynucleotide probes or primers may also have sugar mimetics such as cyclobutyl moieties in place of the pentofuranosyl sugar.

Polynucleotide primers may also include modifications or substitutions to the nucleobase. As used herein, “unmodified” or “natural” nucleobases include the purine bases adenine (A) and guanine (G), and the pyrimidine bases thymine (T), cytosine (C) and uracil (U).

Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808; The Concise Encyclopedia Of Polymer Science And Engineering, (1990) pp 858-859, Kroschwitz, J. L, ed. John Wiley & Sons; Englisch et al., Angewandte Chemie, Int. Ed., 30:613 (1991); and Sanghvi, Y. S., (1993) Antisense Research and Applications, pp 289-302, Crooke, S. T. and Lebleu, B., ed., CRC Press. Certain of these nucleobases are particularly useful for increasing the binding affinity of the polynucleotide probes of the disclosure. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability.

One skilled in the art recognizes that it is not necessary for all positions in a given polynucleotide probe or primer to be uniformly modified. The present disclosure, therefore, contemplates the incorporation of more than one of the aforementioned modifications into a single polynucleotide probe or even at a single nucleoside within the probe or primer.

One skilled in the art also appreciates that the nucleotide sequence of the entire length of the polynucleotide probe or primer does not need to be derived from the target sequence. Thus, for example, the polynucleotide probe may comprise nucleotide sequences at the 5′ and/or 3′ termini that are not derived from the target sequences. Nucleotide sequences which are not derived from the nucleotide sequence of the target sequence may provide additional functionality to the polynucleotide probe. For example, they may provide a restriction enzyme recognition sequence or a “tag” that facilitates detection, isolation, purification or immobilization onto a solid support. Alternatively, the additional nucleotides may provide a self-complementary sequence that allows the primer/probe to adopt a hairpin configuration. Such configurations are necessary for certain probes, for example, molecular beacon and Scorpion probes, which can be used in solution hybridization techniques.

The polynucleotide primers can incorporate moieties useful in detection, isolation, purification, or immobilization, if desired. Such moieties are well-known in the art (see, for example, Ausubel et al., (1997 & updates) Current Protocols in Molecular Biology, Wiley & Sons, New York) and are chosen such that the ability of the probe to hybridize with its target sequence is not affected.

Examples of suitable moieties are detectable labels, such as radioisotopes, fluorophores, chemiluminophores, enzymes, colloidal particles, and fluorescent microparticles, as well as antigens, antibodies, haptens, avidin/streptavidin, biotin, haptens, enzyme cofactors/substrates, enzymes, and the like.

A label can optionally be attached to or incorporated into a probe or primer polynucleotide to allow detection and/or quantitation of a target polynucleotide representing the target sequence of interest. The target polynucleotide may be the expressed target sequence RNA itself, a cDNA copy thereof, or an amplification product derived therefrom, and may be the positive or negative strand, so long as it can be specifically detected in the assay being used. Similarly, an antibody may be labeled.

In certain multiplex formats, labels used for detecting different targets may be distinguishable. The label can be attached directly (e.g., via covalent linkage) or indirectly, e.g., via a bridging molecule or series of molecules (e.g., a molecule or complex that can bind to an assay component, or via members of a binding pair that can be incorporated into assay components, e.g. biotin-avidin or streptavidin). Many labels are commercially available in activated forms which can readily be used for such conjugation (for example through amine acylation), or labels may be attached through known or determinable conjugation schemes, many of which are known in the art.

Labels useful in the disclosure described herein include any substance which can be detected when bound to or incorporated into the biomolecule of interest. Any effective detection method can be used, including optical, spectroscopic, electrical, piezoelectrical, magnetic, Raman scattering, surface plasmon resonance, colorimetric, calorimetric, etc. A label is typically selected from a chromophore, a lumiphore, a fluorophore, one member of a quenching system, a chromogen, a hapten, an antigen, a magnetic particle, a material exhibiting nonlinear optics, a semiconductor nanocrystal, a metal nanoparticle, an enzyme, an antibody or binding portion or equivalent thereof, an aptamer, one member of a binding pair, and combinations thereof. Quenching schemes may be used, wherein a quencher and a fluorophore as members of a quenching pair may be used on a probe, such that a change in optical parameters occurs upon binding to the target to introduce or quench the signal from the fluorophore. One example of such a system is a molecular beacon. Suitable quencher/fluorophore systems are known in the art. The label may be bound through a variety of intermediate linkages. For example, a polynucleotide may comprise a biotin-binding species, and an optically detectable label may be conjugated to biotin and then bound to the labeled polynucleotide. Similarly, a polynucleotide sensor may comprise an immunological species such as an antibody or fragment, and a secondary antibody containing an optically detectable label may be added.

Chromophores useful in the methods described herein include any substance that can absorb energy and emit light. For multiplexed assays, a plurality of different signaling chromophores can be used with detectably different emission spectra. The chromophore can be a lumophore or a fluorophore. Typical fluorophores include fluorescent dyes, semiconductor nanocrystals, lanthanide chelates, polynucleotide-specific dyes and green fluorescent protein.

Polynucleotides from the described target sequences may be employed as probes for detecting target sequences expression, for ligation amplification schemes, or may be used as primers for amplification schemes of all or a portion of target sequences. When amplified, either strand produced by amplification may be provided in purified and/or isolated form.

Complements may take any polymeric form capable of base pairing to the species recited herein, including nucleic acids such as RNA or DNA, or may be a neutral polymer such as a peptide nucleic acid. Polynucleotides of the disclosure can be selected from the subsets of the recited nucleic acids described herein, as well as their complements.

### Preparation of Primers

The polynucleotide primers of the present disclosure can be prepared by conventional techniques well-known to those skilled in the art. For example, the polynucleotide primers can be prepared using solid-phase synthesis using commercially available equipment. As is well-known in the art, modified oligonucleotides can also be readily prepared by similar methods. The polynucleotide probes can also be synthesized directly on a solid support according to methods standard in the art.

### Sequencing and Analysis

In embodiments, the methods disclosed herein involve sequencing genomic DNA obtained from biological samples. In embodiments, the method for sequencing the genomic DNA does not involve culturing a cell (e.g., bacterial cell) comprising the DNA prior to amplifying and sequencing.

In embodiments of the methods provided herein, next-generation sequencing (NGS) of genomic DNA from cells from a sample allows for capture of alterations in the sequence relative to the sequence of, e.g., a reference genome. The methods of the disclosure enable disease monitoring for patients in the clinic or in a hospital setting at regular intervals. Methods of this disclosure further include third-generation sequencing of genomic DNA. For example, using a sequencing platform sold under the trade name Pacific Biosciences or Oxford Nanopore Technologies. Third generation sequencing technologies are useful for constructing whole genome sequences, as such technologies can generate long sequence reads (e.g., greater than 300 base pairs).

Any suitable method for isolation of DNA may be used in the methods of the disclosure (e.g., proteinase K-based purification methods). Various kits are commercially available for the purification of polynucleotides from a sample and are suitable for use in the methods of the disclosure (e.g., an Arcturus PicoPure DNA Extraction Kit, Thermo Fisher Scientific). In an embodiment, the genomic DNA is purified using a proteinase K digestion-based technique (e.g., Arcturus PicoPure DNA Extraction Kit, Thermo Fisher Scientific)

The extracted DNA may be sequenced using any high-throughput platform. Methods of sequencing oligonucleotides and nucleic acids are well known in the art (see, e.g., WO93/23564, WO98/28440 and WO98/13523; U.S. Pat. App. Pub. No. 2019/0078232; U.S. Pat. Nos. 5,525,464; 5,202,231; 5,695,940; 4,971,903; 5,902,723; 5,795,782; 5,547,839 and 5,403,708; Sanger et al., Proc. Natl. Acad. Sci. USA 74:5463 (1977); Drmanac et al., Genomics 4:114 (1989); Koster et al., Nature Biotechnology 14:1123 (1996); Hyman, Anal. Biochem. 174:423 (1988); Rosenthal, International Patent Application Publication 761107 (1989); Metzker et al., Nucl. Acids Res. 22:4259 (1994); Jones, Biotechniques 22:938 (1997); Ronaghi et al., Anal. Biochem. 242:84 (1996); Ronaghi et al., Science 281:363 (1998); Nyren et al., Anal. Biochem. 151:504 (1985); Canard and Arzumanov, Gene 11:1 (1994); Dyatkina and Arzumanov, Nucleic Acids Symp Ser 18:117 (1987); Johnson et al., Anal. Biochem. 136:192 (1984); and Elgen and Rigler, Proc. Natl. Acad. Sci. USA 91(13):5740 (1994), all of which are expressly incorporated herein by reference).

Identification of low frequency or rare mutations involves, in some embodiments, high average read depth, such that a low frequency mutation is distinguished from an error as the number of correct reads outnumbers any individual errors that may occur, rendering them statistically irrelevant. Sequencing depth typically ranges from 80× to up to thousands, or even millions-fold coverage (e.g., 100, 1,000, 10,000, 20,000, 50,000, 100,000, 250,000, 500,000, 1,000,000, 250,000,000).

Identification of low frequency or rare mutations involves, in some embodiments, the use of deep sequencing. In some embodiments, accuracy of variant calling is affected by sequence quality, uniformity of coverage and the threshold of false-discovery rate that is used. Sequence depth influences the accuracy by which rare events can be quantified in RNA sequencing, chromatin immunoprecipitation followed by sequencing (ChIP-seq) and other quantification-based assays. Deep sequencing and related technologies are known in the art and described, for example, by Sims et al., Nature Reviews Genetics 15:121-132, 2014; Petrackova https://doi.org/10.3389/fonc.2019.00851; Shendure and Ji. “Next-generation DNA sequencing”, Nature Biotechnology, 26(10):1135-1145 (2008)).

In some embodiments, the terms “next-generation DNA sequencing” (“NGS”), “high-throughput sequencing”, “massively parallel sequencing” and “deep sequencing” refer to a method of sequencing a plurality of nucleic acids in parallel. See e.g., Bentley et al, Nature 2008, 456:53-59. The leading commercially available platforms produced by Roche/454 (Margulies et al, 2005a), Illumina/Solexa (Bentley et al, 2008), Life/APG (SOLiD) (McKernan et al, 2009) and Pacific Biosciences (Eid et al, 2009) may be used for deep sequencing.

The sequencing of a polynucleotide can be carried out using any suitable commercially available sequencing technology. In another embodiment, the sequencing of a polynucleotide is carried out using chain termination method of DNA sequencing (e.g., Sanger sequencing). In yet another embodiment, commercially available sequencing technology is a next-generation sequencing technology, including as non-limiting examples combinatorial probe anchor synthesis (cPAS), DNA nanoball sequencing, droplet-based or digital microfluidics, heliscope single molecule sequencing, nanopore sequencing (e.g., Oxford Nanopore technologies), GeneGap sequencing, massively parallel signature sequencing (MPSS), microfluidic Sanger sequencing, microscopy-based techniques (e.g., transmission electronic microscopy DNA sequencing), RNA polymerase (RNAP) sequencing, single-molecule real-time (SMRT) sequencing, SOLiD sequencing, ion semiconductor sequencing, polony sequencing, Pyrosequencing (454), sequencing by hybridization, sequencing by synthesis (e.g., Illumina™ sequencing), sequencing with mass spectrometry, and tunneling currents DNA sequencing. In embodiments, the polynucleotide is sequenced using HiSeq2500 or Novaseq6000.

RNA sequencing (RNA-Seq) is a powerful tool for transcriptome profiling. In embodiments, to mitigate sequence-dependent bias resulting from amplification complications to allow truly digital RNA-Seq, a set of unique molecular marker identification sequences can be used to ensure that every cDNA molecule prepared from an mRNA sample is uniquely labeled. In other embodiments, a molecular barcode is used (see, e.g., Shiroguchi K, et al. Proc Natl Acad Sci USA. 2012 Jan. 24; 109(4):1347-52). After PCR, paired-end deep sequencing can be applied. Rather than counting the number of reads, RNA abundance can be measured based on the number of unique sequences observed for a given cDNA sequence. The barcodes may be optimized to be unambiguously identifiable.

In embodiments, the amplicon sequencing is to a coverage of about or at least about 10×, 20×, 30×, 40×, 50×, 60×, 70×, 80×, 90×, 100×, 200×, 500×, 1000×, 2000×, or more, where a sequencing coverage of 0.01 indicates that a DNA sample has been sequenced such that the amount of DNA sequenced is equivalent in size to about 1% of the corresponding amplicon from which the DNA sample is derived. In embodiments, the sequencing is to a coverage of no more than about 0.001, 0.01, 0.05, 0.1, 0.2, 0.3, 0.4, 0.5, 0.75, 1, 2, 3, 4, 5, 6, 7, 8, 9, 10, 20, 30, 40, 50, 60, 70, 80, 90, or 100×.

In some embodiments, methods of this disclosure involve identifying microbial nucleic acids from a biological sample containing mostly human nucleic acids. For example, in some instances the amount of human nucleic acids present in the sample is at least 1000-fold greater than the amount of microbial nucleic acids present. Methods for identifying microbial nucleic acids from biological samples containing mostly human nucleic acids can involve targeted amplification. For example, in some embodiments, methods involve binding primers having sequences specific to microbial nucleic acids, e.g., DNA sequences flanking a resistance mutation, and performing one or more PCR reactions to amplify the microbial nucleic acid. Using PCR, the microbial nucleic acids can be amplified substantially. For example, in some embodiments, the microbial nucleic acid is amplified 1, 2, 3, 4, 5, 6, 7, 8, 9, 10, 20, 30, 40, 50, 60, 70, 80, 90, 100, 200, 500, 1000-fold relative to the human nucleic acids present in the sample. The amplified nucleic acids can then be sequenced, providing for deep sequencing of target amplicons.

In embodiments, the methods of the disclosure further involve analyzing sequence data obtained through the sequencing of a polynucleotide and/or sequencing library. The analysis can involve the detection of clinically relevant events, such as mutations, single nucleotide variation, and/or chromosomal rearrangements associated with antibiotic resistance.

The sequence data obtained according to the methods of the disclosure allows for the detection of genetic alterations in genomic DNA of, for example, pathogens (e.g., bacteria) present in a biological sample of a subject undergoing antibiotic therapy, or present in another cell or organism undergoing selective pressure.

### Hardware and Software

The present disclosure also relates to a computer system involved in carrying out the methods of the disclosure relating to both computations and sequencing. The methods described herein, analyses can be performed on general-purpose or specially-programmed hardware or software. One can then record the results (e.g., characterization of a mutation) on tangible medium, for example, in computer-readable format such as a memory drive or disk or simply printed on paper, displayed on a monitor (e.g., a computer screen, a smart device, a tablet, a television screen, or the like), or displayed on any other visible medium. The results also could be reported on a computer screen.

In aspects, the analysis is performed by an algorithm. The analysis of sequences will generate results that are subject to data processing. Data processing can be performed by the algorithm. One of ordinary skill can readily select and use the appropriate software and/or hardware to analyze a sequence.

In aspects, the analysis is performed by a computer-readable medium. The computer-readable medium can be non-transitory and/or tangible. For example, the computer readable medium can be volatile memory (e.g., random access memory and the like) or non-volatile memory (e.g., read-only memory, hard disks, floppy discs, magnetic tape, optical discs, paper table, punch cards, and the like).

Data can be analyzed with the use of a programmable digital computer. The computer program analyzes the sequence data to indicate alterations (e.g., aneuploidy, translocations, and/or MM driver mutations) observed in the data. In aspects, software used to analyze the data can include code that applies an algorithm to the analysis of the results. The software can also use input data (e.g., sequence) to characterize mutations.

A computer system (or digital device) may be used to receive, transmit, display and/or store results, analyze the results, and/or produce a report of the results and analysis. A computer system may be understood as a logical apparatus that can read instructions from media (e.g. software) and/or network port (e.g. from the internet), which can optionally be connected to a server having fixed media. A computer system may comprise one or more of a CPU, disk drives, input devices such as keyboard and/or mouse, and a display (e.g. a monitor). Data communication, such as transmission of instructions or reports, can be achieved through a communication medium to a server at a local or a remote location. The communication medium can include any means of transmitting and/or receiving data. For example, the communication medium can be a network connection, a wireless connection, or an internet connection. Such a connection can provide for communication over the World Wide Web. It is envisioned that data relating to the present disclosure can be transmitted over such networks or connections (or any other suitable means for transmitting information, including but not limited to mailing a physical report, such as a printout) for reception and/or for review by a receiver. The receiver can be but is not limited to an individual, or electronic system (e.g. one or more computers, and/or one or more servers).

In some embodiments, the computer system may comprise one or more processors. Processors may be associated with one or more controllers, calculation units, and/or other units of a computer system, or implanted in firmware as desired. If implemented in software, the routines may be stored in any computer readable memory such as in RAM, ROM, flash memory, a magnetic disk, a laser disk, or other suitable storage medium. Likewise, this software may be delivered to a computing device via any known delivery method including, for example, over a communication channel such as a telephone line, the internet, a wireless connection, etc., or via a transportable medium, such as a computer readable disk, flash drive, etc. The various steps may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

A client-server, relational database architecture can be used in embodiments of the disclosure. A client-server architecture is a network architecture in which each computer or process on the network is either a client or a server. Server computers are typically powerful computers dedicated to managing disk drives (file servers), printers (print servers), or network traffic (network servers). Client computers include PCs (personal computers) or workstations on which users run applications, as well as example output devices as disclosed herein. Client computers rely on server computers for resources, such as files, devices, and even processing power. In some embodiments of the disclosure, the server computer handles all of the database functionality. The client computer can have software that handles all the front-end data management and can also receive data input from users.

A machine readable medium which may comprise computer-executable code may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The subject computer-executable code can be executed on any suitable device which may comprise a processor, including a server, a PC, or a mobile device such as a smartphone or tablet. Any controller or computer optionally includes a monitor, which can be a cathode ray tube (“CRT”) display, a flat panel display (e.g., active matrix liquid crystal display, liquid crystal display, etc.), or others. Computer circuitry is often placed in a box, which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard, mouse, or touch-sensitive screen, optionally provide for input from a user. The computer can include appropriate software for receiving user instructions, either in the form of user input into a set of parameter fields, e.g., in a GUI, or in the form of preprogrammed instructions, e.g., preprogrammed for a variety of different specific operations.

A computer can transform data into various formats for display. A graphical presentation of the results of a calculation (e.g., sequencing results) can be displayed on a monitor, display, or other visualizable medium (e.g., a printout). In some embodiments, data or the results of a calculation may be presented in an auditory form.

### Kits

The disclosure also provides kits for use in characterizing a biological sample from a subject. Kits of the instant disclosure may include one or more containers comprising an agent for characterization of mutations (e.g., antibiotic resistance mutations). In some embodiments, the kits further include instructions for use in accordance with the methods of this disclosure. In some embodiments, these instructions comprise a description of use of the agent to characterize antibiotic resistance mutations. In some embodiments, the instructions comprise a description of how to isolate polynucleotides from a sample, to carry out deep sequencing on amplicons, or to select an appropriate antibiotic therapy. The kit may further comprise a description of how to analyze and/or interpret data.

Instructions supplied in the kits of the instant disclosure are typically written instructions on a label or package insert (e.g., a paper sheet included in the kit), but machine-readable instructions (e.g., instructions carried on a magnetic or optical storage disk) are also acceptable. Instructions may be provided for practicing any of the methods described herein.

The kits of this disclosure are in suitable packaging. Suitable packaging includes, but is not limited to, vials, bottles, jars, flexible packaging (e.g., sealed Mylar or plastic bags), and the like. Kits may optionally provide additional components such as buffers and interpretive information. Normally, the kit comprises a container and a label or package insert(s) on or associated with the container.

The practice of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the disclosure, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the disclosure, and are not intended to limit the scope of what the inventors regard as their invention.

### EXAMPLES

### Example 1. Prospective Study of P. aeruginosa Populations During Acute Respiratory Infections

A prospective study was conducted of mechanically ventilated patients with clinical evidence of acute respiratory tract infection in the pediatric or cardiac intensive care unit at Boston Children's Hospital. Eighty-seven patients were screened to identify 49 patients that met the inclusion criteria, of which 31 patients consented to enrollment (FIG. 1A). Endotracheal or tracheal aspirates (referred here throughout as sputum samples) were collected at the onset of symptoms (‘sputum day 1’), with serial samples (‘sputum follow-up’) collected when possible. First, a small pilot study was conducted to assess the genomic diversity of P. aeruginosa in two patients, patients A and E*, who were sampled only at day 1. After confirming population growth and detectable diversity, short-term infection dynamics were studied in 7 patients whose serial samples were collected 4-11 days after day 1 and exhibited P. aeruginosa growth at both time points as the predominant pathogen (FIG. 1B; FIG. 5; Table 1).

TABLE 1
Exemplary short-term infection dynamics of P. aeruginosa

| | | | | | Time from most | | Duration of |
|---|---|---|---|---|---|---|---|
| | | | | Time from 1st | recent | | any artificial |
| | Age | | P. | P. aeruginosa | P. aeruginosa | Type of artificial | airway |
| | (approx., | | aeruginosa | infection to | infection to | airway at Day 1 | before Day 1 |
| Patient | years)* | Chronic conditions | history | Day 1 sample | Day 1 sample | sample | sample |
| A | 2-5 | Lysosomal storage disease, | New | N/A | N/A | Tracheostomy | 6 months |
| | | cardiomyopathy | | | | | |
| B | 0.5-2 | Epilepsy, brain malformation | New | N/A | N/A | ETT | 1 day |
| | | (Dandy Walker) | | | | | |
| C | 0.5-2 | Trisomy 21, congenital heart | New | N/A | N/A | Tracheostomy | 6 weeks |
| | | disease, tracheomalacia | | | | | |
| D | 2-5 | Congenital heart disease | New | N/A | N/A | ETT | 3 days |
| E | 0.08-0.5 | Repaired esophageal [symbol] | | <2 months | 11 days | ETT | 3 weeks |
| | | and [symbol] | | | | | |
| F | >21 | Congenital heart disease | Prior | Unknown | 18 days | Tracheostomy | 8 weeks |
| G | 2-5 | Pulmonary hypertension, | Prior | 2 years | 5 weeks | Tracheostomy | 10 weeks |
| | | immunodeficiency | | | | | |
| H | 2-5 | Multiple congenital [symbol] , | Prior | 3 months | Unknown | Tracheostomy | 12 weeks |
| | | congenital heart disease. | | (estimated) | | | |
| | | hypoxic [symbol] encephalopathy | | up to 4 years | | | |
| I | 6-12 | Epilepsy, brain malformation | Prior | 8 years | [symbol] months | ETT | 3 day |
| | | ( [symbol] ) | | | | | |

Exemplary short-term infection dynamics of P. aeruginosa

| | Duration of | P. | | | |
|---|---|---|---|---|---|
| | tracheostomy | aeruginosa | | | Days of |
| | before | load on | PMN on | | ventilation |
| | Day 1 | Day 1 | Day 1 | Infection | after |
| Patient | sample | sample¹ | sample² | type | Day 1³ |
| A | 6 months | Few | Abundant | Pneumonia | 50 |
| | | | | and RSV | |
| B | N/A | Moderate | Moderate | Pneumonia | >300 |
| C | 3 weeks | Few | Few/Moderate⁵ | Tracheitis | >265 |
| D | N/A | | Moderate | Tracheitis | 11 |
| E | N/A | Moderate | Moderate | Tracheitis | 14 |
| F | 2 weeks | Moderate | Few | Tracheitis | 17 |
| G | 7 weeks | Abundant | Abundant | Tracheitis | >7 |
| H | 9 weeks | Abundant | Moderate | Tracheitis | 16 |
| | | | | (2nd sample) | |
| I | N/A | Moderate | Moderate | Tracheitis and | 13 |
| | | | | RSV | |

Abbreviations:
ETT, endotracheal tube;
PMN, polymorphonuclear leukocyte. [symbol] 55% female.
¹For semiquantitative culture results, abundant is approximately ≥ 10\hat{ }6 CFU/mL: moderate is ≥10\hat{ }5 CFU/mL. few is ≥ 10\hat{ }4 CFU/mL, and rare is ≥ 10\hat{ }3 CFU/mL.
²For semiquantitative Gram stain results, abundant = >25,moderate = 10-25, few = 1-9, and rare = <1 PMN per low power field.
³Days of ventilation were not available for some patients transferred to other hospitals (listed as > x days).
⁴Presumed (culture at outside hospital 11 days prior to 1st sample grew mixed bacteria. not spostated).
⁵Moderate PMN on sample sent 4 days later. [symbol]
indicates data missing or illegible when filed

In addition, as GI tract carriage is thought to be a source of intra-patient infection, stool was also collected if available, of which only 2 of 4 available samples exhibited P. aeruginosa growth. Among the 9 patients (7 serially sampled and 2 pilot study patients), 4 had no history of P. aeruginosa infection (patients A-D) while 5 had a documented history of prior P. aeruginosa infection (denoted by an asterisk, patients E*-I*). In total, 18 sputum and 2 stool samples were collected across 9 patients from the onset of infection.

### Example 2. Maximizing the Detection of Genomic Diversity by Constructing Patient-Specific Reference Genomes

To capture the full extent of genomic diversity in pathogens, both long-read and short-read sequencing were used to characterize the P. aeruginosa populations in each patient. P. aeruginosa has a flexible pangenome with variations in gene content across strains by up to 50%. A poor choice in the reference genome would impact the alignment rate of short reads and therefore, the fraction of usable reads for identifying within-patient polymorphisms. Thus, a complete patient-specific reference genome using long-read sequencing of a single P. aeruginosa colony per patient (FIG. 1C) was assembled, which supported that each patient was infected with a unique strain based on gene content (FIG. 6A). To capture within-patient diversity, 24 additional cultured isolates from each sputum or stool (n=420 total) were collected, sequenced their whole genomes with short-reads, and aligned these reads to the patient-specific reference genomes (average alignment rate >99%, FIG. 6B) in order to identify within-patient single nucleotide polymorphisms (SNPs) and short insertions and deletions (indels) (FIG. 6C; Table 1). The within-patient variants (SNPs and indels) were used to construct patient-specific phylogenies of P. aeruginosa populations and to infer the most recent common ancestor (MRCA) in each patient.

### Example 3. New Infections Start with Clonal Founders

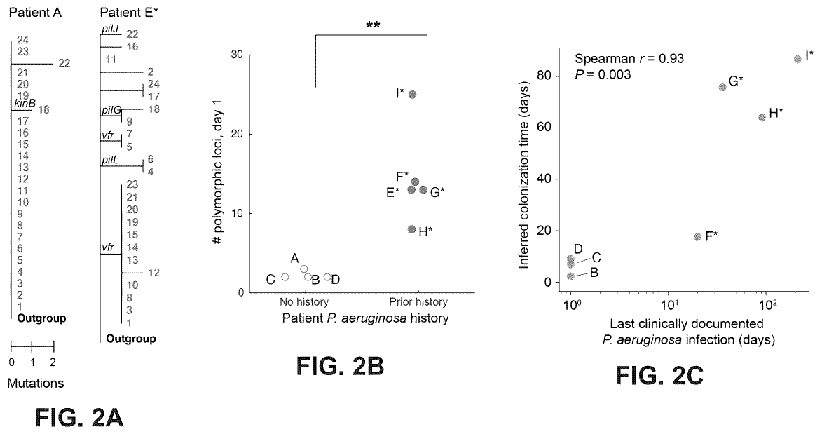

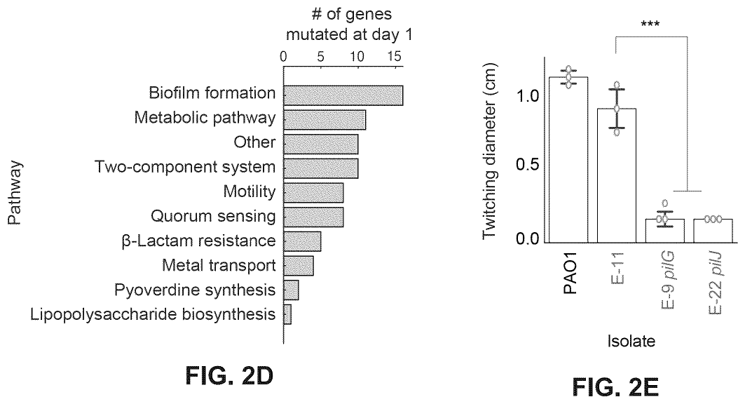

The diversity of pathogens at the onset of infection depended on the infection history of patients. Comparing the initial diversity of pathogens in the pilot patients A and E* suggested two contrasting day 1 populations. In the case of a presumed new infection in patient A, the population was nearly clonal that was consistent with recent colonization by a single founder (FIG. 2A, left). In contrast, the day 1 population was polymorphic in patient E*, who had a documented history of P. aeruginosa infection (FIG. 2A, right). Testing whether initial pathogen diversity—defined as the frequency of polymorphisms at day 1—differed based on infection history across all patients indeed revealed that patients with prior infection had higher initial diversity (FIG. 2B; P=0.007, two-sided t-test). Suspecting that pathogens were maintained in patients from prior infections, the inferred time of colonization within each patient based on genomic data was compared to the time since the last clinically documented P. aeruginosa infection, which showed a significant relationship (FIG. 2B; Spearman r=0.93, P=0.003) provided evidence that pathogen reservoirs were maintained between symptomatic episodes that resembles a chronic infection. Consistently, mutations at day 1 were found in genes and pathways important for colonization, such as biofilm formation (FIG. 2D; Table 2) and impairment in motility (FIG. 2E, P<0.0001). Altogether, these results show that new infections are colonized by a single clonal founder, and that once colonized, pathogen reservoirs can be maintained in patients between symptomatic episodes that resembles sub-chronic infection.

TABLE 2
Exemplary gene and pathway mutations and colonization of patients
All within-patient mutations.

| Patient | Type | Locustag | Pos | Ref | Mut | AAmut | Gene | Annot |
|---|---|---|---|---|---|---|---|---|
| A | N | PABCH05_ | 251 | C | T | A251V | PA2707 | MoxR family ATPase AAA, |
| | | 02289 | | | | | | modulator of stress response |
| A | N | PABCH05_ | 141 | G | T | G141C | glcD | Glycolate oxidase subunit |
| | | 05578 | | | | | | |
| A | N | PABCH05_ | 393 | G | T | G393V | kinB | Alginate biosynthesis sensor protein |
| | | 05714 | | | | | | |
| B | N | PABCH13_ | 141 | G | A | G141S | sulP | Sulfate transporter |
| | | 03537 | | | | | | |
| B | S | PABCH13_ | 206 | A | G | G206 | | Uracil-DNA glycosylase |
| | | 05028 | | | | | | |
| B | INDEL | | 1:3129522 | AT | A | | | |
| C | INDEL | PABCH14_ | 47 | A | AG | R47 | wbpL | glycosyl transferase |
| | | 02010 | | | | | | |
| C | INDEL | PABCH14_ | 250 | G | GA | E250 | | hypothetical protein |
| | | 01385 | | | | | | |
| D | N | PABCH09_ | 67 | G | A | R67H | csgA | short chain dehydrogenase |
| | | 00923 | | | | | | |
| D | N | PABCH09_ | 60 | C | A | A60E | trmJ | tRNA (cytidine/uridine-2′-O-)- |
| | | 01186 | | | | | | methyltransferase |
| D | N | PABCH09_ | 137 | C | G | N137K | PA3470 | 8-oxo-dGTP pyrophosphatase/DNA |
| | | 01523 | | | | | | mismatch repair protein |
| D | P | | -99- | G | A | | | DUF4124 domain-containing protein |
| | | | PABCH09_ | | | | | |
| | | | 04592 | | | | | |
| D | S | PABCH09_ | 31 | A | G | L31 | msbA | Lipid A export ATP-binding/permease |
| | | 05421 | | | | | | protein |
| D | N | PABCH09_ | 232 | G | A | R232H | mutY | A/G-specific adenine glycosylase |
| | | 05572 | | | | | | |
| D | INDEL | PABCH09_ | 400 | A | AC | Q400 | oprD | outer membrane porin OprD |
| | | 04255 | | | | | | |
| D | INDEL | PABCH09_ | 157 | CA | A | H157 | ampD | beta-lactamase expression regulator |
| | | 04792 | | | | | | AmpD |
| D | INDEL | PABCH09_ | 177 | G | GTC | V177 | sltB1 | soluble lytic transglycosylase B |
| | | 01001 | | | | | | |
| E | N | PABCH45_ | 1984 | C | T | Q1984* | pilL | Chemotactic signal transduction protein |
| | | 00431 | | | | | | |
| E | N | PABCH45_ | 163 | G | A | A163T | vfr | Transcriptional regulator Vfr, global |
| | | 00703 | | | | | | virulence factor |
| E | N | PABCH45_ | 355 | T | C | F355L | czcA | Heavy metal efflux protein |
| | | 02996 | | | | | | |
| E | P | | -69: | T | C | | | Hydrolase/non-heme chloroperoxidase |
| | | | PABCH45_ | | | | | |
| | | | 03200 | | | | | |
| E | N | PABCH45_ | 232 | G | A | W232* | PA3052 | hypothetical protein |
| | | 03661 | | | | | | |
| E | N | PABCH45_ | 149 | A | G | S149G | | UDP-glucose/GDP-mannose |
| | | 03814 | | | | | | dehydrogenase family |
| E | N | PABCH45_ | 390 | C | T | Q390* | cyaB | Adenylate cyclase for cAMP synthesis |
| | | 03873 | | | | | | |
| E | I | | 1:4479447 | A | G | | | |
| E | N | PABCH45_ | 484 | C | T | Q484* | | hypothetical protein |
| | | 04514 | | | | | | |
| E | INDEL | PABCH45_ | 79 | A | TA | E79 | vfr | Transcriptional regulator Vfr, global |
| E | | 00703 | | | | | | virulence factor |
| E | INDEL | PABCH45_ | 27 | G | GA | L27 | pilG | Twitching motility protein PilG |
| | | 00426 | | | | | | |
| E | INDEL | | 1:4694472 | G | GC | | | |
| E | INDEL | PABCH45_ | 205 | GGT | G | S205 | pilJ | Twitching motility protein PilJ |
| | | 00429 | | | | | | |
| F | N | PABCH42_ | 135 | G | A | D135N | ampR | HTH-type transcriptional activator |
| | | 00239 | | | | | | AmpR |
| F | N | PABCH42_ | 130 | A | C | T130P | mexR | Multidrug resistance operon repressor |
| | | 00712 | | | | | | |
| F | N | PABCH42_ | 131 | G | T | R131L | mexR | Multidrug resistance operon repressor |
| | | 00712 | | | | | | |
| F | S | PABCH42_ | 137 | C | A | L137 | wbpW | Phosphomannose isomerase/GDP- |
| | | 01410 | | | | | | mannose |
| F | N | PABCH42_ | 85 | C | T | Q85* | dipA | Bifunctional diguanylate |
| | | 01867 | | | | | | cyclase/phosphodiesterase |
| F | N | PABCH42_ | 776 | A | C | T776P | retS | Type III and type VI secretion switch |
| | | 02035 | | | | | | regulator |
| F | S | PABCH42_ | 150 | C | A | L150 | | putative cadaverine/lysine antiporter |
| | | 02091 | | | | | | |
| F | S | PABCH42_ | 85 | C | T | N85 | dapB | Dihydrodipicolinate reductase |
| | | 02138 | | | | | | |
| F | N | PABCH42_ | 164 | C | T | R164C | ampD | Beta-lactamase expression regulator |
| | | 02520 | | | | | | |
| F | S | PABCH42_ | 171 | C | G | R171 | petD | Cytochrome b |
| | | 02613 | | | | | | |
| F | N | PABCH42_ | 327 | C | A | R327S | bifA | Cyclic-di-GMP phosphodiesterase |
| | | | | | | | | inversely regulating biofilm |
| | | 02677 | | | | | | formation and swarming motility |
| F | N | PABCH42_ | 120 | T | A | L120Q | PA0810 | Haloacid dehalogenase, type II |
| | | 02989 | | | | | | |
| F | S | PABCH42_ | 228 | G | A | L228 | shaC | Na(+)/H(+) antiporter subunit D |
| | | 03246 | | | | | | |
| F | N | PABCH42_ | 206 | C | T | A206V | lasR | Transcriptional activator protein LasR |
| | | 03585 | | | | | | |
| F | N | PABCH42 0 | 231 | G | A | A231T | lasR | Transcriptional activator protein LasR |
| | | 03585 | | | | | | |
| F | N | PABCH42_ | 179 | 0 | T | R179C | zipA | Cell division protein ZipA |
| | | 03770 | | | | | | |
| F | P | | 1:4241407 | G | A | | | -122-PABCH42_03973 |
| | | | | | | | | -231-PABCH42_03974 |
| F | N | PABCH42_ | 240 | T | C | V240A | mtlY | Xylulose kinase |
| | | 04798 | | | | | | |
| F | N | PABCH42_ | 212 | T | C | V212A | PA2712 | EamA family transporter |
| | | 05418 | | | | | | |
| F | N | PABCH42_ | 257 | C | T | P257S | | Putative serine protease |
| | | 05480 | | | | | | |
| F | S | PABCH42_ | 129 | G | A | L129 | yqaA | DedA family protein |
| | | 05492 | | | | | | |
| F | N | PABCH42_ | 344 | A | G | Y344C | | hypothetical protein |
| | | 05916 | | | | | | |
| F | N | PABCH42_ | 154 | C | A | Q154K | | Oligosaccharide repeat unit polymerase |
| | | 06134 | | | | | | |
| F | S | PABCH42_ | 41 | G | T | V41 | | LrgB family protein |
| | | 06205 | | | | | | |
| F | P | | 1:6686533 | A | G | | | -113-PABCH42 06250 |
| | | | | | | | | 67-PABCH42_06251 |
| F | S | PABCH42_ | 146 | G | C | G146 | pilN | Type IVB pilus formation outer |
| | | 06689 | | | | | | membrane protein |
| F | INDEL | PABCH42_ | 56 | AC | A | Y56 | lasR | Transcriptional activator protein LasR |
| | | 03585 | | | | | | |
| F | INDEL | PABCH42_ | 8 | T | CT | L8 | purT | phosphoribosylglycinamide |
| | | 06526 | | | | | | formyltransferase 2 |
| F | INDEL | PABCH42_ | 440 | G | TG | G440 | | polysaccharide biosynthesis protein |
| | | 05916 | | | | | | |
| F | INDEL | PABCH42_ | 30 | TG | T | L30 | lasR | Transcriptional activator protein LasR |
| | | 03585 | | | | | | |
| G | S | PABCH01_ | 430 | C | A | I430 | spul | Gamma-glutamylputrescine synthetase |
| | | 00311 | | | | | | |
| G | N | PABCH01_ | 40 | C | A | S40L* | PA0365 | hypothetical twin-arginine translocation |
| | | 00384 | | | | | | pathway signal protein |
| G | N | PABCH01_ | 119 | T | C | L119P | pilH | twitching motility protein PilH |
| | | 00430 | | | | | | |
| G | N | PABCH01_ | 66 | T | A | W66R | anmK | Anhydro-N-acetylmuramic acid kinase |
| | | 00673 | | | | | | |
| G | N | PABCH01_ | 8 | C | T | A8V | rpsG | 30S ribosomal protein S7 |
| | | 00698 | | | | | | |
| G | S | PABCH01_ | 235 | C | T | T235 | gldF | ABC-2 family transporter protein |
| | | 00983 | | | | | | |
| G | N | PABCH01_ | 215 | C | T | Y215* | ladS | Lost Adherence Sensor |
| | | 01045 | | | | | | |
| G | N | PABCH01_ | 624 | C | T | E624* | ladS | Lost Adherence Sensor |
| | | 01045 | | | | | | |
| G | N | PABCH01_ | 246 | G | A | G246D | PA3886 | HAD hydrolase, IIA family protein |
| | | 01156 | | | | | | |
| G | N | PABCH01_ | 166 | G | A | A166T | cheA | Chemotaxis protein CheA |
| | | 01345 | | | | | | |
| G | N | PABCH01_ | 11 | A | C | N11T | nalD | Transcriptional repressor of multidrug |
| | | 01475 | | | | | | efflux pump MexAB-OprM |
| G | N | PABCH01_ | 91 | G | A | D91N | czcR | Transcriptional activator involved in |
| | | 02816 | | | | | | metal and drug resistance |
| G | S | PABCH01_ | 196 | C | T | A196 | | Succinylglutamate desuccinylase |
| | | 04663 | | | | | | Cyclic-di-GMP phosphodiesterase |
| G | N | PABCH01_ | 29 | C | A | R29S | bifA | inversely regulating biofilm formation |
| | | 05182 | | | | | | and swarming motility |
| G | P | | -40: | T | C | | | FAD-dependent oxidoreductase |
| | | | PABCH01_ | | | | | |
| | | | 06340 | | | | | |
| G | INDEL | PABCH01_ | 29 | C | CG | A29 | sltB1 | soluble lytic transglycosylase B |
| | | 01020 | | | | | | |
| G | INDEL | | 1:1106337 | AG | A | | | |
| G | INDEL | PABCH01_ | 112 | G | GC | A112 | | hypothetical protein |
| | | 04794 | | | | | | |
| G | INDEL | PABCH01_ | 138 | AG | A | R138 | | hypothetical protein |
| | | 01275 | | | | | | |
| G | INDEL | PABCH01_ | 250 | G | GA | E250 | | hypothetical protein |
| | | 01275 | | | | | | |
| H | N | PABCH46_ | 793 | C | G | T793S | impA | Immunomodulating metalloprotease |
| | | 00617 | | | | | | |
| H | N | PABCH46_ | 113 | C | T | P113L | PA4401 | Glutathione S-transferase |
| | | 01540 | | | | | | |
| H | I | | 1:1858814 | T | A | | | -215-PABCH46_01663 |
| | | | | | | | | 5968-PABCH46_01669 |
| H | I | | 1:1858871 | T | C | | | -272-PABCH46_01663 |
| | | | | | | | | 5911-PABCH46_01669 |
| H | I | | 1:1858875 | C | T | | | -276-PABCH46_01663 |
| | | | | | | | | 5907-PABCH46_01669 |
| H | I | | 1:1858899 | A | G | | | -300-PABCH46_01663 |
| | | | | | | | | 5883-PABCH46_01669 |
| H | I | | 1:1858977 | G | A | | | -378-PABCH46_01663 |
| | | | | | | | | 5805-PABCH46_01669 |
| H | S | PABCH46_ | 67 | C | T | S67 | | HTH-type transcriptional regulator |
| | | 02458 | | | | | | MalT |
| H | N | PABCH46_ | 3819 | C | T | A3819V | pvdL | Pyoverdine chromophore precursor |
| | | 03543 | | | | | | synthetase PvdL |
| H | N | PABCH46_ | 14 | T | C | L14P | pvdS | Sigma factor PvdS controling pyoverdin |
| | | 03545 | | | | | | biosynthesis |
| H | N | PABCH46_ | 80 | G | A | R80H | pvdS | Sigma factor PvdS controling pyoverdin |
| | | 03545 | | | | | | biosynthesis |
| H | N | PABCH46_ | 493 | G | A | G493E | oprM | Outer membrane protein OprM |
| H | | 04084 | | | | | | |
| H | N | PABCH46_ | 112 | C | A | R112L | rhlR | Transcriptional regulator RhlR involved |
| H | | 04836 | | | | | | in quorum sensing |
| H | S | PABCH46_ | 290 | T | A | P290 | algP | Transcriptional regulator protein AlgP |
| | | 05857 | | | | | | |
| H | INDEL | | 1:2919846 | T | TG | | | |
| H | INDEL | | 1:5864879 | C | CCTG | | | |
| H | INDEL | PABCH46_ | 107 | C | GC | G107 | PA5248 | putative Frt1-like Fe2+/Pb2+ permease |
| | | 05851 | | | | | | |
| I | N | PABCH10_ | 131 | C | A | Q131K | | Hypothetical protein |
| | | 00099 | | | | | | |
| I | S | PABCH10_ | 38 | C | T | D38 | tufA | Translation elongation factor Tu |
| | | 00693 | | | | | | |
| I | N | PABCH10_ | 159 | C | T | A159V | phzE | phenazine biosynthesis protein |
| | | 00767 | | | | | | |
| I | N | PABCH10_ | 341 | A | C | H341P | bphP | Bacteriophytochrome |
| | | 00872 | | | | | | |
| I | N | PABCH10_ | 221 | C | T | T221I | eutB | Ethanolamine ammonia-lyase heavy |
| | | 01109 | | | | | | chain |
| I | I | | 1:1282596 | C | T | | | |
| I | I | | 1:1359878 | C | T | | | |
| I | S | PABCH10_ | 296 | C | T | S296 | hscA | Heat shock protein |
| | | 01331 | | | | | | |
| I | N | PABCH10_ | 244 | T | G | V244G | PA3508 | Transcriptional regulator IcIR family |
| | | 01718 | | | | | | |
| I | N | PABCH10_ | 266 | G | C | R266P | yfiS | Major facilitator superfamily (MFS) |
| | | 01761 | | | | | | transporter permease |
| I | N | PABCH10_ | 16 | G | A | A16T | amrZ | Alginate and motility regulator Z |
| | | 01843 | | | | | | |
| I | N | PABCH10_ | 97 | | C | V97G | PA3093 | Carbon-nitrogen hydrolase family |
| | | 02156 | | | | | | protein |
| I | S | PABCH10_ | 391 | C | T | V391 | pell | Pellicle/biofilm biosynthesis |
| | | 02187 | | | | | | glycosyltransferase |
| I | I | | 1:2712699 | T | C | | | |
| I | S | PABCH10_ | 3506 | G | C | R3506 | pvdI | Pyoverdine sidechain non-ribosomal |
| | | 03159 | | | | | | peptide synthetase |
| I | S | PABCH10_ | 11 | T | C | S11 | PA2225 | Putative lipoprotein |
| | | 03331 | | | | | | |
| I | I | | 1:3593069 | G | T | | | |
| I | S | PABCH10_ | 66 | G | C | T66 | fecI | RNA polymerase sigma factor, sigma-70 |
| | | 03489 | | | | | | family protein |
| I | N | PABCH10_ | 494 | G | A | R494H | PA2044 | Tranglutaminase-like cysteine protease |
| | | 03537 | | | | | | |
| I | S | PABCH10_ | 245 | G | A | E245 | | Patatin-like phospholipase family |
| | | 03962 | | | | | | protein |
| I | N | PABCH10_ | 141 | G | T | G141C | PA1638 | Glutaminase |
| | | 03964 | | | | | | |
| I | I | | 1:4427579 | G | T | | | |
| I | N | PABCH10_ | 129 | C | A | P129T | fliO | Flagellar biosyntheses protein |
| | | 004209 | | | | | | |
| I | S | PABCH10_ | 328 | C | G | L328 | btuB | TonB-dependent vitamin B12 receptor |
| | | 04396 | | | | | | |
| I | N | PABCH10_ | 162 | C | A | L162I | braG | High-affinity branched-chain amino acid |
| | | 04608 | | | | | | transport ATP-binding protein |
| I | S | PABCH10_ | 71 | C | T | R71 | | Hypothetical protein |
| | | 04692 | | | | | | |
| I | S | PABCH10_ | 7 | A | G | R7 | rsmA | Carbon storage protein |
| | | 04842 | | | | | | |
| I | N | PABCH10_ | 334 | G | A | A334T | PA0881 | PrpD/MmgE family protein for |
| | | 04866 | | | | | | propanoate metabolism |
| I | S | PABCH10_ | 229 | T | C | T229 | | Hypothetical protein |
| | | 04938 | | | | | | |
| I | N | PABCH10_ | 153 | G | A | G153D | fimU | Type IV fimbrial biogenesis protein |
| | | 05377 | | | | | | |
| I | S | PABCH10_ | 333 | A | G | L333 | ccsA | Cytochrome c551 peroxidase |
| | | 05411 | | | | | | |
| I | N | PABCH10_ | 105 | C | T | T105I | waaF | ADP-heptose:LPS heptosyltransferase II |
| | | 05934 | | | | | | |
| I | N | PABCH10_ | 139 | A | G | T139A | PA5232 | HlyD family secretion protein/ |
| | | 06190 | | | | | | Multidrug resistance efflux pump |
| I | N | PABCH10_ | 531 | G | T | E531* | kinB | Alginate biosynthesis sensor protein |
| | | 06452 | | | | | | |
| I | INDEL | | 1:2785824 | T | TG | | | |
| I | INDEL | PABCH10_ | 816 | C | AC | P816 | | type I restriction endonuclease subunit R |
| | | 04929 | | | | | | |
| I | INDEL | PABCH10_ | 74 | GAGA | G | K75 | infC | Translation initiation factor IF-3 |
| | | 02632 | | | | | | |
| I | INDEL | | 1:3637805 | TA | T | | | |
| I | INDEL | PABCH10_ | 50 | G | GA | R50 | pilC | Type IV fimbrial assembly protein PilC |
| | | 05350 | | | | | | |

### Example 4. Mutations that Alter Clinical Phenotypes are Accrued Over Days

Pathogen populations diversified in all patients by the emergence of single point mutations. Mutations accumulated significantly over days in most patients, as quantified by the increased distance to the most recent common ancestor (d_{MRCA}; FIG. 3, horizontal bar plots insets, permutation test of <d_{MRCA}>), although the frequencies of individual mutations could both increase or decrease (FIG. 7A). Notably, stool and sputum populations within each patient, where observed, were indistinguishable (FIG. 7C, D), indicating either gut carriage as the source of respiratory colonization, or more simply, that stool samples reflect the passage of ingested sputum through the gastrointestinal tract.

To assess whether mutations could reflect diversifying selection, clinically relevant phenotypes of a subset of non-synonymous mutations that increased in frequency (FIG. 3A) were characterized or those that occurred recurrently in genes that appeared to be under selection (FIG. 7B). A causal genotype-phenotype relation was estimated by focusing on singleton mutations in order to compare isolates with and without only that mutation that were otherwise genetically identical, i.e. clinically observed isogenic controls (based on >99% alignment rate of isolate genomes, FIG. 6B). Point mutations impacted a wide range of clinically important phenotypes, including those in wbpL and wzy that altered lipopolysaccharide (LPS) and O-antigen presentation thereby affecting sensitivity to human serum (FIG. 7C-F), and those in biofilm-related genes encoding BifA and KinB that impacted swarming, biofilm formation, and alginate production (FIG. 7G-K). Altogether, these findings show that the evolution of P. aeruginosa over days leads to the diversification of clinically important phenotypes.

### Example 5. Measuring the In Vivo Frequencies of Resistance Mutations

Mapping the antibiotic resistance profiles of isolates to their genomes revealed mutations associated with resistance (FIG. 3A-G, gray symbols; FIG. 8). The frequencies of these mutations appeared to change across days, based on cultured isolates, with some resistant mutants observed only in later time points. For example, nalD (a repressor of MexAB-OprM), anmK (involved in peptidoglycan recycling), and sltB1 (a lytic transglycosylase) were found in a sublineage of patient G* that appeared to emerge after day 1 (FIG. 3E). Another set of linked mutations—oprD, ampD, and sltB1 in patient D—conferring resistance to meropenem and ceftazidime (FIG. 3C) were found at low-frequencies in only the second time point.

To accurately capture the dynamics of resistance mutations in patients without culture-based growth bias, a scheme was designed to measure the mutation frequencies directly from intact sputum samples by developing “resistance-targeted deep amplicon sequencing” (RETRA-Seq) in which the mutated loci from total DNA extracted from sputum for deep amplicon sequencing was amplified (FIG. 4A). In order to control for amplification bias and reliably measure the number of unique genomes across thousands of single cells that correspond to each allele, unique molecular identifiers (UMIs) were incorporated in the primers, and sequence at a saturating depth such that allele frequencies are resolved to the sequencing error rate (FIG. 1A-C; Table 3).

TABLE 3
Exemplary antimicrobial resistance genes and associated antimicrobial resistance profiles

| Patient | Type | Locustag | Pos | Ref | Mut | AAmut | Gene | Annot | KEGG pathway |
|---|---|---|---|---|---|---|---|---|---|
| G | N | PABCH01_00430 | 119 | T | C | L119P | pilH | twitching motility protein | Motility; Two-component system; Biofilm formation |
| | | | | | | | | PilH | |
| G | N | PABCH01_00698 | 8 | C | T | A8V | rpsG | 30S ribosomal protein S7 | Other |
| G | S | PABCH01_00983 | 235 | C | T | T235 | PA4038 | ABC-2 family transporter protein | Other |
| G | N | PABCH01_01045 | 215 | C | T | Y215* | ladS | Lost Adherence Sensor | Biofilm formation |
| G | N | PABCH01_01045 | 624 | C | T | E624* | ladS | Lost Adherence Sensor | Biofilm formation |
| G | N | PABCH01_01156 | 246 | G | A | G246D | PA3886 | HAD hydrolase, IIA family | Other |
| | | | | | | | | protein | |
| G | N | PABCH01_01345 | 166 | G | A | A166T | cheA | Chemotaxis protein CheA | Motility |
| G | N | PABCH01_01475 | 11 | A | C | N11T | nalD | Transcriptional repressor of | beta-Lactam resistance |
| | | | | | | | | multidrug efflux pump | |
| | | | | | | | | MexAB-OprM | |
| G | N | PABCH01_02816 | 91 | G | A | D91N | czcR | Transcriptional activator | Two-component system |
| | | | | | | | | involved in metal and drug | |
| | | | | | | | | resistance | |
| G | S | PABCH01_04663 | 196 | C | T | A196 | PA0891 | Succinylglutamate | Metabolic pathway |
| | | | | | | | | desuccinylase | |
| G | N | PABCH01_05182 | 29 | C | A | R29S | bifA | Cyclic-di-GMP | Biofilm formation |
| | | | | | | | | phosphodiesterase inversely | |
| | | | | | | | | regulating biofilm formation | |
| | | | | | | | | and swarming motility | |
| G | P | | -40:PABCH01_06340 | T | C | | pauB4 | FAD-dependent | Metabolic pathway |
| | | | | | | | | oxidoreductase | |
| G | INDEL | | 1:1106337 | AG | A | | | could be phage integrase; | |
| | | | | | | | | A > AG insertion; likely | |
| | | | | | | | | intergenic | |
| A | N | PABCH05_02289 | 251 | C | T | A251V | PA2707 | MoxR family ATPase AAA, | Other |
| | | | | | | | | modulator of stress response | |
| A | N | PABCH05_05578 | 141 | G | T | G141C | glcD | Glycolate oxidase subunit | Metabolic pathway |
| A | N | PABCH05_05714 | 393 | G | T | G393V | kinB | Alginate biosynthesis sensor | Two-component system |
| | | | | | | | | protein | |
| D | N | PABCH09_00923 | 67 | G | A | R67H | csgA | short chain dehydrogenase | Metabolic pathway |
| D | S | PABCH09_05421 | 31 | A | G | L31 | msbA | Lipid A export ATP- | Other |
| | | | | | | | | binding/permease protein | |
| I | N | PABCH10_00099 | 131 | C | A | Q131K | | Hypothetical protein | Other |
| I | N | PABCH10_00767 | 159 | C | T | A159V | phzE1 | phenazine biosynthesis | Quorum sensing |
| | | | | | | | | protein | |
| I | I | | 1:1282596 | C | T | | | 172-PABCH10_01227 | |
| | | | | | | | | 21-PABCH10_01228 | |
| I | I | | 1:1359878 | C | T | | | -1390-PABCH10_01302 | |
| | | | | | | | | 111-PABCH10_01303 | |
| I | S | PABCH10_01331 | 296 | C | T | S296 | hscA | Heat shock protein | Other |
| I | N | PABCH10_01761 | 266 | G | C | R266P | yfiS | Major facilitator superfsmily | Other |
| | | | | | | | | (MFS) transporter permease | |
| I | N | PABCH10_01843 | 16 | G | A | A16T | amrZ | Aliginate and motility | Motility |
| | | | | | | | | regulator Z | |
| I | N | PABCH10_02156 | 97 | A | C | V97G | PA3093 | Carbon-nitrogen hydrolase | Metablic pathway |
| | | | | | | | | family protein | |
| I | S | PABCH10_02187 | 391 | C | T | V391 | pelF | Pellicle/biofilm biosynthesis | Biofilm formation |
| | | | | | | | | glycosyltransferase | |
| I | I | | 1:2712699 | T | C | | | 12-PABCH10_02569 | |
| | | | | | | | | 264-PABCH10_02570 | |
| I | S | PABCH10_03159 | 3506 | G | C | R3506 | pvdI | Pyoverdine sidechain non- | Pyoverdine synthesis |
| | | | | | | | | ribosomal peptide synthetase | |
| I | I | | 1:3593069 | G | T | | | 36-PABCH10_03358 | |
| | | | | | | | | 403-PABCH10_03359 | |
| I | S | PABCH10_03489 | | G | C | T66 | fecI | RNA polymerase sigma | Metal transport |
| | | | | | | | | factor, sigma-70 family protein | |
| I | I | | 1:4427579 | G | T | | | -455-PABCH10_04123 | |
| | | | | | | | | 334-PABCH10_04124 | |
| I | N | PABCH10_04209 | 129 | C | A | P129T | fliO | Flagellar biosynthesis protein | Motility |
| I | S | PABCH10_04396 | 328 | C | G | L328 | PA1271 | TonB-dependent vitamin B12 | Metal transport |
| | | | | | | | | receptor | |
| I | N | PABCH10_04608 | 162 | C | A | L162I | braG | High-affinity branched-chain | Quorum sensing |
| | | | | | | | | amino acid transport ATP- | |
| | | | | | | | | binding protein | |
| I | S | PABCH10_04842 | 7 | A | G | R7 | rsmA | Carbon storage regulator | Two-component system; |
| | | | | | | | | | Biofilm formation |
| I | N | PABCH10_05377 | 153 | G | A | G153D | fimU | Type IV fimbrial biogenesis | Motility |
| | | | | | | | | protein | |
| I | S | PABCH10_05411 | 333 | A | G | L333 | ccpR | Cytochrome c551 peroxidase | Metabolic pathway |
| | | | | | | | | HlyD family secretion protein/ | |
| | | | | | | | | multidrug resistance efllux | |
| I | N | PABCH10_06190 | 139 | A | G | T139A | PA5232 | pump | Other |
| I | N | PABCH10_06452 | 531 | G | T | E531* | kinB | Alginate biosynthesis sensor protein | Two-component system |
| I | INDEL | | 1:2785824 | T | TG | | | might be protein check again | |
| I | INDEL | PABCH10_04929 | 5302739 | C | AC | | | type I restriction endonuclease | |
| | | | | | | | | subunit R | |
| I | INDEL | PABCH10_02632 | 2755529 | GAGA | G | | infC | Translation initiation factor IF-3 | Metabolic pathway |
| B | S | PABCH13 05028 | 206 | A | G | G206 | | Uracil-DNA glycosylase | |
| | | | | | | | | glycosyltransferase? Undecapreny1- | |
| | | | | | | | | phosphate glucose | |
| B | INDEL | | 1:3129522 | AT | A | | | phosphotransferase? | |
| | INDEL | PABCH14_02010 | 47 | A | AG | | wbpL | glycosyl transferase | Lipopolysaccharide |
| C | | | | | | | | | biosynthesis |
| C | INDEL | PABCH14_01385 | 250 | G | GA | | | hypothetical protein | |
| F | N | PABCH42 00239 | 135 | G | A | D135N | ampR | HTH-type transcriptional activator | beta-Lactam resistance |
| | | | | | | | | AmpR | |
| F | N | PABCH42 00712 | 130 | A | C | T130P | mexR | Multidrug resistance operon repressor | beta-Lactam resistance |
| F | N | PABCH42_00712 | 131 | G | T | R131L | mexR | Multidrug resistance operon repressor | beta-Lactam resistance |
| F | S | PABCH42 02138 | 85 | C | T | N85 | dapB | Dihydrodipicolinate reductase | Metabolic pathway |
| F | N | PABCH42_02677 | 327 | C | A | R327S | bifA | Cyclic-di-GMP phosphodiesterase | Biofilm formation |
| | | | | | | | | inversely regulating biofilm | |
| | | | | | | | | formation and swarming motility | |
| F | S | PABCH42 03246 | 228 | G | A | L228 | shaC | Na(+)/H(+) antiporter subunit D | Metabolic pathway |
| F | N | PABCH42 03585 | 206 | C | T | A206V | lasR | Transcriptional activator protein | Quorum sensing; Biofilm |
| | | | | | | | | LasR | formation |
| F | N | PABCH42 03585 | 231 | | A | A231T | lasR | Transcriptional activator protein | Quorum sensing; Biofilm |
| | | | | | | | | LasR | formation |
| F | N | PABCH42 04798 | 240 | T | C | V240A | mtlY | Xylulose kinase | Metabolic pathway |
| F | N | PABCH42_05480 | 257 | C | T | P257S | | Putative serine protease | Other |
| F | N | PABCH42 05916 | 344 | A | G | Y344C | | hypothetical protein | |
| F | S | PABCH42 06205 | 41 | G | T | V41 | | LrgB family protein | |
| F | INDEL | PABCH42 03585 | 3839911 | AC | A | Y56 | lasR | Transcriptional activator protein | Quorum sensing; Biofilm |
| | | | | | | | | LasR | formation |
| F | INDEL | PABCH42_06526 | 6996855 | T | CT | L8 | purT | phosphoribosylglycinamide | Metabolic pathway |
| | | | | | | | | formyltransferase 2 | |
| E | N | PABCH45_00431 | 1984 | C | T | Q1984* | pilL | Chemotactic signal transduction | Motility; Two-component |
| | | | | | | | | protein | system; Biofilm formation |
| E | N | PABCH45_00703 | 163 | G | A | A163T | vfr | Transcriptional regulator Vfr, global | Two-component |
| | | | | | | | | virulence factor | system; Biofilm |
| | | | | | | | | | formation; Quorum sensing |
| E | N | PABCH45_02996 | 355 | T | C | F355L | czcA | Heavy metal efflux protein | Metal transport |
| E | P | | -69:PABCH45_03200 | T | C | | | -69:PABCH45_03200 | |
| | | | | | | | | Hydrolase/non-heme chloroperoxidase | |
| E | N | PABCH45_03661 | 232 | G | A | W232* | PA3052 | hypothetical protein | Other |
| E | N | PABCH45 03814 | 149 | A | G | S149G | | UDP-glucose/GDP-mannose | Metabolic pathway |
| | | | | | | | | dehydrogenase family | |
| E | N | PABCH45_03873 | 390 | C | T | Q390* | cyaB | Adenylate cyclase for cAMP | Biofilm formation; Other |
| | | | | | | | | synthesis | |
| E | I | | 1:4479447 | | A | G | | | |
| E | N | PABCH45 04514 | 484 | C | T | Q484* | | hypothetical protein | Other |
| E | INDEL | PABCH45_00703 | 758219 | | TA | E79 | vfr | Transcriptional regulator Vfr, global virulence factor | Two-component |
| | | | | | | | | | system; Biofilm |
| | | | | | | | | | formation; Quorum sensing |
| E | INDEL | PABCH45_00426 | 459252 | G | GA | L27 | pilG | Twitching motility protein PilG | Two-component |
| | | | | | | | | | system; Motility; Biofilm |
| | | | | | | | | | formation |
| E | INDEL | | 4694472 | G | GC | | | | |
| E | INDEL | PABCH45_00429 | 461277 | GGT | G | S205 | pilJ | Twitching motility protein PilJ | Two-component |
| | | | | | | | | | system; Motility; Biofilm |
| | | | | | | | | | formation |
| H | I | | 1:1858814 | T | A | | | -215-PABCH46 01663 | |
| | | | | | | | | 5968-PABCH46 01669 | |
| H | I | | 1:1858977 | G | A | | | -378-PABCH46_01663 | |
| | | | | | | | | 5805-PABCH46_01669 | |
| H | N | PABCH46_03543 | 3819 | C | T | A3819V | pvdL | Pyoverdine chromophore precursor | Pyoverdine synthesis |
| | | | | | | | | synthetase PvdL | |
| H | N | PABCH46_04084 | 493 | G | A | G493E | oprM | Outer membrane protein OprM | Quorum sensing; beta-Lactam |
| | | | | | | | | | resistance |
| H | INDEL | | 1:2919846 | T | TG | | | DNA invertase/hypothetical | |
| H | INDEL | | 1:5864879 | C | CCTG | | | murein transglycosylase | |
| H | INDEL | PABCH46 05851 | 107 | C | GC | G107 | PA5248 | putative Frt1-like | Metal transport |
| | | | | | | | | Fe2+/Pb2+ permease | |

RETRA-Seq of select resistance mutations (FIG. 3C-E, gray symbols on branches of trees) revealed three types of in vivo dynamics: (i) ‘pre-existing’ mutations that expanded from low frequencies at day 1 undetected by culture-based colony assay, (ii) presumed ‘de novo’ mutations within sequencing error, and (iii) mutations that went to ‘extinction’ (FIG. 4B-D). Some of these mutations impacted key residues at the interface of multimers, suggesting a loss-of-function (FIG. 4E). The magnitudes of in vivo expansions were striking: for instance, pre-existing mutations in nalD, anmK, and sltB1 started at 7-8% allele frequency and increased to 44-49%, and a presumed de novo mutation in ampD increased to 19%, all over 11 days. Conversely, two independent mexR mutations conferring levofloxacin resistance went to extinction within 5 days. Altogether, our findings show that low-frequency resistance mutations can rapidly expand or contract over large magnitudes within days, suggesting that RETRA-Seq could be utilized during acute infection to accurately survey the in vivo dynamics of resistance mutations.

### Example 6. Relating Dynamics of Low-Frequency Resistance Mutations with Antibiotic Therapy

The expansion and contraction of low-frequency resistance mutations coincided with changes in antibiotic therapy. In several patients, population-wide resistance to β-lactams—cefepime, ceftazidime, piperacillin-tazobactam—changed significantly over time (FIG. 4F, FIG. 8A-F; two-sided Mann-Whitney U-test). Relating the change in β-lactam resistance, using cefepime as an example, to the duration of β-lactam therapy administered to each patient (the fraction between sampled days treated with at least one β-lactam) indicated that resistance increased with treatment (FIG. 4G, Pearson's r=0.936, P=0.002), driven in part by expansions in low-frequency mutations (patients D, F*, G*; FIG. 4B-D). Of note, the oprD mutation may have emerged from meropenem use, which was administered to patient D one day prior to mutant detection (FIG. 1B).

Conversely, changes in therapy were also associated with the contraction of resistance mutations. Patient I* was treated with ceftazidime prior to day 1 but not during the study period (FIG. 5), which coincided with a decrease in cefepime resistance over time (FIG. 4G; FIG. 8D, F). In the case of the aforementioned extinction of levofloxacin-resistant mexR mutations in patient F* (FIG. 4D), the patient had received ciprofloxacin 6 months earlier but was not treated with fluoroquinolones during the study period. Altogether, these findings show that population resistance can shift rapidly based on prior and ongoing choice of antibiotic therapy, in part by the expansion or contraction of low-frequency resistance mutations.

This study also shows that the frequencies of within-population resistance mutations change rapidly with antibiotic therapy, highlighting a potential for deep sequencing-guided, short-term cycling of antibiotics within patients as a possible future therapeutic strategy. As resistance mutations can persist in the population for months following treatment, monitoring low-frequency mutations by deep population profiling can inform which antibiotics should be avoided, or conversely, which should be actively used in the case of compounds that select against a specific type of resistance. While antibiotic cycling has been proposed as a strategy to limit the selective advantage of resistance mutations based on mathematical modeling and experimental evolution studies, to date, there are limited data on its clinical efficacy. The present disclosure provides an approach in acute infections, by cycling drugs over days within individual patients over short time scales, which requires further study.

To inform patient-specific antibiotic cycling strategies, molecular diagnostics that deeply and accurately monitor pathogen diversity throughout infection, particularly at the start of infection, are needed. Current culture-based clinical microbiology practice risks missing low-frequency resistant variants. Furthermore, culture-based assays introduce growth bias that differs from the native context of the human lung, where spatial selection is known to occur on pathogens across different niches. Specific alleles encoding resistance could be detected with next-generation molecular assays, e.g. CRISPR-based diagnostics. To monitor known hotspots of mutated genes, resistance targeted deep amplicon sequencing (RETRA-Seq), using primers that are designed to be suitable across multiple strains can be used, as a highly sensitive method to monitor numerous loci across pathogen genomes.

Patient enrollment. The clinical research described in this disclosure complies with all relevant ethical regulations, and the study protocol was approved by the Institutional Review Board of Boston Children's Hospital. Informed consent was obtained for sample use/collection and medical record review. For pediatric patients, consent was obtained from legal guardians of each patient. Mechanically ventilated patients in the pediatric ICU (via endotracheal tube (ETT) or tracheostomy tube (trach)) were enrolled in the study at the time of suspected infection, defined as when respiratory samples (sputum obtained via endotracheal aspirate or trach aspirate) were ordered by the clinical team for evaluation of suspected infection, with subsequent confirmation of P. aeruginosa growth in the clinical microbiology lab. Patients typically experienced fever or hypothermia, increase in ventilator settings or oxygen requirement, and/or increase in quantity and/or change in color or thickness of respiratory secretions. Patients were classified as having pneumonia if they met these criteria and there was a new and persistent infiltrate on chest radiograph (CXR). Patients were classified as tracheitis if CXR showed no evidence of pneumonia but sputum obtained via ETT aspirate or tracheal aspirate showed few, moderate, or abundant polymorphonuclear leukocytes (PMN) on Gram stain. None of the patients met criteria for a ventilator-associated event (VAE). None of the patients had bacteremia, and all recovered from their infection.

The results described herein above were obtained using the following methods and materials.

Sample collection. Sputum and stool samples were processed within 24-48 hrs of collection from the patient, and solubilized with 10 mM dithiothreitol, frozen in 15% glycerol, and stored at −80° C. until further processing.

Whole genome sequencing of P. aeruginosa isolates. Isolates were cultured from sputum and stool samples as previously described (e.g., see Chung, H. et al. Global and local selection acting on the pathogen Stenotrophomonas maltophilia in the human lung. Nat. Commun. 8, 14078 (2017), incorporated herein by reference). Serial dilutions (100 to 10-4) of each sample in PBS were plated onto cetrimide agar (BD) to identify a dilution plate with growth of 50-300 colonies in total to use for colony picking in order to maximize diversity while minimizing competition between isolates. Colonies (24) were randomly picked by taping a paper pre-marked with 24 random “x” marks to the back of each Petri dish using a clean toothpick, which were placed into 1 mL of LB broth in 96 deep-well plates, then grown overnight at 37° C. with shaking. Half of the saturated cultures were used to make glycerol stocks and the rest were used for DNA extraction (Invitrogen PureLink Pro 96 Genomic DNA Purification Kit). Sequencing libraries of the genomes were prepared as previously described (e.g., see, Baym, M. et al. Inexpensive multiplexed library preparation for megabase-sized genomes. PLoS One 10, e0128036 (2015), incorporated herein by reference) and sequenced using paired-end 100 bp reads on the Illumina HiSeq 2000 platform, targeting an average sequencing coverage of 40× per isolate.

Constructing patient-specific reference genomes with long-reads. A single colony was isolated from a cetrimide agar plate streaked with each patient's day 1 sputum sample, grown overnight at 37° C., and cultured overnight in LB broth with shaking, from which genomes were extracted (Invitrogen PureLink Pro 96 Genomic DNA Purification Kit). Genomes were sequenced on both the PacBio platform (long reads) and on the Illumina HiSeq 2500 platform (short reads) to enable error-correction of assembled contigs. Illumina reads were filtered (min Phred score 15) then trimmed for adapter sequences and assembled de novo using Newbler (v 2.7), with minimum contig size 100 bp and minimum coverage at 50×. PacBio reads were assembled de novo using default HGAP 2.0/HGAP 3.0 parameters in the SMRT Analysis Portal (v. 2.3.0). Overlapping contig ends were removed to circularize individual PacBio contigs, and Illumina data was mapped to circularized contigs to detect/correct errors. Comparative genomic analyses were performed using Geneious (see, Kearse, M. et al. Geneious Basic: an integrated and extendable desktop software platform for the organization and analysis of sequence data. Bioinformatics 28, 1647-1649 (2012), incorporated herein by reference).

Constructing a pangenome of coding sequences across reference genomes. A pangenome of all coding sequences found across the patient reference genomes, and two published strains PAO1 and PA14, was constructed with Roary55 3.8.0 (-i 80; minimum percentage identity for blastp). Serotypes were predicted using the web server of PAst (e.g., see Thrane, S. W., Taylor, V. L., Lund, O., Lam, J. S. & Jelsbak, L. Application of whole-genome sequencing data for 0-specific antigen analysis and in silico serotyping of Pseudomonas aeruginosa isolates. J. Clin. Microbiol. 54, 1782-1788 (2016), incorporated herein by reference).

Identifying within-patient mutations and short indels. Short reads (Illumina platform) of individual isolate genomes were adapter trimmed (cutadapt v 1.8.3), filtered (sickle, quality cutoff 25, length cutoff 50), and aligned to the corresponding patient-specific reference genome (bowtie2 v 2.2.4 paired-end, maximum fragment length 2,000 bp, no-mixed, dovetail, very-sensitive, n-ceil 0, 0.01). Within-patient single nucleotide polymorphisms (SNPs) were determined by first identifying variant positions of individual isolates with respect to patient-specific references (SAMtools v 1.3 (see, Li, H. et al. The Sequence Alignment/Map format and SAMtools. Bioinformatics 25, 2078-2079 (2009), incorporated herein by reference), FQ<=−30), combining the list of variant positions across all isolates of a patient, which were then filtered to high-quality SNP positions. High-quality SNPs were defined as nucleotides at which any two isolates disagreed in the called nucleotide, with both calls meeting a patient-specific FQ threshold that was set based on the distribution of all FQ scores within each patient2. Short insertions and deletions (indels) were identified with platypus58 (v 0.8.1, getVariantsFromBAMs=1, genSNPs=0, genlndels=1, minMapQual=30), using a QD (ratio of variant quality to read depth) threshold set for each patient based on the distribution of all QD values. All short indels were confirmed by visual inspection of the aligned reads. A genotype matrix (isolates by positions) based on SNPs and indels were constructed for each patient's pathogen population used for downstream analysis.

Within-patient phylogenetic trees. A maximum parsimony phylogenetic tree was constructed for each patient, using the genotype matrix of within-patient SNPs and indels, with dnapars v 3.696 (PHYLIP package)(see, Baum, B. R. PHYLIP: Phylogeny inference package. Version 3.2. Joel Felsenstein. Q. Rev. Biol. 64, 539-541(1989), incorporated herein by reference). Indels were treated as a mutational event, with “I” or “D” designating an insertion or deletion. To root the tree, an “Outgroup” for each patient was created by using the most likely ancestral nucleotide state at each polymorphic locus; this was identified by querying a 101 bp sequence (50 bp upstream and downstream from each mutated locus) against all Pseudomonas aeruginosa genomes in the NCBI database with BLASTN. For all polymorphic loci, only one state was found in the database, which was designated as the ancestral state based on its prior observation, while the other state was interpreted as a de novo mutation. All phylogenetic trees were plotted with Toytree v 2.0.1 (see, Eaton, D. A. R. Toytree: A minimalist tree visualization and manipulation library for Python. Methods Ecol. Evol. 11, 187-191 (2020), incorporated herein by reference).

Estimating patient colonization time. Bayesian phylogenetic analysis (BEAST 1.10.461) was conducted on the genotype matrix of each patient to estimate the time to the ancestral node in days. Input files were generated with BEAUTi v. 10.4, and BEAST 1.10.4 was run under a tree prior of coalescent expansion growth model and otherwise default parameters. Analyses were run using CIPRES (e.g., see Miller, M. A., Pfeiffer, W. & Schwartz, T. Creating the CIPRES Science Gateway for inference of large phylogenetic trees. in 2010 Gateway Computing Environments Workshop (GCE) (IEEE, 2010), incorporated herein by reference).

Pathway analysis of day 1 mutations. Mutations within day 1 pathogen populations across all patients that were found in annotated coding genes (50 of 81 mutations total) were used to identify associated KEGG pathways on The Pseudomonas Genome Database (Winsor, G. L. et al. Enhanced annotations and features for comparing thousands of Pseudomonas genomes in the Pseudomonas genome database. Nucleic Acids Res. 44, D646-53 (2016), incorporated herein by reference).

Twitching motility assay. Assay was conducted as previously reported (e.g., see O'May, C. & Tufekji, N. The swarming motility of Pseudomonas aeruginosa is blocked by cranberry proanthocyanidins and other tannin-containing materials, Appl. Environ. Microbiol. 77, 3061-3067 (2011), incorporated herein by reference). Frozen isolates were streaked onto LB-agar plates and grown at 37° C. o/n. Individual colonies were selected with a toothpick and stabbed to the bottom of the twitching assay plate (1% tryptone (Sigma Aldrich), 0.5% yeast extract (Sigma-Aldrich), 0.5% NaCl (Sigma)); plates were incubated at 37° C. for 20 hrs. Agar was carefully removed, then plates were stained with 0.1% of Crystal Violet (Sigma) in DI water for 15 min and rinsed with DI water once, then dried. The diameter of the circle was measured in cm.

Permutation test for shift in <dMRCA> over time. The distance to the most recent common ancestor (dMRCA), inferred by the maximum parsimony tree of each patient, was calculated for each isolate within a patient population. Mean <dMRCA> of each sputum sample, <dMRCA>t1 for day 1 and <dMRCA>t2 for follow-up sputum, was calculated within each patient. To test whether the observed difference in means, <dMRCA>t2−<dMRCA>t1 was significant, a null model was constructed by permuting the sputum sample assignment across all sputa isolates and recalculating the difference in means across 1000 permutations, from which a one-tailed p-value was calculated.

Pro-Q gel for lipopolysaccharide. Colonies from an overnight grown Luria Agar plate were resuspended in Luria Broth, normalized to an OD600 of 2.0, then pelleted. LPS was prepared as previously documented65, and 15 μL of each LPS sample was loaded into each well, then separated by SDS-PAGE in a 10% Mini-PROTEAN TGX gel (Bio-Rad) along with CandyCane glycoprotein ladder (Thermo Fisher). LPS was stained using Pro-Q Emerald 300 LPS Gel Stain (Thermo Fisher) according to the manufacturer's instructions with slight modifications (the initial fixation step was repeated twice and each washing step was repeated three times).

O6 serotype Western blot. Colonies from an overnight grown Luria Agar plate were resuspended in Luria Broth, normalized to an OD600 of 2.0, then pelleted. LPS was prepared as previously documented (Davis, M. R., Jr & Goldberg, J. B. Purification and visualization of lipopolysaccharide from Gram-negative bacteria by hot aqueous-phenol extraction. J. Vis. Exp. (2012), which is incorporated herein by reference), and 15 μL of each LPS sample was loaded into each well, then separated by SDS-PAGE in a 10% Mini-PROTEAN TGX gel (Bio-Rad) along with Precision Plus All Blue Protein ladder (Bio-Rad). The LPS was then transferred to a PVDF membrane and blocked for 1 hr, at room temperature, in PBST-5% milk. O6 primary antibody was incubated in a 1:2,500 dilution (Group G, Accurate Chemical & Scientific) in PBST-3% BSA overnight at 4° C. Secondary α-rabbit-HRP IgG (Sigma) was incubated in a 1:10,000 dilution in PBST-3% BSA for 1 hr at room temperature. Blot was visualized using Pierce ECL Western Blotting Substrate (Thermo) according to the manufacturer's instructions.

Serum killing assay. Isolates were streaked onto TSA plates and incubated at 37° C. o/n, then resuspended in 10 mL PBS+ (PBS, 1% proteose peptone, 1 mM CaCl2), 1 mM MgCl2) to an OD600 of 0.25, and diluted 1:23 fold to a final concentration of 5×105 CFU/100 μL. 100 μL of the diluted culture was mixed with 50% serum (Human Serum, male AB plasma, Sigma-Aldrich H4522; diluted 1:2 with PBS+) in a 96-well round bottom plate in triplicate. Serum assay plates were incubated at 37° C. with shaking at 100 r.p.m. for 1 hr, then plated onto TSA, incubated at 37° C. o/n, and quantified for colony forming units (CFU). The PAO1 strain was used as a negative control (not serum sensitive) and PAO1 galU mutant (Priebe, G. P. et al. The galU Gene of Pseudomonas aeruginosa is required for corneal infection and efficient systemic spread following pneumonia but not for infection confined to the lung. Infect. Immun. 72, 4224-4232 (2004, incorporated herein by reference) was used as a positive control (serum sensitive).

Swarming motility assay. Swarming assays were performed as previously reported (e.g., see Ha, D.-G., Kuchma, S. L. & O'Toole, G. A. Plate-based assay for swarming motility in Pseudomonas aeruginosa. Methods Mol. Biol. 1149, 67-72 (2014), incorporated herein by reference). Swarming medium contained 0.52% agar with M8 medium supplemented with casamino acids (0.5%), glucose (0.2%) and MgSO4 (1 mM). Swarming plates were inoculated with 2.5 μL of an overnight culture grown in LB at 37° C. Plates were incubated at 37° C. for 16 hrs. The “Total Swarm Area” is a measure of the number of pixels calculated using ImageJ by first selecting the swarm area, converting images to grayscale (Image→Type→8-bit), thresholding the image (converting to a black and white image where swarm area is black), and analyzing the particles in the swarm (the number of pixels).

Biofilm and Psl assay. Biofilm assays were performed as previously described (O'Toole, G. A. Microtiter dish biofilm formation assay. J. Vis. Exp. (2011), incorporated herein by reference). Overnight cultures (1.5 μL) were inoculated in 100 μL swarming medium and incubated at 37° C. for 24 hrs. Plates were then stained with 0.1% crystal violet. Absorbance was read at OD550. Psl ELISA was conducted following published methods (Ha, D.-G., Kuchma, S. L. & O'Toole, G. A. Plate-based assay for swarming motility in Pseudomonas aeruginosa. Methods Mol. Biol. 1149, 67-72 (2014), incorporated herein by reference). Briefly, 96-well flat-bottom ELISA plates were coated with bacteria overnight at 4° C. Diluted anti-Psl monoclonal antibody (Cam-003; gift from Antonio DiGiandomenico) was added to PBS+1% BSA (PBS-B)—blocked plates for 1 hr, washed with PBS supplemented with 0.1% Tween 20 (PBS-T), and treated with alkaline phosphatase-conjugated anti-human IgG secondary antibodies (Sigma #A1543) at 1:1000 for 1 hr, followed by development with PNP substrate (Sigma).

AlgD promoter activity assay. Strains carrying the lacZ fusion were streaked on PIA or PIA supplemented with 0.1 mM uracil at 37° C. for 24 hrs. The colonies were then scraped into 4 mL 1×PBS and then diluted to OD600 0.3-0.7. Triplicates of 100 μL of the sample were added to 900 μL of Z-Buffer and 20 μL toluene in a 1.5 mL elution tube. After mixing by inverting 4-5 times tubes were placed with tops open in a shaking incubator at 37° C. for 40 min. After, 200 μL of ortho-Nitrophenyl-β-galactoside (ONPG) (4 mg/mL) (Thermo Scientific, Waltham, MA) was added and the time of color change was recorded the reaction was stopped by adding 500 μL of 1M Na2CO3 (Fisher Scientific, Waltham, MA) after 20 min. OD420 and OD550 were measured using a SpectraMax i3x (Molecular Devices, Downingtown, PA) plate reader. Miller units were calculated using the following formula: 1000×[OD420−(1.75×OD550)]/[color change time (min.)×Sample volume×OD600]. In-frame deletion of kinB in strain PA14 was conducted using pEX100T-NotI-ΔkinB through a two-step allelic exchange procedure (see Damron, F. H., Qiu, D. & Yu, H. D. The Pseudomonas aeruginosa sensor kinase KinB negatively controls alginate production through AlgW-dependent MucA proteolysis. J. Bacteriol. 191, 2285-2295 (2009), incorporated herein by reference). Single-crossover merodiploid strains were selected based on sensitivity to sucrose (sacB) and resistance to carbenicillin. Selected merodiploid strains were then grown in LB broth at 37° C. Double-cross over strains were selected based on sensitivity to carbenicillin and confirmed through PCR amplification of the flanking region of target gene.

Antibiotic susceptibility measurements. Minimum inhibitory concentrations (MICs) or zones of inhibition were measured for each isolate in the Infectious Diseases Diagnostic Laboratory at Boston Children's Hospital, using the Vitek-2 instrument (liquid culture assay) or disk diffusion assay, respectively.

Preparation of amplicon sequencing library. Total genomic DNA was extracted from each sputum following previously published methods (see, Terranova, L. et al. How to process sputum samples and extract bacterial DNA for Microbiota analysis. Int. J. Mol. Sci. 19, (2018), incorporated herein by reference). Briefly, sputum was mixed with 1 mM dithiothreitol (DTT), incubated at 30° C. for 30 min with 0.18 mg/mL lysostaphin and 3.6 mg/mL lysozyme. DNA was purified using the High Pure PCR Template Preparation Kit (Roche) according to the manufacturer's instructions and eluted in 30 μL of sterile water. A two-step PCR reaction was used to amplify select loci and add adapter sequences as previously documented72. First PCR. PCR mix was the following: 2 μL DNA template, 10 μL Q5 Hot-Start High-Fidelity 2× Master Mix, 1 μL (NEB #M0494S), 1 μL locus-specific forward primer with UMIs, 1 μL locus-specific reverse primer with UMIs (primers in Supplementary Data 3), 6 μL PCR grade sterile water. Cycling program: hot start 30 s at 98° C., 20× cycles of [10 s at 98° C., 15 s at 67° C., 15 s at 72° C.], then final extension 2 min at 72° C. Dilute PCR1 products 1:10 in PCR grade water. Second PCR. PCR mix was the following: 2 μL 1:10 diluted PCR1 product, 10 μL Q5 Hot-Start High-Fidelity 2× Master Mix, 1 μL universal forward primer, 1 μL sample-specific barcoded reverse primer, 6 μL PCR grade sterile water. Cycling program: hot start 30 s at 98° C., 20× cycles of [10 s at 98° C., 30 s at 72° C.], then final extension 2 min at 72° C. Pool and clean up PCR reaction using a column (Zymo Research #D4013). Amplicon libraries were assessed for correct fragment sizes (350-400 bp) on a 2% agarose gel and quantified using Qubit. Libraries were sequenced on a MiSeq v 2 300 cycle kit (Illumina #MS-102-2002) with Read 1: 150 cycles, Index 1: 8 cycles, Read 2: 150 cycles, sequenced at a minimum saturating depth defined as 1/Illumina sequencing error rate, estimated as 0.5% (Stoler, N. & Nekrutenko, A. Sequencing error profiles of Illumina sequencing instruments. NAR Genom Bioinform 3, lqab019 (2021), incorporated herein by reference).

Sequences useful in the methods are shown in Table 4, Table 5, and Table 6.

TABLE 4
Exemplary PCR primers for use in methods of the disclosure.

| | | Product |
|---|---|---|
| Name | Sequence | Size |
| P01_nalD_ | CCTACACGACGCTCTTCCGATCT NNNNNNNN CTGGAAGTGCCAGTAGACGG | 257 |
| FW | (SEQ ID NO: 1) | |
| P01_nalD_ | GAGTTCAGACGTGTGCTCTTCCGATCT NNNNNNNN | |
| RV | AAATACTTCGAGTCCGCCCG (SEQ ID NO: 2) | |
| P01_anmK_ | CCTACACGACGCTCTTCCGATCT NNNNNNNN GGTTCATGCCGGATGGTTTG | 262 |
| FW | (SEQ ID NO: 3) | |
| P01_anmK_ | GAGTTCAGACGTGTGCTCTTCCGATCT NNNNNNNN | |
| RV | GATCGAGCAAGGTGATCGCA (SEQ ID NO: 4) | |
| P01_sltB1_ | CCTACACGACGCTCTTCCGATCT NNNNNNNN CAAGTACTGCGTACATGGGC | 228 |
| FW | (SEQ ID NO: 5) | |
| P01_sltB1_ | GAGTTCAGACGTGTGCTCTTCCGATCT NNNNNNNN | |
| RV | GCATCGAGGATCGACTGCTT (SEQ ID NO: 6) | |
| P09_oprD_ | CCTACACGACGCTCTTCCGATCT NNNNNNNN CGACGGTACCAAGGTCGATT | 259 |
| FW | (SEQ ID NO: 7) | |
| P09_oprD_ | GAGTTCAGACGTGTGCTCTTCCGATCT NNNNNNNN | |
| RV | CGTTGCCTGTCGGTCGATTA (SEQ ID NO: 8) | |
| P09_ampD_ | CCTACACGACGCTCTTCCGATCT NNNNNNNN TTGGAATCGGTCAATCCGGC | 241 |
| FW | (SEQ ID NO: 9) | |
| P09_ampD_ | GAGTTCAGACGTGTGCTCTTCCGATCT NNNNNNNN | |
| RV | CAACGACTTCTCCCTTGGCA (SEQ ID NO: 10) | |
| P09_sltB1_ | CCTACACGACGCTCTTCCGATCT NNNNNNNN ATCATCGGCGTGGAAACCTT | 228 |
| FW | (SEQ ID NO: 11) | |
| P09_sltB1_ | GAGTTCAGACGTGTGCTCTTCCGATCT NNNNNNNN | |
| RV | CTCGGCATGAATTGTGGCAG (SEQ ID NO: 12) | |
| P13_sulP_ | CCTACACGACGCTCTTCCGATCT NNNNNNNN CTGGTGGACCTGTCGATGAT | 256 |
| FW | (SEQ ID NO: 13) | |
| P13_sulP_ | GAGTTCAGACGTGTGCTCTTCCGATCT NNNNNNNN | |
| RV | ATGGATGTGCAGCAGGTTCT (SEQ ID NO: 14) | |
| P14_wbpL_ | CCTACACGACGCTCTTCCGATCT NNNNNNNN CGCTCTTTCCCTGCTGTTGA | 216 |
| FW | (SEQ ID NO: 15) | |
| P14_wbpL_ | GAGTTCAGACGTGTGCTCTTCCGATCT NNNNNNNN | |
| RV | GGATACCAGTGGCCAGATCC (SEQ ID NO: 16) | |
| P42_ampR_ | CCTACACGACGCTCTTCCGATCT NNNNNNNN CGAAGCGGATCGCGTAGTC | 244 |
| FW | (SEQ ID NO: 17) | |
| P42_ampR_ | GAGTTCAGACGTGTGCTCTTCCGATCT NNNNNNNN | |
| RV | TGCTGTGTGACTCCTTCGAC (SEQ ID NO: 18) | |
| P42_mexR_ | CCTACACGACGCTCTTCCGATCT NNNNNNNN CTTCCAGCTCTTCCTCACCG | 247 |
| FW | (SEQ ID NO: 19) | |
| P42_mexR_ | GAGTTCAGACGTGTGCTCTTCCGATCT NNNNNNNN | |
| RV | ACCTGAAACGAAAAACGCCG (SEQ ID NO: 20) | |
| P42_ampD_ | CCTACACGACGCTCTTCCGATCT NNNNNNNN CTTGGCATCGAACTGGAGGG | 257 |
| FW | (SEQ ID NO: 21) | |
| P42_ampD_ | GAGTTCAGACGTGTGCTCTTCCGATCT NNNNNNNN | |
| RV | AGCAACACCAGGAACGTCAT (SEQ ID NO: 22) | |
| P42_PA0810_ | CCTACACGACGCTCTTCCGATCT NNNNNNNN GGCTTGTAGTGGCCGAACAG | 216 |
| FW | (SEQ ID NO: 23) | |
| P42_PA0810_ | GAGTTCAGACGTGTGCTCTTCCGATCT NNNNNNNN CTGCTGCAACGCATCACTG | |
| RV | (SEQ ID NO: 24) | |

TABLE 5
Exemplary primer for deep sequencing

| Name | Read 1 sequencing primer |
|---|---|
| PCR2_FW | AATGATACGGCGACCACCGAGATCT |
| | ACACTCTTTCCCTACACGACGCTCTTCCGATCT |
| | (SEQ ID NO: 25) |

TABLE 6
Exemplary primers for deep sequencing

| | Read 2 Seq primer partial: | | |
|---|---|---|---|
| Name | [P7][barcode][Read 2 Seq Primer, partial] | Barcode | Output |
| BC1_01SP9_nalD | CAAGCAGAAGACGGCATACGAGATTATCCAGTGTGACTGGAGTTCAGACGTGTGCTCTT | TATCCA | ACTGGA |
| | C (SEQ ID NO: 26) | GT | TA |
| BC2_01SP9_anmK | CAAGCAGAAGACGGCATACGAGATGAGATAACGTGACTGGAGTTCAGACGTGTGCTCTT | GAGATA | GTTATCT |
| | C (SEQ ID NO: 27) | AC | C |
| BC3_01SP9_sltB1 | CAAGCAGAAGACGGCATACGAGATCATGGAATGTGACTGGAGTTCAGACGTGTGCTCTT | CATGGA | ATTCCAT |
| | C (SEQ ID NO: 28) | AT | G |
| BC4_01SP10_nalD | CAAGCAGAAGACGGCATACGAGATCAGTTCCAGTGACTGGAGTTCAGACGTGTGCTCTT | CAGTTCC | TGGAAC |
| | C (SEQ ID NO: 29) | A | TG |
| BC5_01SP10_anmK | CAAGCAGAAGACGGCATACGAGATCCGTATATGTGACTGGAGTTCAGACGTGTGCTCTT | CCGTAT | ATATAC |
| | C (SEQ ID NO: 30) | AT | GG |
| BC6_01SP10_sltB1 | CAAGCAGAAGACGGCATACGAGATGCTGAAGAGTGACTGGAGTTCAGACGTGTGCTCTT | GCTGAA | TCTTCAG |
| | C (SEQ ID NO: 31) | GA | C |
| BC7_09SP1_oprD | CAAGCAGAAGACGGCATACGAGATCGCAAGCTGTGACTGGAGTTCAGACGTGTGCTCTT | CGCAAG | AGCTTGC |
| | C (SEQ ID NO: 32) | CT | G |
| BC8_09SP1_ampD | CAAGCAGAAGACGGCATACGAGATTATCTGTGGTGACTGGAGTTCAGACGTGTGCTCTT | TATCTGT | CACAGA |
| | C (SEQ ID NO: 33) | G | TA |
| BC9_09SP1_sltB1 | CAAGCAGAAGACGGCATACGAGATGCCGAATGGTGACTGGAGTTCAGACGTGTGCTCTT | GCCGAA | CATTCGG |
| | C (SEQ ID NO: 34) | TG | C |
| BC10_09SP4_oprD | CAAGCAGAAGACGGCATACGAGATTACTGCAGGTGACTGGAGTTCAGACGTGTGCTCTT | TACTGC | CTGCAGT |
| | C (SEQ ID NO: 35) | AG | A |
| BC11_09SP4_ampD | CAAGCAGAAGACGGCATACGAGATCATGTTGAGTGACTGGAGTTCAGACGTGTGCTCTT | CATGTTG | TCAACAT |
| | C (SEQ ID NO: 36) | A | G |
| BC12_09SP4_sltB1 | CAAGCAGAAGACGGCATACGAGATATAGAGTCGTGACTGGAGTTCAGACGTGTGCTCTT | ATAGAG | GACTCTA |
| | C (SEQ ID NO: 37) | TC | T |
| BC13_13SP1_sulP | CAAGCAGAAGACGGCATACGAGATTAACCTCAGTGACTGGAGTTCAGACGTGTGCTCTT | TAACCTC | TGAGGTT |
| | C (SEQ ID NO: 38) | A | A |
| BC14_13SP3_sulP | CAAGCAGAAGACGGCATACGAGATATTAGCTGGTGACTGGAGTTCAGACGTGTGCTCTT | ATTAGCT | CAGCTA |
| | C (SEQ ID NO: 39) | G | AT |
| BC15_14SP1_wbpL | CAAGCAGAAGACGGCATACGAGATTCACGCTCGTGACTGGAGTTCAGACGTGTGCTCTT | TCACGCT | GAGCGT |
| | C (SEQ ID NO: 40) | C | GA |
| BC16_14SP3_wbpL | CAAGCAGAAGACGGCATACGAGATGCATCACGGTGACTGGAGTTCAGACGTGTGCTCTT | GCATCA | CGTGAT |
| | C (SEQ ID NO: 41) | CG | GC |
| BC17_42SP1_ampR | CAAGCAGAAGACGGCATACGAGATAGGACGGTGTGACTGGAGTTCAGACGTGTGCTCTT | AGGACG | ACCGTCC |
| | C (SEQ ID NO: 42) | GT | T |
| BC18_42SP1_mexR | CAAGCAGAAGACGGCATACGAGATGAACGTAGGTGACTGGAGTTCAGACGTGTGCTCTT | GAACGT | CTACGTT |
| | C (SEQ ID NO: 43) | AG | C |
| BC19_42SP1_ampD | CAAGCAGAAGACGGCATACGAGATACCTTGAAGTGACTGGAGTTCAGACGTGTGCTCTT | ACCTTG | TTCAAG |
| | C (SEQ ID NO: 44) | AA | GT |
| BC20_42SP1_PA0810 | CAAGCAGAAGACGGCATACGAGATGTGGTTCCGTGACTGGAGTTCAGACGTGTGCTCTT | GTGGTTC | GGAACC |
| | C (SEQ ID NO: 45) | C | AC |
| BC21_42SP2_ampR | CAAGCAGAAGACGGCATACGAGATTATGGTGTGTGACTGGAGTTCAGACGTGTGCTCTT | TATGGT | ACACCA |
| | C (SEQ ID NO: 46) | GT | TA |
| BC22_42SP2_mexR | CAAGCAGAAGACGGCATACGAGATCGACTGCTGTGACTGGAGTTCAGACGTGTGCTCTT | CGACTG | AGCAGT |
| | C (SEQ ID NO: 47) | CT | CG |
| BC23_42SP3_ampD | CAAGCAGAAGACGGCATACGAGATCTGGCTCTGTGACTGGAGTTCAGACGTGTGCTCTT | CTGGCTC | AGAGCC |
| | C (SEQ ID NO: 48) | T | AG |
| BC24_42SP4_PA0810 | CAAGCAGAAGACGGCATACGAGATGGCTCTTGGTGACTGGAGTTCAGACGTGTGCTCTT | GGCTCTT | CAAGAG |
| | C (SEQ ID NO: 49) | G | CC |
| BC1_01SP9_nalD_ | CAAGCAGAAGACGGCATACGAGATCGAACAGTGTGACTGGAGTTCAGACGTGTGCTCTT | CGAACA | ACTGTTC |
| set2 | C (SEQ ID NO: 50) | GT | G |
| BC2_01SP9_anmK_ | CAAGCAGAAGACGGCATACGAGATGGTTAGCAGTGACTGGAGTTCAGACGTGTGCTCTT | GGTTAG | TGCTAAC |
| set2 | C (SEQ ID NO: 51) | CA | C |
| BC3_01SP9_sltB1_ | CAAGCAGAAGACGGCATACGAGATAGACGCTGGTGACTGGAGTTCAGACGTGTGCTCTT | AGACGC | CAGCGT |
| set2 | C (SEQ ID NO: 52) | TG | CT |
| BC4_01SP10_nalD_ | CAAGCAGAAGACGGCATACGAGATTTGCTAGGGTGACTGGAGTTCAGACGTGTGCTCTT | TTGCTAG | CCTAGC |
| set2 | C (SEQ ID NO: 53) | G | AA |
| BC5_01SP10_anmK_ | CAAGCAGAAGACGGCATACGAGATCTCCGGTTGTGACTGGAGTTCAGACGTGTGCTCTT | CTCCGGT | AACCGG |
| set2 | C (SEQ ID NO: 54) | T | AG |
| BC6_01SP10_sltB1_ | CAAGCAGAAGACGGCATACGAGATACCGCATCGTGACTGGAGTTCAGACGTGTGCTCTT | ACCGCA | GATGCG |
| set2 | C (SEQ ID NO: 55) | TC | GT |
| BC7_09SP1_oprD_ | CAAGCAGAAGACGGCATACGAGATATTACCGAGTGACTGGAGTTCAGACGTGTGCTCTT | ATTACC | TCGGTA |
| set2 | C (SEQ ID NO: 56) | GA | AT |
| BC8_09SP1_ampD_ | CAAGCAGAAGACGGCATACGAGATAGTTGTTGGTGACTGGAGTTCAGACGTGTGCTCTT | AGTTGTT | CAACAA |
| set2 | C (SEQ ID NO: 57) | G | CT |
| BC9_09SP1_sltB1_ | CAAGCAGAAGACGGCATACGAGATACGCTGTCGTGACTGGAGTTCAGACGTGTGCTCTT | ACGCTG | GACAGC |
| set2 | C (SEQ ID NO: 58) | TC | GT |
| BC10_09SP4_oprD_ | CAAGCAGAAGACGGCATACGAGATATTGGCAAGTGACTGGAGTTCAGACGTGTGCTCTT | ATTGGC | TTGCCAA |
| set2 | C (SEQ ID NO: 59) | AA | T |
| BC11_09SP4_ampD_ | CAAGCAGAAGACGGCATACGAGATTAGCACGCGTGACTGGAGTTCAGACGTGTGCTCTT | TAGCAC | GCGTGCT |
| set2 | C (SEQ ID NO: 60) | GC | A |
| BC12_09SP4_sltB1_ | CAAGCAGAAGACGGCATACGAGATTGAAGGATGTGACTGGAGTTCAGACGTGTGCTCTT | TGAAGG | ATCCTTC |
| set2 | C (SEQ ID NO: 61) | AT | A |
| BC13_13SP1_sulP_ | CAAGCAGAAGACGGCATACGAGATTTCAGTAGGTGACTGGAGTTCAGACGTGTGCTCTT | TTCAGTA | CTACTGA |
| set2 | C (SEQ ID NO: 62) | G | A |
| BC14_13SP3_sulP_ | CAAGCAGAAGACGGCATACGAGATCGATACGCGTGACTGGAGTTCAGACGTGTGCTCTT | CGATAC | GCGTATC |
| set2 | C (SEQ ID NO: 63) | GC | G |
| BC15_14SP1_wbpL_ | CAAGCAGAAGACGGCATACGAGATCTGTAAGTGTGACTGGAGTTCAGACGTGTGCTCTT | CTGTAA | ACTTACA |
| set2 | C (SEQ ID NO: 64) | GT | G |
| BC16_14SP3_wbpL_ | CAAGCAGAAGACGGCATACGAGATACGTAGCCGTGACTGGAGTTCAGACGTGTGCTCTT | ACGTAG | GGCTAC |
| set2 | C (SEQ ID NO: 65) | CC | GT |
| BC17_42SP1_ampR_ | CAAGCAGAAGACGGCATACGAGATGGCTTCAAGTGACTGGAGTTCAGACGTGTGCTCTT | GGCTTC | TTGAAG |
| set2 | C (SEQ ID NO: 66) | AA | CC |
| BC18_42SP1_mexR_ | CAAGCAGAAGACGGCATACGAGATTCCACACCGTGACTGGAGTTCAGACGTGTGCTCTT | TCCACA | GGTGTG |
| set2 | C (SEQ ID NO: 67) | CC | GA |
| BC19_42SP1_ampD_ | CAAGCAGAAGACGGCATACGAGATGTTGCTCAGTGACTGGAGTTCAGACGTGTGCTCTT | GTTGCTC | TGAGCA |
| set2 | C (SEQ ID NO: 68) | A | AC |
| BC20_42SP1_ | CAAGCAGAAGACGGCATACGAGATCTCACTTAGTGACTGGAGTTCAGACGTGTGCTCTT | CTCACTT | TAAGTG |
| PA0810_set2 | C (SEQ ID NO: 69) | A | AG |
| BC21_42SP2_ampR_ | CAAGCAGAAGACGGCATACGAGATGCACTAGAGTGACTGGAGTTCAGACGTGTGCTCTT | GCACTA | TCTAGTG |
| set2 | C (SEQ ID NO: 70) | GA | C |
| BC22_42SP2_mexR_ | CAAGCAGAAGACGGCATACGAGATTACAACACGTGACTGGAGTTCAGACGTGTGCTCTT | TACAAC | GTGTTGT |
| set2 | C (SEQ ID NO: 71) | AC | A |
| BC23_42SP3_ampD_ | CAAGCAGAAGACGGCATACGAGATGCGAACACGTGACTGGAGTTCAGACGTGTGCTCTT | GCGAAC | GTGTTCG |
| set2 | C (SEQ ID NO: 72) | AC | C |
| BC24_42SP4_ | CAAGCAGAAGACGGCATACGAGATAGAGTGCGGTGACTGGAGTTCAGACGTGTGCTCTT | AGAGTG | CGCACTC |
| PA0810_set2 | C (SEQ ID NO: 73) | CG | T |

List of Exemplary Genes of Table 2 with Corresponding Amino Acid Sequences

PA2707
(SEQ ID NO: 74)
MKFEGTQSYVATDDLKLAVNAAITLQRPLLVKGEPGTGKTMLAEQLAESFGAKL
ITWHIKSTTKAHQGLYEYDAVSRLRDSQLGVDKVHDVRNYIKKGKLWEAFEAEERVIL
LIDEIDKADIEFPNDLLQELDKMEFYVYETNETIKAKQRPIIIITSNNEKELPDAFLRRCFFH
YIAFPDRETLQKIVDVHYPNIKQSLVSEALDIFFDVRKVPGLKKKPSTSELVDWLKLLMA
DEIGEAVLRERDPTKAIPPLAGALVKNEQDVQLLERLAFMSRRASR
glcD
(SEQ ID NO: 75)
MNILYDERLDGPLPQVDKDGLLAELRLRLPDLELLHAAEDLRPYECDGLSAYRC
TPLLVALPERIEQVQGLLALCHRLKVPVVARGAGTGLSGGALPLENGVLLVMARFRRIL
EIDPLGRFARVQPGVRNLAISQAAAPHGLYYAPDPSSQIACSIGGNVAENAGGVHCLKY
GLTVHNLLQVDIVTLEGERLSLGSSALDSAGFDLLALFTGSEGLLGVVVEVTVRLLPRPP
VAKVLLASFDDVESAGRAVADLIGAGIVPAGLEMMDNLSIRAAEDFIHAGYPVDAAAIL
LCELDGVEADVHEDCERVRELFEAAGATSVRQAQDEAERQRFWAGRKNAFPAVGRISP
DYYCMDGSIPRRELPRVLHGIAELSREYGLRVANVFHAGDGNMHPLILFDANLPGELER
AEALGGRILELCVAVGGSITGEHGVGREKINQMCAQFNADELTLFHAVKAAFDPAGLLN
PGKNVPTLHRCAEFGAMHVHHGRLPFPELERF
kinB
(SEQ ID NO: 76)
MSMPLPMKLRTRLFLSISALITVSLFGLLLGLFSVMQLGRAQEQRMSHHYATIEV
SQQLRQLLGDQLVILLRETPDGQALERSQNDFRRVLEQGRANTVDSAEQAALDGVRDA
YLQLQAHTPALLEAPMADNDGFSEAFNGLRLRLQDLQQLALAGISEAETSARHRAYLV
AGLLGLVGVAILLIGFVTAHSIARRFGAPIETLARAADRIGEGDFDVTLPMTNVAEVGQL
TRRFGLMAEALRQYRKTSVEEVLSGERRLQAVLDSIDDGLVIFDNQGRIEHANPVAIRQL
FVSNDPHGKRIDEILSDVDVQEAVEKALLGEVQDEAMPDLVVDVAGESRLLAWSLYPV
THPGGHSVGAVLVVRDVTEQRAFERVRSEFVLRASHELRTPVTGMQMAFSLLRERLDF
PAESREADLIQTVDEEMSRLVLLINDLLNFSRYQTGMQKLELASCDLVDLLTQAQQRFIP
KGEARRVSLQLELGDELPRLQLDRLQIERVIDNLLENALRHSSEGGQIHLQARRQGDRVL
IAVEDNGEGIPFSQQGRIFEPFVQVGRKKGGAGLGLALCKEIIQLHGGRIAVRSQPGQGA
RFYMLLPV
sulP
(SEQ ID NO: 77)
MPLARWVPGLDSLLHYRRAWFRPDVQAGLSVAAIQIPTAIAYAQIAGFPPQVGL
YACILPMLIYALIGSSRQLMVGPDAATAAMVAAAITPLAAGDPQRLVDLSMIVAIMVGL
FSIVAGLARAGFIASFLSRPILVGYLNGIGLSLLVGQLGKLFGYEAATSGFVAGILALLEN
LLHIHWPTLILGSLSLLLMVLLPRRFPQLPGALCGVLLASLAAALLGLDRYGVELLGEVP
AGLPQLSWPQTSLEELKSLLRDATGITVVSFCSAMLTARSFAARHGYSINPNHEFVALGL
ANIGAGVSQGFAISGADSRTAVNDMVGGKTQLVGVVAALVIAATLLLLNKPLGWVPMP
ALGAVLLLAGWGLIDVQALKGFWKLSRFEFSLCLLTTVGVLSVGVLPGIFVAVSIAVLR
LLYYTYRPSDAVLGWMHGIDGQVELAKYPQATTLPGLVIYRFDAPLLFFNADYFKQRV
LAVVDGSERPNAVLLNAEAMTNLDISGLATLHEVQQILKAQGVHLSLARVTGQTLDLL
QRSSMLGEIKPPLVFSSVRSGVSAYRYWLRQQERLAAQAAATSGNA
N/A
(SEQ ID NO: 78)
MKPKEFVRRLSAVSTKNSFNPYSQVCSTFDVKSADKIRFQLLLDMLEKASRVEV
DAIWIGRDLGYRGGRRTGLALTDEVHAKEYAERWSLCAQRTTKGDPCKERTASVIWDA
LRCIEDNIFLWNVFPLHPHEAGDPFSNRSHNAAERKIGEEILKDLVSMIKPRRLIAVGNDA
VSSIGKIAPNIPSAKVRHPSYGGQNIFLQQIEGLYGVVCQPVIQRELF
wbpL
(SEQ ID NO: 79)
MMNLWLLLPAVAALSLLLTAGLRRYAIARSLIDVPNARSSHQVPTPRGGGVAIV
LSFLLAVLLAAILGAVKPDLATGILGAGIGIALLGFLDDHGHIAARWRLLGHFAGACWL
LYWLGGLPALAFFGLVVDLGWVGHIAAAFYLVWMLNLYNFMDGIDGIASVEAVCVCV
GAALLVVVSGVGSDEASQGVWLAALLAAAVTGFLFWNFPPARIFMGDAGSGFLGVIIG
GLSLQAAWVSPQLFWGWLILLGVFIVDATLTLLRRLLRGDKVYEAHRSHAYQYASRHY
GRHLPVTLAVGGINIFWLLPLALLVAAGKIDGMLALLIGYLPLAFLALRFKAGVLESRAA
N/A
(SEQ ID NO: 80)
MKIKAALIVDDLSLSEWQKRAIEDSSEYLDIQLVLSCRNSATKKSVIKHCGYYFL
NILSLKNDMTRRVQLDSRGSEVIHFDSDYEGAWQRIPEDVCARILDKGIKLVIKFGMSLL
RIDGGLQRLDILSYHHGDPEYYRGRPAGFYEIYENADSVGIIVQKLSNKLDAGEVLVRGY
SKVHHHSYKKTSRNFYLNSVVLLRKALVNYSRGEQVVLEKLGKNYRLPSNFTVFKFFC
KTIFRGLARLSYGAFFEKKWNVVALPYNDIPSLQELSVSAGKIPKVEKGYTFYADPFFSA
DGKLIRLEAQGNRMNACRFE
csgA
(SEQ ID NO: 81)
MHNVLIVGASRGIGLGLADAFLQRGAQVFAVARRPQGSPGLQALAERAGERLQ
AVTGDLNQHDCAERIGEMLGERRIDRLIVNAGIYGPQQQDVAEIDAEQTAQLFLTNAIAP
LRLARALSGRVSRGGVVAFMSSQMASLALGLSATMPLYGASKAALNSLVRSWEGEFEE
LPFSLLLLHPGWVRTEMGGDSAPLSVEESAAGLVAAVEDAAGVNACRFVDYRNQPLP
W
trmJ
(SEQ ID NO: 82)
MLDRIRVVLVNTSHPGNIGGAARAMKNMGLSQLVLVQPESFPHGDAVARASGA
TDILDAARVVDTLEEALSGCSVVLGTSARDRRIPWPLLDPRECATTCLEHLEANGEVAL
VFGREYAGLTNEELQRCQFHVHIPSDPEFGSLNLAAAVQVLTYEVRMAWLAAQGKPTK
MEKFESTSMLNTELVTADELELYYAHLERTLIDIGFLDPEKPRHLMSRLRRLYGRSAISK
LEMNILRGILTETQKVARGLSYKRSDD
PA3470
(SEQ ID NO: 83)
MTDNLLSISAACLFDDQGNLLLVRKRGTQAFMLPGGKREPGETPLAALQRELLE
ELRLPMGASTFEHLGSFQAPAANEANTRVDADIYVARLPHAVCAQAELEELAWLVPGQ
AQPDNLAPLLRDHVLPALARRAAENPETQAEHRTRPDHVR
N/A
(SEQ ID NO: 84)
MRALWFCVALMPVLAQADIYRWTDAQGKVHFSATPPAGAQRVEVRPQVVERD
AATRQREQRTQEYFDARREERTAAAERAGQRQAALAEECGRLRQQLSQLERGGRFYRQ
DAGGGPVYLSDAELDAIRRELASRESERCR
msbA
(SEQ ID NO: 85)
MSDSPQNPGPSSLKIYFRLLGYVKPYIGMFLLSIVGFLIFASTQPMLAGILKYFVD
GLSNPDAALFPNVQWPWLRDLHLVYAVPLLIILIAAWQGLGSFLGNFFLAKVSLGLVHD
LRVALFNKLLVLPNRYFDTHSSGHLISRITFNVTMVTGAATDAIKVVIREGLTVVFLFLY
LLWMNWKLTLVMLAILPVIAVMVTTASRKFRKQSKKIQVAMGDVTHVASETIQGYRV
VRSFGGEAYEEKRFLDASQSNTDKQLRMTKTGAVYTPMLQLVIYVAMAILMFLVLWLR
GDASAGDLVAYITAAGLLPKPIRQLSEVSSTVQRGVAGAESIFEQLDEAAEEDQGTVEKE
RVSGRLEVRNLSFRYPGTDKQVLDDISFIAEPGQMIALVGRSGSGKSTLANLVPRFYQHN
NGKILLDGVEVEDYRLRNLRRHIALVTQQVTLFNDSVANNIAYGDLAGAPREEIERAAK
AANAKEFIDNLPQGFDTEVGENGVLLSGGQRQRLAIARALLKDAPLLILDEATSALDTES
ERHIQAALDEVMKGRTTLVIAHRLSTIEKADLILVMDQGQIVERGSHAELLAQNGHYAR
LHAMGLDEQAPAPVG
mutY
(SEQ ID NO: 86)
MTPEGFNGAVLDWYDRHGRKDLPWQQGITPYRVWVSEIMLQQTQVSTVLGYF
DRFMAALPDVEALAAAAEDEVLHLWTGLGYYSRARNLHKTAQIVVERHAGEFPRDVE
QLAELPGIGRSTAGAIASLSMGLRAPILDGNVKRVLARYLAQDGYPGEPKVARALWEA
AERFTPHARVNHYTQAMMDLGATLCTRSKPSCLLCPLVSGCRAHLLGREADYPQPKPR
KALPQKRTLMPILANRDGAILLYRRPSSGLWGGLWSLPELDDLDGLEPLAARHSLALGE
RRELSGLTHTFSHFQLAIEPWLVAVESAPRAVAEGDWLWYNLATPPRLGLAAPVKKLL
KRAEQELGRGTAA
oprD
(SEQ ID NO: 87)
MKVMKWSAIALAVSAGSTQFAVADAFVSDQAEAKGFIEDSSLNLLLRNYYFNR
DGKEGRGDRVDWTQGFLTTYESGFTQGTVGFGVDAFGYLGLKLDGTSDKTGTGNLPV
MNDGKPRDDYSRAGGAVKVRISKTMLKWGEMQPTAPVFAAGGSRLFPQTATGFQLQS
SEFEGLDLEAGHFTEGKEPTTVKSRGELYATYAGQTAKSADFAGGRYAITDNLSASLYG
AELKDIYRQYYLNTNYTIPLASDQSLGFDFNIYRTTDEGKSKAGDISNTTWSLAGAYTLD
AHTFTLAYQQVHGDEPFDYIGFGGNGSGAGGDSIFLANSVQYSDFNGPGEKSWQARYD
LNLASYGVPGLTFMLRYINGKDIDGTKVDSSSSYAGLYGEDGKHHETNLEAKYVVQSG
PAKDLSFRIRQAWHRANADQGEGDQNEFRLIVDYPLSIL
ampD
(SEQ ID NO: 88)
MHFDSVTGWVRGVRHCPSPNFNLRPQGDAVSLLVIHNISLPPGQFGTGKVQAFF
QNRLDPNEHPYFEEIRHLTVSAHFLIERDGAITQFVSCHDRAWHAGVSCFDGREACNDF
SLGIELEGTDTEPYTDAQYTALAGLTRLLRAAFPAITPERIQGHCDIAPERKTDPGEAFDW
SRYRAGLTDSKEET
sltB1
(SEQ ID NO: 89)
MQVLRTWAARGVQWVGVAGVIGLSGAAQAGDYDGSPQVAEFVSEMTRDYGF
AGEQLMGLFRDVNRKQSILDAISRPAERVKQWKEYRPIFISDARISRGVDFWNKHAEDL
ARAEKEYGVPAEIIVSIIGVETFFGRNTGSYRVMDALSTLGFDYPPRADFFRKELREFLLL
AREQQVDPLSLTGSYAGAMGLPQFMPSSFRAYAVDFDGDGHINIWSDPTDAIGSVASYF
KQHGWVTGEPVVSVAEINDESAESAVTRGVDPTMSLGELRARGWRTHDALRDDQKVT
AMRFVGDKGIEYWVGLPNFYVITRYNRSAMYAMAVYQLAGEIARARGAH
pilL
(SEQ ID NO: 90)
MGDRHDYVALEWVKGEIAETLKQARQALEAFVENPQDPTRMRFCLTYVHQVQ
GTLQMVEFYGAALLAEEMEQLVQALLDGRVPNQGEALEVLMQAILQLPVYLDRIQTAR
RDLPMVVLPLLNDLRAARGEKLLSETSLFAPDLSQRQPQLDGEAIAQLRTDELGGLLRK
LRQTQQMALVGLLRNQDVATSLGYLARVYARLEGLCREAPLGPLWSIASGLVEGLANG
SVVNSASVRTLLRQLDRELKRLVEQGADGLNQAAPDELVKNLLFYVAKAPSQSPRIRAL
KEQYRFDEALPDHETVDAERARLAGPDRDAMRSVVGALCEELVRIKDSLDLFVRSDRG
HPSELDALLAPLKQIADTLAVLGFGQPRKVILDQLDVIHALAQGRREPSDAILMDVAGA
LLYVEATLAGMAGPGDERNSEESRLPTTDVAQIHQLVIKEARNGLEQAKDAIIEFIASQW
NHEHLARVPELLTQVRGGLAMIPLERAATLLETCNRYIQEQLLARKAVPDWQSLDTLAD
AITSVEYYLERLSEDHASQSDLILDVAEDSLANLGYTLKPNSSAPAEPGLSGPAAIESPAA
EPERPEAVVEVAETAEQPPADTASAEAAREDAPLLASDDNWTLGEVVPDAGEPSLDLAL
DLPLDDSAEVPPALPEVVEESGQPQSTPAPARSLDDFSLDEIDLSGLDLPADAAPASGPA
ALADWSLPEQWGLGDDLAQPAQAGETLDLSLEEPALSFDAPLESLEPLPALEPFDGSAE
QELVLDALDPLPLDVALPESEGEVSAWEGSSLEELDLSDLDLPEVQLPEAEAEAPPAAEA
LASEAPALSLAEVMAAPVQPINPPAQNVPVSLLPPPADEEPVDEELREVFIEEAGEVLETI
GRYLPAWKADHDDREALTEVRRAFHTLKGSGRMVRALVIGELAWSIENLFNRVLDRSI
AASEPVQRVVDQVVALLPELVEEFAANAQRQRDDVDLLAATAHALAKGEPLPEPPAPD
DGGVPPEAGAEQPSSLDNGVQAPPLADAPQAAAEAQSDVELLDPQLLEIFTNEAETHLE
ALVGFLADCARELPQPVTDALQRALHTLKGSAHMAGILPIAEIATPLEKLVKEYKSNLLA
FDLREAELLHDAEQLFRIGLEQVGAQRPLNPIPGSDALLERIEALHQERIASLEAERYSDA
GERRDPLLIEAFLVEGMDILLDAEDLLERWHEHPQERQELSALREELSTLDRGARHAELP
QVEELCQALLALYDAVEEGRLAVSPAFFEEARQAHEALIGMMDQVAAGLQVTPRPERV
AALQELLEAPAAEAVPFIDPESLGADDFPPEDEEPALPEAVFEEAGTPAEETVPAAPAPAP
GRELDEEMVSIFLEEAVDILESAGQALAQWQAEPGALSSLSALQRDLHTLKGGARMAEI
AEIGDLAHELEALYEGLVDRRYQHSPQLAGLLQACHDRLAEQLDQLSAGQPLADPHDLI
QAIRRFRQGPVAEAATPGEAESPVEELVAPAVEEPAAPAAEAFEERDPELVEIFLEEGFDI
LDSAAAALQRWIDDVDNTIELEALQRDLHTLKGGARMAEIGEIGDLAHELEFLYEGLCG
GRLRASPALFGLLQRCHDELAEMLEAVRGHRSLPDGQALIAEIRRLRSDPDEQLSVPTSV
SLKPLAAKGAAADESEILDIFLEEADDLLENLELALGRWDGGNGDAQPLDDLLRILHTL
KGGARLAGQTELGNLAHDLEQHLTDAQQQGAPWPDSLLLDAQSGLEGLQRQVDLLRE
RLAEDDEAGERPEPAQALVQADDTDRAVASALAELTRLAPAAGAIMAAEAAPPAAPAT
TLPFVRKAQEAAQEAASRRAPQELVKVPAELLENLVNLAGETSIFRGRVEQQVSDVGFT
LGEMESTIERVRDQLRRLDTETQAQILSRHQADAERAGYEEFDPLEMDRYSQLQQLSRA
LFESASDLLDLKETLAAKNRDAETLLLQQARVNTELQEGLMRTRMVPFDRLVPRLRRIV
RQVAGELGKQVEFVVGNADGEMDRTVLERIVAPLEHMLRNAVDHGIESGETRRAAGKP
EHGTIRLNIGREGGDILLTLSDDGAGIRLDAVRRKAIERGLMSADSDLSDHEVLQFVLES
GFSTAEKVTQISGRGVGLDVANSEVKQLGGSVSIQTEPGQGTRFNVRLPFTVSVNRALM
VLSGEDLYAVPLNTIEGIVRVSPYELEALYDQRGEAGLDTPSFEYAGQSYELKYLGELLN
NGQEPKLVGQSLPLPVILVRSSEHAVAVQVDSLAGSREIVVKSLGPQFAGVAGISGATLL
GDGRVVVILDLLATIRSRHALLGQESRRERLALRQEMAASESEQQRPPLVMVVDDSVTV
RKVTTRLLERNGMNVLTAKDGVDAIAQLQEHRPDILLLDIEMPRMDGFEVATLVRHDE
RLGNLPIIMITSRTGEKHRERALGIGVNQYLGKPYQETELLEAIQSLVGQHE
vfr
(SEQ ID NO: 91)
MVAITHTPKLKHLDKLLAHCHRRRYTAKSTIIYAGDRCETLFFIIKGSVTILIEDDD
GREMIIGYLNSGDFFGELGLFEKEGSEQERSAWVRAKVECEVAEISYAKFRELSQQDSEI
LYTLGSQMADRLRKTTRKVGDLAFLDVTGRVARTLLDLCQQPDAMAHPDGMQIKITRQ
EIGRIVGCSREMVGRVLKSLEEQGLVHVKGKTMVVFGTR
czcA
(SEQ ID NO: 92)
MIAKLIRWSVANRFLVLLATAMLTAWGVWGVRSTPVDALPDLSDVQVIIRTNYP
GQAPQIVENQVTYPLATTMLSVPGAKTVRGFSFFGDSFVYVLFEDGTDLYWARSRVLE
YLNQVQGRLPATAKPALGPDATGVGWIFQYALVDRTGKNDLAQLRALQDWFLKFELK
SLPNVAEVASVGGMVKQYQVVLDPIKLASYGLSQAQVRDALMGANQETGGSVLELSG
AEYMVRASGYLKTLDEFREIPLTARGGVPVRLGDVATLQIGPEMRRGIAELDGEGEVAG
GVVVLRSGKNAQETIAAVKAKLAELQGSLPPGVEIVTTYDRSALIERAIRNLTTKLGEEF
LVVALVCALFLWHLRSALVAIISLPLGVMTAFLVMRYQGINANIMSLGGIAIAVGAMVD
AAVVMIENAHKKLEAWQHAHPDQRLQGKERWDVITQAAEEVGPALFFSLLIITLSFIPVF
TLEAQEGRLFGPLAFTKTYAMAAAAGLSVTLIPVLMGYWIRGRIPDEQKNPITRILIAAY
RPALEWVLRRPKATLLIAVLALATTAWPLARLGGEFLPRLDEGDLLYMPSALPGLSAQR
ATELLQLSNRMIKTVPEVDKVFGKAGRAETATDPAPLEMFETTVKLKPREQWRPGMTP
EKLVEELDRAVKIPGLSNIWIPPIRNRIDMLATGIKSPIGVKVTGNDLGVIDRIAAEVEQV
AKGIPGVTSSLAERLTGGRYVDVQIDRVAAGRYGLNIADVQAVIAGAVGGENVSETVE
GLARFPINLRYAREWRDSPQRLAELPIFTPMGQQITLGTVARIAITDGPPMLKSENARPSG
WVYVDVRGRDLASVANELRDAIGQQVKLEPGVSITYSGQFEYMERANARLKVVVPATL
LIIFVLLYLTFARVDEAGLIMATLPFALTGGIWFLYLLNYNLSIATGVGFIALAGVAAEFG
VVMLIYLKQALAERCPDGRNPTREELLDAIREGAVLRVRPKAMTVAVILAGLVPIVWSS
GTGSEVMSRIAAPMLGGMVTAPLLSLFVIPAAYLLMRKPR
N/A
(SEQ ID NO: 93)
MSHYLEVLTPQNSQIIFIDQQPQMAFGVQSIDRQTLKNNVVGLAKAAKVEDIPVT
ITTVETDSFSGPTYPELLAVFPEQKILERTSMNSWDDQNVRDSLAAAGRKKVVVAGLWT
EVCNTTFALCAMLEGGYEIYMVADASGGTSSDAHKYAMDRMVQAGVVPVTWQQVLL
EWQRDWARRDTYDAVMAIAKEHSGAYGMGVDYAYTMVHKAPERVTHGERIGPNPAK
PA3052
(SEQ ID NO: 94)
MSSNSRSNSTFTGRLSALANRLRLGAARSDGRELRERAAALELPFQPLRRPEPSV
WWQAGPPLHTLVDLPRGALSGPVQEDKAEAHAVLKRLVRVSHSTLESVDLKTIEGVCS
REILVQAPCPRLEDLATAEVCRGVRIISYKDFVKALSLALPRFTNGDSIRLRQAAWHGER
LFWAGERQACAFAAAIVYARRRELELKLPAHLERYELEPGALDELEQRYHMLRIPTEA
WSEPTFMSLLLDTGLPYARLALFTPETPECLLLPRNDERADALGEGLRAAGAADVVKYL
KQL
N/A
(SEQ ID NO: 95)
MKDLKVAVVGLGYVGLPLAVEFGKKRTVVGFDINQGRIAELRQGIDSTLEVDAA
ELKEASELSFTFNLQDLQKCNVFIVTVPTPIDEHKQPDLTPLVKASESIGKVLKKGDIVIY
ESTVYPGATEEDCVPVLEKFSGLRFNEDFFAGYSPERINPGDKEHRVSSIKKVTSGSTPEI
AELVDSLYREIITAGTHKASSIKVAEAAKVIENTQRDLNIALINELAIIFNRMGIDTEAVLK
AAGTKWNFMPFRPGLVGGHCIGVDPYYLTHKAQSIGYHPEIILAGRRLNDGMGAYVVS
QLVKAMLKRRIHVDGARVLLMGLTFKENCPDLRNTKVVDIVRELAEYNIQVDVFDPWV
SAEDAMHEYGITPVGTPSHGAYDGIILAVAHSEFKNMGAENIRKLGKAEHVLYDLKYLL
DEDKSDLRL
cyaB
(SEQ ID NO: 96)
MREYYSRVLAYIACGASIAAGTYTQYFSYGILWMVPYALLYPHLAYHLGQRFRQ
HDPRKVTRALLAVDAVHCGLGMALLGFSVVPSLMFLLVLSFTALVIGGLRLLGMALLV
SASSALLVAVLVAPPLLGNTPVEVAAVSILFCGLYICITAFFGHQQGLRLAQVRQEIARE
QEKAARLARNLAKYLSPQVWEMIFSGKKSVRLETQRKKLTVFFSDIRGFTELSEELEAE
ALTDLLNNYLNEMSKIALKYGGTIDKFVGDCVMVFFGDPSTQGAKKDAVAAVSMGIA
MRKHMKVLRQQWRAQGITKPLEIRMGINTGYCTVGNFGADTRMDYTIIGREVNLASRL
ESASEAGEILISHETYSLIKDVIMCRDKGQIAVKGFSRPVQIYQVVDSRRDLGAAPSYVEH
ELPGFSMYLDTNNIQNYDKERVIQALQQAAERLRDKVIL
N/A
(SEQ ID NO: 97)
MKIKAALIVDDLSLSEWQKRAIEDSSEYLDIQLVLSCRNSATKKSVIKHCGYYFL
NILSLKNDMTRRVQLDSRGSEVIHFDSDYEGAWQRIPEDVCARILDKGIKLVIKFGMSLL
RIDGGLQRLDILSYHHGDPEYYRGRPAGFYEIYENADSVGIIVQKLSNKLDAGEVLVRGY
SKVHHHSYKKTSRNFYLNSVVLLRKALVNYSRGEQVVLEKLGKNYRLPSNFTVFKFFC
KTIFRGLARLSYGAFFEKKWNVVALPYNDIPSLQELSVSAGKIPKVEKGYTFYADPFFSA
DGKLIRLEALNASNGLGEIIELKAQSLDFSRVILKGNHFSYPYSFEASGVEYLIPEVASHS
APCLLPPPFALESKKLFQGMEGERILDGTLFEHGGRYYLFCGQAVSGSDNLYLYVGESL
EGPYTSHPCNPVVMNPGSARMGGRIFKEGGKLYRFGQNNSYGYGSSLAVNEIEVLDPEH
YSEKRVANLAFQDARGPHTIDIHGQTMILDFYQDRFSLLAGYRRLVARLLSRG
pilG
(SEQ ID NO: 98)
MEQQSDGLKVMVIDDSKTIRRTAETLLKKVGCDVITAIDGFDALAKIADTHPNIIF
VDIMMPRLDGYQTCALIKNNSAFKSTPVIMLSSKDGLFDKAKGRIVGSDQYLTKPFSKE
ELLGAIKAHVPSFTPVDAVS
pilJ
(SEQ ID NO: 99)
MKKINAGNLFAGMRSSSVIAGLFIVLIVSIVLLFANFAYLNTQSNHDKQYIGHAG
ELRVLSQRIAKNATEAAAGKGEAFKLLKDARNDFEKRWNILVNGDESTSLPPSPEAVKP
QMDVVQQDWDGLRKNADSILASEQTVLSLHQVASTLAETIPQLQVEYEEVVDILLENG
APADQVAVAQRQSLLAERILGSVNKVLAGDENSVQAADSFGRDASLFGRVLKGMQEG
NAAMSISKVTNAEAVDRLNEIAELFEFVSGSVDEILETSPDLFQVREAANNIFSVSQTLLD
KASQLADGFENLAGGRSINLFAGYALGALALASIILIGLVMVRETNRRLAETAEKNDRN
QAAILRLLDEIADLADGDLTVAATVTEDFTGAIADSINYSIDQLRELVETINQTAVQVAA
AAQETQSTAMHLAEASEHQAQEIAGASAAINEMAVSIDQVSANASESSAVAERSVAIAN
KGNEVVHNTITGMDNIREQIQDTSKRIKRLGESSQEIGDIVSLINDIADQTNILALNAAIQA
SMAGDAGRGFAVVADEVQRLAERSSAATKQIEALVKTIQTDTNEAVISMEQTTSEVVRG
ARLAQDAGVALEEIEKVSKTLAALIQNISNAARQQASSAGHISNTMNVIQEITSQTSAGT
TATARSIGNLAKMASEMRNSVSGFKLPEGVEQA
ampR
(SEQ ID NO: 100)
MVRPHLPLNALRAFEASARHLSFTRAAIELCVTQAAVSHQVKSLEERLGVALFK
RLPRGLMLTHEGESLLPVLCDSFDRIAGLLERFEGGHYRDVLTVGAVGIFTVGWLLPRL
EDFQARHPFIDLRLSTHNNRVDIAAEGLDYAIRFGGGAWHGTEALALFEAPLTVLCCPE
VAAQLHSPADLLQHTLLRSYRADEWPLWFQAAGLPAHAPLTRSIVFDTSLAMLEAARQ
GVGVALAPAAMFARQLASESIRRPFATEVSTGSYWLTRLQSRGETSAMLAFRGWLLEM
AAVEARGR
mexR
(SEQ ID NO: 101)
MNYPVNPDLMPALMAVFQHVRTRIQSELDCQRLDLTPPDVHVLKLIDEQRGLNL
QDLGRQMCRDKALITRKIRELEGRNLVRRERNPSDQRSFQLFLTDEGLAIHQHAEAIMSR
VHDELFAPLTPVEQATRVHLLDQCLAAQPLEDI
wbpW
(SEQ ID NO: 102)
MLIPVVLSGGAGTRLWPVSREGQPKPFMRLPDGQTLLGKTYRRAAGLLAGHGEI
VTVTNREHYFQSKDQFQAARLGRHRGHFILEPTGRNTAPAIAVAALALQAEHGDAAVL
VVMPADHLIRNEEAFREAVGHAARLAVAGHLVTFGVVPDAAETGFGYIELGDRLDEQG
AAKVRRFVEKPDEETARRYVESGGFLWNSGMFCFTASTLVDELAQHAPALLEQARACL
AASAAVKMADGIQHELAGEAFAALPDISIDYALMERSARVAVVPAAFDWSDIGSWGA
MSALLDADAEGNRGSGDTLFVDTRNTFVQSDGRLVATVGVDDLVVVDTSDALLIARAD
RVQEVRRVVQRLKDERHEAYRLHRTVNRPWGSYTVLEEGPRFKIKRIVVRPGERLSLQ
MHHHRSEHWIVVQGMARVINGDGARLVNSNESTYIPAGHRHRLENPGVIDLVMIEVQS
GEYLGEDDIVRFEDQYGRVV
dipA
(SEQ ID NO: 103)
MKSHPDAASRSAAEVVTQLPVPSRLGLLRFERLNEPSWALLFLDPACERQLGLPA
TTLCALLDAPYASLMEPEARHRLHEQIQQQLVKRPHYQVSYKLHTPNGVLTMLEFGEAF
QQHGRQLLHGYLMVEERAESAERSEQLLDLESQNLRLKASLDLYQRSQDDHLQHLLRS
RTQQNLIVRLARHRYLSSDPLLEAAQLITQAACEAYGTARAGIWRLLDDQRLEAVTVYR
RDLDQYEKPQSIDASRYPAYLEAVHSGRAIDAHNAQRDPRTQELYKDYLRPLGVNALL
DATIRIGGEVVGVLCLEHAGENRMWQSDEIAFAGELADQYAQVLMNHERRNVSSALHL
FQRAVEQSASAFLLIDRDGVVEYVNPSFTSITQYSADEVRNRRLSELPALENLSELLFDA
RSALTQQNSWQGEFRSRRKNHEPYWGQLSLSKVYDDLGELTHYIGIYEDITQNKLAQQ
HIEKLAYRDNLTGLANRHYFIGALEERLESSGDRPLSLLLVDIDNFKRINDSLGHQTGDK
LLVSLARRLRSCLGDGATLARFASNEFAVLLDDTAVEKGESIAAQVLHMLDKPLFVDN
QLINITGSIGLASAPQHGCDPQTLMKYAGLALHKAKANGKHQVQVFTEALTAEASYKLF
VESNLRRALAQNELAVHYQPKLCLRSGQLLGLEALLRWQHPEKGMIRPDRFISVAEETG
LIVPIGKWVIREACRQARELAEAGLGELQIAINLSPKQFTDPDLVGSIAAILHEENIPASQL
ELELTESLLLDATDDTRQQLERLKSLGLTLAMDDFGTGYSSLSYLKKFPIDVIKIDRSFIK
DIPDSQDDMEITSAVIAMAHNLKLKVVAEGVESAEQLAFLRRNRCDIGQGYLFDRPIPSD
LLNTSLLRYPCRTLH
retS
(SEQ ID NO: 104)
MVRLRIAIGLLVSFLLLLLGPMSPAVADDAGVSSVPLQTTATTPSVNQNWRLLRD
ESAQLRIADVLQRKEQFRPLAKRSFIFPASPQAVWLQVQLPAQKVPSWLWIFAPRVQYL
DYYLVQDGQLVRDQHTGESRPFQERPLPSRSYLFSLPVDGKPMTLYVRMTSNHPLMAW
FDQIDEAGLVGLEKPAYAFGMLLGGMLLLLMYNLIRFAYSRSASSLWLAAVHAALAVC
AAANLGLVAFWLPGLKFNQSLTADLGALGAAVSLLWFACSFFRGTAESRLNRLLQGEA
LLILAVGAIIAFTQQLWFSWLIYLLVILSSLSVPLIAAWHWYRGYQPARLIVAGMIVENA
GFMVFLPVLFGTKQLDPGWLVLGVFSFATLAGLVLSVSLTERQRLIQQLNLQQRTSEAA
HTAELQTKAEFLAKISHEIRTPMNGVLGMTELLLGTPLSAKQRDYVQTIHSAGNELLTLI
NEILDISKLESGQIELDEVQFDLNALIEDCLDIFRVKAEQQRIELISFTQPQVPRVIGGDPTR
LRQVVLSLLDNAFKQTEEGEILLVVALDDQGETPRLRIAVRDSGHPFDAKEREALLTAEL
HSGDFLSASKLGSHLGLIIARQLVRLMGGEFGIQSGSSQGTTLSLTLPLDPQQLENPTADL
DGPLQGARLLVVDDNETCRKVLVQQCSGWGLNVSAVSSGKEALAQLRTKAHLREYFD
VVLLDQDMPGMTGMQLAAKIKEDPNLNHDILLIMLTGISNAPSKIIARNAGIKRILAKPV
AGYTLKATLADELAQRGVSGVTNYLQPAKEAQAPSLPSDFRILVAEDNSISTKVIRGML
NKLNLQPDTASNGQEALSAMKATQYDLVLMDCEMPVLDGFSATEQLRAWEAHEQRPH
TPVVALTAHILSEHKERARLVGMDGHMAKPVELSQLRELIAYWVGERDRRRQGDALPS
N/A
(SEQ ID NO: 105)
MDDNKNKVLRLRDVVLYTVSAMLFMDQIALASSLGPSSLFWWLYVLVLLFLPM
AMMTSELGTAFPANGGVYHWVRSAFGFRWGARVSWMYWVNNALWMPSVYTLFGSM
LGAFYFPELSLWGKIAIGIALALLTAAFNVVALRLGKWLPNLGALLKLLAVLALGVGGL
HFGWNHGFANDFSLDSIVPSSPGQMAALGVMVYGIMGTELACCSAAEMRNARRDIPRA
VLISGLIVGAFNIFGTLGVLAAVPAEETDVTRIFAHTLYNIYGHDGAGGMLADLVGAFVL
FTLFTNMVTWSMGTNRAAVEAAKAGELPALFGVVHSRHGTPIGSAVLASAVSIVLLLLY
GLVAHTAEELFWTLLSIFAMVFMMPYVLMCLAFVRLRRADPRPRPYRMPLGDRLASLW
ALFVALHVLAGICLFVVTPGAPMDWAYAGKIVGGVALALAVGELLIRQAARRRGVMSL
RGAYG
dapB
(SEQ ID NO: 106)
MRRIAVVGAAGRMGKNLIEAVQQTGGAAGLTAAVDRPDSTLVGADAGELAGL
GRIGVPLSGDLGKVCEEFDVLIDFTHPSVTLKNIEQCRKARRAMVIGTTGFSADEKLLLA
EAAKDIPIVFAANFSVGVNLCLKLLDTAARVLGDEVDIEIIEAHHRHKVDAPSGTALRMG
EVVAQALGRDLQEVAVYGREGQTGARARETIGFATVRAGDVVGDHTVLFAAEGERVEI
THKASSRMTFARGAVRAALWLEGKENGLYDMQDVLGLR
ampD
(SEQ ID NO: 107)
MHFDSVTGWVRGVRHCPSPNFNLRPQGDAVSLLVIHNISLPPGHFGTGKVQAFF
QNRLDPNEHPYFEEIRHLTVSAHFLIERDGAITQFVSCHDRAWHAGVSCFDGREACNDF
SLGIELEGTDTEPYTDAQYTALAGLTRLLRAAFPAITPERIQGHCDIAPERKTDPGEAFDW
SRYRAGLTYSKEET
petD
(SEQ ID NO: 108)
MNKFMAWVDARFPATKMWEDHLSKYYAPKNFNFWYFFGSLALLVLVNQILTGI
WLTMSFTPSAEEAFASVEYIMRDVDYGWIIRYMHSTGASAFFIVVYLHMFRGLLYGSYQ
KPRELVWIFGMLIYLALMAEAFMGYLLPWGQMSYWGAQVIISLFGAIPVVGEDLAQWI
RGDFLISGITLNRFFALHVIALPIVLLGLVVLHILALHEVGSNNPDGVDIKKKKDENGVPL
DGIAFHPYYTVKDIVGVVVFLFIFCTVIFFFPEMGGYFLEKPNFEMANQFKTPEHIAPVW
YFTPFYAILRAVPDKLMGVVAMGAAIAVLFVLPWLDRSPVRSIRYKGWLSKLWLVIFA
VSFVILGYYGAQAPSPLGTTLSRVCTVLYFAFFILMPFYTRMEKTKPVPERVTG
bifA
(SEQ ID NO: 109)
MKLDSRHSLSLKLLRVVLLAALAVGVVLSCAQIVFDAYKAKQAVSSDAQRILA
MVRDPSTQAVYSLDREMAMQVLEGLFQHEAVRQASIGHPGEPMLAEKSRPLLDLPTRW
LTDPILGQERTFSIRLIGRPPYSEYYGDLKITLDTAPYGENFVTTSEIIFISGILRALAMGLV
LFLVYHWMLTKPLSKIIEHLVSINPDRPSQHQLPLLKGHERNELGLWVTTANQLLASIES
NSHLRREAEDNLLRISQYDFLTGLPNRQLLQQQLDQILDGAGRQQRRVAVLCLGLDDFK
GINEQYTYQLGDQLLIALADRLRGHSARLGSLARLGGDQFALVQADIEQPYEAAELAQS
ILDGLEAPFEIDQHEVRLRATIGITLFPEDGETTEKLLQKAEQTMTLAKTRSRNRYQFYIA
SVDSEMRRRRELEKDLRDALQRHELHLVYQPQVDYRDHRVVGVEALLRWQHPLHGFV
PPDLFIPLAEQNGSIFSIGEWVLDQACRQLREWHDQGFDDLRMAVNLSTVQLHHNALPR
VVSNLLQVYRLPARSLELEVTETGLMEDISTAAQHLLSLRRAGALIAIDDFGTGYSSLSY
LKSLPLDKIKIDKSFVQDLLQDEDDATIVRAIIQLGKSLGMQVIAEGVETAEQEAYIIAEG
CNEGQGYLYSKPLPARELTQYLKQARRLSQATSSERP
PA0810
(SEQ ID NO: 110)
MRAILFDVFGTLVDWRSSLIEQFQALERELGGTLPCVELTDRWRQQYKPAMDRV
RNGQAPWQHLDQLHRQSLEALAGEFGLALDEALLQRITGFWHRLRPWPDTLAGMHAL
KADYWLAALSNGNTALMLDVARHAGLPWDMLLCADLFGHYKPDPQVYLGACRLLDL
PPQEVMLCAAHNYDLKAARALGLKTAFIARPLEYGPGQSQDLAAEQDWDLIASDLLDL
HRQLAASA
shaC
(SEQ ID NO: 111)
MSHWLILPILLPLFAGSLLLLPLAERWQRGLSLLAALALIPLSLLLIRTAASGDLSV
YALGNWAAPFGIVLMLDRLAALMLLATAVLGSAALIYALRGDDRLGKHFHALFQFQLL
GINGAFLTGDLFNLFVFFEILLIASYALLLHGGGAERVRSGLHYVILNLVGSAFFLIAVGT
LYGLTGTLNMADMAQKIAMADAERAPLLAAAGLLLLVVFALKAALLPLYFWLPRAYA
AASAPVAALFAIMTKVGIYSILRVYTLVFGDAAGELANLAQAWLWPLALATLGLGAIG
ALAARTLQSLLAYLVVVSAGTLLAGVALGSERALAASLYYLLHSTWIAGGLFLLADLV
ARQRGDKAGDLVQGPALQNPRLLGGAFFIGAIAVAGLPPLSGFFGKVMLLQSVAPGSQA
LALWSVVLGSGLVALVALSRAGSTLFWRTGHTVLGSAELDHGRLFACILLLSAGPLLVF
AAKPLLAYVQATAAQLHDLDLYRQIITRGGAA
lasR
(SEQ ID NO: 112)
MALVDGFLELERSSGKLEWSAILQKMASDLGFSKILFGLLPKDSQDYENAFIVGN
YPAAWREHYDRAGYARVDPTVSHCTQSVLPIFWEPSIYQTRKQHEFFEEASAAGLVYGL
TMPLHGARGELGALSLSVEAENRAEANRFMESVLPTLWMLKDYALQSGAGLAFEHPVS
KPVVLTSREKEVLQWCAIGKTSWEISVICNCSEANVNFHMGNIRRKFGVTSRRVAAIMA
VNLGLITL
zipA
(SEQ ID NO: 113)
MDIGLREWLIVIGLIVIAGILFDGWRRMRGGKGKLKFKLDRSFANLPDDDGDSAE
LLGPARVVEHREPSFDEQDLPSVSAREAKERKGGKRQEEPRQGDLDLDDEGLALEADPS
DAAETVEPRKGKSKGRKEKEREKAPAVAAEPAPVDEVLIINVIARDESGFKGPALLQNIL
ESGLRFGDMDIFHRHESMAGNGEILFSMANAVKPGTFDLDDIDNFSTRAVSFFLGLPGPR
HPKQAFDVMVAAARKLAHELNGELKDEQRSVLTAQTIEHYRQRIIDHERRSLMQKR
mtlY
(SEQ ID NO: 114)
MHGLFLGIDCGTQGSKALLLDAGSGRTLGLGSAAQRPPEGRDGRREQDPADWL
EAMASAVRMALEEAAVDGREVRALAVSAQQHGLLLLDAEGRALRPAKLWCDTESAAE
NRELLEALGGPAGSLERLGLVLAPGYTLSKLLWSRRRFPELFARVAHILLPHDYLNHWL
TSRVCSEAGDASGSGYFDVRRRTWASDVLELVEPGGRLAAALPELIEPGACIGNLRPEA
AAALGLAPHTRVACGGGDNMLAAIGTGNIRPGLLTASLGTSGTLSAYAERPLVSPHGEL
ATFCASSGGWLPLACTMNLTGACGLVQDLLHLDLDEFSRLAAQAPVGAEGLLMLPFFD
GERVPALPHASASLHGMTAANLSRANLCRAVLEGTAFGLRYGLDLLRASGLPGEEVRL
VGGAAKNPLWRRTLADLLGLPLVCPRQTEAAALGAALQAAWSLGRESGAGESLEALC
RRCVALDESTRTQPQARQQAAYEQAYRRYLELLPPR
PA2712
(SEQ ID NO: 115)
MFALNKSALAGLASTSLFVLLWSSGAIASKWGLAHSSPFAFLVFRFGIALACLLP
LAPLLRLRAPRSARERGKALLTGLVMLGVYPIFYIFSLKLQVTPGMMATILGVQPILTAVI
LERRQSPARLFGLLLGLAGLVLVVYQGIGLAGMSTAGILCALLALAGVTGGSIMQKGIR
ENPLGTLPLQYLAGLGLCLAFVPFQPFEFEWNAGFLVPALWMGVVVSVGATLLLYRLIA
QGNLVNVTSLFYLVPAVTAIMDFMVFGNRLGWLSLLGMGLIVVGLMFVFRKAG
N/A
(SEQ ID NO: 116)
MMVMAPGANTALGAAQCSWTLECGNPSAFGDYAAVALLPLDDKRHPKGEAAL
FQVSQAWMQWSGGQEKICCNLNLGQLPTGADRVLLVVYTFSAMGPVSDLRLLRLQIDS
QIEFNLNLSDNGESAIIVGEFYCRNHQWKFRALAEGSAYGLAALGRRIGLKIDDAHPHRR
SSSSEQSRPASGATGTGFAVTSTHILTCAHVIEDMKEIHIASFEGRHRAEAVVVDQRNDL
ALLRVQGAPAFKPVAFRDGVGCDLGEPVVALGFPLAGLAGGGVHVTQGGVSALFGLH
NDSSLLQFTAPIQPGSSGSPLFDAAGSVVGMVTSTIPDAQNMNFAVKAGLALAFLDACGI
EPARTPTGKTFSTSQMAREAQQSLWKIEARNP
yqaA
(SEQ ID NO: 117)
MLTDWAAYAGLFLSAFGSATLLPLQSETVLAALLLRGGQSVAWLLALAIVGNVL
GSWVNWWLGRYLEHFRGRRWFPVGEVQLLRAQRHYRRYGRWTLLLSWVPVIGDPLTL
VAGIMREPCWSFLLIVGLAKTLRYLALAALVLGWAG
N/A
(SEQ ID NO: 118)
MLGKHSLVYFLFKSFPAILTLVGLSVFTRLLSPGEYGVYSLTIIVVGFLNTVFLQW
VALGVGRYLPECSDDQARARLLGTARAISFLVSLVIIFVTFLLWEWREEIGFSILYYMVG
FLCLAQAWHDLNLKIQNAILQPLTYGKMLLIKGAGSFFIGVLLVYFGFGVDGLLLGTLVS
LVLATIFFQDAWRGVSWALVDKEQLTRLFAYGAPLTLTFLFAFIVNASDRFFIGAFLGDA
AVGVYSVSYDLAQYSVGTVASVVHLAAFPLVMEKLSKSGLPQTQDQLRKTFIFIFAVVS
PAACGLAMVAPEISGSIMGEEFREGALKIIPLISLSAFLGALKSFYFDYSFQLASATRVQV
VTVAVSAVVDVVFNLILIPEFGIVGAAVSSVMAFSSAILISIFLGRRVFPMPALPGKDAMK
IALSVLLMAVSVASFSLESAFFGLVVKVVLGGGVYLAAMIALDVSGMRTFLKSKLIR
N/A
(SEQ ID NO: 119)
MYAMLTGATLLIFAVAARLLARSAIHPSVAMPITWGLGLIGVSLASLIGFYRVES
DALLIFLFGVMSFSLSAGCFSFLYNGYFRAPSSNFLFDSELRTRALVIFFCLAHIVFLTVIY
RDLSSIAPTLREAAYMARAQSVSGEPVLSSLSMNYLQLGQTVIPLVVLLYLRGKCGVLG
FLAISVPWMGVILLASGRASLMQMLVGLFFIYILVKGSPSLKSLLVIGLAMFLVIAVGAV
ATSKIQFHEGDGISTLFIELYRHVAGYALQGPVLFDRYYQGSIHLEPYWSPLNGFCSILAT
VGLCQKPPLHLDFYEYAPGELGNVYSMFFSMYPHYGALGVIGVMALYGMLCSYAYCK
AKKGSLYFTVLSSYLFSAIVFSLFSDQISTSWWFYVKMTIILGILCFVFRRDRMFVIRLPQ
AG
N/A
(SEQ ID NO: 120)
MSLDKQALFWLAATVGGYLLSRQLYRRVKWYWLSPIVFVPVLLYALAIPTHTR
YADYARDTNWLVALLGPATVAFAIPIWQQRELLMRHWPALLAGMFAGTAVAIGSSWA
LAQALALDGQVTLSLLPRSITTPFAMEMSHDLGGVPELTAAFVMITGVFGAVIGGTLLR
VLRLRTPLARGALFGVGAHGAGTSRAYEFGGEEGSAAGLLMVLTGLFNLLVAPLVAHC
L
pilN
(SEQ ID NO: 121)
MNLKPLIASFLLVATGGCTITNVNDTMRRAEVASESAEGLAASMRSRQDTPARP
TVRYSDTPWVSTRPIDLKIDGIPDALNCDITYSPTVDVDIFQVGQEITKWCGIPVRVTPDV
TLTGSSTSAISLPSLNDGAQSSAANPASSMGLPPLPALPQGGSALGSSGGRNLTISGLKW
KGGPAKGLLDMATVRLGLSWKYSAAENLVTIFYVDTKTFRFYAIPSVTDMTSVVQSGT
TTAAGVSNSGTSSSSSGGGISGNSGSSQSTGVTINTDITKDIGNSVQSMLTPGVGRMSMSS
STGTMTVTDTPEVLARVGDFLNGENSNITKQVLLNVKVLSVTLTDKDDLGIDWNLVYK
AVNGKWGLGWKNVTQTDAAAVQGSVSILDTSSQWAGSNLLVKALAQQGRVSTITSPS
VTTLNLQPVPVQVARQTSYLASIQTTNTADVGSTTSLTPGTVTSGFNMNLLPYVMPGKE
LLLRYSINLSALKQIRQVSSGDNTIEIPEVDNRIFSQMVKLRSGETLVLSGFEQSVDNGSK
AGVGSASNWLMGGSLKRDNSKDVIVVLITPIVEG
purT
(SEQ ID NO: 122)
MTRIGTPLSPSATRVLLCGSGELGKEVAIELQRLGCEVIAVDRYGNAPAMQVAH
RSHVISMLDGAALRAVIEQEKPHYIVPEIEAIATATLVELEAEGYTVVPTARAAQLTMNR
EGIRRLAAEELGLPTSPYHFADTFEDYRRGVERVGYPCVVKPIMSSSGKGQSVLKGPDD
LQAAWDYAQEGGRAGKGRVIVEGFIDFDYEITLLTVRHVDGTTFCAPIGHRQVKGDYH
ESWQPQAMSAQALAESERVARAVTEALGGRGLFGVELFVKGDQVWFSEVSPRPHDTG
LVTLISQDLSEFALHARAILGLPIPVIRQLGPSASAVILVEGKSRQVAFANLGAALSEADT
ALRLFGKPEVDGQRRMGVALARDESIDAARAKATRAAQAVRVEL
spuI
(SEQ ID NO: 123)
MSVPQRAVQLTEPSEFLKEHPEVQFVDLLIADMNGVVRGKRIERNSLNKVFEKGI
NLPASLFALDITGSTVESTGLGLDIGDADRICYPIPGTLSMEPWQKRPTAQLLMTMHELE
GEPFFADPREVLRQVVARFTEMELTIVAAFELEFYLIDQENVNGRPQPPRSPISGKRPQSV
QVYSIDDLDEYVECLQDIIDGARAQGIPADAIVAESAPAQFEVNLNHVNDALKACDHAV
LLKRLVKNIAYDHEMDTTFMAKPYPGQAGNGLHVHISLLDKHGNNIFTSEDPEQNAAL
RHAIGGVLETLPASMAFLCPNVNSYRRFGSQFYVPNAPSWGLDNRTVALRVPTGSPDAV
RLEHRVAGADANPYLLLAAVLAGVHHGLINKVEPGAPIEGNSYEQMEPSLPNNLRDAL
RELDESEIMAKYIDPKYIDIFVACKESELEEFEHSISDLEYNWYLHTV
PA0365
(SEQ ID NO: 124)
MSDTTLESAGLSRRSLMKVGLIGGAFLATAGVTASLTGCSAEKPASGLEKVRES
DLPFLRALLPVMLLGAVSAEQMPKAVEGAIQSLDHNLARLSPEMFKLTQQLFDVLALPL
TRGPLTGIWGSWENASGDDVRAFLSRWENSFIGLLRMGHSSLMQLAMMAWYARPEA
WAHCGYPGPPKIA
pilH
(SEQ ID NO: 125)
MARILIVDDSPTEMYKLTAMLEKHGHQVLKAENGGDGVALARQEKPDVVLMDI
VMPGLNGFQATRQLTKDAETSAIPVIIVTTKDQETDKVWGKRQGARDYLTKPVDEETLL
KTINAVLAG
anmK
(SEQ ID NO: 126)
MPRYLGLMSGTSLDGMDIVLIEQGDRTTLLASHYLPMPAGLREDILALCVPGPDE
IARAAEVEQRWVALAAQGVRELLLQQQMSPDGVRAIGSHGQTIRHEPARHFTVQIGNPA
LLAELTGIDVVADFRRRDVAAGGQGAPLVPAFHQALFGDGDASRAVLNIGGFSNVSLLS
PGKPVRGFDCGPGNVLMDAWIHHQRGEHFDRDGAWAASGQVNHALLASLLADEFFAA
RGPKSTGRERFNLPWLQEHLARHPALPAADIQATLLELSARSISESLLDAQPDCEEVLVC
GGGAFNTALMKRLAMLMPEARVASTDEYGIPPAWMEGMAFAWLAHRFLERLPGNCPD
VTGALGPRTLGALYPA
rpsG
(SEQ ID NO: 127)
MPRRRVAAKREVLADPKYGSQILAKFMNHVMESGKKAVAERIVYGALDKVKE
RGKADPLETFEKALDAIAPLVEVKSRRVGGATYQVPVEVRPSRRNALAMRWLVDFARK
RGEKSMALRLAGELLDAAEGKGAAVKKREDVHRMAEANKAFSHYRF
gldF
(SEQ ID NO: 128)
MTAIGTIFRRELGSYFATPLAYVFTLVFLVLSGVATFYLGDFFERGQADLAPFFSS
LPWLYLLLIPALAMRLWAEERKSGSIEMLMTLPVSRATLVTGKFLAAWFCAGLALLLTF
PMPLTVNYLGSPDNGAIIAGYLAGWLLSGGYLAIGSCMSALAKNQIIAFALTVLVCLLFV
GAGTPHVQQALSGWLPQWLLDGIASLSVLVRFEALGRGVLDVRDLAYFCSLIVAWLVA
TTIVIDLKKAA
ladS
(SEQ ID NO: 129)
MRHWLILFLIALPCLAGAVSFNEQVERLPLGQSIDVFEDVRGSADINDITSRAIDSS
FRRHDKDVLNAGYSRSVFWLRLDLDYRPVASSDPRTWLLELAYPPLDKLDLYLPDGQG
GYRLAQRTGDTLPFASRPIRQNNYLFELGLEPNKPQRVYLRLESQGSIQAPLTLWSPKAY
LEEQPERIYVLGIIYGVLLVMLIYNLFIFLSVRDTSYLYYILYIASFGLYQVSVNGAGIEYF
WPDSPWWANAATPFLIGSAALFGCQFARSFLHTRDHSVWVDRGLLALMAVGALVML
MALTMSYAVALRLATYLALAFTGLIFAAGILAWLRGMRVARYFIIAWTAFLLGGIVNTL
MVLGYLPNMFLTMYASQIGSALEVGLLSLALADRINAMKEERARILQESSRKLEALNQE
LANSNRLKDEFLATVTHELRTPMSGVIGSLELMQTVPMDVELAEYQRTAAGSARDMM
RMVNDILALIELQAGKLYPRREPFSLRGLFDSLRAQYAPRAEEKGLRFALQLDDSLPDTL
EGDAGKLAQALGYLVDNAIKFTARGSVTLRVAAGRTHDGVALRVEVIDTGIGFDMAAG
SDLYQRFVQADSSLTRGYGGLGIGLALCRKLVELLGGELTHESRPGQGSRFLLRLQLTQP
AQGLAPPPRRAGGQAVRRPEECTVLVVEDNAINQLVTRGMLLKLGYRVRTADNGSEAL
ELLARERPDGVLLDCQMPVMDGFATCRAIRALPGCAELPVLALTAHSHSGDRERCLAA
GMSDYMAKPVKFEELQTLLHDWLLCQPIVTKSA
PA3886
(SEQ ID NO: 130)
MSDAPTSPRFSAATSTRLLDHAQLEALCADYDGFLLDLWGVVMDGTEAFPGAL
AWLARRHAEGRPVWFLSNSSSSVVEMSAGLERLGIRRDWFAGITTSGQLTIDALLQTAE
YRRGGIYLAGVGLAQQSWPAEIRERFVEDIAQAALIVGVGSFPQDELEQRFAPLRGATDL
PFLCANPDRVVVSGGRTVYGAGMLAELFSEEGGQVSWYGKPDPAAFRIAQRQLEARGA
RHILFVGDSLVTDVPGALAARIDTLWLGATGIHREALGAEFNGALDEERVRSLLHGYPIR
PHFAAPGLV
cheA
(SEQ ID NO: 131)
MTPDQMRDASLLELFRLEAEAQTQVLNAGLMALERSPTQADQLEACMRAAHSL
KGAARIVGLDAGVRVAHVMEDCLVEAQDGRLLLQSEHIDALLQGCDLLLRIGTPPAGD
AGWAEGAGREEIDGLVLRLEGLVRSGLPLARAELPATTPGLPEAVPEAPPAASAAASDD
NDEEPAGQAGGEQAEERRSRVLRVTAERLDRLLDISSKSLVEFQRIKPLADSLQRLRRLQ
SSASRALDVVRETVQETALDPQAQAMLGEARQLIGECQQMLVQHIADLDEFAWQGGQ
RAQVLYDAALASRMRPFADVLSGQARMVRDLGRSLGKQVRLLVEGESTQVDRDVLEK
LEAPLTHLLRNAVDHGIEAPETRLAAGKPAEGRITIRARHHAGMLVLELSDDGGGIDLQ
RLRETVLNRQFATAETVAQLSEEELLAFLFLPGFSMREQVTEVSGRGVGLDAVQHMVR
QLRGGVRMEQRQGQGALFHVEVPLTLSVVRSLVVEIGEEAYAFPLAHIERMCELEAEEI
VQLEGRQHFWYEGRHVGLVSAAQLLQRPESSRTEGAIPVVVVRDRDAVYGVAVERFV
GERTLVVMPLDPRLGKVRDVSAGALLDDGSPVLILDVEDLLHSVGKLLSSGRLERIDRS
RRQAGGAQRKRILVVDDSLTVRELERKLLLGRGYDVAVAVDGMDGWNALRSEHFDLL
ITDIDMPRMDGIELVTLVRRDSRLQSLPVMVVSYKDREEDRRRGLDAGADYYLAKASF
HDEALLDAVVVLIGEAQG
nalD
(SEQ ID NO: 132)
MRRTKEDSEKNRTAILLAAEELFLEKGVSHTSLEQIARAAGVTRGAVYWHFQNK
AHLFNEMLNQVRLPPEQLTERLSGCDGSDPLRSLYDLCLEAVQSLLTQEKKRRILTILMQ
RCEFTEELREAQERNNAFVQMFIELCEQLFARDECRVRLHPGMTPRIASRALHALILGLF
NDWLRDPRLFDPDTDAEHLLEPMFRGLVRDWGQASSAP
czcR
(SEQ ID NO: 133)
MRILIIEDEVKTADYLHQGLTESGYIVDRANDGIDGLHMALQHPYELVILDVNLP
GIDGWDLLRRLRERSSARVMMLTGHGRLTDKVRGLDLGADDFMVKPFQFPELLARVRS
LLRRHDQAPMQDVLRVADLELDASRHRAFRGRVRINLTTKEFALLHLLMRRNGDVITR
TQIISLIWDMNFDNDSNVVEVAICRLRAKIDDGFDLKLIHTIRGVGYVLEARR
N/A
(SEQ ID NO: 134)
MERIDHLLPWSTLGSEKRLSVFRFGCGARKVYIQSSLHADELPGMRTAWELKQR
LRLLEAEGRLRGTVELVPVANPVGLGQMIQALHQGRFEMSSGRNFNRDFPDLLDAVIDS
VGERLGSDPAANVALVRQTLRAALDALPPATSELEGMQRLLYRHACDADLVLDLHCDF
EAAIHLYTLPQQWPAFASLAARLGAAVGLLAEESGGGSFDEACSVPWLRLSRLYPRAEL
PLACLATTVELGGQADTTVQQAEANAAAILAFLAEQGFVEGEWPAAPEACCEGLPFEGT
EYVHAPHTGVVSFLRRPGEWVEAGEPLFQVIDPLADRASTVCAGVSGVLFAIERMRYA
QPGLWLAKVAGRQPIRQGRLLSD
bifA
(SEQ ID NO: 135)
MKLDSRHSLSLKLLRVVLLAALAVGVVLRCAQIVFDAYKAKQAVSSDAQRILA
MVRDPSTQAVYSLDREMAMQVLEGLFQHEAVRQASIGHPGEPMLAEKSRPLLDLPTRW
LTDPILGQERTFSIRLIGRPPYSEYYGDLKITLDTAPYGENFVTTSEIIFISGILRALAMGLV
LFLVYHWMLTKPLSKIIEHLVSINPDRPSQHQLPLLKGHERNELGLWVTTANQLLASIES
NSHLRREAEDNLLRISQYDFLTGLPNRQLLQQQLDQILDGAGRQQRRVAVLCLGLDDFK
GINEQYTYQLGDQLLIALADRLRGHSARLGSLARLGGDQFALVQADIEQPYEAAELAQS
ILDGLEAPFEIDQHEVRLRATIGITLFPEDGETTEKLLQKAEQTMTLAKTRSRNRYQFYIA
SVDSEMRRRRELEKDLRDALQRHELHLVYQPQVDYRDHRVVGVEALLRWQHPLHGFV
PPDLFIPLAEQNGSIFSIGEWVLDQACRQLREWHDQGFDDLRMAVNLSTVQLHHNALPR
VVSNLLQVYRLPARSLELEVTETGLMEDISTAAQHLLSLRRAGALIAIDDFGTGYSSLSY
LKSLPLDKIKIDKSFVQDLLQDEDDATIVRAIIQLGKSLGMQVIAEGVETAEQEAYIIAEG
CNEGQGYLYSKPLPARELTQYLKQARRLSQATSSERP
N/A
(SEQ ID NO: 136)
MNARVHQPVHTAQHAPSYYAATLNRRIECPPLAGEEQADVCVVGGGFSGVNTA
LELAQRGFSVVLLEAHRIGWGASGRNGGQLIRGVGHDVEQFLPVIGADGVKALKLMGL
EAVEIVRRRVEQYAIDCDLRWGYCDLANKPGDYQGFREDMEELQALGYRHEMRLVPA
AEMRSVVGSDRYVGGLVDMGSGHLHPLNLVLGEAAAAQSLGVRLFERSPVTRIDYGTE
VQVHTATGKVRAKTLVLGCNAYMNDLNPLLGGKVLPAGSYVIATEPLDEKLARQLLPQ
NMAVCDQRVALDYYRLSADNRLLFGGACHYSGRDPSDIAAYMRPKMLEVFPQLANVR
IDYQWGGMIGIGANRLPQIGRLPGQPNVYFAQAYSGHGVNATHLAGQLLAEAIGGQQS
DGFDLFAKVPHITFPGGKLLRSPLLALGMAWYRLKEKLGS
N/A
(SEQ ID NO: 137)
MAEWNRNETLWRQGLLLASDAVEALGLHHPESPERTLVIVASHDCDLAQSPEKE
PDIEVVIGRLALEKDGNSTHAKNARKLHIEFTGADTFWAEFEATAKSKVGKLELNRHAP
RSGATLSPECHAVFQMWLASRYRRSAFPDEFERRLTSKDFKLHERISKAVKPHGDLIAG
VFFDVDEGVEINRNGADDTYTLDIIIMHSADPNFEEAEKAAESAAATITQAFKEKLFSPTS
TWQHIELRSCDAVSESVLTYQQFKQLKRWRLEHLSLAADPQQPVLAE
N/A
(SEQ ID NO: 138)
MKIKAALIVDDLSLREWQKRAIEDSSEYLDIQLVLSCRNSATKKSVIKHCGYYFL
NILSLKNDMTRRVQLDSRGSEVIHFDSDYEGAWQRIPEDVCARILDKGIKLVIKFGMSLL
RIDGGLQRLDILSYHHGDPEYYRGRPAGFYEIYENADSVGIIVQKLSNKLDAGEVLVRGY
SKVHHHSYKKTSRNFYLNSVVLLRKALVNYSRGEQVVLEKLGKNYRLPSNFTVFKFFC
KTIFRGLARLSYGAFFEKKWNVVALPYNDIPSLQELSVSAGKIPKVEKGYTFYADPFFSA
DGKLIRLEALNASNGLGEIIELKAQSLDFSRVILKGNHFSYPYSFEASGVEYLIPEVASHS
APCLLPPPFALESKKLFQGMEGERILDGTLFEHGGRYYLFCGQAVSGSDNLYLYVGESL
EGPYTSHPCNPVVMNPGSARMGGRIFKEGGKLYRFGQNNSYGYGSSLAVNEIEVLDPEH
YSEKRVANLAFQDARGPHTIDIHGQTMILDFYQDRFSLLAGYRRLVARLLSKG
impA
(SEQ ID NO: 139)
MSRSPIPRHRALLAGFCLAGALSAQAATQEEILDAALVSGDSSQLTDSHLVALRL
QQQVERIRQTRTQLLDGLYQNLSQAYDPGAASMWVLPANPDNTLPFLIGDKGRVLASL
SLEAGGRGLAYGTNVLTQLSGANAAHAPLLKRAVQWLVNGDPGAATAKDFKVSVVG
VDKTATLNGLKSAGLQPADAACNALTDASCASASKLLVLGNGASAASLSATVRARLQA
GLPILFVHTNGWNQSSTGQQILSGLGLQEGPYGGNYWDKDAVPSSRTRARSVELGGAY
GQDPALVQQIVDGSWRTDYDWSKCTSYVGRTTCDDVPGLSDFSKRVDVLKGALDAYN
QKAQNLFALPGTTSLRLWLLWADAVRQNIRYPMDKAADTARFQETFVADAIVGYVRE
AGAAQKELGSYAGQRQQSMPVSGSEETLTLTLPSAQGFTAIGRMAAPGKRLSIRIEDAG
QASLAVGLNTQRIGSTRLWNTRQYDRPRFLKSPDIKLQANQSVALVSPYGGLLQLVYSG
ATPGQTVTVKVTGAASQPFLDIQPGEDSSQAIADFIQALDADKADWLEIRSGSVEVHAK
VEKVRGSIDKDYGGDVQRFIRELNEVFIDDAYTLAGFAIPNQAKTPAIQQECAVRGWDC
DSETLHKLPGTQHINVDQYAQCGGGCSGNPYDQTWGLNPRGWGESHELGHNLQVNRL
KVYGGRSGEISNQIFPLHKDWRVLREFGQNLDDTRVNYRNAYNLIVAGRAEADPLAGV
YKRLWEDPGTYALNGERMAFYTQWVHYWADLKNDPLQGWDIWTLLYLHQRQVDKS
DWDANKAALGYGTYAQRPGNSGDASSTDGNDNLLLGLSWLTQRDQRPTFALWGIRTS
AAAQAQVAAYGFAEQPAFFYANNRTNEYSTVKLLDMSQGSPAWPFP
PA4401
(SEQ ID NO: 140)
MALTIVIGNRNDSSWSLRGWLALRMSGAAFDEILVPLGRPDSRERILQYSPTGKV
PLLKSEDGDIWDSLAIAEYLAERFPEAHLWPRGEAARALARSVCAEMHSGFAALRGEPP
MDLRRQQPLVELSEATRQDIQRICEAWADCRRRFGQDGPFLFGHASLADAFYAPVAAR
FRSYAVELPDIARTYVETIYQWPAFRAWYDAALREQAGS
N/A
(SEQ ID NO: 141)
MTLLDIEKLLDMTHHMSVHIPIELIISWEQSEEAWSVNDNCLNFVYVNRRYTELIT
PRFGQKNSLLSPFSASIEEHDKLVIQTGKRIEALALLRPDDYPAPCCLYFERMPLYDRRG
NRTGVIAHAKTLTSVAPRGFIATDGIGTFTFTPPSELFTSREWDVIYLLLSGLSEKEIAEQIS
RSLSTVKFHKSNIFQKVGCSCIGAFKALARQKKWNFYIPPTFASAKYIINH
pvdL
(SEQ ID NO: 142)
MMDAFELPTTLVQALRRRAVQEPERLALRFLAEDDGEGVVLSYRDLDLRARSIA
AALQAHAQLGDRAVLLFPSGPDYVAAFFGCLYAGVIAVPAYPPESARRHHQERLLSIIA
DAEPRLVLTTADLREPLLQMNAQLSAANAPQLLCVDQLDPAVAEAWDEPQVRPEHIAF
LQYTSGSTALPKGVQVSHGNLVANEVLIRRGFGIGADDVIVSWLPLYHDMGLIGGLLQPI
FSGVPCVLMSPRYFLERPVRWLEAISQYGGTVSGGPDFAYRLCSERVAESALQRLDLSG
WRVAFSGSEPIRQDSLERFAEKFAASRFDASSFFACYGLAEATLFVTGGQRGQGIPALAV
DGEALARNRIAEGAGSVLMCCGRSQPEHAVLIVDAVSGEALGDDNVGEIWAAGPSIAH
GYWRNPEASAKTFVERDGRTWLRTGDLGFLRDGELFVTGRLKDMLIVRGHNLYPQDIE
RTVESEVPSARKGRVAAFAVTVDGEEGIGIAAEIGRGVQKSVPAQELIDSIRQAVAEAYQ
EAPKVVALLNPGALPKTSSGKLQRSACRLRLEDGSLDSYALFPGLQAVQEAQPPAGDDE
LLARIGEIWKARLGVAQVAPRDHFFLLGGNSIGAAQVVAQVRDSLGVALDLRQLFEAPT
LHAFSATVARQLAAGLPAEAPMAHLPRGVDLPQSAAQQRLWLTWQIDPQSAAYNIPGG
LRLRGELDEAALRASFQRLVERHEALRTRFLERDGAALQRIDERGEFAWQFVDLAALAE
HERAAAAAQRREAEAQQPFDLEKGPLLRISLVRLDEQEHQLWVTLHHIVADGWSLNLL
LDEFSRLYAEACGGQPADLAPLELHYAEFAAWQRQWLDAGEGARQLAYWRERLGDA
APVLELATDHPRTARQASPAARYSLRVDEALARAIREAALDHEASVFMWLLAAFQALL
HRHSGQGEIRIGVPSANRQRLDTQGLVGFFINTLVLRGTPRARQPFAALLGEAREATLGA
QANQDLPFDQVLAACGQGGQLFQVLFNHQQRDLSALRRLPGLLADELPWHSREAKFDL
QLQSEEDARGRLTLNFDYAADLFDEASIRRFAAQYLELLRQVAEDPQRCLGDIALVDAE
QAARLAEWGSAPCEPARAWLPELLERQLAQSAERVALEWDGGSLGYAELHARANRLA
HYLRDKGVGPDVRVAICAERSPQLLVGLLAIVKAGGAYVPLDPDYPSERLAYMLADSG
VELLLTQAHLFERLPGAEGVTPICLDSLKLDNWPSQAPGLHLHGDNLAYVIYTSGSTGQP
KGVGNTHAALAERLQWMQATYALDGDDVLMQKAPVSFDVSVWECFWPLVTGCRLVL
AAPGEHRDPARLVELVRQFGVTTLHFVPPLLQLFIDEPGVAACGSLRRLFSGGEALPAEL
RNRVLQRLPAVALHNRYGPTETAINVTHWQCRAEDGERSPIGRPLGNVVCRVLDAEFN
LLPAGVAGELCIGGLGLARGYLGRPALSAERFVADPFSADGERLYRTGDRARWNADGV
LEYLGRLDQQVKLRGFRIEPEEIQARLLAQPGVAQAVVVIREGVAGSQLVGYYTGAVG
AEAEAEQNQRLRAALQAELPEYMVPAQLMRLAQMPLGPSGKLDTRALPEPVWQQREH
VEPRTELQRRIAAIWSEVLGLPRVGLRDDFFELGGHSLLATRIVSRTRQACDVELPLRAL
FEASELEAFCEQVRAAQAAGRTDSHGAIRRIDREQPVPLSYSQQRMWFLWQLEPDSPAY
NVGGLARLSGPLDVARFEAALQALVQRHETLRTTFPSVDGVPVQRVHGDGGLHMDWQ
DFSALDRDSRQQHLQTLADSEAHRPFDLESGPLLRVCMVKMAEREHYLVVTLHHIVTE
GWAMDIFARELGALYEAFLDDRESPLEPLPVQYLDYSVWQREWLESGERQRQLDYWK
AQLGNEHPLLELPGDRPRPPVQSHQGDLYRFDLSPELAERVRRFNAARGLTMFMTMTA
TLAALLYRYSGQQDLRIGAPVANRIRPESEGLIGAFLNTQVLRCRLDGQMSVGELLEQV
RQTVIDGQSHQDLPFDHLVEALQPPRSAAYNPLFQVMCNVQRWEFQQTRQLAGMTVE
YIANDARATKFDLNLEVTDLDQRLGCCLTYSRDLFDEPRIARMAGHWQNLLEALLGDP
QRRIAELPLFAAEERKQLLLAGTAGEAGLQDTLHGLFAARVAASPQAPALTFAGQTLSY
AELDARSNRLARVLRSHGVGPEVRVGLALERSLEMVVGLLAILKAGGAYVPLDPEYPLE
RLQYMIEDSGVRLLLSHAALFEALGELPAGVARWCLEEDGPALDAEDPAPLAALSGPQH
QAYLIYTSGSTGKPKGVAVSHGEIAMHCAAVIERFGMRAEDCELHFYSINFDAASERLL
APLLCGARVVLRAQGQWGAEEICELIRAEGVSILGFTPSYGSQLAQWLESQGRQLPVRM
CITGGEALTGEHLQRIRQAFAPASFFNAYGPTETVVMPLACLAPERLEEGAASVPIGSVV
GARVAYILDADLALVPQGASGELYVGGAGLARGYHERPALSAERFVPDPFAAEGGRLY
RTGDLVRLCDNGQVEYVGRIDHQVKIRGFRIELGEIEARLLEHPQVREALVLALDSPSGK
QLAGYVASAVAEQDEDAQAALREALKTHLKQQLPDYMVPAHLLLLASLPLTANGKLD
RRALPAPDPALNRQAYEAPRSVLEQQLAGVWREVLNVERVGLGDNFFELGGDSILSIQV
VSRARQLGIHFSPRDLFQHQTVQSLAAVARHSQASQAEQGPVQGDSALTPIQHWFFDLP
LARREHWNQSLLLQPRQALDLGLLRKSLQRLVEQHDALRLAFRQVDGEWLAQHRPLRE
QELLWHVPVQSFDECAELFAKAQRSLDLEQGPLLRAVLVDGPAGEQRLLLAIHHLVVD
GVSWRVLLEDLQQVYRQFAEGAEPALPAKTSAFRDWAGRLQAYAGSESLREELGWWQ
ARLGGQSAEWPCDRPQGDNREALAESVSLRLDPQRTRQLLQQAPAAYRTQVNDLLLAA
LARVLCRWSGQPSTLVQLEGHGREALFDDIDLTRSVGWFTSAYPLRLTPAQSPGESIKAI
KEQLRAVPHKGLGYGVLRYLADPAVRQAMAALPTAPITFNYLGQFDQSFADALFQPLD
QPTGPIHDEQAPLPNELSVDGQVYGGELVLRWTYSRERYDARTVNELAQAYLAELQALI
EHCLEDGAGGLTPSDFPLAQLSQAQLDALAVPVGEIEDVYPLTPMQEGLLLHTLLEPGT
GIYYMQDRYRIDSPLDPERFAAAWQAVVARHEALRASFVWNAGETMLQVIHKPGRTRI
EFLDWSELPEDGHEERLQALHKREREAGFDLLEQPPFHLRLIRLGEARYWFMMSNHHILI
DAWCRGLLMNDFFEIYGALGEGRPANLPTPPRYRDYIAWLQRQDLEQSRRWWSESLRG
FERPTLVPSDRPFLREHAGESGGMIVGDRYTRLDAADGARLRELAQRYQLTVNTFAQA
AWALTLRRFSGERDVLFGVTVAGRPVGMPEMQRTVGLFINSIPLRVQMPAAGQRCTVR
EWLNRLFERNLELREHEHLPLVAIQESSELPKGQPLFDSLFVFENAPVEVSVLDRAQSLN
ASSDSGRTHTNFPLTVVCYPGDDLGLHLSYDQRYFEAPTVERLLGEFKRLLLALADGFH
GELEALPLLGEDERDFLLDGCNRSARDYPLEQGYVRLFEAQVAAHPQRIAASCLEQRWS
YAELNRRANRLGHALRAAGVGIDQPVALLAERGLDLLGMIVGSFKAGAGYLPLDPGHP
TQRLTRIVELSRTPVLVCTQACREQALALFDELGCVDRPRLLVWDEIQQGEGAEHDPQV
YSGPQNLAYVIYTSGSTGLPKGVMVEQASMLNNQLSKVPYLELDENDVIAQTASQSFDI
SVWQFLAAPLFGARVAIVPNAIAHDPQGLLAHVGEQGITVLESVPSLIQGMLAEERQAL
DGLRWMLPTGEAMPPELARQWLKRYPRIGLVNAYGPAECSDDVAFFRVDLASTESTYL
PIGSPTDNNRLYLLGAGADDAFELVPLGAVGELCVAGTGVGRGYVGDPLRTAQAFVPH
PFGAPGERLYRTGDLARRRADGVLEYVGRIDHQVKIRGFRIELGEIEARLHERADVREA
AVAVQEGANGKYLVGYLVPGETPRSSADSPAGLMVEQGAWFERIKQQLRADLPDYMV
PLHWLVLDRMPLNANGKLDRKALPALDIGQMQNQAYQAPRNELEETLARIWAEVLKV
ERVGVFDNFFELGGHSLLATQIASRVQKALQRNVPLRAMFECTTVEELASYIESLAPSEIS
EQKAERLNDLMSKLEML
pvdS
(SEQ ID NO: 143)
MSEQLSTRRCDTPLLQAFVDNRTILVKIAARITGCRSRAEDVVQDAFFRLQSAPQI
TSSFKAQLSYLFQIVRNLAIDHYRKQALEQKYSGPEEEGLNVVIQGASPETSHINYATLE
HIADALTELPKRTRYAFEMYRLHGVPQKDIAKELGVSPTLVNFMIRDALVHCRKVTAER
QGDNVTHLSARR
oprM
(SEQ ID NO: 144)
MKTSFAFTRPARTLAPLALAAALAGCSMAPKYDRPAAPIDTAYPSGAAYVELAA
ATPDDAITAEIGWRDFFRDPLLQQLIGISLENNRDMHKAALNVEAAQALYRIQRAEMLP
NLGVSARGASERVPADLSTTGQSDVLRRYDVAGVTAAWELDLWGRIRSLNDRALASYL
ALDETRIATQMSLVSEVASAYLTLRADQELLRLTSDTLATQKRSYDLTTQLVEAGNSTQ
LDLRRAEIALRTAEANRAAYTRQAAKDRNALVLLLGQPLTPELSRQLDEAVALPDDIVP
TDLPSGLPSELLARRPDIRAAEQMLIGANANIGAARAAFFPTISLTGSAGTASASLDGLFD
SGSRAWSFLPQITLPIFRGGALRANLDVAQVQKRIEIANYEKSIQAAFAEVADGLAGKRT
LDEQIRSEQLLVAASQKAYQLAEQRFQEGVDDNLTLLDAQRTQYGAQQTLVRTRLTRL
NNLIHLYKALGGGWTEHTVQSGATGQPSARSPG
rhlR
(SEQ ID NO: 145)
MRNDGGFLLWWDGLRSEMQPIHDSQGVFAVLEKEVRRLGFDYYAYGVRHTIPF
TRPKTEVHGTYPKAWLERYQMQNYGAVDPAILNGLRSSEMVVWSDSLFDQSRMLWNE
ARDWGLCVGATLPIRAPNNLLSVLSVARDQQNISSFEREEIRLRLRCMIELLTQKLTDLE
HPMLMSNPVCLSHREREILQWTADGKSSGEIAIILSISESTVNFHHKNIQKKFDAPNKTLA
AAYAAALGLI
algP
(SEQ ID NO: 146)
MSANKKPVTTPLHLLQQLSHSLVEHLEGACKQALVDSEKLLAKLEKQRGKAQE
KLHKARTKLQDAAKAGKTKAQAKARETISDLEEALDTLKARQADTRTYIVGLKRDVQE
SLKLAQGVGKVKEAAGKALESRKAKPATKPAAKAAAKPAVKTVAAKPAAKPVAKTA
AAKPAAKPAAKPAAKPAAKPAAKTAAAKPAAKPVAKPAAKPAAKPAAKTAAAKPAA
KSAAKPVAKPAAKPAAKTAAAKPAAKPAVKPVAKPAAKPAAKTAAAKPAAKPAAKPV
AKPAPAAAKPAAKPAAKPAAKPVAKPAAKPVAAKPAAAKPATAPAAKPAATPSAPAA
ASSAASATPAAGSNGAAPTSAS
PA5248
(SEQ ID NO: 147)
MGGKAKGQFNLFHSHFVFAMHPPKILLWLLPLVCAFSLGAIADTAVDPSQALHL
LSYLAADYPPTVADGKIVDPSEYQEQVEFVGNLQALVLTLPMRPERAELERGGASLRQA
IEQRLPGRDVALQARNLEARVADIYQVVQTPAITPDPSRAAPIYAQQCAICHGDAGKGD
GPAGIGLEPPPANLTDRQRLDHLSLYDLRNVIGLGVAGTDMPAFADQLDERQRWDLAS
YVAGLSAGSAQPDKAHAYPLATLATQTPAEVAEHDGEAAAESFRALRAHPPLEQRGPG
QLIDYTAATLDKSFAVYREGDRDQAYDLSVAAYLEGFELVESSLDNVDADLRRSTEKQ
LMAYRQALRDGLPETQVAQQLELAKGKLAEAAKQLGGDSLSFSISFVSALLILLREGVE
AILVLAAILAFLRNTGQESAVRGVHVGWGLAFVAGFATWALAAYVIDIGGAQRELMEG
FTSLFACVMVLWLGVWMHDRRHAAAWQDYIRSSLVGGGGRFGFAVLAFFSVYRELFE
VILFYETLWLQAGPAGHNAVIGGAATAVVLLIGLAWVILRGSAKLPLGLFFSINAALLCA
LSVVFAGHGVIALQEAGVIGTRPVPFFDFDWLGIKADAYSLSAQAMALVAIALLYGRSRI
VERRRAAANAAD
N/A
(SEQ ID NO: 148)
MSDTPAEVCENLLARALTNDDLVAFLVGEQPYFIESHSGEEEPQDVCRAIERCLL
PCWQSGRFPYLPRRFADALLKILATYPDRNRAIYVAQNWIWYYRFCLSKKRANPRGPY
GDLFEVDLGAVAVALKRQLEANKDELIRDARWAGATWNSSNGLWGPLLRTSTTVRDK
LDGPDFVPDTP
tufA
(SEQ ID NO: 149)
MAKEKFERNKPHVNVGTIGHVDHGKTTLTAALTKVCSDTWGGSARAFDQIDNA
PEEKARGITINTSHVEYDSAVRHYAHVDCPGHADYVKNMITGAAQMDGAILVCSAADG
PMPQTREHILLSRQVGVPYIVVFLNKADMVDDAELLELVEMEVRDLLNTYDFPGDDTPII
IGSALMALEGKDDNGIGVSAVQKLVETLDSYIPEPVRAIDQPFLMPIEDVFSISGRGTVVT
GRVERGIIKVQEEVEIVGIKATTKTTCTGVEMFRKLLDEGRAGENVGILLRGTKREDVER
GQVLAKPGTIKPHTKFECEVYVLSKEEGGRHTPFFKGYRPQFYFRTTDVTGNCELPEGV
EMVMPGDNIKMVVTLIAPIAMEDGLRFAIREGGRTVGAGVVAKIIE
phzE
(SEQ ID NO: 150)
MNALPTSLLQRLLERPAPFALLYRPESNGPGLLDVIRGETLELHGLADLPLDEPGP
GLPRHDLLALIPYRQIAERGFEALDDGTPLLALKVLEQELLPLEQALALLPNQALELSEE
AFDLDDEAYAEVVGRVIADEIGRGEGANFVIKRRFQARIDGYATASALSFFRQLLLREK
GAYWTFIVHTGERTLVGASPERHISVRDGLAVMNPISGTYRYPPAGPNLAEVMEFLDNR
KEADELYMVVDEELKMMARICEDGGRVLGPYLKEMAHLAHTEYFIEGQTSRDVREVL
RETLFAPTVTGSPLESACRVIRRYEPQGRGYYSGVAALIGGDGQGGRTLDSAILIRTAEIE
GDGRLRIGVGSTIVRHSDPLGEAAESRAKASGLIAALKSQAPQRLGSHPHVVAALASRN
APIADFWLRGASERQQLQADLSGREVLIVDAEDTFTSMIAKQLKSLGLTVTVRGFQEPY
SFDGYDLVIMGPGPGNPTEIGQPKIGHLHLAIRSLLSERRPFLAVCLSHQVLSLCLGLDLQ
RRQEPNQGVQKQIDLFGAAERVGFYNTFAARALQDRIEIPEVGPIEISRDRETGEVHALR
GPRFASMQFHPESVLTREGPRIIADLLRHALVERRP
bphP
(SEQ ID NO: 151)
MTSITPVTLANCEDEPIHVPGAIQPHGALIALRADGMVLAASENIHALLGFVASPG
SYLAPEQVGPEVLRMLEEGLTGNGPWSNSVETRIGEHLFDVIGHSYKEVFYLEFEIRTAD
TLSITSFTLNAQRIIAQVQLHNDTASLLSNVTDELRRMTGYDRVMAYRFRHDDSGEVVA
ESRREDLESYLGQRYPASDIPAQARRLYIQNPIRLIADVAYTPMRVFPALNPETNESFDLS
YSVLRSVSPIHCEYLTNMGVRASMSISIVVGGKLWGLFSCHHMSPKLIPYPVRMSFQIFS
QVCSAIVERLEQGRIAELLRVPTERRLALARRARDADDLFGALAHPDDGIAALIPCDGAL
VMLGGRTLSIRGDFERQAGNVLQRLQRDPERDIYHTDNWPQPSEDSPDGGDCCGVLAIR
FHRQESGWIFWFRHEEVHRIRWGGKPEKLLTIGPSGPRLTPRGSFEAWEEVVRGHSTPW
SETDLAIAEKLRLDLMELCLNHAAEVDRMRQRLIAVLGHDLRNPLQSISMAAALLSSSD
TRTTELRQHISASSSRMERLVSQILDMSRLQSGIGLTVNPVDTDVSQLVQQIVCETDVAY
PGLVIEIAIDPQVRAVVDPDRYAQVAANLLSNARHHGLPGRPVLVTLTRQGDEVCLSVL
NETSGLSEAQLANLFEPFKRESADNQRNRNGLGIGLYISQAIAQAHQGRIDVDCRDDVIT
FCLRLPVRQAETGSSS
eutB
(SEQ ID NO: 152)
MARFTHSVGGETYRFDSLKDVMAKASPARSGDFLAGVAASNDGERVAAQMAL
ADIPLKHFLDEALIPYEDDEVTRLIIDTHQRDAFAPVSHLTVGGFRDWLLGDAADEASLR
ALAPGLTPEMAAAVSKIMRVQDLVLVAQKIRVVTRFRNTLGLRGRLSTRLQPNHPTDDP
AGIAASILDGLLFGNGDAMLGINPATDSMASICALLEMLDAIIQRYEIPTQACVLTHVTSS
IEAINRGVPLDLVFQSIAGTEAANASFGISLKILQEGYEAGLSQKRGTLGNNLMYFETGQ
GSALSANAHHGVDQQTCETRAYAVARHFKPFLVNTVVGFIGPEYLYNGKQIIRAGLEDH
FCGKLLGVPMGCDICYTNHAEADQDDMDMLLTLLGVAGINFIMGIPGSDDVMLNYQTT
SFHDALYARQTLGLKPAPEFEDWLQRMGIFTQADGRIRFGDELPPAFRQALAQLA
hscA
(SEQ ID NO: 153)
MALLQIAEPGQSPKPHERRLAVGIDLGTTNSLVAAVRSGVAEPLPDAQGRLILPS
AVRYHAERAEVGESARAAAAKDPFNTIISVKRLMGRGLEDVKQLGEQLPYRFRQGESH
MPFIETVQGLKSPVEVSADILRELRQRAETTLGGELVGAVITVPAYFDDAQRQATKDAA
RLAGLNVLRLLNEPTAAAVAYGLDKGAEGLVAIYDLGGGTFDISILRLTRGVFEVLATG
GDTALGGDDFDHAIAGWVIEQAGLSADLDPGSQRQLLQIACAAKERLTDEASVRVAYG
DWSGELSRATLDELIEPFVARSLKSCRRAVRDSGVDLEEIRSVVMVGGSTRVPRVRTAV
GELFGCEPLTDIDPDQVVAIGAAIQADALAGNKRGEELLLLDVIPLSLGLETMGGLMEK
VIPRNTTIPVARAQEFTTYKDGQTAMMIHVLQGERELVKDCRSLARFELRGIPPMVAGA
AKIRVTFQVDADGLLGVSARELSSGVEASIQVKPSYGLTDGEIARMLKDSFDYAGDDKA
ARALREQQVEAQRLLEAVQSALDVDGERLLDEEERLAIAAQMDTLRELAGGSDTAAIE
NQIKRLSQVTDAFAARRMDATVKAALSGRRLNEIEE
PA3508
(SEQ ID NO: 154)
MDKSDDSQDKYIVPGLERGLLLLCEFSRKDRTLTAPELARRLKLPRSTIFRLLTTL
EAMGFVTRNGNEYRLGMAVLRLGFEYLASLELTELGQPLLNRLCDEIRYPCNLVVRDG
RSIVYVAKVSPSTPLSSSVNVGTRLPAHATVLGRILLQDLSLGELRELYPEEQLEQFSPNT
PRSVLELFDMVQGDRQRGFVQGEGFFEASISTVAAPVRDHSGRVIAAMGATIAAGHIDP
ERIEGLVSRVRSSADELSYLLDYRADGQDNVTPIFRSRSHETV
yfiS
(SEQ ID NO: 155)
MLQSVSPKHDLPLKPEGQAAKPERTGTWAPFSIQAFRIIWICNLFANLGTWAQSV
AAAWVVTDAHASPLMVAMIQVAAALPLVLLSILSGVIADNHDRRKIMLWGLSFEMTGA
MFATLLAFLGYLDPVLLIISILWISLGGSVTIPAWQAAVNEQVPARMVSDAVLLNSVNYN
VARAAGPALGGLLLSAVGPAWVFLFNSFCYMALIWAIWQWRRDVPKRSLPPEGILEGV
TAALRFTQYSTVTRLVMMRSFAFGLSASAVWALLRLLAHRNPDGDAAIYGYMLGALG
LGAILGSTQVSRLRQRIGSSRLISLAGFTLALILLTLGLVDNLWVLFPVLILGGGCWIGAL
ATYNSAVQILVPDWIKARALALYQTALYGGLALGSFLWGHLAETMTVHGALLAAGCL
LLASVILLYNSRLPEMDAASISRAPASMPGQPSFVFNTRRGMVLVSIEYRIPAERTRDFVR
AAQPLRRLRLRNGAERWSLFRDVSNPEVWQELFLVDNWIQHLRMLDRMTLADKIVIDN
VTALHAGDGPPQIRHCVSYEASSYDTPLVKSATPPANDEEGATAGN
amrZ
(SEQ ID NO: 156)
MRPLKQATPTYSSRTADKFVVRLPEGMREQIAEVARSHHRSMNSEIIARLEQSLL
QEGALQDNLGVRLDSPELSLHERELLQRFRQLTHRQQNALVALIAHDAELAQA
PA3093
(SEQ ID NO: 157)
MRKLIFLILITLFAAYAGWAERRPVGHYLSDLRSQVSVVQGQPGERGNLLAVQP
ELFTPDYQSAERLQLKFHAYLENARRQGLLNERTVVVFPEHVVTWLVASGEKPEVYAA
ADWPTAMDWMAASNPLKVARGWITARGEQRLTDTLFRMKAVDMAHDYQTLFGGLA
HDFKVTVVAGSIVLPDPEVEDGELRPGTGQLYNVSLTFGPDGRPLGQPQRKVFPTRHEL
SYLNNGRGERLQVLDTPAGRLGVLIGTDSWYPDTYATLVEQRVELLAIPAALNQSGRW
QQPWPGFDAELVPGDVRLAPNSLSNAEAWQRLAVGERLLASGARGAAVAFAHSRLWN
VTEDGQSLLGSPQGMRQANPGGGAQLVNLWL
pelF
(SEQ ID NO: 158)
MTEHTAPTAPVADVCLLLEGTWPYVRGGVSSWVNQLILGLPDLTFSVFFIGGQK
DAYSKRHYPIPDNVLHIEEHFLETAWSSPNPQTRQGSSETEKALRDLHRFFHYPETPDVE
EGDALLDLLAEGRIGREDFLHSKASWEAITVGYERYCTDPSFVNYFWTLRSMQAPVFM
LAEAARRMPRARMLHSISTGYAGLLGCILQRRWGCRYLLSEHGIYTKERKIDLAQASWI
AENPDEQLSTGLDAEVSYIRRLWIRFFERVGLLTYRAANPIVALYEGNRQRQVLDGAEP
WRTRVIPNGIDLDAWAGALERRPPGIPPVVGLVGRVVPIKDVKTFIRAMRGVVSAMPEA
EGWIVGPEEEDPDYASECRSLVASLGLQDKVKFLGFRRIGEVLPQLGLMVLTSISEAQPL
VILEAWAAGTPVVSSDVGSCRELIEGADAEDRALGRAGEVVAIADPQATSRAILALLRN
PQRWQVAQAVGLQRVERYYTEALMLGRYRGLYREATEIA
pvdI
(SEQ ID NO: 159)
MNAEDSLKLARRFIELPVEKRRVFLETLRGEGIDFSLFPIPAGVSSAERDRLSYAQ
QRMWFLWHLEPQSGAYNLPSAVRLNGPLDRQALERAFASLVQRHETLRTVFPRGADDS
LAQAPLQRPLEVAFEDCSGLPEAEQEARLREEAQRESLQPFDLCEGPLLRVRLIRLGEER
HVLLLTLHHIVSDGWSMNVLIEEFSRFYSAYATGAEPGLPALPIQYADYALWQRSWLEA
GEQERQLEYWRGKLGERHPVLELPTDHPRPAVPSYRGSCYEFSIEPALAEALRGTARRQ
GLTLFMLLLGGFNILLQRYSGQTDLRVGVPIANRNRAEVEGLIGLFVNTQVLRSVFDGRT
SVATLLAGLKDTVLGAQAHQDLPFERLVEAFKVERSLSHSPLFQVMYNHQPLVADIEAL
DSVAGLSFGQLDWKSRTTQFDLSLDTYEKGGRLYAALTYATDLFEARTVERMARHWQ
NLLRGMLENPQASVDSLPMLDAEERYQLLEGWNATAAEYPLQRGVHRLFEEQVERTPT
APALAFGEERLDYAELNRRANRLAHALIERGVGADRLVGVAMERSIEMVVALMAILKA
GGAYVPVDPEYPEERQAYMLEDSGVELLLSQSHLKLPLAQGVQRIDLDRGAPWFEDYS
EANPDIHLDGENLAYVIYTSGSTGKPKGAGNRHSALSNRLCWMQQAYGLGVGDTVLQ
KTPFSFDVSVWEFFWPLMSGARLVVAAPGDHRDPAKLVALINREGVDTLHFVPSMLQA
FLQDEDVASCTSLKRIVCSGEALPADAQQQVFAKLPQAGLYNLYGPTEAAIDVTHWTC
VEEGKDAVPIGRPIANLGCYILDGNLEPVPVGVLGELYLAGRGLARGYHQRPGLTAERF
VASPFVAGERMYRTGDLARYRADGVIEYAGRIDHQVKLRGLRIELGEIEARLLEHPWVR
EAAVLAVDGRQLVGYVVLESESGDWREVLAAHLATSLPEYMVPAQWLALERMPLSPN
GKLDRKALPAPEVSVAQAGYSAPRNAVERTLAEIWQDLLGVERVGLDDNFFSLGGDSI
VSIQVVSRARQAGLQLSPRDLFQHQNIRSLALAAKAGAATTEQGPASGEVALAPVQRW
FFERAIPNRQHWNQSLLLQARQPLDGDRLGRALERLQAQHDALRLRFREERGAWHQAY
AEQAGEPLWRRQAGSEEALLALCEEAQRSLDLEQGPLLRALLVDMADGSQRLLLVIHH
LAVDGVSWRILLEDLQRLYADLDADLGPRSSSYQAWSRHLHEQAGARLDELDYWQAQ
LHDAPHALPCENPHGALENRHERKLVLTLDAERTRQLLQEAPAAYRTQVNDLLLTALA
RATCRWSGDASVLVQLEGHGREDLGEAIDLSRTVGWFTSLFPLRLTPAADLGESLKAIK
EQLRGVPDKGVGYGLLRYLAGEEAAARLAALPQPRITFNYLGRFDRQFDGAALLVPAT
ESAGAAQDPCAPLANWLSIEGQVYGGELSLHWSFSREMFAEATVQRLVDDYARELHVL
IEHCCQEGNVGATPSDFPLATLRQEQLDRLPLALIEDIYPLSPMQHGMLFHSLYEQASGD
YLNQLRVDVHGLDPARFRAAWQAALDSHDILRAGFLWQGDLEQPLQVIHKHLELPFAE
HDWRGREALAEALDELAASERRRGFELEQAPLLRLVLVRMDEERYHLVYTHHHILLDG
WSSAQLLGEVLARYTGEQAERTGGRYRDYIAWLQAQDKRVSEAFWKEQLAELLEPTRL
AQAVAAEREQVGSGQFQRSLPPARTARLKTFAQRHAVTLNTLVQAAWSLLLQRYTGQ
DTVVFGATVAGRPAELAGIERQIGLFINTLPVVATPQPGMRLTDWLQEVQARSLALREQ
EHTPLFEIQRWAGLGEALFDSLLVFENYPVAEALEKGSPGGVRFGPVSNHEQTNYPLTV
ALGVGDSLSLQYSYDRQAFSDAAVEQLDRHLLNLLEGFVDNAERTLVELSLLDAEERAL
IDSLWNRSESGFPASPLIHQRVAERARLAPDAPAVLFDDQVLSFAELDSRANRLAHALIA
RGVGPEVRVAIAMQRSAEIMIAFLAVLKSGGAYVPLDIEYPRERLLYMMQDSRAHLLLT
QSHLLDRLPIPDGLSCLCLDREQEWAGFPAHDPEVALHGDNLAYVIYTSGSTGMPKGVA
VSHGPLAAHIVATGERYEMTPADCELHFMSFAFDGSHEGWMHPLINGARVLIRDDSLW
LPEQTYAQMHRHGVTVAVFPPVYLQQLAEHAERDGNPPAARVYCFGGDAVAQASYDL
AWRALRPQYLFNGYGPTETVVTPLLWKARPDDPCGAAYMPIGTLLGNRSGYILDAQLN
LLPVGVAGELYLGGEGVARGYLERPALTAERFVPDPFGAPGSRLYRSGDLTRGRADGV
VDYLGRVDHQVKIRGFRIELGEIEARLREQAAVREAVVVAQAGASGQQLVGYVVPQDP
ALAEDVGAQAACRDALRKALKERLPEYMLPAHLLFLACMPLTPNGKLDRKGLPKPSAD
QQQRDYQAPRSEVERQLATIWAEVLKLEQVGLADNFFEIGGDSIISLQVVSRARQLGIHF
TPKMLFEAQTIGALAPLAESGTQVLAIDQGPVTGVTPLLPIQQGFFAEEVAERHWWNQS
VLLEAREPLDARLLEQALRGVLAHHDALRLSFTREAAGWTARHRGVEEGAAALLRVAR
VADLAALRALADEVQRSLDLADGPLLRALLATFDDGSQRLLLVIHHLVVDGVSWRILFE
DLQTAYRQLLAGQAVELPAKTSAFRDWAERLQAFAGDGGLDGELAYWQGQLQGASSD
LPCLDPQGDQSNRHARSVSCGLDAEATRQLLQEAPAAYRTQVNDLLLTALARVICRWT
GQVDALIQLEGHGREELFAEIDLTRTVGWFTSLFPLRLTPAEGIAASIKGIKEQLRAVPNK
GIGFGALRYLGSAASQAALAGLPVPRITFNYLGQFDGSFAMEEGALFAPAGERAGDDQS
PDAPLANWLALNGRIYGGELRIDWSFSGECFEIASIQRLADAYRDELLALIAHCRVAEGQ
GLTPSDFPLARLDQARLDQLPLAPCEVEDLYPLSPMQQGMLFHSLYQQEAGDYINQLRV
DIDGLHPESFRAAWQAALDEHDVLRSGFLWQGAFETPLQVVRKRVEVPFSVLDWRGRE
DLAAALDELAAGEGRLGFDLSEAPLLRLVLVRTDEERYHLIYTNHHILMDGWSNSQLLG
EVLQRYRGETPPRSGGRYRDYIAWLQRQDAALAEAFWLPRLRQLDEPTRLGQSIAQAK
QRGKGYAERLRELDGEQTRRLAELAREQKITVNTLVQAAWLMLLQRYTGQDSVAFGA
TVAGRPAELNGIEEQIGLFINTLPVIASPLPQQSLASWLQAVQGENLALREFEHTPLYDIQ
RWAGQGGEALFDNILVFENYPVSQMLQQQASQGLAFGAVGNHEQTNYPLTLSVSLGQR
LELQFAYDREHFDDASVARLDRHLTHLLAQMVERPASTCLAEFQLLEAAERRQAIFDW
GRNPGRYPDERSVEQLFASRAAMEPERVALLFEERQLSYGELNAQANRLAHRLIELGVG
PDVLVGIAVERGLEMIVSLLAVLKAGGAYVPLDPEYPQERLGYMIEDSGIALLLSQSHLL
QRLPAASGIACLALDQARDWQDRPASDPQLRAHPQNLAYVMFTSGSTGRPKGVGISRES
LSRHTHVSLEFFGIGPDDRVLQFSTFNFDGFVEQLYPPLACGASVVLRGTEIWDSETLYR
EIVERRITTVDLTTAYWNMLAKDFANQGVRDYGALRQVHAGGEAMPPESLVAWKAAG
LEHVRLLNTYGPTEATVTVTTLDCAPYVDGSKAIPATMPIGKVLPGRAIYLLDDAGQPA
PVGAVGELVIGAELLARGYFKRPDLTAARFIPDPFDEQGGGRLYRTGDLARYGADGVIE
YVGRVDHQVKVRGFRIELGEIEACLGEHPAVREALVIAIEGTAGAQLVAYLVPQAEALA
SATLEVQAALRNELKALLRDSLPEYMVPAHLLFLERLPLSPNGKVDRKALPAPDASLLQ
EAYVAPRSELECQVAAIWQEVLKLQRVGLDDHFFELGGHSLLAINVISRIQLELGMKLTP
QLLFQFPTLGLFVSNLEKAGGQVDTSKLNKLEALLDEMEEV
PA2225
(SEQ ID NO: 160)
MRIVCLLVLLSLIGGCSHSVTLQERAEQEAIFIQEFRQEFAKRLRYPLFAPGDEIPE
ADVVVFFRFASSSGRISSCRVQYGEGNVKSRSTLDEVFARRVIATCREPDLPVAPPALLD
GGGGFGFKQRVMFRKEVERPRF
ecI
(SEQ ID NO: 161)
MDSQGSIKNTLIAQLFQQNYDWLCKKLSYQTGCSHSAEDLAAEAFLQVWMLPD
PASIRSPRAFLATIAQRLMYESWRRKDLERAYLQILAEAPEAVQPSPHEQWMLIESLQAI
DRLLDGLSGQARAVFLMSQLEGLTYVQIGERLGLSLGRIHQLMKDALHCCYRGFQE
PA2044
(SEQ ID NO: 162)
MPRHPFSPSFLAASVFCAATPALAALQPIAEAPLAGETEFRCQFNADNSTDCVST
YSFTILKPSGREMLSRIDRSYAETDSLIVEKAELTQPGGKPVPLDQSQIDTRTAPNPDQGF
LRERQTSLAFPNLRVGTRISYTLREHFTAKPLSTQFHYILSRPPMPVRDDRFVAEFKAERP
IFVRSELMDAYRIEQSADKKTLKVSLKKPQYTNYINEAGNAYLRHTPRLELGSSLDLQD
NFGPFAARYNEILAAELPKGAAAAVAAVKGKPAREQVAGLMQYINDTYRYLGDWRAS
ERGYVPFSLAEIERNGYGDCKDLAILLAAMLKAAGIKAEPTLVSRGDVVWDLLVPGMY
APNHAIVRAEVDGKTWWLDPTNPVFAPGRTMPDIQQRWALVLGADGKVRRDEIPLEAP
GDTLRVTRSEHYTHDGEARVESRVELSNAPLMQLSVADRQRGRTSTDQDLCRNFAKEG
SDCVLERDDSQFVLPPSYTISARLTDRRALDRLGGEYFYNRQDLASQWDAFAKYRSEGQ
LAELYLGEPQITSYDISLSGGKTDEPAHSCEIRSPWFDIDLQAEPAKDGGYHYRYREVQK
TSWLNHDEINSAEFGKLIEQSRGCVEQLRLVVKLDKRP
N/A
(SEQ ID NO: 163)
MSKQVALVLGSGGARGYAHIGVIEELEARGYEITCIAGCSMGSVIGGIYAAGKLR
EYREWVESLDYLDVLRLLDVSFRLGAIRGERVFGKIHEILGEVNIEDLSIPYTAVATDLTN
QQEIWFQEGCLHQAMRASAAIPSLFTPVMQGSRMLVDGGLLNPLPIVPVVSAHSDLIIAV
NLNATNQKQYHLPVIERPAALKGRFDSMIESLGHKLSFFRRHGDDNTPPELSADALLHP
VPAESEEPAEPQLQQPAAAPQGKDSPKSASGTTVVESSGPASLLELVNQSFEVMQTSLA
QYKIAGYPPDILINVPKRVCRFFEFYKAPELIALGRQIASDTLDRYEEDNA
PA1638
(SEQ ID NO: 164)
MQQLLNEILDEVRPLIGRGKVADYIPALAGVEPNQLGIAVYSRDGELFHAGDALR
PFSIQSISKVFSLVQAIQHSGEDIWQRLGHEPSGQPFNSLVQLEFERGKPRNPFINAGALVI
CDINQSRFAAPAQSMRDFVRRLCGNPEVVSDSVVARSEYQHRSRNAAAAYLMKSFGNF
HNDVEAVLLSYFHHCALRMSCVDLARAFCFLADKGFCKHSGEQVLNERQTKQVNAIM
ATSGLYDEAGNFAYRVGLPGKSGVGGGIIAVVPGRFTVCVWSPELNAAGNSLAGIAALE
KLSERIGWSIF
fliO
(SEQ ID NO: 165)
MRRYLFAGFLPALASLSAPLCAAEGTTGAAAPTVGAASGAAAQLAQLVLGLGL
VIGLIFLLAWLVRRVQQAGPRGNRLIRTLASQPLGPRDRLVLVQVGEEQILLGLTPGRITP
LHVLKEPVHLPDGEPATPEFAQRLLELLNKDPKGKP
btuB
(SEQ ID NO: 166)
MNRVFLTPAAVALCGASSLSLAEPVSLADQVVTATRTAQTASQSLAAVSVIDRE
DIERSQARSVPELLRQVPGVSLANNGGFGKNTTLFLRGTESDHVLVLIDGIKVGSASAGL
TAFQDLPVELIERIEVVRGPRSSLYGSEAIGGVIQIFTRRGDGQGAKPFFSAGYGTHQTLE
GSAGVSGGAGNGWYSLGVSSFDTAGINTKRAGTAGYEPDRDGYRNLSGNLRGGYRFD
NGLELDGTLLRAKSHNDYDQVFGNSGFNANADGEQNLVGGRARFTPFDPWLVTLQAG
RSEDKADAYQDGRFYSRFDTRRDSLSWQNDLTLAEGHVLTLGYDWQKDEISSSEAFSV
DSRLNKGWFAQYLGQYGRQDWQLSLRRDDNQQFGVHDTGSAAWGYALSDALRFTVS
YGTAFKAPTFNELYYPDYGNPDLDAETSRSLEVGLSGTHGWGHWAVNAFRINVDDLIG
NDPRPAPGRPWGQPNNIDEARIRGVELVLGSQWLGWDWNANATFLDPQNRSGGVSDG
NELPRRARRMFNLELDRRFERLSLGASVHAEGRRYDDPANKVRLGGYATLDLRSEYRL
NDEWRLQGRIANLFGADYETAYGYNQPGQAVYLSVRYQAL
braG
(SEQ ID NO: 167)
MLSFDKVSTYYGKIQALHDVSVEVKKGEIVTLIGANGAGKSTLLMTLCGSPQAA
SGSIRYEGEELVGLPSSTIMRKSIAVVPEGRRVFSRLTVEENLAMGGFFTDKDDYQVQM
DKVLELFPRLKERYEQRAGTMSGGEQQMLAIGRALMSKPKLLLLDEPSLGLAPIIIQQIFE
IIEQLRREGVTVFLVEQNANQALKLADRAYVLENGRIVMHDTGAALLTNPKVRDAYLG
G
N/A
(SEQ ID NO: 168)
MAIRSEDSDGFWQAFSRQEQDSTNRLLPTTLAAPFILCASSATPCRSASSSNSANR
VRSSRLLANKVSSNRRNAAGSPPVMASSERMSKRLSIGDSEPSAFIQTPSICLKEGHCALR
CPAYGTPGIRRTTRPYMPSGPVIRKQHKGIASASAVQTGEHRLLRLIERPQTALETLMAG
TESTFASRPQPARPLVSGRETPTVSLCEEASSVATA
rsmA
(SEQ ID NO: 169)
MLILTRRVGETLMVGDDVTVTVLGVKGNQVRIGVNAPKEVAVHREEIYQRIQKE
KDQEPNH
PA0881
(SEQ ID NO: 170)
MTDYTQQLAGFLAGLRYQDLPPAVLARMEELFLDWLGSALAGKGQHPIPLFERY
AERMGPADGSAQILPSRRRSSPYFAALVNGAASHVVEQDDLHNSSVLHPAAVVFPAAL
AAAQDLGRSGAELILAAVAGYEAGIRIGEFLGRSHYRVFHTTATVGTLAAAVAVGKLM
DFDRERFVDLLGSAGTQAAGLWEFLRDAADSKQLHTAKAAADGLLAAYLTADGLSGA
RHILEGEQGMAAGMSSDADPERLVDRLGSRWALLETSFKFHASCRHTHPAADALLALM
QREGLDHSQIAAVTARVHQGAIDVLGRVVEPQTVHQAKFSMGTVLGLIAVYGKAGLGE
FHRHALSDPRVAAFRERVEMRLDPEVDAAYPQRWLGRVEVLDREGRRHTAAIDEPKGD
PGNTLSRDELADKFRRLLAFSGAATGAEAEILIQRAWGLRQAPSVTPLI
N/A
(SEQ ID NO: 171)
MEQPFDLAAELAKQPHLLEIAGNLLMKGGPEDYIGAVLCLRGTLYFKEAHTPLV
RESLCQCFDEFKRLAEPHLTRLWREEPAQGKPLTAYRDTQPLREMMGAMDEDDHLSFC
YTSGKKSRDAGAWLFDIYGKRSWQAKMGHDLSVLEFSVPLLYQEQQPLDFLQLFIDFA
RRLEPEQGYAGHAYNLSPTSWDNDEPSEAFMAARMPGLDVGTACLLANTPEFKPTRIK
TVSWLTLLNNERLALAGGLDALRAQLPSSHFAFYRYGDGVVIQAGAYPYIAGDAEDSR
PAPYVLLNHALKGIRYETIGSLHGGSHDGELRLVGWAADQWLKRLDVEDSEIPRWRDK
LLSAEPCLDATNTLPERL
fimU
(SEQ ID NO: 172)
MSYRSNSTGFTLIELLIIVVLLAIMASFAIPSFKQLTERNELQSAAEELNAMLQYA
RSEAVSQRRAISIQALKDKDWGKGLSIGVLASGSIAAPLRKHDGFRAATLTAKEKSAVE
HLTFTANGTLVPPTERTFAICQNGKTDGGRVLSISQAGRIQLEPSSKAPQSCY
ccsA
(SEQ ID NO: 173)
MQSSQLLPLGSLLLSFATPLAQADALHDQASALFKPIPEQVTELRGQPISEQQREL
GKKLFFDPRLSRSHVLSCNTCHNVGTGGADNVPTSVGHGWQKGPRNSPTVFNAVFNAA
QFWDGRAKDLGEQAKGPIQNSVEMHSTPQLVEQTLGSIPEYVDAFRKAFPKAGKPVSFD
NMALAIEAYEATLVTPDSPFDLYLKGDDKALDAQQKKGLKAFMDSGCSACHNGINLGG
QAYFPFGLVKKPDASVLPSGDKGRFAVTKTQSDEYVFRAAPLRNVALTAPYFHSGQVW
ELKDAVAIMGNAQLGKQLAPDDVENIVAFLHSLSGKQPRVEYPLLPASTETTPRPAE
waaF
(SEQ ID NO: 174)
MRILIVGPSWVGDMVMAQTLFQCLRQRHPECVIDVLAPEWSRPILERMPEVRQA
LSFPLGHGVMDVATRRRIGRGLRGQYEQAILLPNSLKSALVPWFAGIPKRTGWRGEMR
YGLLNDIRKLDKQRYPLMIERFMALAFEPGVELPKPYPQPRLRIDDGSRQAALDKFALSL
DRPVLALCPGAEFGEAKRWPAEHYAAVAEAKIRAGWQVWLFGSKNDHPGGEEIRQRLI
PGLREESFNLAGETSLAEAIDLMSCAGAVVSNDSGLMHVAAALDRPLVGVYGSTSPQFT
PPLADRVEIVRLGLECSPCFERTCRFGHYNCLRELPPGLVLQALERLVGDPAEVAG
PA5232
(SEQ ID NO: 175)
MKQESKRWLSRALIVAALLGVGVLVWQVSRPTGLGEGFASGNGRIEATEVDVA
AKLPGRVAEIKVDEGDFVKAGEIVARMDTQVLEAQLAQAQAQVRQAENAKLTATSLV
AQRESEKSTAQAVVAQRQAELTAAQKRFTRTEALVKRNALPQQQLDDDRATLQSAQA
ALSAARSQVISAQAAIEAGRSQVIEAQSAIEAAKASVARLQADIDDSLLKAPRNGRVQYR
VAQPGEVLPAGGKLLNMVDLADVYMTFFLPSMQAGRVGLGQEVRLVIDAVPDYVIPA
KVSYVASVAQFTPKTVETANEREKLMFRVKARLDPALLEKYITYVKTGVPGMAYLRLD
PEVEWPANLQIKVPQ
kinB
(SEQ ID NO: 176)
MSMPLPMKLRTRLFLSISALITVSLFGLLLGLFSVMQLGRAQEQRMSHHHATIEV
SQQLRQLLGDQLVILLREKPDGQALERSQNDFRRVLEQGRANTVDGAEQAALDGVRDA
YLQLQAHTPALLKAPMADNDGFSEAFNTLRLRLQDLQQLALAGISDAETSARHRAYLV
AGLLGLVGVAILLIGFVTAHSIARRFGAPIETLARAADRIGEGDFDVTLPMTNVAEVGQL
TRRFGLMAEALRQYRKTSVEEVLSGERRLQAVLDSIDDGLVIFDNQGRIEHANPVAIRQL
FVSNDPHGKRIDEILSDVDVQEAVEKALLGEVQDEAMPDLVVDVAGESRLLAWSLYPV
THPGGHSVGAVLVVRDVTEQRAFERVRSEFVLRASHELRTPVTGMQMAFSLLRERLDF
PAESREADLIQTVDEEMSRLVLLINDLLNFSRYQTGMQKLELASCDLVDLLTQAQQRFT
PKGEARRVSLQLELGDELPRLQLDRLQIERVIDNLLENALRHSSEGGQIHLQARRQGDRV
LIAVEDNGEGIPFSQQGRIFEPFVQVGRKKGGAGLGLALCKEIIQLHGGRIAVRSQPGQG
ARFYMLLPV
N/A
(SEQ ID NO: 177)
MTEDQLEQETLGWLTELGYAYLYGPDIAHDGDNPERESYRDVLLTMRLRTAIAR
LNPQIPLAAREDALRQVLELGVPVQLSANRLLHRLLVGGVPVQYQKDGETRGDFVRLID
WVDVQANEWLAVNQFSIQGPKHTRRPDIILFVNGLPLVLLELKNPADVNADLVKAFDQ
LQTYKEQIPDVFHYNEILVISDGSEARMGSLSADIERFTRWRTIDGATVDPLGEFNELETL
VRGVLQPAMLLDYLRYFVLFEDDGRLVKKIAGYHQFHAVRAAIQQVVSASRPGGTHKG
GVVWHTQGSGKSITMTCFAARVMQEAAMENPTIVVITDRNDLDGQLFGVFSLSQDLLR
EQPVQVATRGDLREKLANRPSGGIVFATIQKFMPGEDEDSFPVLSTRSNIVVVADEAHRT
QYGFSASLKVPDLKVAEASARYQVGYAQHLRDALPNATFVAFTGTPVSSEDRDTRAVF
GDYIHVYDMQQAKEDGATVAIYYESRLAKLSLKDSELAHIDDEVDELAEDEEEDQQSR
LKSRWAALEKVVGAEPRIKSVAADLVAHFEERNQAQNGKAMVVAMSREICVHLYNEII
ALRPEWHAEDPEKGAVKIVMTGSASDKALLRPHIYPGQVKKRLEKRFKDPADPLQLVIV
RDMWLTGFDAPCVHTLYVDKPMKGHNLMQAIARVNRVFKDKQGGLVVDYIGIANELK
AALKEYTASKGRGRPTVDAHEAYAVLEEKLDVLRSLLYGFDYGDFLTGGHKLLAGAA
NHVLGLEDGKKRFADNALAMSKAFTLCCTLDEAKAVREEVAFLQAIKVLLIKRDISAQK
KPTKNVSWRSARSSAMP
infC
(SEQ ID NO: 178)
MRQDKRAQPKPPINENISAREVRLIGADGQQVGVVSIDEAIRLAEEAKLDLVEISA
DAVPPVCRIMDYGKHLFEKKKQAAVAKKNQKQAQVKEIKFRPGTEEGDYQVKLRNLV
RFLSEGDKAKVSLRFRGREMAHQELGMELLKRVEADLVEYGTVEQHPKLEGRQLMMV
IAPKKKK
pilC
(SEQ ID NO: 179)
MADKALKTCVFVWEGTDKKGAKVKGELAGQNTMLVKAQLRKQGINPLKVRK
KGITLLGKGKRVKPMDIALFTRQMATMMGAGVPLLQSFDIISEGFDNPNMRKLVDEIKQ
EVSAGNSLANSLRKKPLYFDDLYCNLVDAGEQSGALETLLDRVATYKEKTESLKAKIKK
AMTYPIAVVLVAIIVSAILLIKVVPQFQSVFSSFGAELPAFTMMVINLSNLLQEWWLVVLI
GLFSASFAIKESHKRSVNFRNTVDRYMLKIPIIGGILYKSAVARYARTLSTTFAAGVPLVE
ALDSVSGATGNVVFRNAVSKIKQDVSTGMQLNFSMRTTNVFPNMAIQMTAIGEESGSL
DDMLGKVAAFYEEEVDNAVDNLTTLMEPMIMAVLGVLVGGLIIAMYLPIFQLGSVV

Analysis of amplicon sequencing data. Paired-end reads were trimmed for adapter sequences and filtered with cutadapt (pair-filter q30), then merged across overlapping regions of Read 1 and Read 2 with vsearch v 2.15.2, and aligned to the coding sequence of mutated genes (bowtie2—local). From each merged and aligned read, both the sequence at the profiled locus (wild-type vs. mutant) and the unique UMI sequence (from both forward and reverse) were extracted, which were used to count the number of unique UMI corresponding to each allele type. Uncertainty of each allele frequency was calculated using the Wilson Score interval based on UMI counts using the statsmodels package (proportion_confint).

Mapping mutations onto protein structure. Protein sequences of mutated genes were queried in the Protein Data Bank (PDB) to find the closes homolog structures: NalD (PDB ID: 5DAJ, 94% identity), AnmK (3QBW, 99% identity), MexR (1LNW, 99% identity), AmpR (5MMH, 100% identity), PA0810 (3UMC, 93% identity).

Statistical analyses. Statistical analyses using Mann-Whitney U-test (ranksum) and Kolmogorov-Smirov test (kstest2) were conducted using built-in packages in MATLAB (R2017b). ANOVA tests for phenotype assays were conducted in Prism (GraphPad). Permutation test for <dMRCA> were conducted in python, with code available at GitHub https://github.com/hattiechung/Paeruginosa_acute_infection.

Data Availability. The patient-specific reference genomes constructed from PacBio sequencing in this study have been deposited to Sequence Read Archive (SRA) under accession code PRJNA638217 [https://www.ncbi.nlm.nih.gov/bioproject/PRJNA638217]. The raw FASTQ files of Illumina sequencing of the 420 isolates generated in this study have been deposited to SRA under accession code PRJNA622605 [https://www.ncbi.nlm.nih.gov/bioproject/PRJNA622605]. The list of all within-patient pathogen variants is available in Supplementary Data 1. The processed data of genomic variants used to construct phylogenetic trees and the data on antibiotic resistance susceptibility profiles of all 420 isolates are available on GitHub [https://github.com/hattiechung/Paeruginosa_acute_infection]. Source data are provided with this paper. Protein structure data are available at the Protein Data Bank under the following IDs: 5DAJ [https://www.rcsb.org/structure/5DAJ], 3QBW [https://www.rcsb.org/structure/3QBW], 1LNW [https://www.rcsb.org/structure/1LNW], 5MMH [https://www.rcsb.org/structure/5MMH], 3UMC [https://www.rcsb.org/structure/3UMC].

Code Availability. Code used for analyses are available on GitHub [https://github.com/hattiechung/Paeruginosa_acute_infection].

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated herein by reference to the same extent as if each independent patent and publication was specifically and individually indicated to be incorporated herein by reference.

What is claimed is:

1. A method for characterizing low-frequency mutations associated with resistance in a pathogen or characterizing a bacterial infection in a subject, the method comprising:
(a) contacting a nucleic acid molecule derived from a biological sample from a subject with a primer pair, wherein at least one member of the primer pair comprises a unique molecular identifier, and wherein the primer pair binds a complementary sequence within or adjacent to at least one of an antimicrobial resistance polynucleotide, an antimicrobial resistance gene, or a regulator of the antimicrobial resistance gene, present in a pathogen genome;
(b) amplifying at least a portion of the antimicrobial resistance polynucleotide, the antibiotic resistance gene, or the regulator of the antibiotic resistance gene to obtain an amplicon; and
(c) deep sequencing the amplicon to identify an alteration in the antimicrobial resistance polynucleotide or an alteration in the antimicrobial resistance gene or the regulator of the antimicrobial resistance gene.

2. A method for characterizing low-frequency mutations associated with resistance to selection in a nucleic acid molecule derived from an organism, the method comprising:
(a) contacting the nucleic acid molecule with a primer pair, wherein at least one member of the primer pair comprises a unique molecular identifier, and wherein the primer pair binds a complementary sequence within or adjacent to a gene, or a regulator of the gene, associated with resistance to selection present in the nucleic acid molecule;
(b) amplifying at least a portion of the gene or the regulator of the gene to obtain an amplicon; and
(c) deep sequencing the amplicon to identify an alteration in the gene or the regulator of the gene.

3. The method of claim 1, wherein the antimicrobial resistance gene is a gene listed in Table 3 and/or wherein the regulator is a gene promoter or an enhancer.

4. The method of claim 1, wherein the antimicrobial resistance gene is NalD, OprD, MexR, AnmK, AmpD, SltB1, or PA0810.

5. The method of claim 1, wherein the pathogen is a bacteria, a virus, a fungus, or a protozoa.

6. The method of claim 5, wherein the pathogen is a bacteria selected from Helicobacter pylori, Borrelia burgdorferi, Legionella pneumophilia, Mycobacteria species, Staphylococcus aureus, Neisseria gonorrhoeae, Neisseria meningitidis, Listeria monocytogenes, Streptococcus pyogenes, Streptococcus agalactiae, Streptococcus, Enterococcus faecalis, Streptococcus bovis, Streptococcus, Streptococcus pneumoniae, pathogenic Campylobacter sp., Salmonella species, Shigella species, Yersinia species, Enterococcus species, Haemophilus influenzae, Bacillus anthracis, Erysipelothrix rhusiopathiae, Clostridium perfringers, Clostridium tetani, Clostridioides difficile, Pasteurella multocida, Bacteroides sp., Fusobacterium species, Streptobacillus moniliformis, Treponema pallidium, Treponema pertenue, Leptospira, Rickettsia, Actinomyces israelli, Pseudomonas aeruginosa, Escherichia coli, Klebsiella species, Enterobacter species, Acinetobacter species, Stenotrophomonas maltophilia, Burkholderia cepacia complex, Achromobacter species, or Burkholderia pseudomallei.

7. The method of claim 1, wherein the subject was previously treated with at least one antimicrobial.

8. The method of claim 1, wherein the alteration is a missense mutation, an insertion, or a deletion.

9. The method of claim 2, wherein the selection involves an antimicrobial, a chemotherapeutic, or another therapeutic agent.

10. The method of claim 1, wherein the method involves (d) determining the frequency of occurrence of the alteration in the population.

11. The method of claim 10, wherein a change in frequency of occurrence of the alteration is carried out over the course of time.

12. The method of claim 11, wherein a first biological sample is collected at a first time point and a second biological sample is collected at a second time point that is hours, days, or weeks later than the first time point.

13. A method of treating a bacterial infection in a subject, the method comprising administering to the subject an effective amount of an antimicrobial selected for efficacy in the subject, wherein the antimicrobial is selected by characterizing a bacteria present in a biological sample of the subject according to the method of claim 1.

14. The method of claim 13, wherein the bacteria comprises one or more antimicrobial resistance mutations.

15. A method of monitoring antimicrobial therapy in a subject, the method comprising:
(a) collecting two or more biological samples from the subject prior to or during the course of antimicrobial therapy;
(b) analyzing the samples according to the method of claim 1.

16. The method of claim 15, wherein a first biological sample is collected prior to commencing a therapy and wherein a second biological sample is collected 1, 2, or 3 days after the therapy is commenced.

17. The method of claim 15, wherein the antimicrobial resistance gene is a gene listed in Table 3.

18. The method of claim 15, wherein the regulator of the gene is a gene promoter or an enhancer.

19. The method of claim 15, wherein the antimicrobial resistance gene is NalD, OprD, MexR, AnmK, AmpD, SltB1, or PA0810.

20. A kit for characterizing antimicrobial resistance in a bacteria for use in the method of claim 1, the kit comprising one or more primers from among those listed in Table 4.
